(19) United States
(12) Patent Application Publication
(10) Pub. No.: US 2022/0290242 A1
(43) Pub. Date: Sep. 15, 2022

(54) METHOD FOR DIAGNOSING A CANCER AND ASSOCIATED KIT

(71) Applicants: CENTRE HENRI BECQUEREL, ROUEN (FR); UNIVERSITE DE ROUEN-NORMANDIE, MONT-SAINT-AIGNAN (FR); INSERM (INSTITUT NATIONAL DE LA SANTÉ ET DE LA RECHERCH MÉDICALE), PARIS (FR)

(72) Inventors: Philippe RUMINY, Rouen (FR); Vinciane MARCHAND, Rouen (FR); Ahmad ABDEL SATER, Rouen (FR); Pierre-Julien VIAILLY, Rouen (FR); Marie Delphine LANIC, Bihorel (FR); Fabrice JARDIN, Rouen (FR); Marick LAE, Rouen (FR); Mathieu VIENNOT, Rouen (FR)

(21) Appl. No.: 17/291,407

(22) PCT Filed: Nov. 5, 2019
(86) PCT No.: PCT/FR2019/052617

§ 371 Date: Oct. 4, 2021

(30) Foreign Application Priority Data
Nov. 5, 2018 (FR) 18 60174
Aug. 2, 2019 (FR) 19 08905

(51) Int. Cl. C12Q 1/6886

(52) U.S. Cl. CPC C12Q 1/6886; C12Q 2600/156

(57) ABSTRACT

The invention concerns a method for diagnosing a cancer in a subject, comprising a step of RT-MLPA on a biological sample obtained from the subject, in which the RT-MLPA step is carried out using at least one pair of probes comprising at least one probe chosen among the probes with SEQ ID NO: 1 to 13, and/or the probes with SEQ ID NO: 96 to 99, and/or the probes with SEQ ID NO: 866 to 938, and/or the probes with SEQ ID NO: 940 to 1104, and/or SEQ ID NO: 211 to 1312, and/or the probes with SEQ ID NO: 96 to 99, and/or the probes with SEQ ID NO: 1105 to 1107 and/or the probe with SEQ ID NO: 939 and/or the probes with SEQ ID NO: 1108 to 1123, each of the probes being fused, at at least one end, with a priming sequence, and at least one of the probes of the pair comprising a molecular barcode sequence.

### BACKGROUND OF THE INVENTION

### Field of the Invention

This invention relates to a method for diagnosing cancer and a kit useful for implementing such a method. The invention also relates to a method implemented by computer in order to analyze the results obtained after implementing this method, in particular carried out in the context of a cancer diagnosis.

### Description of the Related Art

Cancers are due to an accumulation of genetic abnormalities, by tumor cells. Among these abnormalities are numerous chromosomal rearrangements (translocations, deletions, and inversions) which result in the formation of fusion genes which encode abnormal proteins. These rearrangements also lead to imbalances in the expression of exons located at 5′ and 3′ of genomic breakpoints (5′-3′ expression imbalances), the expression of the former remaining under the control of the natural transcriptional regulatory regions of the gene while that of the latter falls under the control of the transcriptional regulatory regions of the partner gene. These abnormalities also include mutations at splice sites that disrupt normal RNA maturation, resulting in particular in exon skipping. Fusion genes, exon skipping, and 5′-3′ expression imbalances, which are important diagnostic markers, are usually investigated by different techniques. Some of these genetic abnormalities are very difficult to detect/analyze, particularly those involved in the development of sarcomas, which are very heterogeneous and can involve a very large number of genes. In addition, the amounts of RNA obtained from sarcoma biopsies are often very low, of poor quality. Chromosomal rearrangements in the context of sarcomas are discussed in particular in the Nakano and Takahashi article (Int. J. Mol. Sci. 2018, 19, 3784; doi:10.3390/ijms19123784).

Fusion genes are often associated with particular forms of tumor, and their detection can significantly contribute to making the diagnosis and choosing the most suitable treatment (The impact of translocations and gene fusions on cancer causation. Mitelman F, Johansson B, Mertens F, Nat Rev Cancer. 2007 April; 7(4):233-45). They are also often used as molecular markers to monitor the efficacy of treatments and follow the course of the disease, for example in acute leukemia (Standardized RT-PCR analysis of fusion gene transcripts from chromosome aberrations in acute leukemia for detection of minimal residual disease. Report of the BIOMED-1 Concerted Action: Investigation of minimal residual disease in acute leukemia. van Dongen J J, Macintyre E A, Gabert J A, Delabesse E, Rossi V, Saglio G, Gottardi E, Rambaldi A, Dotti G, Griesinger F, Parreira A, Gameiro P, Diaz M G, Malec M, Langerak A W, San Miguel J F, Biondi A. Leukemia. 1999 December; 13(12):1901-28).

The four main techniques which are commonly used to search for fusion genes are conventional cytogenetics, molecular cytogenetics (fluorescent in situ hybridization), immunohistochemistry, and molecular genetics (RT-PCR, RNAseq, or RACE).

Conventional cytogenetics consists of establishing the karyotype of cancer cells in order to look for possible abnormalities in the number and/or structure of the chromosomes. It has the advantage of providing an overall view of the entire genome. However, it is relatively insensitive, its effectiveness being highly dependent on the percentage of tumor cells in the sample to be analyzed and on the possibility of obtaining viable cell cultures. Another of its disadvantages is its low resolution, which does not allow detecting certain rearrangements (in particular small inversions and deletions). Finally, some tumors are associated with major genomic instability which masks pathognomonic genetic abnormalities. This is the case for example in solid tumors such as lung cancer. Karyotype analysis, when possible, is therefore difficult and can only be carried out by personnel with exceptional expertise, which entails significant costs.

Molecular cytogenetics, or FISH (Fluorescent In Situ Hybridization), consists of hybridizing fluorescent probes on the chromosomes of tumor cells in order to visualize their structural abnormalities. It makes it possible to detect chromosomal rearrangements with better resolution than conventional cytogenetics, and therefore to detect rearrangements of smaller size. It also makes it possible to uncover abnormalities in tumors with high genomic instability, by precisely targeting the genes likely to be involved. Its major disadvantage is that each abnormality must be investigated individually, using specific probes. It therefore incurs significant costs, and, due to the great diversity of the abnormalities which have been described and the small amount of tumor material available for diagnosis, only a few abnormalities can be investigated. For example, in practice, in a context of diagnosing a lung carcinoma, only the rearrangement of the ALK gene is commonly investigated by this method, the search for other recurrent rearrangements in these tumors remaining highly exceptional.

Immunohistochemistry (or IHC) consists of using antibodies to investigate the overexpression of an abnormal protein. This is a simple and rapid method, but also requires searching for each abnormality individually and its specificity is often low, as certain genes can be overexpressed in a tumor without any rearrangement.

RT-PCR, RNAseq, and RACE are methods of molecular genetics carried out using RNA extracted from tumor cells. RT-PCR has excellent sensitivity, far superior to cytogenetics. This sensitivity makes it the benchmark technique for analyzing biological samples where the percentage of tumor cells is low, for example in order to monitor the effectiveness of treatments or to anticipate possible relapses very early on. Its main limitation is linked to the fact that it is extremely difficult to multiplex this type of analysis. As with molecular cytogenetics, in general each translocation must be investigated by a specific test, and only a few recurrent fusions among the very many which are currently known are therefore tested for in routine diagnostic laboratories. RT-PCR also requires having RNAs of good quality, which is rarely the case for solid tumors where, in order to facilitate pathological diagnosis, the samples are fixed in formalin and embedded in paraffin the moment the biopsy sample is obtained. This highly sensitive technique can be very useful in diagnosing a sarcoma. Nevertheless, it is necessary to perform numerous independent tests, at a minimum for the most frequent recurrent fusion genes, which incurs additional costs and lengthens the time required. RNAseq, which consists of analyzing all the RNAs expressed by the tumor by next-generation sequencing (NGS), theoretically allows detecting all abnormal fusion transcripts expressed. However, it also requires having RNAs of good quality and is therefore difficult to implement from biopsies fixed with formalin. Its application is also very complex, since many steps are required to generate the sequencing libraries. In addition, the sequencing generates a very large amount of data (since all the genes are studied) which makes the analysis particularly complex. RACE, which has recently been adapted to NGS, is a simplification of the RNAseq technique but allows targeting small panels of genes likely to be involved in fusions. It has the advantage of being able to be applied to biopsies fixed with formalin. However, although the amount of data generated is reduced compared to RNAseq, it is still significant. Unlike the method described in the present invention which only detects abnormal RNAs, RACE results in obtaining sequences which correspond to all of the targeted genes in the panel, even when they are in a germinal configuration. The vast majority of the sequences obtained therefore correspond to normal transcripts, expressed naturally by tumor cells and by the cells in their environment. The sequence files must therefore be filtered to identify the fusion transcripts. Finally, similarly to RNAseq, RACE is a long and complex technique to implement, where many steps are necessary in order to obtain the sequencing libraries, which increases the time required to deliver results.

Exon skipping generally results in the expression of an abnormally short protein which is involved in the tumor process. For example, skipping of exon 14 of the MET gene is involved in the development of lung carcinoma, and skipping of exons 2 to 7 of the EGFR gene is involved in the development of certain brain tumors, in particular glioblastoma. They are often due to point mutations which affect the exon splicing sites (3′ donor sites, 5′ acceptors, as well as intronic or exonic enhancers), or to internal deletions of genes. Today, it is particularly difficult to uncover these abnormalities in order to diagnose cancers, since neither cytogenetics nor FISH are informative. RT-PCR could be an alternative, but it is severely limited due to the formalin fixation of tumor biopsies that is necessary for pathological diagnosis. These abnormalities are therefore currently tested for primarily by next-generation sequencing of genomic DNA or of RNA, which are expensive and complex techniques.

5′-3′ expression imbalances, which require quantitatively evaluating the expression of exons, are only very rarely tested for when diagnosing a cancer. They can be analyzed either by RNAseq or by dedicated kits such as those offered by the Nanostring company (for example the “nCounter® Lung Fusion Panel” test).

International application PCT/FR2014/052255 describes a method for diagnosing cancer by detecting fusion genes. Said method comprises a RT-MLPA step using probes fused, at at least one end, with a primer sequence.

The article by Ruminy et al. describes the detection of fusion genes by RT-MLPA in the context of acute leukemia (Multiplexed targeted sequencing of recurrent fusion genes in acute leukaemia; Leukemia, 2016 March; 30(3):757-60).

The article by Piton et al. describes the detection by RT-MLPA of rearrangement linked to the ALK, ROS and RET genes in the context of lung adenocarcinomas (Ligation-dependent-RT-PCR: a new specific and low-cost technique to detect ALK, ROS and RET rearrangements in lung adenocarcinoma; Lab Invest. 2018 March; 98(3):371-379).

Techniques are therefore currently known which allow detecting fusion genes, exon skipping, or 5′-3′ expression imbalances, but they have disadvantages.

The limitations of existing methods are essentially linked to: (i) the large number of abnormalities to be tested for (this is one of the most significant limitations of IHC, FISH, and RT-PCR techniques); (ii) the sensitivity required to detect genetic abnormalities using small tumor biopsies that are fixed and embedded in paraffin (this is one of the most significant limitations of next-generation sequencing techniques); (iii) the interpretation of the results (it is necessary to define thresholds for IHC, there are significant artifacts for FISH, RNAseq and RACE generate a very large amount of data which is difficult to analyze); (iv) the implementation complexity (the large number of steps to be carried out increases the risk of error, the technical time required increases operator costs and has a strong impact on the quality of the results generated and the times required for delivery).

The method described in international application PCT/FR2014/052255 is more specific, simple, and quick to implement compared to existing techniques for detecting fusion genes.

However, there is still a need for fusion gene diagnostic techniques capable of detecting a very wide variety of abnormalities, in specific, sensitive, and reliable ways, while remaining simple and quick to implement.

International application PCT/FR2014/052255 also describes specific probes for types of translocation observed in cancers. However, new genetic abnormalities have since been uncovered and cannot be detected by the method described in the international application referenced above.

There is therefore a need for a diagnostic method which allows detecting new genetic abnormalities.

Furthermore, the techniques which currently make it possible to detect exon skipping require performing complex additional tests. These techniques are therefore expensive, long to implement, and difficult to interpret.

There is therefore a need for a technique which allows detecting exon skipping that is sensitive, reliable, simple, economical, and quick to implement.

There is also a need for a technique which allows detecting 5′-3′ expression imbalances which is sensitive, reliable, simple, economical, and quick to implement.

As the techniques for detecting fusion genes, exon skipping, and 5′-3′ expression imbalances are different, there is also a need for a method that allows detecting these three types of genetic abnormalities simultaneously.

Finally, as the surgical tumor biopsies available for the diagnosis of solid cancers are often very small, fixed in formalin, and embedded in paraffin, there is a need for a method that allows detecting a large number of abnormalities simultaneously, in a small amount of low-quality genetic material.

### SUMMARY OF THE INVENTION

The invention thus aims to meet these different needs. The invention is in fact based on the results of the Inventors who (i) have identified new genetic abnormalities linked to the RET, MET, ALK, and/or ROS genes in carcinomas (both fusion genes and exon skipping), and (ii) have developed a technique to identify them. The invention is also based on (iii) the results of the inventors which have identified new probes, in particular which allow diagnosing sarcomas, brain tumors, gynecological tumors, or tumors of the head and neck, or (iv) 5′-3′ imbalances (for example 5′-3′ imbalances of the ALK gene). The invention is also based on (v) the use of probes comprising at least one molecular barcode, which makes it possible to significantly improve the sensitivity and specificity of the detection.

The invention thus provides a method which makes it possible to simultaneously detect fusion genes, exon skipping, and 5′-3′ expression imbalances. The invention also has the advantage of being specific, sensitive, reliable, but also simple, economical, and quick to implement. Typically, by means of the technique according to the invention, the results can be obtained within two or three days after the sample is received by the analysis laboratory, compared to several weeks for conventional techniques. It also offers the advantage of being applicable to fixed tissues, such as those used in pathology laboratories. The invention thus makes it possible to identify genetic abnormalities from a small amount of poor-quality genetic material. Finally, its very high sensitivity (it allows detecting less than ten abnormal molecules in a sample), coupled with its very high specificity (the results obtained are DNA sequences, meaning qualitative data, which does not induce interpretation bias the way quantitative IHC-type methods can), make this a very efficient method. The invention thus makes it possible to have a treatment plan adapted to each patient. Indeed, the invention makes it possible to diagnose with accuracy and to guide the choice of treatment by identifying patients eligible for targeted treatments.

### DESCRIPTION OF THE PREFERRED EMBODIMENTS

In a first aspect, the invention thus relates to a method for diagnosing cancer in a subject, comprising an RT-MLPA step on a biological sample obtained from said subject, wherein the RT-MLPA step is carried out using at least one pair of probes comprising at least one probe selected from:
the probes SEQ ID NO: 1 to 13, and/or
the probes SEQ ID NO: 96 to 99,
   each of the probes being fused, at at least one end, with a primer sequence, and at least one of the probes of said pair comprising a molecular barcode sequence.

In this first aspect, the invention also relates to a method for diagnosing cancer in a subject, comprising an RT-MLPA step on a biological sample obtained from said subject, wherein the RT-MLPA step is carried out using at least one pair of probes comprising at least one probe selected from:
the probes SEQ ID NO: 866 to 938, and/or SEQ ID NO: 940 to 1104, and/or
the probes SEQ ID NO: 1105 to 1107, and/or SEQ ID NO: 939, and/or
the probes SEQ ID NO: 1108 to 1123,
   each of the probes being fused, at at least one end, with a primer sequence, and at least one of the probes of said pair comprising a molecular barcode sequence.

In this first aspect, the invention also relates to a method for diagnosing cancer in a subject, comprising an RT-MLPA step on a biological sample obtained from said subject, wherein the RT-MLPA step is carried out using at least one pair of probes comprising at least one probe selected from the probes SEQ ID NO: 1211 to 1312,

each of the probes being fused, at at least one end, with a primer sequence, and at least one of the probes of said pair comprising a molecular barcode sequence.

In a first aspect, the invention thus relates to a method for diagnosing cancer in a subject, comprising an RT-MLPA step on a biological sample obtained from said subject, wherein the RT-MLPA step is carried out using at least one pair of probes comprising at least one probe selected from:
the probes SEQ ID NO: 1 to 13, and/or 866 to 938, and/or SEQ ID NO: 940 to 1104, and/or SEQ ID NO: 1211 to 1312, and/or
the probes SEQ ID NO: 96 to 99, and/or SEQ ID NO: 1105 to 1107, and/or SEQ ID NO: 939, and/or
the probes SEQ ID NO: 1108 to 1123,
   each of the probes being fused, at at least one end, with a primer sequence, and at least one of the probes of said pair comprising a molecular barcode sequence.

According to the invention, the term “MLPA” means Multiplex Ligation-Dependent Probe Amplification, which allows the simultaneous amplification of several targets of interest that are adjacent to one another, using one or more specific probes. In the context of the invention, this technique is very advantageous for determining the presence of translocations, which are frequent in malignant tumors.

According to the invention, the term “RT-MLPA” means Multiplex Ligation-Dependent Probe Amplification preceded by a Reverse Transcription (RT), which, in the context of the invention, allows starting with the RNA from a subject to amplify and characterize fusion genes, exon skippings of interest, and/or 5′-3′ expression imbalances. According to the invention, the RT-MLPA step is carried out in multiplex mode. The multiplex mode saves time because it is faster than several monoplex assays, and is economically advantageous. It also makes it possible to simultaneously search for a much higher number of abnormalities than the other techniques currently available. The RT-MLPA step is derived from MLPA, described in particular in U.S. Pat. No. 6,955,901. It allows the detection and simultaneous assay of a large number of different oligonucleotide sequences. The principle is as follows (see FIG. 1 which illustrates the principle with a fusion gene): the RNA extracted from tumor tissue is first converted into complementary DNA (cDNA) by reverse transcription. This cDNA is then incubated with the mixture of appropriate probes, each of which can then hybridize to the sequences of the exons to which they correspond. If one of the fusion transcripts or one of the transcripts corresponding to a searched-for exon skipping is present in the sample, two probes attach side by side to the corresponding cDNA. A ligation reaction is then carried out using an enzyme with DNA ligase activity, which establishes a covalent bond between the two adjacent probes. A PCR (Polymerase Chain Reaction) reaction is then carried out, using primers corresponding to the primer sequences, which makes it possible to specifically amplify the two ligated probes. Obtaining an amplification product after the RT-MLPA step indicates that one of the translocations or an exon skipping being searched for is present in the analyzed sample. Sequencing this amplification product allows identifying the genes involved.

According to the invention, the term “subject” means an individual who is healthy or is likely to be affected by cancer or is seeking screening, diagnosis, or follow-up.

According to the invention, the term “biological sample” means a sample containing biological material. More preferably, it means any sample containing RNA. This sample may come from a biological sample taken from a living being (human patient, animal). Preferably, the biological samples of the invention are selected among blood and a biopsy, obtained from a subject, in particular a human subject. The biopsy is in particular tumoral, in particular from a section of fixed tissue (for example fixed with formalin and/or embedded in paraffin) or from a frozen sample.

According to the invention, the term “cancer” means a disease characterized by abnormally high cell proliferation within normal tissue of the organism, such that the survival of the organism is threatened. In a preferred embodiment of the method according to the invention, the cancer is linked to a genetic abnormality, preferably the formation of a fusion gene and/or an exon skipping and/or a 5′-3′ imbalance. In a preferred embodiment of the method according to the invention, the cancer is linked to a genetic abnormality, preferably a fusion gene or an exon skipping. In a preferred embodiment of the method according to the invention, the cancer involves at least one gene selected among RET, MET, ALK and/or ROS, and in particular is associated with the formation of a fusion gene and/or an exon skipping, more particularly a skipping of an exon of the MET gene and/or a 5′-3 imbalance, more particularly a 5′-3′ imbalance of the ALK gene. According to the invention, and in a first aspect, the cancer is preferably a carcinoma. Carcinomas are malignant tumors that develop at the expense of epithelial tissue. More particularly, the cancer is a lung carcinoma, more particularly a bronchopulmonary carcinoma, even more particularly a lung carcinoma associated with a genetic abnormality of the RET, MET, ALK and/or ROS genes. In another preferred embodiment of the method according to the invention, the 5′-3′ expression imbalance is more particularly understood to mean an expression imbalance of the ALK gene. According to another aspect of the invention, and in a second aspect, the cancer is preferably a sarcoma, a brain tumor, a gynecological tumor, or a tumor of the head and neck. Sarcomas are tumors of the soft tissue and bone. Brain tumors are tumors that grow in the brain, such as gliomas or medulloblastomas. Gynecologic tumors are tumors of the female reproductive system, such as cervical cancer, endometrial cancer, and ovarian cancer. Cancers of the head and neck are cancers of the upper respiratory tract, such as squamous cell carcinoma of the throat (larynx, pharynx) and mouth, cancer of the cavum (or nasopharynx), cancer of the salivary glands (parotid, palate), or cancer of the thyroid gland. In another preferred embodiment of the method according to the invention, exon skipping also means a skipping of an exon of the EGFR gene, and more particularly a skipping of exons 2 to 7 of the EGFR gene. Thus, according to the invention, exon skipping is understood to mean a skipping of an exon or exons of the MET and/or EGFR gene.

According to the invention, the term “probe” means a nucleic acid sequence of a length between 15 and 55 nucleotides, preferably between 15 and 45 nucleotides, and complementary to a cDNA sequence derived from RNA of the subject (endogenous). It is therefore capable of hybridizing with said cDNA sequence derived from RNA of the subject. The term “pair of probes” means a set of two probes (i.e. a “Left” probe and a “Right” probe): one located at 5′ (see in particular “L” in Table 1) of the translocation of the fusion gene, of the skipping of an exon or exons whose expression is evaluated in order to detect a 5′-3′ expression imbalance, the other located at 3′ (see in particular “R” in Table 1) of the translocation of the fusion gene, of the skipping of an exon or exons whose expression is evaluated in order to detect a 5′-3′ expression imbalance. Preferably, said pair of probes consists of two probes hybridizing side by side during the RT-MLPA step. Preferably, a pair of probes according to the invention is formed at least of probes of SEQ ID NO: 1 to 13, and/or probes of SEQ ID NO: 96 to 99 and/or probes of SEQ ID NO: 14 to 91. Even more particularly, a pair of probes according to the invention is formed at least of probes of SEQ ID NO: 1 to 13, of probes of SEQ ID NO: 96 to 99 and of probes of SEQ ID NO: 14 to 91. Preferably, a pair of probes according to the invention is formed at least of probes of SEQ ID NO: 866 to 938, and/or probes of SEQ ID NO: 940 to 1104, and/or probes of SEQ ID NO: 1105 to 1107, and/or SEQ ID NO: 939, and/or probes SEQ ID NO: 1108 to 1123. Even more particularly, a pair of probes according to the invention is formed at least of probes of SEQ ID NO: 866 to 938, probes of SEQ ID NO: 940 to 1104, probes of SEQ ID NO: 1105 to 1107, the probe of SEQ ID NO: 939 and probes SEQ ID NO: 1108 to 1123. Preferably, a pair of probes according to the invention is formed at least of probes of SEQ ID NO: 1211 to 1312. Even more particularly, a pair of probes according to the invention is formed at least of probes of SEQ ID NO: 1 to 13, probes of SEQ ID NO: 96 to 99, probes of SEQ ID NO: 14 to 91, probes of SEQ ID NO: 866 to 938, probes of SEQ ID NO: 940 to 1104, probes of SEQ ID NO: 1105 to 1107, the probe of SEQ ID NO: 939, and probes of SEQ ID NO: 1108 to 1123. Even more particularly, a pair of probes according to the invention is formed at least of probes of SEQ ID NO: 1 to 13, probes of SEQ ID NO: 96 to 99, probes of SEQ ID NO: 14 to 91, probes of SEQ ID NO: 866 to 938, probes of SEQ ID NO: 940 to 1104, probes of SEQ ID NO: 1105 to 1107, the probe of SEQ ID NO: 939, and probes of SEQ ID NO: 1108 to 1123 and probes of SEQ ID NO: 1211 to 1312.

According to the invention, the term “primer sequence” means a nucleic acid sequence of a length between 15 and 30 nucleotides, preferably between 19 and 25 nucleotides, and not complementary to the cDNA sequences obtained from RNA of the subject. It is therefore not complementary to the cDNA corresponding to endogenous RNA. It therefore cannot hybridize with said cDNA sequences. Preferably, in a preferred embodiment of the method according to the invention, the primer sequence is selected from the (pairs of) sequences SEQ ID NO: 92 and SEQ ID NO: 93 or SEQ ID NO: 94 and SEQ ID NO: 95.

According to the invention, the term “index sequence” means a nucleic acid sequence of a length between 5 and 10 nucleotides, preferably between 6 and 8 nucleotides, in particular 8 nucleotides, and not complementary to the sequences of cDNA obtained from RNA of the subject. It is therefore not complementary to the cDNA corresponding to endogenous RNA. It therefore cannot hybridize with said cDNA sequences. Preferably, the index sequence is represented by the sequence SEQ ID NO: 836. Said index sequence is composed of bases (A, T, G, or C). In a preferred embodiment of the method according to the invention, said index sequence can be fused to a primer sequence, in particular at the 3′ end of the primer sequence. The index sequence is specific to each subject/patient whose sample is tested. Each pair of probes used in the PCR step comprises a different index sequence which allows identifying the sequences linked to each of the patients analyzed.

According to the invention, the term “molecular barcode” means a nucleic acid sequence of length between 5 and 10 nucleotides, preferably between 6 and 8 nucleotides, in particular 7 nucleotides, and not complementary to the cDNA sequences from RNA of the subject. It is therefore not complementary to the cDNA corresponding to endogenous RNA. It therefore cannot hybridize with said cDNA sequences. Preferably, the molecular barcode sequence is represented by the sequence SEQ ID NO: 100. Said molecular barcode sequence is a random sequence, composed of random bases (A, T, G, or C). The use of this sequence provides information on the exact number of cDNA molecules detected by ligation, while avoiding the bias associated with PCR amplification. According to the invention, at least one of the probes of said pair comprises a molecular barcode sequence. In other words, at least one of the probes of said pair is fused at one end with a molecular barcode sequence. In an embodiment that is preferred, and particularly preferred, a molecular barcode sequence is added at 5′ of the “F” or “Forward” probe, also called “L” or “Left”. In a preferred embodiment, each of the probes can comprise a molecular barcode sequence, in particular the probes SEQ ID NO: 14 to 91 and the probes SEQ ID NO: 96 and 98, preferably the probes SEQ ID NO: 14 to 91.

According to the invention, the term “extension sequence” refers to the sequences which can be present at the ends of the primers used during the PCR step, and which allow analysis of the PCR products on an Illumina-type next-generation sequencer. An “extension” sequence corresponds to any suitable sequence enabling analysis of the PCR products on a next-generation sequencer. An extension sequence is a nucleic acid sequence of a length between 5 and 20 nucleotides, preferably between 5 and 15 nucleotides, and not complementary to the cDNA sequences derived from RNA from the subject. It is therefore not complementary to the cDNA corresponding to endogenous RNA. It therefore cannot hybridize with said cDNA sequences. It is in particular represented by SEQ ID NO: 865. The knowledge of persons skilled in the art easily allows them to adapt these extension sequences.

According to the invention, the term “sensitivity” means the proportion of positive tests in subjects suffering from cancer and actually carrying the searched-for abnormalities (calculated by the following formula: number of true positives/(number of true positives plus number of false negatives)).

According to the invention, the term “specificity” means the proportion of negative tests in subjects not suffering from cancer and not carrying the searched-for abnormalities (calculated by the following formula: number of true negatives/(number of true negatives plus number of false positives)).

The inventors of the invention have identified specific probes for new genetic abnormalities observed in certain cancers. This identification is based on analysis of the intron/exon structure of genes involved in translocations, as shown in FIG. 1, or exon skippings, as shown in FIG. 2 or FIG. 9, or even 5′-3′ expression imbalances as shown in FIG. 13. In particular, with regard to FIG. 1, the breakpoints likely to lead to expression of functional chimeric proteins are searched for (FIG. 1A). From these results, DNA sequences of 25 to 50 base pairs are defined, which exactly correspond to the 5′ and 3′ ends of the exons of the two juxtaposed genes after splicing the hybrid transcripts (FIG. 1A). A set of probes is then defined as follows: a primer sequence (S_{A} in FIG. 1B) of about twenty base pairs, is added at 5′ of all the probes complementary to the exons of the genes forming the 5′ part of the fusion transcripts (S₁ in FIG. 1B). A second primer sequence (S_{B} in FIG. 1B), also about twenty base pairs but different from S_{A}, is added to the 3′ ends of all the probes complementary to the exons of the genes forming the 3′ part of the fusion transcripts (S₂ in FIG. 1B). At least one molecular barcode sequence (S_{A}′ in FIG. 1B) is added, for example at 5′ of the probe complementary to the exons of the genes forming the 5′ part of the fusion transcripts. These probes are then grouped together in a mixture, and contain all the elements necessary for the detection of one or more fusion transcripts, produced by one or more translocations. The probes used in the invention are therefore capable of hybridizing either with the last nucleotides of the last exon at 5′ of the translocation, or with the first nucleotides of the first exon at 3′ of the translocation. Preferably, the probes used according to the invention, capable of hybridizing with the first nucleotides of the first exon at 3′ of the translocation, are phosphorylated at 5′ before their use. The same principle applies when the genetic abnormality is an exon skipping. FIG. 2 represents the strategy which allows detecting a skipping of exon 14 of the MET gene, by means of the invention. FIG. 2A shows that in a normal situation, the splicing of the transcripts of the MET gene induces junctions between exons 13 and 14, and 14 and 15. In a pathological situation, for example if a mutation destroys the splice donor site of exon 14, the tumor cells express an abnormal transcript, resulting from the junction of exons 13 and 15. A set of probes is thus defined as follows: a primer sequence (S_{A} in FIG. 2B) of about twenty base pairs, is added at 5′ of all probes complementary to the exon 13 forming the 5′ part of the fusion transcripts (S_{13L} in FIG. 2B). A second primer sequence (S_{B} in FIG. 2B), also about twenty base pairs but different from S_{A}, is added to the 3′ ends of all probes complementary to the exon 15 forming the 3′ part of the fusion transcripts (S_{15R} in FIG. 2B). At least one molecular barcode sequence (S_{A}′ in FIG. 2B) is added, for example at 5′ of the probe complementary to the exons forming the 5′ part of the exon skipping, in particular exon 13 of the MET gene. The same principle applies for the skipping of exons 2 to 7 of the EGFR gene, which is often due to an internal deletion of the gene at the genomic DNA level and which results in the loss of these exons.

According to the invention, at least one of the probes of a pair used comprises a molecular barcode sequence, in particular the “L” probe. This means that the molecular barcode sequence is fused to the probe sequence at one of its ends, preferably 5′. When it is present, said molecular barcode sequence is preferably inserted between the primer sequence and the probe complementary to the exons of the genes. According to the invention, a preferred embodiment may also comprise a primer sequence at 5′ of a molecular barcode sequence, said barcode sequence itself being added at 5′ of the probe complementary to the exon of the gene forming the 5′ part of the fusion transcripts or of the transcript corresponding to an exon skipping, optionally 5′-3′ expression imbalances. According to the invention, an alternative embodiment may also comprise a primer sequence added to the 3′ end of a molecular barcode sequence, said barcode sequence itself being added at 3′ of the probe complementary to the exon of the gene forming the 3′ part of the fusion transcripts or of the transcript corresponding to an exon skipping, optionally 5′-3′ expression imbalances. According to the invention, one particular embodiment can thus comprise a primer sequence at 5′ of a molecular barcode sequence, said barcode sequence itself being added at 5′ of the probe complementary to the exon of the gene forming the 5′ part of the fusion transcripts or of the transcript corresponding to an exon skipping, optionally 5′-3′ expression imbalances, as well as a primer sequence added to the 3′ end of a molecular barcode sequence, said barcode sequence itself being added at 3′ of the probe complementary to the exon of the gene forming the 3′ part of the fusion transcripts or of the transcript corresponding to an exon skipping, optionally 5′-3′ expression imbalances.

An example of the various translocations (fusion genes) identified according to the invention is illustrated in FIG. 4. An example of exon skipping identified according to the invention is illustrated in FIG. 2 or FIG. 9. An example of a 5′-3′ imbalance is illustrated in FIG. 13. Example 6 also illustrates fusions associated with pathologies.

In a preferred embodiment of the method according to the invention, the probes SEQ ID NO: 14 to 91 are also used for the RT-MLPA step. In this aspect, each of the probes is also fused, at at least one end, with a primer sequence, and at least one of the probes preferably comprises a molecular barcode sequence. According to an even more particular embodiment, each of the “L” probes of the pair comprises a molecular barcode sequence.

In a preferred embodiment of the method according to the invention, the RT-MLPA step is carried out using pairs of probes each comprising a probe selected from probes SEQ ID NO: 1 to 13, optionally probes SEQ ID NO: 14 to 91, each of the probes being fused, at at least one end, with a primer sequence, and at least one of the probes of said pair comprising a molecular barcode sequence.

In a preferred embodiment of the method according to the invention, the RT-MLPA step is carried out using pairs of probes each comprising a probe selected from probes SEQ ID NO: 96 to 99, each of the probes being fused, at at least one end, with a primer sequence, and at least one of the probes of said pair comprising a molecular barcode sequence.

In a preferred embodiment of the method according to the invention, the RT-MLPA step is carried out using pairs of probes each comprising a probe selected from probes SEQ ID NO: 1 to 13 and probes SEQ ID NO: 96 to 99, each of the probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 1 to 13, probes SEQ ID NO: 96 to 99, and probes SEQ ID NO: 14 to 91, each of the probes being fused, at at least one end, with a primer sequence, and at least one of the probes of said pair comprising a molecular barcode sequence, in particular probes SEQ ID NO: 14 to 91 and optionally probes SEQ ID NO: 96 and 98.

In a preferred embodiment of the method according to the invention, the RT-MLPA step is carried out using pairs of probes comprising the probes selected from probes SEQ ID NO: 866 to 938 and SEQ ID NO: 940-1104, each of the probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 1211 to 1312, each of the probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 1105 to 1107 and SEQ ID NO: 939, each of the probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 1108 to 1123, each of the probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 866 to 938, and/or SEQ ID NO: 940 to 1104, and/or probes SEQ ID NO: 1105 to 1107, and/or SEQ ID NO: 939, and/or SEQ ID NO: 1108 to 1123, each of probes being fused, at at least one end, with a primer sequence, and at least one of the probes of said pair comprising a molecular barcode sequence.

In a preferred embodiment of the method according to the invention, the RT-MLPA step is carried out using pairs of probes comprising the probes selected from probes SEQ ID NO: 866 to 938, SEQ ID NO: 940 to 1104, SEQ ID NO: 1105 to 1107, SEQ ID NO: 939, SEQ ID NO: 1108 to 1123, each of the probes being fused, at at least one end, with a primer sequence, and at least one of the probes of said pair comprising a molecular barcode sequence.

In a preferred embodiment of the method according to the invention, the RT-MLPA step is carried out using pairs of probes each comprising the probes selected from probes SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824 and SEQ ID NO: 825, each of the probes being fused, at at least one end, with a primer sequence, and at least one of the probes of said pair comprising a molecular barcode sequence.

In a preferred embodiment of the method according to the invention, the RT-MLPA step is carried out using pairs of probes each comprising the probes selected from probes SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824, SEQ ID NO: 825, SEQ ID NO: 866 to 938, SEQ ID NO: 940 to 1104, SEQ ID NO: 1105 to 1107, SEQ ID NO: 939, and SEQ ID NO: 1108 to 1123, each of the probes being fused, at at least one end, with a primer sequence, and at least one of the probes of said pair comprising a molecular barcode sequence.

In a preferred embodiment of the method according to the invention, the RT-MLPA step is carried out using pairs of probes each comprising the probes selected from probes SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824, SEQ ID NO: 825, SEQ ID NO:866 to 938, SEQ ID NO: 940 to 1104, SEQ ID NO: 1105 to 1107, SEQ ID NO: 939, SEQ ID NO: 1108 to 1123, and SEQ ID NO: 1211 to 1312, each of the probes being fused, at at least one end, with a primer sequence, and at least one of the probes of said pair comprising a molecular barcode sequence.

In a preferred embodiment of the method according to the invention, the cancer associated with the formation of a fusion gene is diagnosed using at least one pair of probes comprising at least one probe selected from probes SEQ ID NO: 1 to 13, optionally probes SEQ ID NO: 14 to 91, and each of the probes is fused, at at least one end, with a primer sequence, preferably selected from the sequences SEQ ID NO: 92 and SEQ ID NO: 93, and at least one of the probes of said pair comprises a molecular barcode sequence.

In a preferred embodiment of the method according to the invention, the cancer associated with the formation of a fusion gene is diagnosed using at least one pair of probes comprising at least one probe selected from probes SEQ ID NO: 866 to 938 and/or SEQ ID NO: 940 to 1104, and each of the probes is fused, at at least one end, with a primer sequence, preferably selected from the sequences SEQ ID NO: 92 and SEQ ID NO: 93, and at least one of the probes of said pair comprises a molecular barcode sequence.

In a preferred embodiment of the method according to the invention, the cancer associated with the formation of a fusion gene is diagnosed using at least one pair of probes comprising at least one probe selected from probes SEQ ID NO: 1211 to 1312, and each of the probes is fused, at at least one end, with a primer sequence, preferably selected from the sequences SEQ ID NO: 92 and SEQ ID NO: 93, and at least one of the probes of said pair comprises a molecular barcode sequence.

In a preferred embodiment of the method according to the invention, the cancer associated with the formation of a fusion gene is diagnosed using at least one pair of probes comprising at least one probe selected from probes SEQ ID NO: 1 to 13, and/or SEQ ID NO: 14 to 91, and/or SEQ ID NO: 866 to 938 and/or SEQ ID NO: 940 to 1104, and each of the probes is fused, at at least one end, with a primer sequence, preferably selected from the sequences SEQ ID NO: 92 and SEQ ID NO: 93, and at least one of the probes of said pair comprises a molecular barcode sequence. Preferably, all the probes of SEQ ID NO: 1 to 13, SEQ ID NO: 14 to 91, SEQ ID NO: 868 to 938, and SEQ ID NO: 940 to 1104 are used.

In a preferred embodiment of the method according to the invention, the cancer associated with the formation of a fusion gene is diagnosed using at least one pair of probes comprising at least one probe selected from probes SEQ ID NO: 1 to 13, and/or SEQ ID NO: 14 to 91, and/or SEQ ID NO: 866 to 938 and/or SEQ ID NO: 940 to 1104, and/or SEQ ID NO: 1211 to 1312, and each of the probes is fused, at at least one end, with a primer sequence, preferably selected from the sequences SEQ ID NO: 92 and SEQ ID NO: 93, and at least one of the probes of said pair comprises a molecular barcode sequence. Preferably, all the probes of SEQ ID NO: 1 to 13, SEQ ID NO: 14 to 91, SEQ ID NO: 868 to 938, SEQ ID NO: 940 to 1104 and SEQ ID NO: 1211 to 1312 are used.

Alternatively and in another preferred embodiment of the method according to the invention, the cancer associated with an exon skipping is diagnosed using at least one pair of probes comprising at least one probe selected from probes SEQ ID NO: 96 to 99, and each of the probes is fused, at at least one end, with a primer sequence, preferably selected from the sequences SEQ ID NO: 94 and SEQ ID NO: 95, and optionally at least one of the probes of said pair comprises a molecular barcode sequence. More particularly according to this embodiment, the cancer is associated with a skipping of an exon of the MET gene, more particularly a skipping of exon 14 of the MET gene.

Alternatively and in another preferred embodiment of the method according to the invention, the cancer associated with an exon skipping is diagnosed using at least one pair of probes comprising at least one probe selected from probes SEQ ID NO: 1105 to 1107 and/or SEQ ID NO: 939, and each of the probes is fused, at at least one end, with a primer sequence, preferably selected from the sequences SEQ ID NO: 94 and SEQ ID NO: 95, and optionally at least one of the probes of said pair comprises a molecular barcode sequence. More particularly according to this embodiment, the cancer is associated with a skipping of exons of the EGFR gene, more particularly a skipping of exons 2 to 7 of the EGFR gene.

Alternatively and in another preferred embodiment of the method according to the invention, the cancer associated with an exon skipping is diagnosed using at least one pair of probes comprising at least one probe selected from probes SEQ ID NO: 96 to 99, and/or SEQ ID NO: 1105 to 1107 and/or SEQ ID NO: 939, and each of the probes is fused, at at least one end, with a primer sequence, preferably selected from the sequences SEQ ID NO: 94 and SEQ ID NO: 95, and optionally at least one of the probes of said pair comprises a molecular barcode sequence. Preferably, all the probes SEQ ID NO: 96 to 99, SEQ ID NO: 1105 to 1107 and SEQ ID NO: 939 are used.

Alternatively and in another preferred embodiment of the method according to the invention, the cancer associated with a 5′-3′ imbalance is diagnosed using at least one pair of probes comprising at least one probe selected from probes SEQ ID NO: 1108 to 1123 and each of the probes is fused, at at least one end, with a primer sequence, preferably selected from the sequences SEQ ID NO: 94 and SEQ ID NO: 95, and optionally at least one of the probes of said pair comprises a molecular barcode sequence. Preferably, all the probes SEQ ID NO: 1108 to 1123 are used.

In a preferred embodiment, the invention thus relates to a method for diagnosing a carcinoma in a subject, comprising an RT-MLPA step on a biological sample obtained from said subject with at least probes SEQ ID NO: 1 to 13, optionally probes SEQ ID NO: 14 to 91, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a carcinoma in a subject, comprising an RT-MLPA step on a biological sample obtained from said subject with at least probes SEQ ID NO: 1294 to 1312, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a carcinoma in a subject, comprising an RT-MLPA step on a biological sample obtained from said subject with at least probes SEQ ID NO: 1 to 13, and probes SEQ ID NO: 1294 to 1312, optionally probes SEQ ID NO: 14 to 91, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a sarcoma in a subject, comprising an RT-MLPA step on a biological sample obtained from said subject with at least probes SEQ ID NO: 866 to 938 and probes SEQ ID NO: 940 to 1054, optionally SEQ ID NO: 1148, and/or SEQ ID NO: 1149, and/or SEQ ID NO: 1178 and/or SEQ ID NO: 1179, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a sarcoma in a subject, comprising an RT-MLPA step on a biological sample obtained from said subject with at least probes SEQ ID NO: 1228 to 1291, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a sarcoma in a subject, comprising an RT-MLPA step on a biological sample obtained from said subject with at least probes SEQ ID NO: 866 to 938 and probes SEQ ID NO: 940 to 1054, and probes SEQ ID NO: 1228 to 1291, optionally SEQ ID NO: 1148, and/or SEQ ID NO: 1149, and/or SEQ ID NO: 1178 and/or SEQ ID NO: 1179, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a tumor of the head and neck in a subject, comprising an RT-MLPA step on a biological sample obtained from said subject with at least probes SEQ ID NO: 866 to 938 and probes SEQ ID NO: 940 to 1054, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a tumor of the head and neck in a subject, comprising an RT-MLPA step on a biological sample obtained from said subject with at least probes SEQ ID NO: 1211 to 1227, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a tumor of the head and neck in a subject, comprising an RT-MLPA step on a biological sample obtained from said subject with at least probes SEQ ID NO: 866 to 938 and probes SEQ ID NO: 940 to 1054 and probes SEQ ID NO: 1211 to 1227, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a gynecological tumor in a subject, comprising an RT-MLPA step on a biological sample obtained from said subject with at least probes SEQ ID NO: 866 to 938 and probes SEQ ID NO: 940 to 1054, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a brain tumor in a subject, comprising an RT-MLPA step on a biological sample obtained from said subject with at least probes SEQ ID NO: 1040 to 1104, optionally probes of SEQ ID NO: 124-125, SEQ ID NO: 456, SEQ ID NO: 1209-1210, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a brain tumor in a subject, comprising an RT-MLPA step on a biological sample obtained from said subject with at least probes SEQ ID NO: 1292 to 1293, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment, the invention thus relates to a method for diagnosing a brain tumor in a subject, comprising an RT-MLPA step on a biological sample obtained from said subject with at least probes SEQ ID NO: 1040 to 1104 and probes SEQ ID NO: 1292 to 1293, optionally the probes of SEQ ID NO: 124-125, SEQ ID NO: 456, SEQ ID NO: 1209-1210, each of the probes being fused, at at least one end, with a primer sequence, preferably selected from the sequences SEQ ID NO: 92 and SEQ ID NO: 93, and at least one of the probes of said pair comprises a molecular barcode sequence.

In a preferred embodiment of the method according to the invention, said RT-MLPA step comprises at least the following steps:

a) extraction of RNA from the biological sample from the subject,
b) conversion of the RNA extracted in a) into cDNA by reverse transcription,
c) incubation of the cDNA obtained in b) with a pair of probes comprising at least one probe selected from:
the probes SEQ ID NO: 1 to 13, and/or
the probes SEQ ID NO: 96 to 99,
   each of the probes being fused, at at least one end, with a primer sequence, and at least one of the probes of said pair comprising a molecular barcode sequence,
   d) addition of a DNA ligase to the mixture obtained in c), in order to establish a covalent bond between two adjacent probes,
   e) PCR amplification of the covalently bound adjacent probes obtained in d), in order to obtain amplicons.

In a preferred embodiment of the method according to the invention, said RT-MLPA step also comprises at least the following steps:

a) extraction of RNA from the biological sample from the subject,
b) conversion of the RNA extracted in a) into cDNA by reverse transcription,
c) incubation of the cDNA obtained in b) with a pair of probes comprising at least one probe selected from:
the probes SEQ ID NO: 866 to 938, and/or SEQ ID NO: 940 to 1104, and/or
the probes SEQ ID NO: 1105 to 1107 and/or SEQ ID NO: 939, and/or
the probes SEQ ID NO: 1108 to 1123,
   each of the probes being fused, at at least one end, with a primer sequence, and at least one of the probes of said pair comprising a molecular barcode sequence,
   d) addition of a DNA ligase to the mixture obtained in c), in order to establish a covalent bond between two adjacent probes,
   e) PCR amplification of the covalently bound adjacent probes obtained in d), in order to obtain amplicons.

In a preferred embodiment of the method according to the invention, said RT-MLPA step also comprises at least the following steps:

a) extraction of RNA from the biological sample from the subject,
b) conversion of the RNA extracted in a) into cDNA by reverse transcription,
c) incubation of the cDNA obtained in b) with a pair of probes comprising at least one probe selected from the probes SEQ ID NO: 1211 to 1312,
each of the probes being fused, at at least one end, with a primer sequence, and at least one of the probes of said pair comprising a molecular barcode sequence,
d) addition of a DNA ligase to the mixture obtained in c), in order to establish a covalent bond between two adjacent probes,
e) PCR amplification of the covalently bound adjacent probes obtained in d), in order to obtain amplicons.

In a preferred embodiment of the method according to the invention, said RT-MLPA step comprises at least the following steps:

a) extraction of RNA from the biological sample from the subject,
b) conversion of the RNA extracted in a) into cDNA by reverse transcription,
c) incubation of the cDNA obtained in b) with a pair of probes comprising at least one probe selected from:
the probes SEQ ID NO: 1 to 13, and/or SEQ ID NO: 866 to 938, and/or SEQ ID NO: 940 to 1104, and/or
the probes SEQ ID NO: 96 to 99, and/or SEQ ID NO: 1105 to 1107 and/or SEQ ID NO: 939,
the probes SEQ ID NO: 1108 to 1123,
   each of the probes being fused, at at least one end, with a primer sequence, and at least one of the probes of said pair comprising a molecular barcode sequence,
   d) addition of a DNA ligase to the mixture obtained in c), in order to establish a covalent bond between two adjacent probes,
   e) PCR amplification of the covalently bound adjacent probes obtained in d), in order to obtain amplicons.

In a preferred embodiment of the method according to the invention, said RT-MLPA step comprises at least the following steps:

a) extraction of RNA from the biological sample from the subject,
b) conversion of the RNA extracted in a) into cDNA by reverse transcription,
c) incubation of the cDNA obtained in b) with a pair of probes comprising at least one probe selected from:
the probes SEQ ID NO: 1 to 13, and/or SEQ ID NO: 866 to 938, and/or SEQ ID NO: 940 to 1104, and/or SEQ ID NO: 1211 to 1312, and/or
the probes SEQ ID NO: 96 to 99, and/or SEQ ID NO: 1105 to 1107 and/or SEQ ID NO: 939,
the probes SEQ ID NO: 1108 to 1123,
   each of the probes being fused, at at least one end, with a primer sequence, and at least one of the probes of said pair comprising a molecular barcode sequence,
   d) addition of a DNA ligase to the mixture obtained in c), in order to establish a covalent bond between two adjacent probes,
   e) PCR amplification of the covalently bound adjacent probes obtained in d), in order to obtain amplicons.

Typically, the extraction of RNA from the biological sample according to step a) is carried out according to conventional techniques, well known to those skilled in the art. For example, this extraction can be carried out by cell lysis of the cells obtained from the biological sample. This lysis may be chemical, physical or thermal. This cell lysis is generally followed by a purification step which allows separating the nucleic acids from other cellular debris and concentrating them. For the implementation of step a), commercial kits of the QIAGEN and Zymo Research type, or those marketed by Invitrogen, can be used. Of course, the relevant techniques differ depending on the nature of the biological sample tested. The knowledge of the person skilled in the art will allow said person to easily adapt these steps of lysis and purification to said biological sample tested.

Preferably, the RNA extracted in step a) is then converted by reverse transcription into cDNA; this is step b) (see FIG. 1B). This step b) can be carried out using any reverse transcription technique known from the prior art. It can in particular be carried out using the reverse transcriptase marketed by Qiagen, Promega, or Ambion, according to the standard conditions of use, or alternatively using M-MLV Reverse Transcriptase from Invitrogen.

Preferably, the cDNA obtained in step b) is then incubated with at least the probes SEQ ID NO: 1 to 13 and/or SEQ ID NO: 96 to 99, preferably also the probes SEQ ID NO: 14 to 91, each of the probes being fused, at at least one end, with a primer sequence, and at least one of the probes of said pair comprising a molecular barcode sequence, preferably the probes of SEQ ID NO: 14 to 91 and optionally the probes of SEQ ID NO: 96 and 98. This is the probe hybridization step c) (see FIG. 1B). Indeed, the probes which are complementary to a portion of cDNA will hybridize with this portion if the portion is present in the cDNA. As shown in FIG. 1B, due to their sequence, the probes will therefore hybridize:
either with the portion of cDNA corresponding to the last nucleotides of the last 5′ exon of the translocation. These are then probes that are also called “L” or “Left”;
or with the portion of cDNA corresponding to the first nucleotides of the first 3′ exon of the translocation. These are then probes that are also called “R” or “Right”.

Preferably, the cDNA obtained in step b) is then incubated with at least the probes SEQ ID NO: 866 to 938 and/or SEQ ID NO: 940 to 1104 and/or SEQ ID NO: 1105 to 1107 and/or SEQ ID NO: 939 and/or SEQ ID NO: 1108 to 1123, each of the probes being fused, at at least one end, with a primer sequence, and at least one of the probes of said pair comprising a molecular barcode sequence. This is probe hybridization step c) (see FIG. 1B). Indeed, the probes which are complementary to a portion of cDNA will hybridize with this portion if the portion is present in the cDNA. As shown in FIG. 1B, due to their sequence, the probes will therefore hybridize:
either with the portion of cDNA corresponding to the last nucleotides of the last 5′ exon of the translocation. These are then “L” or “Left” probes;
or with the portion of cDNA corresponding to the first nucleotides of the first 3′ exon of the translocation. These are then also “R” or “Right” probes.

Preferably, the cDNA obtained in step b) is then incubated with at least the probes SEQ ID NO: 1211 to 1312, each of the probes being fused, at at least one end, with a primer sequence, and at least one of the probes of said pair comprising a molecular barcode sequence. This is probe hybridization step c) (see FIG. 1B). Indeed, the probes which are complementary to a portion of cDNA will hybridize with this portion if the portion is present in the cDNA. As shown in FIG. 1B, due to their sequence, the probes will therefore hybridize:
either with the portion of cDNA corresponding to the last nucleotides of the last 5′ exon of the translocation. These are then “L” or “Left” probes;
or with the portion of cDNA corresponding to the first nucleotides of the first 3′ exon of the translocation. These are then also “R” or “Right” probes.

Preferably, the probes SEQ ID NO: 1 to 13, 97 and 99 are “R” probes and the probes SEQ ID NO: 96 and 98 are “L” probes, as are the probes SEQ ID NO: 14 to 91.

Preferably, the probes SEQ ID NO: 870-873, 877-878, 882, 889-892, 894-895, 901-902, 912-914, 920-921, 924-926, 930, 937, 939, 943, 946, 950-968, 970-971, 973-983, 988, 991-994, 997-998, 1000, 1002-1004, 1007, 1009-1010, 1017, 1021, 1022, 1035-1040, 1042-1043, 1048-1054, 1056-1059, 1063, 1065, 1067-1068, 1070, 1079-1081, 1088-1089, 1092, 1094, 1096, 1099-1102, 1104, 1106, 1109, 1111, 1113, 1115, 1117, 1119, 1121, 1123 are “R” probes, and the probes SEQ ID NO: 866-869, 874-876, 879-881, 883-888, 893, 896-900, 903-911, 915-919, 922-923, 927-929, 931-936, 938, 940-942, 944-945, 947-949, 969, 972, 984-987, 989-990, 995-996, 999, 1001, 1005-1006, 1008, 1011-1016, 1018-1020, 1023-1034, 1041, 1044-1047, 1055, 1060-1062, 1064, 1066, 1069, 1071-1078, 1082-1087, 1090-1091, 1093, 1095, 1097-1098, 1103, 1105, 1107-1108, 1110, 1112, 1114, 1116, 1118, 1120, 1122 are “L” probes.

Preferably, the probes SEQ ID NO: 1211, 1214, 1215, 1216, 1217, 1222, 1224, 1227, 1230, 1235, 1237, 1239, 1242, 1245, 1248-1249, 1251, 1253, 1260-1265, 1269-1270, 1272, 1273, 1278, 1280, 1282, 1284-1288, 1290, 1295, 1299, 1303-1305, 1310-1312 are “R” probes, and the probes SEQ ID NO: 1212, 1213, 1218-1221, 1223, 1225-1226, 1228-1229, 1231-1234, 1236, 1238, 1240-1241, 1243-1244, 1246-1247, 1250, 1252, 1254-1259, 1266-1268, 1271, 1274-1277, 127, 1281, 1283, 128, 1291-1294, 1296-1298, 1300-1302, 1306-1309 are “L” probes.

At the end of step c), the probes hybridized to the cDNA are adjacent, if and only if the translocation (fusion gene) or the exon skipping has taken place. This step c) is typically carried out by incubating the cDNA and the mixture of probes at a temperature of between 90° C. and 100° C. in order to denature the secondary structures of the nucleic acids, for a period of 1 to 5 minutes, then leaving this to incubate for a period of at least 30 minutes, preferably 1 hour, at a temperature of about 60° C. to allow hybridization of the probes. This can be carried out using the commercial kit sold by the MRC-Holland company (SALSA MLPA Buffer) or using a buffer offered by the NEB company (Buffer U).

At the end of step c), a DNA ligase is typically added in order to covalently bind only the adjacent probes; this is step d) (see FIGS. 1B and 2B). The DNA ligase is in particular ligase 65, sold by MRC-Holland, Amsterdam, Netherlands (SALSA Ligase-65), or the thermostable ligases (Hifi Taq DNA Ligase or Taq DNA ligase) sold by the NEB company. It is typically carried out at a temperature between 50° C. and 60° C., for a period of 10 to 20 minutes, then for a period of 2 to 10 minutes at a temperature between 95° C. and 100° C.

At the end of step d), each pair of adjacent probes L and R is covalently bound, and the primer sequence of each probe is still present in 5′ and 3′, as well as the molecular barcode sequence.

Preferably, the method also comprises a step e) of PCR amplification of the adjacent covalently bound probes obtained in d) (see FIGS. 1B and 2B). This PCR step is done using a pair of primers, one of the primers being identical to the 5′ primer sequence, the other primer being complementary to the 3′ primer sequence. Preferably, the PCR amplification of step e) is carried out using the pair of primers SEQ ID NO: 101 and 92 to detect fusion genes, or the pair of primers SEQ ID NO: 102 and 94 to detect skipping of exons of the MET and EGFR genes.

PCR is typically carried out using commercial kits, such as the ready-to-use kits sold by Eurogentec (Red′y′Star Mix) or NEB (Q5 High fidelity DNA polymerase). Typically, the PCR takes place with a first phase of initial denaturation at a temperature between 90° C. and 100° C., typically around 94° C., for a time of 5 to 8 minutes; then a second phase of amplification comprising several cycles, typically 35 cycles, each cycle comprising 30 seconds at 94° C., then 30 seconds at 58° C., then 30 seconds at 72° C.; and a last phase of returning to 72° C. for approximately 4 minutes. At the end of the PCR, the amplicons are preferably stored at −20° C. According to the invention, the amplicons correspond to the fusion transcripts or to the transcripts corresponding to an exon skipping present in the sample from the patient/subject to be tested, or possibly to a 5′-3′ imbalance.

According to the invention, in one particular embodiment, and when it is present, the index sequence is in particular introduced during the PCR step at the 3′ end of a primer sequence, in particular the “R” primer sequence.

According to the invention, in one particular embodiment, a first extension sequence can be introduced at 5′ of a primer sequence, and a second extension sequence can be introduced at 3′ of the index sequence.

According to the invention, in one particular embodiment, each pair of probes used in the PCR step comprises a different index sequence which makes it possible to identify the patients. PCR is typically carried out using commercial kits, such as the ready-to-use kits sold by Eurogentec (Red′y′Star Mix) or NEB (Q5 High fidelity DNA polymerase). Typically, the PCR takes place in a first phase of initial denaturation at a temperature between 90° C. and 100° C., typically around 94° C., for a period of 5 to 8 minutes; then a second amplification phase comprising several cycles, typically 35 cycles, each cycle comprising 30 seconds at 94° C., then 30 seconds at 58° C., then 30 seconds at 72° C.; and a last phase of returning to 72° C. for approximately 4 minutes. At the end of the PCR, the amplicons are preferably stored at −20° C.

In a preferred embodiment of the method according to the invention, the RT-MLPA step also comprises a step f) of analyzing the results of the PCR of step e), preferably by sequencing. According to the invention, the sequencing step is preferably a step of capillary sequencing or next-generation sequencing. For this purpose, it is possible to use a capillary sequencer (for example such as the AB13130 Genetic Analyzer, Thermo Fisher) or a next generation sequencer (for example the MiSeq System, Illumina, or the ion S5 System, Thermo Fisher). Several sequences are analyzed simultaneously, the index sequence thus making it possible to associate any identified genetic abnormality with a tested subject.

This analysis step allows immediately reading the result, and indicates directly whether the sample from the subject carries a specific translocation, identified or not, and/or exon skipping such as the skipping of exon 14 of the MET gene or the skipping of exons of the EGFR gene, or possibly a 5′-3′ imbalance.

In a preferred embodiment of the method according to the invention, the RT-MLPA step also comprises a step g) of determining the level of expression of the amplicons that are obtained at the end of the PCR step. Determining the level of expression of the amplicons allows ensuring in particular that the ligations obtained are indeed representative of a fusion transcript or of a transcript corresponding to exon skipping, and do not correspond to a ligation artifact. According to the invention, this step g) is implemented in particular by computer. This determining of the level of expression is implemented by the following steps: (1) demultiplexing the results obtained at the end of the PCR step (i.e. step e)) in order to isolate the sequences obtained for a given subject, thanks to the index sequences, (2) determining the number of DNA or RNA fragments present in the sample from the patient to be tested (before amplification) thanks to the molecular barcodes, and optionally (3) supplying an expression matrix for each fusion transcript or transcript corresponding to an exon skipping or to a 5′-3′ imbalance identified for the tested subject. This determining of the level of expression of the amplicons obtained at the end of a PCR step makes it possible to add more precision to the results of the PCR step, and in particular to the sequencing errors that may occur (see step f) indicated above). Ultimately, determining the level of expression of the amplicons obtained at the end of a PCR step makes it possible to add more precision to the diagnosis of cancer according to the invention.

According to an even more particular embodiment, step g) is a step of analyzing the amplicons obtained at the end of the PCR step, which is implemented by computer, in particular by an arrangement of bioinformatic algorithms. More particularly, this step g) comprises the following steps: (1) a step of demultiplexing based on the identification of the indexes, (2) a step of identifying the pairs of probes, (3) a step of counting the reads (results) and molecular barcode sequences (Barcodes: UMI sequence (Unique Molecular Index)), and optionally (4) a step of evaluating the quality of the sequencing of the sample. The sequences as analyzed by the software are shown in FIG. 7.

In a preferred embodiment of the method according to the invention, if, for a biological sample from a subject, a PCR amplification is obtained in step e) following hybridization with a pair of probes targeting fusion genes and/or exon skipping, then the subject is a carrier of the cancer linked to the genetic abnormality corresponding to the pair of probes identified. Preferably, this abnormality is typically analyzed in step f) and/or g) as mentioned above.

In a preferred embodiment of the method according to the invention, the PCR amplification of step e) is carried out using the pair of primers SEQ ID NO: 101 and 92 or SEQ ID NO: 102 and 94.

In a preferred embodiment of the method according to the invention, a cancer is thus identified and allows the patient (meaning the subject to whom the tested biological sample belongs) to benefit from a targeted therapy. According to the invention, “targeted therapy” means any anticancer therapy, such as chemotherapy, radiotherapy, or immunotherapy, but preferably means pharmacological inhibitors of the ALK, ROS, RET, EGFR, and MET proteins.

The invention also relates to a kit comprising at least the probes SEQ ID NO: 1 to 13, and/or the probes SEQ ID NO: 96 to 99, preferably further comprising the probes SEQ ID NO: 14 to 91, each of the probes preferably being fused, at at least one end, with a primer sequence, and at least one of the probes of said pair preferably comprising a molecular barcode sequence, in particular the probes SEQ ID NO: 14 to 91 and optionally SEQ ID NO: 96 and 98.

The invention also relates to a kit comprising at least the probes SEQ ID NO: 868 to 938 and/or the probes SEQ ID NO: 940 to 1104 and/or the probes SEQ ID NO: 1105 to 1107 and/or the probe SEQ ID NO: 939 and/or the probes SEQ ID NO: 1108 to 1123, each of the probes preferably being fused, at at least one end, with a primer sequence, and at least one of the probes of said pair preferably comprising a molecular barcode sequence.

The invention also relates to a kit comprising at least the probes SEQ ID NO: 1211 to 1312, each of the probes preferably being fused, at at least one end, with a primer sequence, and at least one of the probes of said pair preferably comprising a molecular barcode sequence.

The invention also relates to a kit comprising at least the probes SEQ ID NO: 1 to 13, and/or the probes SEQ ID NO: 96 to 99 and/or the probes SEQ ID NO: 866 to 938 and/or the probes SEQ ID NO: 940 to 1104 and/or the probes SEQ ID NO: 1105 to 1107 and/or the probe SEQ ID NO: 939 and/or the probes SEQ ID NO: 1108 to 1123, each of the probes preferably being fused, at at least one end, with a primer sequence, and at least one of the probes of said pair preferably comprising a molecular barcode sequence.

The invention also relates to a kit comprising at least the probes SEQ ID NO: 1 to 13, and/or the probes SEQ ID NO: 96 to 99 and/or the probes SEQ ID NO: 866 to 938 and/or the probes SEQ ID NO: 940 to 1104 and/or the probes SEQ ID NO: 1105 to 1107 and/or the probe SEQ ID NO: 939 and/or the probes SEQ ID NO: 1108 to 1123, and/or the probes SEQ ID NO: 1211 to 1312, optionally the probes SEQ ID NO: 1148, 1149, 1178, 1179, 1209 and/or 1210, each of the probes preferably being fused, at at least one end, with a primer sequence, and at least one of the probes of said pair preferably comprising a molecular barcode sequence.

The invention also relates to a kit comprising at least the following probes: SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824 and SEQ ID NO: 825, each of the probes being preferably fused, at at least one end, with a primer sequence, and at least one of the probes of said pair preferably comprising a molecular barcode sequence.

The invention also relates to a kit comprising at least the following probes: SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824, SEQ ID NO: 825, SEQ ID NO: 866 to 938, SEQ ID NO: 940 to 1104, SEQ ID NO: 1105 to 1107, SEQ ID NO: 939 and SEQ ID NO: 1108 to 1123, each of the probes being preferably fused, at at least one end, with a primer sequence, and at least one of the probes of said pair preferably comprising a molecular barcode sequence.

The invention also relates to a kit comprising at least the following probes: SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 826 to 835, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824, SEQ ID NO: 825, SEQ ID NO: 866 to 938, SEQ ID NO: 940 to 1104, SEQ ID NO: 1105 to 1107, SEQ ID NO: 939, SEQ ID NO: 1108 to 1123, and SEQ ID NO: 1211 to 1312, optionally the probes SEQ ID NO: 1148, 1149, 1178, 1179, 1209 and/or 1210, each of the probes preferably being fused, at at least one end, with a primer sequence, and at least one of the probes of said pair preferably comprising a molecular barcode sequence.

Determining the level of expression of the amplicons that are obtained at the end of a PCR step (for example carried out according to step e) above) is very advantageous because it allows ensuring that the obtained results are reliable. It allows in particular determining the number of RNA molecules (in particular the fusion transcripts or the transcripts corresponding to exon skipping or the transcripts of the genes whose 5′-3′ imbalance is to be analyzed) present in the sample to be tested. This adds more precision to the diagnosis performed.

In this aspect, the invention thus relates to a method for determining the level of expression of the amplicons that are obtained at the end of a PCR step, said method being implemented by computer and comprising the following steps:

(a) providing a sample to be tested, said sample comprising amplicons obtained at the end of a PCR step, and
(b) determining the level of expression of the amplicons.

In one particular embodiment of the method implemented by computer according to the invention, the determination of the level of expression of the amplicons aims in particular to:

(1) demultiplex the results of amplicons obtained at the end of a PCR step,
(2) determine the number of DNA or RNA fragments present in the sample of the patient to be tested (before amplification), and optionally
(3) provide an expression matrix for each fusion transcript or transcript corresponding to exon skipping identified for the patient being tested.

This determination of the level of expression of the amplicons that are obtained at the end of a PCR step allows adding more precision to the results. Analysis of the amplicons and their quantification can also be carried out very quickly.

In one particular embodiment, the method implemented by computer comprises the following steps:

(1) a step of demultiplexing the results of amplicons obtained at the end of a PCR step,
(2) a step of searching for pairs of probes used during the PCR step,
(3) a step of counting the reads (results, i.e. fusion transcripts or exon skippings) and molecular barcode sequences (UMI sequence (Unique Molecular Index)), optionally the index sequence, and optionally
(4) a step of evaluating the quality of sequencing of the sample.

The software according to the invention requires three files for its execution: a FASTQ, an index file and a marker file.

FASTQ: During a sequencing experiment, the raw data are generated in the form of a standard file called FASTQ. This FASTQ format will group, for each read sequenced by the device: (1) a unique sequence identifier, (2) the sequence of the read, (3) the read direction, (4) an ASCII sequence grouping the quality scores per base for each base that is read. An example of a read in FASTQ format is shown in FIG. 8. A FASTQ file is therefore composed of this repetition of 4 lines for each sequenced read. A high-throughput sequencing experiment generates hundreds of millions of sequences. The FASTQ file is the raw file required to launch the software according to the invention.

Marker file: This file groups all the sequences of each probe as well as their name. It brings together all the pairs of probes used during a diagnosis. It is specific to each kit (expression measurement, searching for fusion transcripts, for exon skipping, for imbalance, etc.).

Index file: This file groups the list of sequences used to identify the subjects tested. It gathers together all the index sequences used during a diagnosis. Each sequence will correspond to a tested subject and will allow reassigning the sequenced reads. This file is specific to each experiment.

According to the invention, the term “step of demultiplexing” means the step which aims to identify the various index sequences used during construction of the library to identify the reads for each of the subjects tested. This search is carried out by an exact and inexact matching algorithm for comparing sequences to allow taking into account the sequencing errors linked to the method of acquisition by high-throughput sequencing. According to the invention, a “library” is understood to mean the construction comprising at least an index sequence, a left probe and a right probe that are characteristic of a genetic abnormality, and optionally a molecular barcode sequence.

According to the invention, the term “step of searching for pairs of probes” means the step which aims to identify, for each sequence of the FASTQ file, whether there is a pair of probes in the marker file that allow attributing it to an entity that was to be measured (fusion transcripts, exon skipping . . . ). A data structure in the algorithm allows associating with each sequence a tag bearing the name of the two probes, left (“L”) and right (“R”). This search is carried out as an exact search by comparing sequences (e.g. the Hamming and Levenshtein distance calculation) and by an approximate method tolerating ‘k’ errors. This ‘k’ parameter can be changed when launching the tool. For the expression measurement, each pair of probes (right and left) is specific to an entity whose expression is to be measured. To measure the expression of a gene, two probes are used which hybridize strictly one behind the other to this gene. These probes will then be assembled during the ligation step, then amplified and read. Sequences having no logical tag during the search for probes are stored, in order to perform a search for chimeras. Indeed, it is possible that certain probes cross-hybridize during the hybridization, ligation, and amplification steps during construction of the library, leading to the appearance of hybrid sequences (for example a right probe of gene A with a left probe of gene B). Here again, these sequences are detected by exact and inexact matching of sequences. For the search for fusion transcripts, it is not known which probes will hybridize together and be amplified. The search for the probes is therefore carried out without preconceptions, by comparison of all pairs of possible right/left sequences.

According to the invention, the term “a step of counting the reads (results) and molecular barcode sequences” means the step occurring when the FASTQ file is scanned and the pairs of probes identified (markers and chimeras). The algorithm will proceed to count them. These counts are of two types: (1) quantifying the number of sequences read by the sequencer, and (2) the number of unique molecular barcode (UMI) sequences assigned to the marker. Sequence counting is done based on the data structure previously described during identification of the markers. The number of tags assigned for each marker will be determined by traversing the data structure. Counting the IMUs is more complex. It involves a step of extracting the UMI of each sequence and a step of correcting sequencing errors in the UMIs. The significant combinatorial analysis of these random sequences, their counts, and the amplification factor of the sample will make it possible to identify the IMUs carrying sequencing errors in order to correct the count data. This correction of the UMIs involves creating a graph structure associating a counter with each unique UMI. The UMIs are then grouped by increasing count with k tolerated errors. The UMIs allow identifying the number of unique sequences read by the sequencer before the amplification step during preparation of the library. They therefore provide information about the number of transcripts actually read and not the number of transcripts read after amplification.

According to the invention, the term “a step of evaluating the quality of sequencing of the sample” means the step which aims to determine the analyzed sequences which are not significant. A quality score indicative of the diversity of the libraries, meaning the number of unique transcripts read, has been implemented in the algorithm so as to provide an indication of the richness of the sample analyzed and to eliminate samples that would be considered as failures (i.e. having a score <5000).

Preferably, the method implemented by computer according to the invention makes it possible to calculate the level of expression of a large number of fusion transcripts or transcripts corresponding to exon skipping (in particular greater than 1000) for a large number of samples (in particular greater than 40), and to do so in a very short time (in particular 5 to 10 minutes).

According to one particular embodiment, the method implemented by computer can make it possible to correct sequencing errors which arise during sequencing of the amplicons, for example the correction of sequencing errors in molecular barcode sequences (UMI) (see for example ‘Method called Directional & Reference: Smith, T., Heger, A., & Sudbery, I. (2017). UMI-tools: modeling sequencing errors in Unique Molecular Identifiers to improve quantification accuracy. Genome Research, 27(3), 491-499. http://doi.org/10.1101/gr.209601.116))

Tables 1 and 2 below provide details concerning the sequences of the invention.

TABLE 1

| | |
|---|---|
| SEQ ID NO: 1 | SEQ ID NO: 52 |
| TGTCA | ATTG |
| CCCACCCCGGAGCCA | CTGTGGGAAATAATG |
| (R) | ATGTAAAG |
| SEQ ID NO: 2 | SEQ ID NO: 53 |
| AGCCC | GCAG |
| TGAGTACAAGCTGAG | CATGTCAGCTTCGTA |
| CAAGCTCCGC (R) | TCTCTCAA (L) |
| SEQ ID NO: 3 | SEQ ID NO: 54 |
| TGTAC | AAGA |
| CGCCGGAAGCACCAG | ACTAGTCCAGCTTCG |
| GAG (R) | AGCACAAG (L) |
| SEQ ID NO: 4 | SEQ ID NO: 55 |
| TGGAA | CAGG |
| GCAAGCAATTTCTTC | ACCTGGCTACAAGAG |
| AACC (R) | TTAAAAAG (L) |
| SEQ ID NO: 5 | SEQ ID NO: 56 |
| ATCTG | GAAC |
| GGCAGTGAATTAGTT | AGCTCACTAAAGTGC |
| CGCTACG (R) | ACAAACAG (L) |
| SEQ ID NO: 6 | SEQ ID NO: 57 |
| ATCAG | AGAA |
| TTTCCTAATTCATCT | GAGGGCATTCTGCAC |
| CAGAACGGTT (R) | AGATTG (L) |
| SEQ ID NO: 7 | SEQ ID NO: 58 |
| ATCCA | GAAA |
| CTGTGCGACGAGCTG | GGGAGTTTGGTTCTG |
| TGC (R) | TAGATG (L) |
| SEQ ID NO: 8 | SEQ ID NO: 59 |
| GAGGA | GTTG |
| TCCAAAGTGGGAATT | CTCCTATTGCAACAA |
| CCCT (R) | CAAACTCAG (L) |
| SEQ ID NO: 9 | SEQ ID NO: 60 |
| ATGTG | GGAT |
| GCCGAGGAGGCGGGC | CTTCGTAGCATCAGT |
| (R) | TGAAGCAG (L) |
| SEQ ID NO: 10 | SEQ ID NO: 61 |
| CTGG | TTTT |
| AGTCCCAAATAAACC | CTTACCACAACATGA |
| AGGCAT (R) | CAGTAGTG (L) |
| SEQ ID NO: 11 | SEQ ID NO: 62 |
| ATGA | AGGC |
| TTTTTGGATACCAGA | TGTGGAGTGGCAGCA |
| AACAAGTTTCA (R) | GAAG (L) |
| SEQ ID NO: 12 | SEQ ID NO: 63 |
| TCTG | GAGG |
| GCATAGAAGATTAAA | AACAGACTAAGAAGG |
| GAATCAAAAAA (R) | CTCAGCAAG (L) |
| SEQ ID NO: 13 | SEQ ID NO: 64 |
| TACT | GCTG |
| CTTCCAACCCAAGAG | TATCTCCATGCCAGA |
| GAGATTGAA (R) | GCAG (L) |
| SEQ ID NO: 14 | SEQ ID NO: 65 |
| CAAC | AAAG |
| ATTCAACTCCCTACT | CAGACCTTGGAGAAC |
| TTGTCCATCAG (L) | AGTCAG (L) |
| SEQ ID NO: 15 | SEQ ID NO: 66 |
| AGCC | CAGT |
| CAAGCTTCCCATCAC | GOATATTAGTGGACA |
| AG (L) | GOACTTAGTAG (L) |
| SEQ ID NO: 16 | SEQ ID NO: 67 |
| ACAG | GGTG |
| GCTGTGTGCATGCAC | GTACTGGCCCAAGGT |
| CAAAG (L) | AAAAAAG (L) |
| SEQ ID NO: 17 | SEQ ID NO: 68 |
| GAAG | CAGT |
| ATTGCCCGAGAGCAA | ATGAAAAAAAGCTTA |
| AAAG (L) | AATCAACCAAA (L) |
| SEQ ID NO: 18 | SEQ ID NO: 69 |
| GCAA | ACAT |
| AGCCAGCGTGACCAT | TTCATGGGGCTCCAC |
| C (L) | TAACAG (L) |
| SEQ ID NO: 19 | SEQ ID NO: 70 |
| TGAG | GTGG |
| CTCTCCAGAAAATTG | GAACGTGAAACATCT |
| ATGCAG (L) | GATACAAG (L) |
| SEQ ID NO: 20 | SEQ ID NO: 71 |
| CGAG | AGCT |
| TTCAAGCAGGCCTAT | GTCTGGCTCTGGAGA |
| ATCACCTG (L) | TCTGG (L) |
| SEQ ID NO: 21 | SEQ ID NO: 72 |
| TGGG | TGAG |
| AACATCCCATGGTAT | AGAACGGAGGTCCTG |
| CACA (L) | GCAG (L) |
| SEQ ID NO: 22 | SEQ ID NO: 73 |
| GCCA | GTAC |
| CCCATGCAGCCCACG | CACCTTATCCACAGC |
| (L) | CACAGC (L) |
| SEQ ID NO: 23 | SEQ ID NO: 74 |
| GCCC | GCTG |
| ACTGACGCTCCACCG | CCTGCGTCCCAAAGA |
| AAAG (L) | ACAG (L) |
| SEQ ID NO: 24 | SEQ ID NO: 75 |
| CCAA | ACAT |
| GCAGGATCTGGGCCC | AACCATTAGCAGAGA |
| AG (L) | GGCTCAGG (L) |
| SEQ ID NO: 25 | SEQ ID NO: 76 |
| GGCA | CGCC |
| GCTCAGCAGCTCCTC | TTCCAGCTGGTTGGA |
| AG (L) | G (L) |
| SEQ ID NO: 26 | SEQ ID NO: 77 |
| TGGC | GCAG |
| CAATGTGATCTGGAA | CTGCCCTTAGCCCTC |
| CTTATTAAT (L) | TGG (L) |
| SEQ ID NO: 27 | SEQ ID NO: 78 |
| ATCC | TGTT |
| AGGTCATGAAGGAGT | ACCTCAAGAAGCAGA |
| ACTTGACAAAG (L) | AGAAGAAAACA (L) |
| SEQ ID NO: 28 | SEQ ID NO: 79 |
| CTAC | GAAG |
| AGAGACACAACCCAT | CCTCCAAGCTATGAT |
| TGTTTATG (L) | TCTG (L) |
| SEQ ID NO: 29 | SEQ ID NO: 80 |
| CTAC | GACC |
| TCTGGTCTCTGGCAT | TTCCACCAATATTCC |
| TGCTGGTG (L) | TGAAAATG (L) |
| SEQ ID NO: 30 | SEQ ID NO: 81 |
| CTTC | TTGG |
| ATGAGCTGCAATCTC | CTTAACAGATGATCA |
| ATCACTG (L) | GGTTTCAG (L) |
| SEQ ID NO: 31 | SEQ ID NO: 82 |
| CCCACACCTGGGAAA | CTCAGACTCAAGCAG |
| GGACCTAAAG (L) | GTCAGATTGAAG (L) |
| SEQ ID NO: 32 | SEQ ID NO: 83 |
| GATCTGAATCCTGAA | AGCCTCAACAGTATG |
| AGAGAAATAGAG (L) | GTATTCAGTATTCAG |
| | (L) |
| SEQ ID NO: 33 | SEQ ID NO: 84 |
| TGAAAGAGAAATAGA | TCAGGGAACAGGAAG |
| GATATGCTGGATG (L) | AATTCCTAGGG (L) |
| SEQ ID NO: 34 | SEQ ID NO: 85 |
| TTTAATGATGGCTTC | TGGAAAAGACAATTG |
| CAAATAGAAGTACAG | ATGACCTGGAAG (L) |
| (L) | |
| SEQ ID NO: 35 | SEQ ID NO: 86 |
| GCCATAGGAACGCAC | AAACAACAGGAGTTG |
| TCAGGCAG (L) | CCATTCCATTACATG |
| | (L) |
| SEQ ID NO: 36 | SEQ ID NO: 87 |
| AGCTCTCTGTGATGC | CCGTCAGCCTCTTCT |
| GCTACTCAATAG (L) | CCCCAG (L) |
| SEQ ID NO: 37 | SEQ ID NO: 88 |
| ACTCGGGAGACTATG | GCTGCCAGATATTCC |
| AAATATTGTACT (L) | ACCCATACAG (L) |
| SEQ ID NO: 38 | SEQ ID NO: 89 |
| CAGTGAAAAAATCAG | ACAGAGGATGGCAGG |
| TCTCAAGTAAAG (L) | AGGAGTGCTTGCATG |
| | (L) |
| SEQ ID NO: 39 | SEQ ID NO: 90 |
| AGCATAAAGATGTCA | GTTAAGCCCCGTGGA |
| TCATCAACCAAG (L) | CCAAAGG (L) |
| SEQ ID NO: 40 | SEQ ID NO: 91 |
| AGCGGAAGGTTAATG | GCTGGAAACATTTCC |
| TTCTTCAGAAGAAG (L) | GACCCTG (L) |
| SEQ ID NO: 41 | SEQ ID NO: 92 |
| GGAGAAGACAAAGAA | GTGCCAGCAAGATCC |
| GGCAGAGAGAG (L) | AATCTAGA (L) |
| SEQ ID NO: 42 | SEQ ID NO: 93 |
| ATCAGATAAAGAGCC | TCCAACCCTTAGGGA |
| AGGAGCAGCTG (L) | ACCC (R) |
| SEQ ID NO: 43 | SEQ ID NO: 94 |
| CAAAGCCACTGGAGT | GCCATTGCGGTGACA |
| CTTTACCACAC (L) | CTATAG (L) |
| SEQ ID NO: 44 | SEQ ID NO: 95 |
| AGAAACAAGAAACCC | CCCTATAGTGAGTCG |
| TACAAGAAGAAATAA | TCGTCGC (R) |
| (L) | |
| SEQ ID NO: 45 | SEQ ID NO: 96 |
| AGCTTAAGAATGAAC | CTGTGGCTGAAAAAG |
| CGACCACAAGAA (L) | AGAAAGCAAATTAAA |
| | G (L) |
| SEQ ID NO: 46 | SEQ ID NO: 97 |
| CAAGTACTTGGATAA | ATCTGGGCAGTGAAT |
| GGAACTGGCAGGAAG | TAGTTCGCTACG (R) |
| (L) | |
| SEQ ID NO: 47 | SEQ ID NO: 98 |
| ACACAAGTGGGGAAA | GAATCTGTAGACTAC |
| TCAAAGTATTACAAG | CGAGCTACTTTTCCA |
| (L) | GAAG (L) |
| SEQ ID NO: 48 | SEQ ID NO: 99 |
| CCCACCTGAGCCTGC | ATCAGTTTCCTAATT |
| CGACT (L) | CATCTCAGAACGGTT |
| | C (R) |
| SEQ ID NO: 49 | SEQ ID NO: 100 |
| GCAAATCACAGATCG | NNNNNNNNNN |
| AAGAGACAG (L) | |
| SEQ ID NO: 50 | SEQ ID NO: 101 |
| TGCTGAGGGCTGGGA | GGGTTCCCTAAGGGT |
| AGAAG (L) | TGGA (L) |
| SEQ ID NO: 51 | SEQ ID NO: 102 |
| TTAGTTAATCACGAT | GCGACGACGACTCAC |
| TTCTCTCCTCTTGAG | TATAGGG (L) |
| (L) | |
| SEQ ID NO: 866 | SEQ ID NO: 1001 |
| CCGTCCACACCCGCC | GGTCACAGCCCCCAT |
| GCCAG (L) | TCCAG (L) |
| SEQ ID NO: 867 | SEQ ID NO: 1002 |
| ACCGCGAGAAGATGA | TGATGTCCTTGCATT |
| CCCAG (L) | GCCCATTTTTA (R) |
| SEQ ID NO: 868 | SEQ ID NO: 1003 |
| CTAAGCAGTGATGAA | GGGGCTCCAGGACCC |
| GAGGAGAATGAACAG | CTGCC (R) |
| (L) | |
| SEQ ID NO: 869 | SEQ ID NO: 1004 |
| CGCTCGCCCGGACCC | AGACCGAGGCAAAGG |
| CTCAG (L) | CCCTTTT (R) |
| SEQ ID NO: 870 | SEQ ID NO: 1005 |
| GAAGAAGAGCTGAGA | CAGGAACAAAGGCTG |
| AAAGCCATTTTAGTG | CTCCAGCT (L) |
| (R) | |
| SEQ ID NO: 871 | SEQ ID NO: 1006 |
| GAAGTGGTCCTGTAC | ATGACCTTCTTTCTG |
| TGCTTAGAGAACAAG | CCACAAAACGTAAAG |
| (R) | (L) |
| SEQ ID NO: 872 | SEQ ID NO: 1007 |
| GCGAGTATAGTGTTG | GCGAAGCTGGAGAAG |
| GAAACAAGCACC (R) | TCACTGGAG (R) |
| SEQ ID NO: 873 | SEQ ID NO: 1008 |
| TGCCGGAAGCTGCCC | CCACCAGGGAGCTCC |
| AGTGA (R) | TGCAG (L) |
| SEQ ID NO: 874 | SEQ ID NO: 1009 |
| GTTTACAGAAAAAGC | GAAACTGGGCATCTC |
| AAAGGAAACCGTTCT | TGTGGCC (R) |
| (L) | |
| SEQ ID NO: 875 | SEQ ID NO: 1010 |
| CTGACAGCGAAGACT | GATGGACATGGTAGA |
| CCGAAACAG (L) | GAATGCAGATAGTTT |
| | (R) |
| SEQ ID NO: 876 | SEQ ID NO: 1011 |
| GCAGCCCTGCTTCTT | GAGCTCTGGGCCCTG |
| CACAGTT (L) | GCGAG (L) |
| SEQ ID NO: 877 | SEQ ID NO: 1012 |
| TCCATGGCATCAAGT | GGGCCTCAGCGTGGA |
| GGACC (R) | CTCAG (L) |
| SEQ ID NO: 878 | SEQ ID NO: 1013 |
| GAGCTGGCGGCAGCG | CACTGGCCAGAGGTA |
| TGCAT (R) | CTTCCTCAA (L) |
| SEQ ID NO: 879 | SEQ ID NO: 1014 |
| GTGAAGCGGCCCAGG | GCAGTATCCCAGCCA |
| TGAGG (L) | AATCTCG (L) |
| SEQ ID NO: 880 | SEQ ID NO: 1015 |
| TCCACCCTCAAGGGC | CCAAATCCCACTCCC |
| CCCAG (L) | GACAG (L) |
| SEQ ID NO: 881 | SEQ ID NO: 1016 |
| CAGCAAGTATCCAAT | GACTTCAGACATGCA |
| GGGTGAAGAAG (L) | GGGTGACG (L) |
| SEQ ID NO: 882 | SEQ ID NO: 1017 |
| GTAAGACTCGGACCA | ATGAAAAAAAAGATA |
| AGGACAAGTACCG (R) | TTGACCATGAGACAG |
| | (R) |
| SEQ ID NO: 883 | SEQ ID NO: 1018 |
| GCAAACAGCAGCCCA | GGACAAACCTGACTC |
| GCAGA (L) | CTTCATGG (L) |
| SEQ ID NO: 884 | SEQ ID NO: 1019 |
| GTCGAGGGCCAAGAC | CAGCTCTGCTACCCC |
| GAAGACA (L) | AAGACAG (L) |
| SEQ ID NO: 885 | SEQ ID NO: 838 |
| CAGTAACCTTATGCC | NNNNNNNNNN |
| TAGCAACATGCCAAT | |
| (L) | |
| SEQ ID NO: 886 | SEQ ID NO: 1020 |
| ATCCCACTATTATTT | CATGGATCTGACTGC |
| TGGCACAACAGGAAG | CATCTACGAG (L) |
| (L) | |
| SEQ ID NO: 887 | SEQ ID NO: 1021 |
| AGAACCATTGGCTCT | CAGGCACCGCCCCTG |
| CACTGAAACAG (L) | GGGCT (R) |
| SEQ ID NO: 888 | SEQ ID NO: 1022 |
| AATGTGAAAAGGTTT | CCACTCGGGCGAGAA |
| GCGCTCCTG (L) | GCCGC (R) |
| SEQ ID NO: 889 | SEQ ID NO: 1023 |
| AGGACCTGGTGCAGA | CGGGTGGACATTCCC |
| TGCCT (R) | CTCAG (L) |
| SEQ ID NO: 890 | SEQ ID NO: 1024 |
| AAATTACAGGGGACA | GTGGGCCTCCTGGGC |
| TCAGGGCCACT (R) | CTCAG (L) |
| SEQ ID NO: 891 | SEQ ID NO: 1025 |
| CCCCAGTGGACCACC | TCCCTGGAATGAAGG |
| TGCAT (R) | GACACAGA (L) |
| SEQ ID NO: 892 | SEQ ID NO: 1026 |
| AAACTGCAGGGATCA | ATGGCAAAACTGGCC |
| GGCCC (R) | CCCCT (L) |
| SEQ ID NO: 893 | SEQ ID NO: 1027 |
| GGCACTGCACTGTGT | TCCCTGGACCTAAAG |
| GCGAG (L) | GTGCTGCT (L) |
| SEQ ID NO: 894 | SEQ ID NO: 1028 |
| TTGCTATAGCCCAAG | AAGCAGGCAAACCTG |
| GTGGAACAATC (R) | GTGAACAG (L) |
| SEQ ID NO: 895 | SEQ ID NO: 1029 |
| CTGCCACTGGTGACA | TCCAGGGCCTAAGGG |
| TGCCAAC (R) | TGACAGA (L) |
| SEQ ID NO: 896 | SEQ ID NO: 1030 |
| GCCTGACGCGGGCCG | CTGGTGCCCCTGGTG |
| CGCGG (L) | ACAAG (L) |
| SEQ ID NO: 897 | SEQ ID NO: 1031 |
| CCGACCTCACCCTGT | CTGGACCCCCTGGCC |
| CGCGG (L) | CCATT (L) |
| SEQ ID NO: 898 | SEQ ID NO: 1032 |
| GAGGAGCCTGTTCCC | AGGGTCCCCCTGGCC |
| CTGAG (L) | CTCCT (L) |
| SEQ ID NO: 899 | SEQ ID NO: 1033 |
| TGATGGCTTGTGCCC | CTGGTCCTGCTGGTC |
| AAACAG (L) | CCCGA (L) |
| SEQ ID NO: 900 | SEQ ID NO: 1034 |
| AGACAGCAGTGAGCA | CTGGCGAGCCTGGAG |
| TGGCG (L) | CTTCA (L) |
| SEQ ID NO: 901 | SEQ ID NO: 1035 |
| ATCAAGATGACTGTG | ATGTCACCGGGTGCG |
| CTCCTGTGGGA (R) | CATCAAT (R) |
| SEQ ID NO: 902 | SEQ ID NO: 1036 |
| ATATTGATGAGTGCC | CTACAAGAGACTGTG |
| AACTGGGGGAG (R) | AAAAGGAAGTTGGAA |
| | (R) |
| SEQ ID NO: 903 | SEQ ID NO: 1037 |
| GGTCAAATTTCAGCC | CATCCCAGTGACTGC |
| ATCAGCAA (L) | ATCCCTC (R) |
| SEQ ID NO: 904 | SEQ ID NO: 1038 |
| AGGACTGGGCGCTGC | GGGGACCCCATTCCC |
| TGCAG (L) | GAGGA (R) |
| SEQ ID NO: 905 | SEQ ID NO: 1039 |
| GTAAAAGTAGCAGTG | GTTTCAAAGTCACCC |
| GTTCAGOACACTTTG | TCCCACCTTT (R) |
| (L) | |
| SEQ ID NO: 906 | SEQ ID NO: 1040 |
| TCAGACGAAGAACCT | GTCCCGTGGCTGTCA |
| CTCTCCCAG (L) | TCAGTG (R) |
| SEQ ID NO: 907 | SEQ ID NO: 1041 |
| CAGTGCCATCAGCAG | CCCTGGCGAGCCCCT |
| CATAGCAAG (L) | TGCAG (L) |
| SEQ ID NO: 908 | SEQ ID NO: 1042 |
| GCTCGACTGTGGGGA | ACACTAACAGCACAT |
| AACCATAAG (L) | CTGGAGACCCG (R) |
| SEQ ID NO: 909 | SEQ ID NO: 1043 |
| GCCACCACCACTCCG | GTCTCGGTGGCTGTG |
| TGGAG (L) | GGCCT (R) |
| SEQ ID NO: 910 | SEQ ID NO: 1044 |
| CCAGCAGCCACTGCA | TGTCCTCCTTGAAGG |
| CCTACAAG (L) | GCTCCAG (L) |
| SEQ ID NO: 911 | SEQ ID NO: 1045 |
| TATGGACAGAGTAAC | CCTCCACTGAAGAAG |
| TACAGTTATCCCCAG | CTGAAACAAGAG (L) |
| (L) | |
| SEQ ID NO: 912 | SEQ ID NO: 1046 |
| CCCTGACCGAGAAGT | GAGAGTCTGGATGGA |
| TTAATCTGCCT (R) | CATTTGCAGG (L) |
| SEQ ID NO: 913 | SEQ ID NO: 1047 |
| TCTTGAAAGCGCCAC | TGCGAAGCCACCTCT |
| AAGCA (R) | CGCAG (L) |
| SEQ ID NO: 914 | SEQ ID NO: 1048 |
| ATGCTCTCCCCTCCT | GCTCTCCACAGATAG |
| CGGAGGA (R) | AGAACATCCAGC (R) |
| SEQ ID NO: 915 | SEQ ID NO: 1049 |
| GGAGAGGAGCACCAC | CTGAACAGATGGGTA |
| CCCAG (L) | AGGATGGCAG (R) |
| SEQ ID NO: 916 | SEQ ID NO: 1050 |
| GTGTCCCTATCTCTG | GGACCAACCACTTCC |
| ATACCATCATCCCAG | TACCCCAG (R) |
| (L) | |
| SEQ ID NO: 917 | SEQ ID NO: 1051 |
| CTCCTTCAGACAATG | GCCCCAGGTGTACCC |
| CAGTGGTCTTAACAA | ACCAC (R) |
| (L) | |
| SEQ ID NO: 918 | SEQ ID NO: 1052 |
| GCACACCTCTTAGAG | GCCTCACCTGCAGAT |
| GAAGACAGAAAACAG | GCCCC (R) |
| (L) | |
| SEQ ID NO: 919 | SEQ ID NO: 1053 |
| GAAGTGGTCATTTCA | GCAACCTCCAAGTCC |
| GATGTGATTCATCTA | CAGATCATGT (R) |
| (L) | |
| SEQ ID NO: 920 | SEQ ID NO: 1054 |
| CTCCTCACCCTCTGC | GGAGTTCCTGGTCGG |
| CGAGTCTCAAT (R) | CTCCG (R) |
| SEQ ID NO: 921 | SEQ ID NO: 1055 |
| GAGTGCGCCGGTCTC | CTTACCGTGACGTCC |
| GGGGA (R) | ACCGAC (L) |
| SEQ ID NO: 922 | SEQ ID NO: 1056 |
| TGGTGGCTATGAACC | GAGAGAGCCTTGAAC |
| CAGAGGT (L) | TCTGCCAGC (R) |
| SEQ ID NO: 923 | SEQ ID NO: 1057 |
| AGTCTGTGGCTGATT | TTTAAGGAGTCGGCC |
| ACTTCAAGCAGATTG | TTGAGGAAGC (R) |
| (L) | |
| SEQ ID NO: 924 | SEQ ID NO: 1058 |
| CCCATCTCTGGGATT | GTGCCAGGCCCACCC |
| CCCAG (R) | CCAGG (R) |
| SEQ ID NO: 925 | SEQ ID NO: 1059 |
| CTGAAGTCTGAGCTG | GTAAAGGCGACACAG |
| GACATGCTG (R) | GAGGAGAACC (R) |
| SEQ ID NO: 926 | SEQ ID NO: 1060 |
| GATCCCCTGTTGGGG | CCTCTGTGTTTGCCG |
| ATGCT (R) | CCTGG (L) |
| SEQ ID NO: 927 | SEQ ID NO: 1061 |
| CTGAAGGATGCTGTA | TGTTGAAGAGATTGG |
| CCACAGACG (L) | CTGGTCCTATACAG (L) |
| SEQ ID NO: 928 | SEQ ID NO: 1062 |
| GGACGACTTTATGAC | ACACATTCATTCATA |
| CAAGAGCTGAACAAG | ACACTGGGAAAACAG |
| (L) | (L) |
| SEQ ID NO: 929 | SEQ ID NO: 1063 |
| CTGCATACGGCAGGA | ATAAACCTCTCATAA |
| GGGAAAG (L) | TGAAGGCCCCCG (R) |
| SEQ ID NO: 930 | SEQ ID NO: 1064 |
| GAACCAACCGGTGAG | CCTGCAGCCCCCATA |
| CCCTC (R) | GCAG (L) |
| SEQ ID NO: 931 | SEQ ID NO: 1065 |
| TGAACCCCACCAACA | CTCGCAACGCCCTGG |
| CAGTTTTTG (L) | TGGTC (R) |
| SEQ ID NO: 932 | SEQ ID NO: 1066 |
| GGCCAACGGGTCTAA | GTGGCCTTGACCTCC |
| AGCAG (L) | AACCAG (L) |
| SEQ ID NO: 933 | SEQ ID NO: 1067 |
| AACCTATGTTGCCCT | GGGCTGCTGGAGTCC |
| GAGTTACATAAATAG | TCTGC (R) |
| (L) | |
| SEQ ID NO: 934 | SEQ ID NO: 1068 |
| CCGCAGCAGCACTCC | GCATAGAGAAGGAGA |
| GACAG (L) | CGTGCCAGAAG (R) |
| SEQ ID NO: 935 | SEQ ID NO: 1069 |
| GGGAGGTTCAAGATT | CGGGTCCTGAACGCT |
| CTTATGAAGCTTATG | GTGAAAT (L) |
| (L) | |
| SEQ ID NO: 936 | SEQ ID NO: 1070 |
| GCAGAAGTTAGCGCT | ATTATGGAACTGCAG |
| TCTCTCTCG (L) | CGAATGACATC (R) |
| SEQ ID NO: 937 | SEQ ID NO: 1071 |
| GCCGTGGTGGCTGGT | GCCCAGAGATCGCAG |
| TCCCT (R) | CATATCAAA (L) |
| SEQ ID NO: 938 | SEQ ID NO: 1072 |
| CGACTCATTCATCGC | GATGAGATTCTTCCA |
| CCTCCAG (L) | AGGAAAGACTATGAG |
| | (L) |
| SEQ ID NO: 940 | SEQ ID NO: 1073 |
| TGCGGGGCCAGGTGG | GGTCAAGCTGCTGCT |
| CCAAG (L) | GCTCG (L) |
| SEQ ID NO: 941 | SEQ ID NO: 1074 |
| CTGGACTTCCAGAAG | GGGGACCTAATTACA |
| AACATCTACAGTGAG | CCTCCGGTTATG (L) |
| (L) | |
| SEQ ID NO: 942 | SEQ ID NO: 1075 |
| GAGAATCTTTTAGGA | CAGCCTACATCGGAT |
| CAAGCACTGACGAAG | GCCCA (L) |
| (L) | |
| SEQ ID NO: 943 | SEQ ID NO: 1076 |
| CTCCAGGGTTCCTTG | CGGCCAACAATCCCT |
| AAAAGAAAACAGG (R) | GCAGT (L) |
| SEQ ID NO: 944 | SEQ ID NO: 1077 |
| TAAAAAGCGAAAGAA | CGACGGGTCCATTGC |
| TAAAAACCGGCACAG | CAAG (L) |
| (L) | |
| SEQ ID NO: 945 | SEQ ID NO: 1078 |
| GGGGACAACAGCAGT | GCCTGTCGGGGGTAC |
| GAGCAAG (L) | CACAG (L) |
| SEQ ID NO: 946 | SEQ ID NO: 1079 |
| GCCACTCAATGACAA | GACTTGATTAGAGAC |
| AAATAGTAACAGTGG | CAAGGATTTCGTGG (R) |
| (R) | |
| SEQ ID NO: 947 | SEQ ID NO: 1080 |
| TCCACGGACGACTCA | GATCAACCACAGGTT |
| GAGCAAG (L) | TGTCTGCTACC (R) |
| SEQ ID NO: 948 | SEQ ID NO: 1081 |
| AATGAAGTTAGAAGA | AAAACACTTGGTAGA |
| AAGCGAATTCCATCA | CGGGACTCGAGT (R) |
| (L) | |
| SEQ ID NO: 949 | SEQ ID NO: 1082 |
| CGGGGCAGATCCAGG | AGCTAAAAGGACAGC |
| TTCAG (L) | AGGTGCTACCA (L) |
| SEQ ID NO: 950 | SEQ ID NO: 1083 |
| TTTACAGCTGACCTT | TTTGCAGAAACACTC |
| GACCAGTTTGATCAG | CAATTTATAGATTCT |
| (R) | (L) |
| SEQ ID NO: 951 | SEQ ID NO: 1084 |
| GATTACCTGAGCTGG | GCCTACCCTTCTCTC |
| AATTGGAAGCAAT (R) | CCTCGCAG<L) |
| SEQ ID NO: 952 | SEQ ID NO: 1085 |
| CCTGGCAGTGAGCTG | GAAATTAAATACGGT |
| GACAACT (R) | CCCCTGAAGATGCTA |
| | (L) |
| SEQ ID NO: 953 | SEQ ID NO: 1086 |
| CTTTTAATAACCCAC | ACCACCCTTACTGAA |
| GACCAGGGCAACT (R) | GAAAATCAAACAAGA |
| | G (L) |
| SEQ ID NO: 954 | SEQ ID NO: 1087 |
| GAATGATTGGTAACA | CGCCTGTGGCAGATG |
| GTGCTTCTCGG (R) | CACCG (L) |
| SEQ ID NO: 955 | SEQ ID NO: 1088 |
| CATCCTGCCTATAGA | GAGGAGCAAAATAGA |
| CCAGGCGTCTTTT (R) | GGCAAGCCC (R) |
| SEQ ID NO: 956 | SEQ ID NO: 1089 |
| GGCCATCTGAATTAG | GCAGAAGGAGAAGAC |
| AGATGAACATGGG (R) | AGCCTGAAGA (R) |
| SEQ ID NO: 957 | SEQ ID NO: 1090 |
| CCCGACCCTGCCCGC | CCCGCCCAAGGGCCC |
| CCTGG (R) | AG (L) |
| SEQ ID NO: 939 | SEQ ID NO: 1091 |
| GTAATTATGTGGTGA | GCTCACCCAGTCCCC |
| CAGATCACGGCTCG (R) | ACCAG (L) |
| SEQ ID NO: 958 | SEQ ID NO: 1092 |
| CTGAGGATTTGTGAC | AACTGTTCCCCCTCA |
| TGGACCATGAATC (R) | TCTTCCCG (R) |
| SEQ ID NO: 959 | SEQ ID NO: 1093 |
| TCCTGGTACCTGGGC | AAGAGGATGGATTCG |
| TAGCTTGGT (R) | ACTTAGACTTGACCT |
| | (L) |
| SEQ ID NO: 960 | SEQ ID NO: 1094 |
| GTGGGAGGCCGCACC | CTTCTTTTTCAGAAG |
| ATGCT (R) | ACACCCTAAAAAAAG |
| | (R) |
| SEQ ID NO: 961 | SEQ ID NO: 1095 |
| AGAGCACGGATAACT | CTGATTCCAGAGAGC |
| TTATCTTGT (R) | TAAAGCCGATG (L) |
| SEQ ID NO: 962 | SEQ ID NO: 1096 |
| TTGACGAAGTGAGTC | AAAGCCAAACTTGGC |
| CCACACCTCCT (R) | CCTGCT (R) |
| SEQ ID NO: 963 | SEQ ID NO: 1097 |
| ATGAACAGCAAAGAT | CACCTGCAAGATGGG |
| GTTCAGTATTGTGCT | GCTGG (L) |
| (R) | |
| SEQ ID NO: 964 | SEQ ID NO: 1098 |
| CATCTGCATTGCCGG | ATCTCCTGTGTGCCC |
| GACCG (R) | AGAAGACCT (L) |
| SEQ ID NO: 965 | SEQ ID NO: 1099 |
| GTTCATGGAGTTTGA | GTGCAAACCCAAATT |
| GGCTGAGGAGA (R) | ATCCTGATGTAATTT |
| | (R) |
| SEQ ID NO: 966 | SEQ ID NO: 1100 |
| TGTACATTCCGAAGA | GTCTATGCTGTGGTG |
| AGGCAGCCT (R) | GTGATTGCGTC (R) |
| SEQ ID NO: 967 | SEQ ID NO: 1101 |
| CATACCCAGCGCTGG | ATTTCTCATGGTTTG |
| GACCG (R) | GATTTGGGAAAGTA (R) |
| SEQ ID NO: 968 | SEQ ID NO: 1102 |
| GAATCTTTCTGAACC | GCCCAGCCTCCGTTA |
| TGTCATGACCTATAG | TCAGC (R) |
| (R) | |
| SEQ ID NO: 969 | SEQ ID NO: 1103 |
| GGCGGCGGTGCAGCG | AAATTAAATACGGTC |
| CTCCG (L) | CCCTGAAGATGCTA (L) |
| SEQ ID NO: 970 | SEQ ID NO: 1104 |
| GCCTGATCACTTGAA | GCAGAAGGAGAAGAC |
| CGGACATATCAAG (R) | AGCCTGAAGA (R) |
| SEQ ID NO: 971 | SEQ ID NO: 1105 |
| ACCTGCAATGCTTCT | GTCGGGCTCTGGAGG |
| TTTGCCACC (R) | AAAAGAAAG (L) |
| SEQ ID NO: 972 | SEQ ID NO: 1106 |
| TCTTACCAGCCCACA | TTTGCCAAGGCACGA |
| TCTATTCCACAAG (L) | GTAACAAG (R) |
| SEQ ID NO: 973 | SEQ ID NO: 1107 |
| GCGGAAGAGACGGAA | CCTGCGTGAAGAAGT |
| TTTCAACAA (R) | GTCCCC (L) |
| SEQ ID NO: 974 | SEQ ID NO: 1108 |
| ACGGAAAAGGCGTAA | ACCGATCAAGAGCTC |
| CTTCAGTAAACAG (R) | TCCATGTGAG (L) |
| SEQ ID NO: 975 | SEQ ID NO: 1109 |
| TTGACCTGGATAGGC | CTCCGAATGTCCTGG |
| TCAATGATGAT (R) | CTCATTCG (R) |
| SEQ ID NO: 976 | SEQ ID NO: 1110 |
| CAGCCCCATCCGGAT | GCCAGCCACCGACAC |
| GTTTG (R) | CTACAG (L) |
| SEQ ID NO: 977 | SEQ ID NO: 1111 |
| GCCCCCCCAGGATGC | CATCTCGGGCTACGG |
| AATGG (R) | AGCTGC (R) |
| SEQ ID NO: 978 | SEQ ID NO: 1112 |
| GTTGCCTCTTGGTGC | GGCAATTCCGGAGCC |
| TGCCT (R) | GCAG (L) |
| SEQ ID NO: 979 | SEQ ID NO: 1113 |
| ATTGGCCAAAATGGG | GTGGTGGAGGTGGCT |
| AAGGATTGG (R) | GGAATG (R) |
| SEQ ID NO: 980 | SEQ ID NO: 1114 |
| TCCCAGGACATCAAA | GCATCCTGTACACCC |
| GCTCTGCAG (R) | CAGCTTTAAAAG (L) |
| SEQ ID NO: 981 | SEQ ID NO: 1115 |
| GTGAAAAAACACGTG | TGATGGAAGGCCACG |
| CGCAGCTTC (R) | GGGAA (R) |
| SEQ ID NO: 982 | SEQ ID NO: 1116 |
| GAGATATCTCTGTGA | CCCCTGCAAGTGGCT |
| GTATTTCAGTATCAA | GTGAAG (L) |
| (R) | |
| SEQ ID NO: 983 | SEQ ID NO: 1117 |
| GACATGAGCACAGTA | ACGCTGCCTGAAGTG |
| TATCAGATTTTTCCT | TGCTCTG (R) |
| (R) | |
| SEQ ID NO: 984 | SEQ ID NO: 1118 |
| GTGCCCCAAAGATGC | CCTCATGGAAGCCCT |
| AAACG (L) | GATCATCAG (L) |
| SEQ ID NO: 985 | SEQ ID NO: 1119 |
| AAGTATTTGGCTGAG | CAAATTCAACCACCA |
| GAGTTTTCAATCCCA | GAACATTGTTCG (R) |
| (L) | |
| SEQ ID NO: 986 | SEQ ID NO: 1120 |
| AAGCACAAGACCAAG | GGGATGGCCCGAGAC |
| ACAGCTCAACAG (L) | ATCTACAG (L) |
| SEQ ID NO: 987 | SEQ ID NO: 1121 |
| CTCAGTTCATTGCCA | GGCGAGCTACTATAG |
| GAGAGCCAT (L) | AAAGGGAGGCTG (R) |
| SEQ ID NO: 988 | SEQ ID NO: 1122 |
| CACCCCAGCCCTATC | CAAGAACTGCCCTGG |
| CCTTTACGT (R) | GCCTGT (L) |
| SEQ ID NO: 989 | SEQ ID NO: 1123 |
| CATGGAGACCCATTC | ATACCGGATAATGAC |
| AGATAACCCACTAAG | TCAGTGCTGGC (R) |
| (L) | |
| SEQ ID NO: 990 | SEQ ID NO: 996 |
| ACCATGTCAGCAAAA | GTTTCAGCAGTTCAG |
| CTTCTTTTGGG (L) | CTCCACCAG (L) |
| SEQ ID NO: 991 | SEQ ID NO: 997 |
| GTTCTCCAAACCTAT | ATGTTGGATGACAAT |
| CCCCGAATCCG (R) | AACCATCTTATTCAG |
| | (R) |
| SEQ ID NO: 922 | SEQ ID NO: 998 |
| ACCTGCAGCCAGTTA | GTATCAGCAGATGTT |
| CCTACTGCGAG (L) | GCACACAAACTTG (R) |
| SEQ ID NO: 993 | SEQ ID NO: 999 |
| ATGTAAAATGGGGTA | GCGGCCCTACGGCTA |
| AACTGAGAGATTATC | TGAACAG (L) |
| (L) | |
| SEQ ID NO: 994 | SEQ ID NO: 1000 |
| AGGTACCAATCTTGG | AGCCAACACAGATCT |
| GAAAAAGAAGCAACA | ATAGATTTCTTCGAA |
| (L) | (R) |
| SEQ ID NO: 995 | SEQ ID NO: 865 |
| GACCTCCTCCAGCGG | NNNNNNNNNNNNNNN |
| GACAG (L) | NNNNN |
| SEQ ID NO: 1209 (R) | SEQ ID NO: 1210 (L) |
| TCTGGCATAGAAGAT | TGGAAAAGACAATTG |
| TAAAGAATCAAAAAA | ATGACCTGGAAG |
| SEQ ID NO: 1211 (R) | SEQ ID NO: 1212 (L) |
| GATAGCTAGCGGCCA | TGACTTCTGGATTCT |
| GGAGAAATACAGT | CCTCTTGAGTAAAAG |
| SEQ ID NO: 1213 (L) | SEQ ID NO: 1214 (R) |
| CGAACATGGCACGAA | TTTGGACATCACATT |
| AGAGATCAAG | TCACAGTCAGAAGG |
| SEQ ID NO: 1215 (R) | SEQ ID NO: 1216 (R) |
| ACCAAGCCACCCTGG | ACAGGTGATTTGGCT |
| TAGAACAAGTAA | TCTGCACAGTTAG |
| SEQ ID NO: 1217 (R) | SEQ ID NO: 1218 (L) |
| ATGGTGCTCCAAGAG | CCTTATTGGAGATTT |
| GCAGCTT | TACATTGTGCTATAG |
| SEQ ID NO: 1219 (L) | SEQ ID NO: 1220 (L) |
| CTGGCTGGAAAAAGA | TGGGAGAAGCAGCAG |
| GGAAAGATTTCTG | CGCAAG |
| SEQ ID NO: 1221 (L) | SEQ ID NO: 1222 (R) |
| GCCAAGAGGCAGACC | CTCCAGAAACATGAC |
| TAGGAAATGG | AAGGAGGACTTTC |
| SEQ ID NO: 1223 (L) | SEQ ID NO: 1224 (R) |
| TGGCGAAGCGGAGGC | CTGTCTGCGAGCCTG |
| CGGAG | GCTGTG |
| SEQ ID NO: 1225 (L) | SEQ ID NO: 1226 (L) |
| CAAGTTGTTCAGAAG | AGATGGTGCAGAAGA |
| AAGCCTGCTCAG | AGAACGCG |
| SEQ ID NO: 1227 (R) | SEQ ID NO: 1228 (L) |
| GGTACGAAGCCAGCC | GGAACTGCCAGTGTA |
| TCATACATGC | GAGGGAATTCTAAG |
| SEQ ID NO: 1229 (L) | SEQ ID NO: 1230 (R) |
| GCCTTTTTGAAGAAA | GATGAGCAATTCTTA |
| CTCCACGAAGAG | GGTTTTGGCTCAGAT |
| SEQ ID NO: 1231 (L) | SEQ ID NO: 1232 (L) |
| GCTGGAAACATTTCC | AAGGAGAAGGGGTTG |
| GACCCTG | AAATTGTTGATAGAG |
| SEQ ID NO: 1233 (L) | SEQ ID NO: 1234 (L) |
| ATCAAGTCCTTTGAC | GCAAGAGTGGTGATC |
| AGTGCATCTCAAG | GTGGTGAGACT |
| SEQ ID NO: 1235 (R) | SEQ ID NO: 1236 (L) |
| TTTTTTTGAAGAAGC | TCTTATCCTTTGTCG |
| AGGATGCTGATCTAA | CAGAGACTATCTGAG |
| SEQ ID NO: 1237 (R) | SEQ ID NO: 1238 (L) |
| GGCTATTGAGTGGCC | AGGTTGTTACCGTGG |
| AGACTTCCC | GCAACTCTG |
| SEQ ID NO: 1239 (R) | SEQ ID NO: 1240 (L) |
| GTGGTGGAGGTGGCT | CCAGAAAAAAAGACC |
| GGAATG | AGGCCACAG |
| SEQ ID NO: 1241 (L) | SEQ ID NO: 1242 (R) |
| GCCTTCTACCCCATG | CAGCAGCCAGTAAGG |
| AGAAAGACCAG | AGGAGAAGG |
| SEQ ID NO: 1243 (L) | SEQ ID NO: 1244 (L) |
| GAGTTCAGGACCAGC | GTGGAAAAGGCTTTA |
| TCATTGAAAAGA | GCCATGGACAG |
| SEQ ID NO: 1245 (R) | SEQ ID NO: 1246 (L) |
| AGATCTGTCTTACAA | CCAAGGCTTGACCCT |
| CCTATTAGAAGATTT | CGTTTTG |
| SEQ ID NO: 1247 (L) | SEQ ID NO: 1248 (R) |
| AAACAGCAAGAACTG | ACAAGTCATCAATTG |
| CTTCGGCAG | CTGGCTCAGAA |
| SEQ ID NO: 1249 (R) | SEQ ID NO: 1250 (L) |
| GGTCAAGAAAGTGAC | GTCCTCCGACAGTGC |
| TCATCAGAGACCTCT | TTGGCA |
| SEQ ID NO: 1251 (R) | SEQ ID NO: 1252 (L) |
| AAGATGAATCCGGCC | CGGAGTCAGCTGCCA |
| TCGGC | AGAGACAG |
| SEQ ID NO: 1253 (R) | SEQ ID NO: 1254 (L) |
| GTGCTATACTTGGTA | GACCATCATCCAGGG |
| GATCAGAAACTCAGG | CATCCTG |
| SEQ ID NO: 1255 (L) | SEQ ID NO: 1256 (L) |
| TGACACGCTTCCCTG | CAGCTCCTGACCAAC |
| GATTGG | CCCAAG |
| SEQ ID NO: 1257 (L) | SEQ ID NO: 1258 (L) |
| ACAGGGACGCCATCG | TGAAATCCGACACTA |
| AATCCG | CTGATTCTAGTCAAG |
| SEQ ID NO: 1259 (L) | SEQ ID NO: 1260 (R) |
| TTGGAGAAGATCTAT | GTTACTCTGGAAGAA |
| GGGTCAGACAGAATT | GTCAACTCCCAAATA |
| SEQ ID NO: 1261 (R) | SEQ ID NO: 1262 (R) |
| AACTCGAAAATTAAT | GACTGGGAGGTGCTG |
| GCTGAAAATAAGGCG | GTCCTAGG |
| SEQ ID NO: 1263 (R) | SEQ ID NO: 1264 (R) |
| TTTAAGGCTGCAAGC | AATCATCGGACTCAG |
| AGTATTTACAACAGA | GTACATCTGTGAGTG |
| SEQ ID NO: 1265 (R) | SEQ ID NO: 1266 (L) |
| GCCTGTGCAGTGGGA | GTTCAAAAACTGAAG |
| CTGATTG | GACTCTGAAGCTGAG |
| SEQ ID NO: 1267 (L) | SEQ ID NO: 1268 (L) |
| CGCCAATTGTAAACA | CCTTATTGATTGGCC |
| AAGTGGTGACAC | AACAATCAACAG |
| SEQ ID NO: 1269 (R) | SEQ ID NO: 1270 (R) |
| CCCAGCCCTGGGGAG | CCGTAGCTCCATATT |
| CCCCT | GGACATCCC |
| SEQ ID NO: 1271 (L) | SEQ ID NO: 1272 (R) |
| CCCTGAGAATCTGGG | TGTGTGCCTCCTGAC |
| ACCTCAACAG | GAAGCC |
| SEQ ID NO: 1273 (R) | SEQ ID NO: 1274 (L) |
| GCCACAGTGGAGACC | GCCAAGAGGAGCTCA |
| AGTCAGC | TGAGGCAG |
| SEQ ID NO: 1275 (L) | SEQ ID NO: 1276 (L) |
| TCTCTAGCAGTTACT | AACTCACAACGGTAG |
| ATGGATGACTTCCGG | GAGAGAAACCTGAAG |
| SEQ ID NO: 1277 (L) | SEQ ID NO: 1278 (R) |
| AGCCCGGGACCGTTT | AAATGTGGAGCCCAG |
| AAAAAACTG | GAGGAAGG |
| SEQ ID NO: 1279 (L) | SEQ ID NO: 1280 (R) |
| AATGGTCAGAAACCC | GATGCAATTCGAAGT |
| TCCATAACCTGAAG | CACAGCGAAT |
| SEQ ID NO: 1281 (L) | SEQ ID NO: 1282 (R) |
| CGGACGCATCACTTG | AGCTGATAGACACAC |
| CACTTCTAGAA | ACCTTAGCTGGATAC |
| SEQ ID NO: 1283 (L) | SEQ ID NO: 1284 (R) |
| CTTTGCTGAATGCTC | CTTGTAATCTGGATG |
| CAGCCAAG | TGATTCTGGGGTTT |
| SEQ ID NO: 1285 (R) | SEQ ID NO: 1286 (R) |
| GAAAGCCCTTCTTGT | GTAACAGTATCGGGA |
| ATGTCAATGCC | CCCTTACTGCACAT |
| SEQ ID NO: 1287 (R) | SEQ ID NO: 1288 (R) |
| ACATTACTGGTTATA | CTCAAGCTTTTAAAA |
| GAATTACCACAACCC | TCGAGACCACCCC |
| SEQ ID NO: 1289 (L) | SEQ ID NO: 1290 (R) |
| AGCCCCAGTCCCAGC | AATGCAGCTCTTCAG |
| CCCAG | CATCTGTTTATTCG |
| SEQ ID NO: 1291 (L) | SEQ ID NO: 1292 (L) |
| CGAGGGTGTTCTTGA | CTCCGCCCCACAGTC |
| CGATTAATCAACAG | CACGAG |
| SEQ ID NO: 1293 (L) | SEQ ID NO: 1294 (L) |
| GTGGCGGAATCGGTG | CGCCATCATCCTCAT |
| GTAGAG | CATCATCATAG |
| SEQ ID NO: 1295 (R) | SEQ ID NO: 1296 (L) |
| AGATCATCACTGGTA | ACAGTCTCTTGCAAT |
| TGCCAGCCTC | CGGCTAAAAAAAAGA |
| SEQ ID NO: 1297 (L) | SEQ ID NO: 1298 (L) |
| CTATCAGAAGAAAAT | AGAAAACTCTTAAAG |
| CGGCACCTGAGA | AATGCAGCAGCTTGG |
| SEQ ID NO: 1299 (R) | SEQ ID NO: 1312 (R) |
| GACACTGGGGTTGGG | GGTCCTGTCGGGGAA |
| AAATCAAGC | CCCTCT |
| SEQ ID NO: 1300 (L) | SEQ ID NO: 1301 (L) |
| CCCAGCGCTACCTTG | CAGTTTGCTGTGTGT |
| TCATTCAG | TTGCTCAAACAG |
| SEQ ID NO: 1302 (L) | SEQ ID NO: 1303 (R) |
| TACTTGGACTAGTTT | GACATGAACAAGCTG |
| ATATGAAATTTGTGG | AGTGGAGGCGGCG |
| SEQ ID NO: 1304 (R) | SEQ ID NO: 1305 (R) |
| CTACATCTACATCCA | CCTTGCCTCCCCGAT |
| CCACTGGGACAAG | TGAAAG |
| SEQ ID NO: 1306 (L) | SEQ ID NO: 1307 (L) |
| GTGCCACGGTGTCCG | ATTTTAATGAAAACA |
| GATATG | CAGCAGCACCTAGAG |
| SEQ ID NO: 1308 (L) | SEQ ID NO: 1309 (L) |
| ATGAAGGAAATGCTA | TGCCATCTCCAGGCC |
| AAGCGATTCCAAG | TTGCAG |
| SEQ ID NO: 1310 (R) | SEQ ID NO: 1311 (R) |
| GCCCGGCTGTGCTGG | TCCCGGCCAGTGTGC |
| CTCCA | AGCTG |

Description of sequences 1 to 102 and 866 to 1123 and 1209 to 1312 according to the invention

TABLE 2

| | Number of probes described |
|---|---|
| Number of probes | in international patent |
| in the invention | application PCT/FR2014/052255 |
| SEQ ID NO: 103 to 127 | SEQ ID NO: 1 to 25 |
| SEQ ID NO: 128 | SEQ ID NO: 30 |
| SEQ ID NO: 129 | SEQ ID NO: 31 |
| SEQ ID NO: 130 to 137 | SEQ ID NO: 113 to 120 |
| SEQ ID NO: 138 to 168 and | SEQ ID NO: 374 to 405 |
| SEQ ID NO: 825 | |
| SEQ ID NO: 169 to 194 and | SEQ ID NO: 524 to 559 |
| SEQ ID NO: 826 to 835 | |
| SEQ ID NO: 195 to 198 | SEQ ID NO: 26 to 29 |
| SEQ ID NO: 199 to 245 | SEQ ID NO: 66 to 112 |
| SEQ ID NO: 246 to 344 | SEQ ID NO: 121 to 219 |
| SEQ ID NO: 345 to 403 | SEQ ID NO: 616 to 674 |
| SEQ ID NO: 404 to 428 | SEQ ID NO: 750 to 774 |
| SEQ ID NO: 429 to 436 | SEQ ID NO: 734 to 741 |
| SEQ ID NO: 437 to 479 | SEQ ID NO: 438 to 480 |
| SEQ ID NO: 480 to 504 | SEQ ID NO: 35 to 59 |
| SEQ ID NO: 505 | SEQ ID NO: 64 |
| SEQ ID NO: 506 | SEQ ID NO: 65 |
| SEQ ID NO: 507 to 514 | SEQ ID NO: 267 to 274 |
| SEQ ID NO: 515 to 546 | SEQ ID NO: 406 to 437 |
| SEQ ID NO: 547 to 582 | SEQ ID NO: 560 to 595 |
| SEQ ID NO: 583 to 586 | SEQ ID NO: 60 to 63 |
| SEQ ID NO: 587 to 633 | SEQ ID NO: 220 to 266 |
| SEQ ID NO: 634 to 732 | SEQ ID NO: 275 to 373 |
| SEQ ID NO: 733 to 791 | SEQ ID NO: 675 to 733 |
| SEQ ID NO: 792 to 816 | SEQ ID NO: 775 to 799 |
| SEQ ID NO: 817 to 824 | SEQ ID NO: 742 to 749 |

Correspondence between sequences 103 to 835 and the sequences described in international application PCT/FR2014/052255. The L/R information for sequences 103 to 835 is indicated in FIGS. 4-5, 7 to 9 of international application PCT/FR2014/052255.

### BRIEF DESCRIPTION OF THE FIGURES

Other features, details, and advantages of the invention will become apparent on reading the appended Figures.

FIG. 1

FIG. 1 shows the diagram of a chromosomal translocation leading to the expression of a fusion transcript detectable by the invention. FIG. 1A (top) shows the obtaining of a fusion mRNA following a chromosomal translocation between gene A and gene B. FIG. 1B (bottom) shows the step of reverse transcription of this fusion mRNA, in order to obtain cDNA. Next there is a step of incubating with the probes and hybridizing them with the complementary portions of cDNA. Probe S1 consists of a sequence complementary to the last nucleotides of exon 2 of cDNA gene A, and probe S2 consists of a sequence complementary to the first nucleotides of exon 2 of cDNA gene B. Probe S1 is fused at 5′ with a barcode sequence SA′ as well as with a primer sequence SA. Probe S2 is fused at 3′ with a primer sequence SB. Due to the adjacency of exons 2 of gene A and gene B, probes S1 and S2 are side by side. Next there is a ligation step by a DNA ligase. The adjacent probes are now bound. S1 and S2 thus form a continuous sequence, with SA and SB. PCR is then performed. Using suitable primers, the bound probes are amplified. In the current case, the primers used are the sequence SA and the complementary sequence of SB (called B′). The results obtained are then analyzed by sequencing.

FIG. 2

FIG. 2 shows the diagram of an exon skipping leading to the expression of a transcript corresponding to an exon skipping detectable by the invention. FIG. 2A (top) shows the cDNA obtained after reverse transcription in the case of normal splicing, and FIG. 2A (bottom) shows the cDNA obtained after reverse transcription in the case of a splicing abnormality. FIG. 2B (top) shows that in the absence of mutation (normal case), after hybridization of the probes, the sequences obtained are as follows: S13L-S14R and S14L-S15R. FIG. 2B (bottom) shows that in the presence of a mutation (abnormal case of exon skipping), after hybridization of the probes, the sequence obtained is as follows: S13L-S15R.

FIG. 3

FIG. 3 shows an example of probe construction according to the invention. FIG. 3A shows the hybridization of the probes after formation of a fusion gene. The number 1 represents the first primer sequence; the number 2 represents the molecular barcode sequence; the number 3 represents the first probe which hybridizes to the left side of the fusion; the number 4 represents the second probe which hybridizes to the right side of the fusion; the number 5 represents the second primer sequence. Probes 3 and 4 represent an example of a pair of probes according to the invention. Each probe consists of a specific sequence capable of hybridizing at the end of an exon and has a primer sequence at its end. Here, a random 7-base molecular barcode is added between the primer sequence and the specific sequence of the left probe. FIG. 3B shows a fusion transcript before analysis with a next-generation sequencer of the Illumina® type. When a fusion transcript is detected, two probes hybridize side by side, enabling their ligation. The ligation product can then be amplified by PCR using primers corresponding to the primer sequences. In FIG. 3B, these primers themselves carry extensions (P5 and P7) which allow analysis of the PCR products on a next-generation sequencer of the Illumina type.

FIG. 4

FIG. 4 shows translocations identified using the invention. The new rearrangements specifically revealed by the probes of the invention are indicated with dark lines. The already known rearrangements, in particular those described in international application PCT/FR2014/052255, are indicated with light lines. Each line represents an abnormal gene junction possibly present in a tumor, between the genes listed on the left of the figure and those listed on the right. The mix shown here makes it possible to simultaneously search for more than 50 different rearrangements that are recurrent in carcinomas. In addition, due to the use of several probes for certain genes targeting different exons, recombinations capable of leading to the expression of hundreds of different transcripts are detectable.

FIG. 5

FIG. 5 shows the number of fusion RNA molecules present in the starting sample tested according to Example 1. This graph shows that 729 fusion RNA molecules were present in the starting sample, and that this result was amplified by a factor of 135.8 during the PCR step. 98,993 sequences were thus obtained at the end of the PCR step.

FIG. 6

FIG. 6 represents one of the strategies which makes it possible to detect a skipping of exon 14 of the METgene, by means of the invention. In FIG. 6A, the selected probes hybridize to the ends of exons 13, 14 and 15 of this gene. In a normal situation, splicing transcripts of this gene induces junctions between exons 13 and 14, and 14 and 15. In a pathological situation, for example if a mutation destroys the splicing donor site of exon 14, the tumor cells express an abnormal transcript, resulting from the junction of exons 13 and 15. The various amplification products obtained by means of the invention are visible in FIG. 6B on a capillary sequencer, after amplification using a pair of primers of which one is labeled with a fluorochrome. These products, which differ in their sequence, can also easily be revealed using a next-generation sequencer.

FIG. 7

FIG. 7 shows the construction of the sequences as analyzed by the software. The terms “Oligo 5′” and “Oligo 3′” represent a pair of probes according to the invention. The term “UMI” represents the molecular barcode sequence. The terms “11” and “12” represent the primer sequences. The term “index” represents the sequence index. The terms “P5” and “P7” correspond to extensions, useful for the use of a next-generation sequencer.

FIG. 8

FIG. 8 shows an example of a read in FASTQ format.

FIG. 9

FIG. 9 shows the diagram of a skipping of exons in the EGFR gene leading to expression of a transcript corresponding to an exon skipping detectable by the invention. FIG. 9A (top) shows the cDNA obtained after reverse transcription in the case of a normal splicing, and FIG. 9B (bottom) shows the cDNA obtained after reverse transcription in the case of a splicing abnormality.

FIG. 9B (top) shows that in the absence of mutation (normal case), after hybridization of probes S1L, S2R, S7L and SBR, the sequences obtained are as follows: S1L-S2R and 57L-S8R. FIG. 2B (bottom) shows that in the presence of a mutation (abnormal case in the presence of exon skipping), after hybridization of the probes, the sequence obtained is as follows: S1L-S8R (deletion of exons 2 to 7 has taken place).

FIG. 10

FIG. 10 shows the number of fusion RNA molecules present in the starting sample tested according to Example 3. This graph shows that 587 fusion RNA molecules were present in the starting sample, and that this result was amplified by a factor of 259.3 during the PCR step. 152,227 sequences were thus obtained at the end of the PCR step.

FIG. 11

FIG. 11 shows the number of fusion RNA molecules present in the starting sample tested according to Example 4. This graph shows that 505 fusion RNA molecules were present in the starting sample, and that this result was amplified by a factor of 123.1 during the PCR step. 62,151 sequences were thus obtained at the end of the PCR step.

FIG. 12

FIG. 12 shows the number of fusion RNA molecules present in the starting sample tested according to Example 5. This graph shows that 965 fusion RNA molecules were present in the starting sample, and that this result was amplified by a factor of 123.5 during the PCR step. 119,161 sequences were thus obtained at the end of the PCR step.

FIG. 13

FIG. 13 shows the diagram of a 5′-3′ expression imbalance leading to the expression of a transcript corresponding to different alleles, detectable by the invention. Expression levels depend on the transcriptional regulatory regions of the rearranged alleles. For example, the expression of alleles I and III is (Sn_Sn+1)=(Sn+2_Sn+3), the expression of alleles I and II is (Sn+4_Sn+5)=(Sn+6_Sn+7). However, when the transcriptional regulatory regions of genes A and B are not equivalent, then the expression of the 5′ exons (Sn_Sn+1) and (Sn+2_Sn+3) is different from the expression of the 3′ exons expressions (Sn+4_Sn+5) and (Sn+6_Sn+7). For example, in lung carcinomas carrying a fusion of the ALK gene (gene B), alleles I and III, whose expression is controlled by the regulatory regions of ALK, are very weakly expressed, while allele II, controlled by the regulatory regions of the partner gene A, is strongly expressed. This therefore results in a 5′-3′ imbalance, with: (Sn+4_Sn+5)=(Sn+6_Sn+7)»(Sn_Sn+1)=(Sn+2_Sn+3).

FIG. 14

FIG. 14 shows an example of the probes which can be used according to the invention, as well as the gene which this probe makes it possible to detect. L/R indicates whether the probe is “Left” or “Right”, as indicated above.

FIG. 15

FIG. 15 shows an example of the probes which can be used according to the invention, as well as the gene which this probe makes it possible to detect. L/R indicates whether the probe is “Left” or “Right”, as indicated above.

FIG. 16

FIG. 16 shows an example of the probes which can be used according to the invention, as well as the gene which this probe makes it possible to detect. L/R indicates whether the probe is “Left” or “Right”, as indicated above.

FIG. 17

FIG. 17 shows an example of the probes which can be used according to the invention, as well as the gene which this probe makes it possible to detect. L/R indicates whether the probe is “Left” or “Right”, as indicated above.

FIG. 18

FIG. 18 shows an example of the probes which can be used according to the invention, as well as the gene which this probe makes it possible to detect. L/R indicates whether the probe is “Left” or “Right”, as indicated above.

FIG. 19

FIG. 19 shows an example of the probes which can be used according to the invention, as well as the gene which this probe makes it possible to detect. L/R indicates whether the probe is “Left” or “Right”, as indicated above.

FIG. 20

FIG. 20 shows an example of the probes which can be used according to the invention, as well as the gene which this probe makes it possible to detect. L/R indicates whether the probe is “Left” or “Right”, as indicated above.

FIG. 21

FIG. 21 shows an example of the probes which can be used according to the invention, as well as the gene which this probe makes it possible to detect. L/R indicates whether the probe is “Left” or “Right”, as indicated above.

FIG. 22

FIG. 22 shows an example obtained during analysis of a splicing abnormality of the MET gene.

FIG. 23

FIG. 23 shows an example obtained during analysis of a splicing abnormality of the MET gene.

FIG. 24

FIG. 24 shows an example obtained during analysis of a splicing abnormality of the EGFR gene.

FIG. 25

FIG. 25 shows an example obtained during analysis of a splicing abnormality of the EGFR gene.

FIG. 26

FIG. 26 shows an example obtained during analysis of a 5′-3′ expression imbalance. FIG. 27

FIG. 27 shows an example obtained during analysis of a 5′-3′ expression imbalance. FIG. 28

FIG. 28 shows novel probes (SEQ ID NO: 1211 to 1312) and illustrates the cancers they detect. The so-called “full” sequences include the primer sequence, the molecular barcode sequence (for the so-called “Left” probes), and the specific sequence of the probe (called SEQ ID NO: 1313 to 1414).

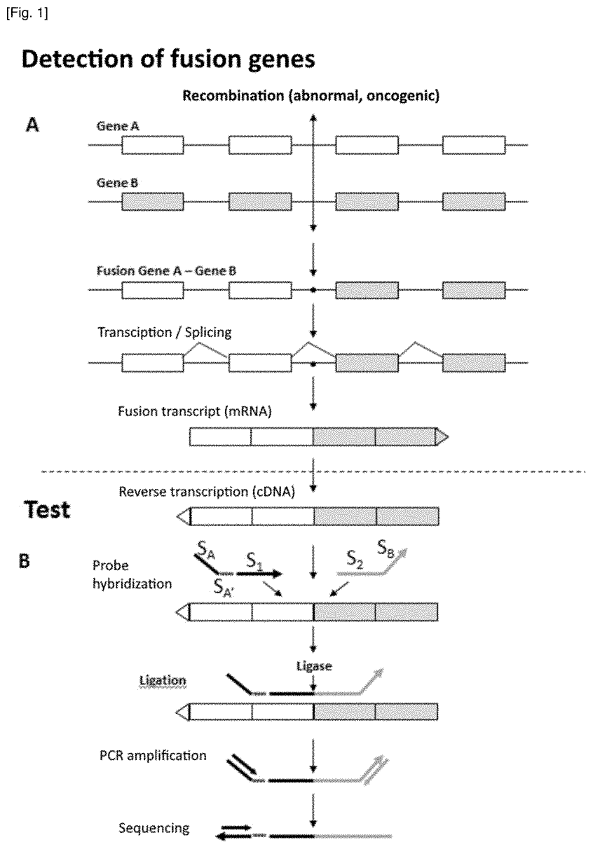

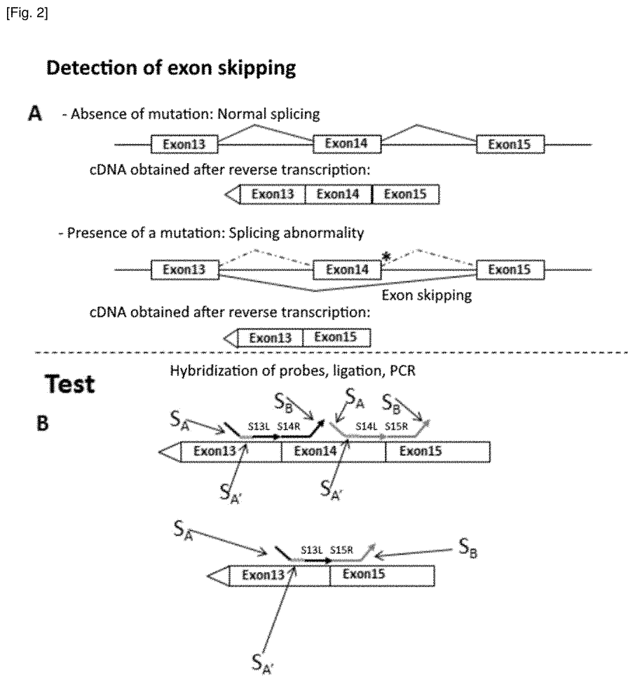

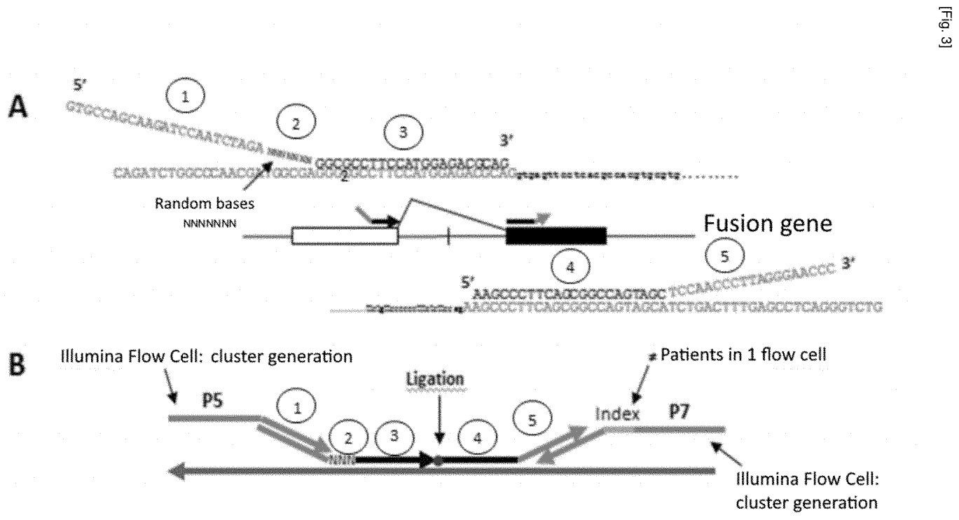

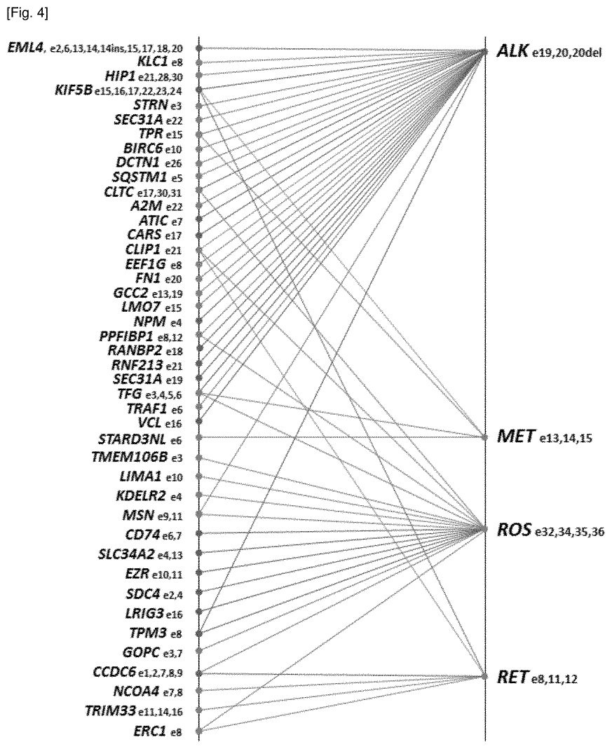

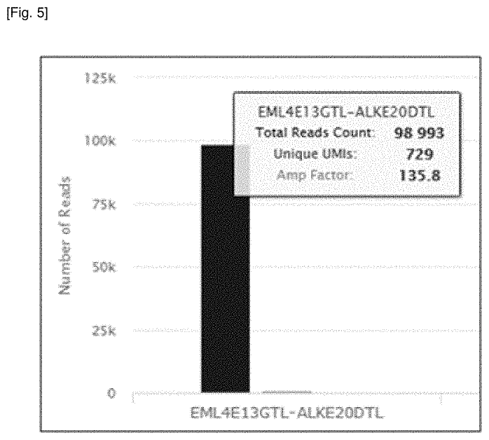

### EXAMPLES

### Example 1: Diagnosing a Carcinoma

The sample from a subject was subjected to an RT-MLPA step according to the invention, using the probes described above (more particularly at least probes SEQ ID NO: 1 to 13 and 14 to 91).

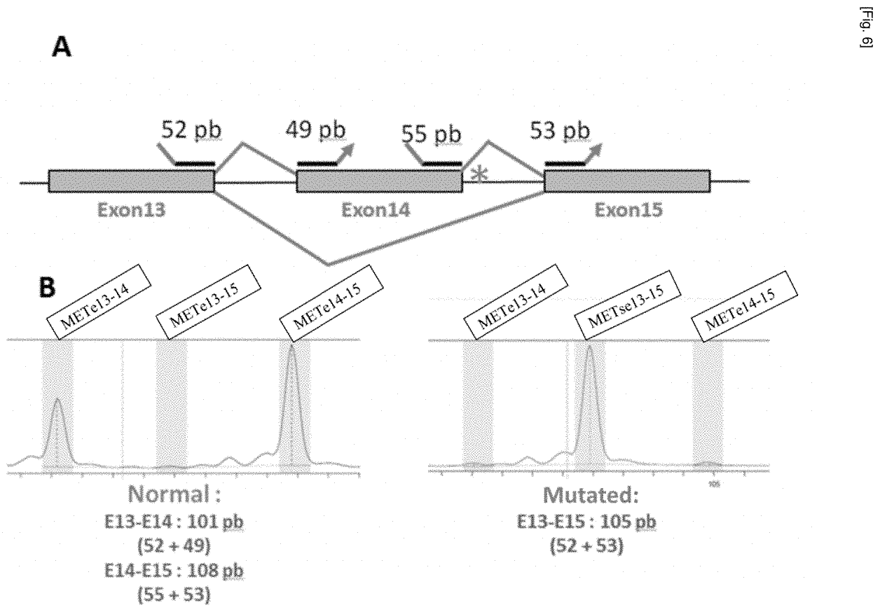

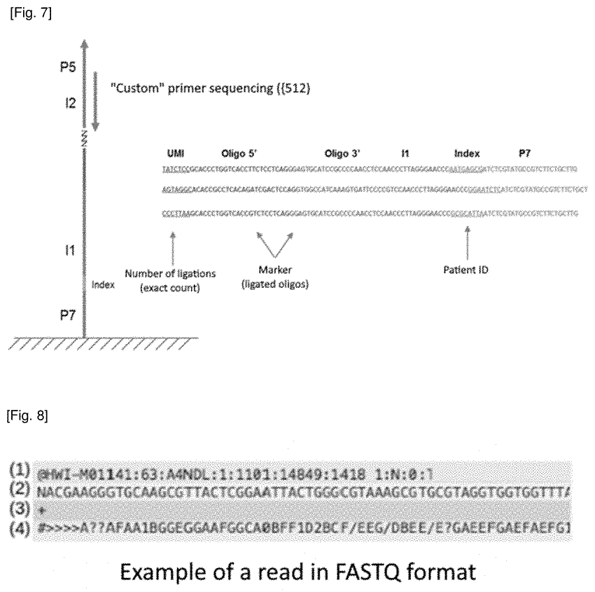

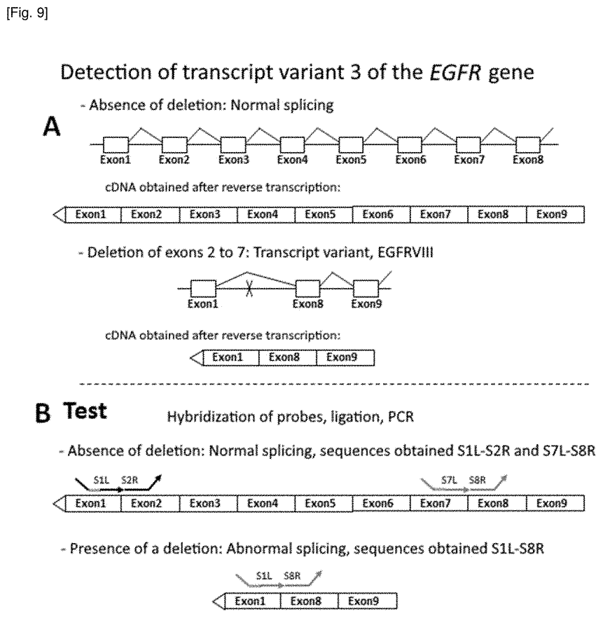

At the end of the PCR step, 98,993 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes allows accurately determining the number of fusion RNA molecules present in the starting sample (in the case tested here: 729, see FIG. 5).

Table 3 shows the results obtained.

TABLE 3

| | Number | | | | |
|---|---|---|---|---|---|
| | of | | | | Sequences |
| Complete sequence | reads | Barcode | Left probe | Right probe | identified |
| AAAAATACCCACACCTGGG | 156 | AAAAATA | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 851) | AG | (SEQ ID NO: 3) | |
| (SEQ ID NO: 837) | | | (SEQ ID | | |
| | | | NO: 31) | | |
| AAAATGACCCACACCTGGG | 72 | AAAATGA | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 852) | AG (SEQ ID | (SEQ ID | |
| (SEQ ID | | | NO: 31) | NO: 3) | |
| NO: 838) | | | | | |
| AAAATGCCCCACACCTGGG | 74 | AAAATGC | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 853) | AG (SEQ ID NO: | (SEQ ID NO: 3) | |
| (SEQ ID NO: 839) | | | 31) | | |
| AAACACTCCCACACCTGGG | 22 | AAACACT | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 854) | AG (SEQ ID NO: | (SEQ ID NO: 3) | |
| (SEQ ID NO: 840) | | | 31) | | |
| AAACGAGCCCACACCTGG | 209 | AAACGA | CCCACACCTGG | TGTACCGCCGGAA | EML4E13GTL- |
| GAAAGGACCTAAAGTGTAC | | G (SEQ ID | GAAAGGACCTAA | GCACCAGGAG | ALKE20DTL |
| CGCCGGAAGCACCAGGAG | | NO: 855) | AG (SEQ ID NO: | (SEQ ID NO: 3) | |
| (SEQ ID NO: 841) | | | 31) | | |
| AAACTGCCCCACACCTGGG | 172 | AAACTGC | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 856) | AG (SEQ ID NO: | (SEQ ID NO: 3) | |
| (SEQ ID NO: 842) | | | 31) | | |
| AAACTGTCCCACACCTGGG | 175 | AAACTGT | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 857) | AG (SEQ ID NO: | (SEQ ID NO: 3) | |
| (SEQ ID NO: 843) | | | 31) | | |
| AAAGAGACCCACACCTGG | 25 | AAAGAG | CCCACACCTGG | TGTACCGCCGGAA | EML4E13GTL- |
| GAAAGGACCTAAAGTGTAC | | A (SEQ ID | GAAAGGACCTAA | GCACCAGGAG | ALKE20DTL |
| CGCCGGAAGCACCAGGAG | | NO: 858) | AG (SEQ ID NO: | (SEQ ID NO: 3) | |
| (SEQ ID NO: 844) | | | 31) | | |
| AAAGATGCCCACACCTGGG | 155 | AAAGATG | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 859) | AG (SEQ ID NO: | (SEQ ID NO: 3) | |
| (SEQ ID NO: 845) | | | 31) | | |
| AAAGGCTCCCACACCTGG | 34 | AAAGGC | CCCACACCTGG | TGTACCGCCGGAA | EML4E13GTL- |
| GAAAGGACCTAAAGTGTAC | | T (SEQ ID | GAAAGGACCTAA | GCACCAGGAG | ALKE20DTL |
| CGCCGGAAGCACCAGGAG | | NO: 860) | AG (SEQ ID NO: | (SEQ ID NO: 3) | |
| (SEQ ID NO: 846) | | | 31) | | |
| AAAGGTACCCACACCTGGG | 68 | AAAGGTA | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 861) | AG (SEQ ID NO: | (SEQ ID NO: 3) | |
| (SEQ ID NO: 847) | | | 31) | | |
| AAAGTCACCCACACCTGGG | 50 | AAAGTCA | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 862) | AG (SEQ ID NO: | (SEQ ID NO: 3) | |
| (SEQ ID NO: 848) | | | 31) | | |
| AAAGTGTCCCACACCTGGG | 149 | AAAGTGT | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 863) | AG (SEQ ID NO: | (SEQ ID NO: 3) | |
| (SEQ ID NO: 849) | | | 31) | | |
| AAAGTTCCCCACACCTGGG | 166 | AAAGTTC | CCCACACCTGG | TGTACCGCCGGAA | EML4E13GTL- |
| AAAGGACCTAAAGTGTACC | | (SEQ ID | GAAAGGACCTAA | GCACCAGGAG | ALKE20DTL |
| GCCGGAAGCACCAGGAG | | NO: 864) | AG (SEQ ID | (SEQ ID | |
| (SEQ ID NO: 850) | | | NO: 31) | NO: 3) | |
| . . . | . . . | . . . | . . . | | . . . |

Example of probes used and results obtained during a diagnosis of carcinoma

Analysis of the sequence corresponding to PCR products makes it possible to identify the two partner genes involved in the chromosomal rearrangement, here the EML4 and ALK genes. The diagnosis of carcinoma was thus confirmed for the patient to be tested.

This rearrangement is recurrent in lung carcinomas, and makes the patient eligible for certain targeted therapies.

### Example 2: Determining a Skipping of Exon 14 of the MET Gene

The sample from a subject was analyzed to confirm or rule out the presence of a skipping of exon 14 of the MET gene. Said sample was subjected to an RT-MLPA step according to the invention, using the probes described above (more particularly at least probes SEQ ID NO: 96 to 99).

In a normal situation, the splicing of the transcripts of this gene induces junctions between exons 13 and 14, and 14 and 15. In a pathological situation, for example if a mutation destroys the splicing donor site of exon 14, tumor cells express an abnormal transcript, resulting from the junction of exons 13 and 15 (FIG. 6A).

The various amplification products obtained by virtue of the invention are visible in FIG. 6B on a capillary sequencer, after amplification using a pair of primers, one of which is labeled with a fluorochrome. These products, which differ in their sequence and in their size, can also easily be revealed using a next-generation sequencer.

### Example 3: Diagnosing a Carcinoma

The sample from a subject was subjected to an RT-MLPA step according to the invention, using the probes described above (more particularly at least probes SEQ ID NO: 1 to 13 and 14 to 91).

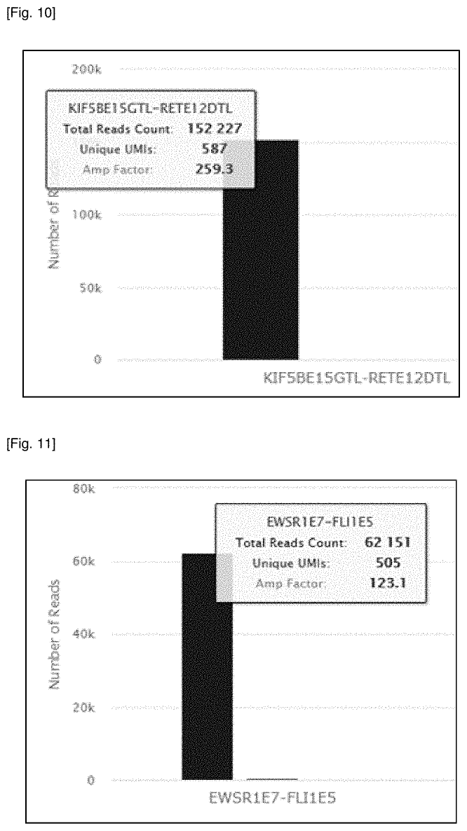

At the end of the PCR step, 152,227 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to accurately determine the number of fusion RNA molecules present in the starting sample (in the case tested here: 587, see FIG. 10).

Table 4 shows the results obtained.

TABLE 4

| | Number | | | | Sequences |
|---|---|---|---|---|---|
| Complete sequence | of reads | Barcode | Left probe | Right probe | identified |
| ATTGCTGTGGGAAATAATG | 1020 | GTATTGC | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 851) | GTAAAG (SEQ | (SEQ ID NO: 8) | |
| (SEQ ID NO: 1124) | | | ID NO: 52) | | |
| ATTGCTGTGGGAAATAATG | 967 | GTGCTCA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1125) | GTAAAG (SEQ | (SEQ ID NO: 8) | |
| (SEQ ID NO: 1124) | | | ID NO: 52) | | |
| ATTGCTGTGGGAAATAATG | 803 | CTAGGGC | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1126) | GTAAAG (SEQ | (SEQ ID NO: 8) | |
| (SEQ ID NO: 1124) | | | ID NO: 52) | | |
| ATTGCTGTGGGAAATAATG | 800 | ATGCTAT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1127) | GTAAAG (SEQ | (SEQ ID NO: 8) | |
| (SEQ ID NO: 1124) | | | ID NO: 52) | | |
| ATTGCTGTGGGAAATAATG | 775 | CTTTGTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1128) | GTAAAG (SEQ | (SEQ ID NO: 8) | |
| (SEQ ID NO: 1124) | | | ID NO: 52) | | |
| ATTGCTGTGGGAAATAATG | 750 | TGACCAA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1129) | GTAAAG (SEQ | (SEQ ID NO: 8) | |
| (SEQ ID NO: 1124) | | | ID NO: 52) | | |
| ATTGCTGTGGGAAATAATG | 740 | AGGTCTT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1130) | GTAAAG (SEQ | (SEQ ID NO: 8) | |
| (SEQ ID NO: 1124) | | | ID NO: 52) | | |
| ATTGCTGTGGGAAATAATG | 731 | TCCATTT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1131) | GTAAAG (SEQ | (SEQ ID NO: 8) | |
| (SEQ ID NO: 1124) | | | ID NO: 52) | | |
| ATTGCTGTGGGAAATAATG | 648 | TCGTTGA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1132) | GTAAAG (SEQ | (SEQ ID NO: 8) | |
| (SEQ ID NO: 1124)) | | | ID NO: 52) | | |
| ATTGCTGTGGGAAATAATG | 592 | GAAAATA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1133) | GTAAAG (SEQ | (SEQ ID NO: 8) | |
| (SEQ ID NO: 1124) | | | ID NO: 52) | | |
| ATTGCTGTGGGAAATAATG | 590 | GCGAGTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1134) | GTAAAG (SEQ | (SEQ ID NO: 8) | |
| (SEQ ID NO: 1124) | | | ID NO: 52) | | |
| ATTGCTGTGGGAAATAATG | 576 | GGGGGTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1135) | GTAAAG (SEQ | (SEQ ID NO: 8) | |
| (SEQ ID NO: 1124) | | | ID NO: 52) | | |
| ATTGCTGTGGGAAATAATG | 572 | TCCAGCC | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1136) | GTAAAG (SEQ | (SEQ ID NO: 8) | |
| (SEQ ID NO: 1124) | | | ID NO: 52) | | |
| ATTGCTGTGGGAAATAATG | 566 | ACGCTTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1137) | GTAAAG (SEQ | (SEQ ID NO: 8) | |
| (SEQ ID NO: 1124) | | | ID NO: 52) | | |
| ATTGCTGTGGGAAATAATG | 554 | TCCTGCG | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1138) | GTAAAG (SEQ | (SEQ ID NO: 8) | |
| (SEQ ID NO: 1124) | | | ID NO: 52) | | |
| ATTGCTGTGGGAAATAATG | 553 | GTGGGCT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1139) | GTAAAG (SEQ | (SEQ ID NO: 8) | |
| (SEQ ID NO: 1124) | | | ID NO: 52) | | |
| ATTGCTGTGGGAAATAATG | 552 | GGCCGGC | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1140) | GTAAAG (SEQ | (SEQ ID NO: 8) | |
| (SEQ ID NO: 1124) | | | ID NO: 52) | | |
| ATTGCTGTGGGAAATAATG | 548 | GGGTCAC | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1141) | GTAAAG (SEQ | (SEQ ID NO: 8) | |
| (SEQ ID NO: 1124) | | | ID NO: 52) | | |
| ATTGCTGTGGGAAATAATG | 521 | CGAGATT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1142) | GTAAAG (SEQ | (SEQ ID NO: 8) | |
| (SEQ ID NO: 1124) | | | ID NO: 52) | | |
| ATTGCTGTGGGAAATAATG | 519 | ACCTGAT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1143) | GTAAAG (SEQ | (SEQ ID NO: 8) | |
| (SEQ ID NO: 1124) | | | ID NO: 52) | | |
| ATTGCTGTGGGAAATAATG | 509 | GCGGCTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1144) | GTAAAG (SEQ | (SEQ ID NO: 8) | |
| (SEQ ID NO: 1124) | | | ID NO: 52) | | |
| ATTGCTGTGGGAAATAATG | 507 | GACGTCT | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1145) | GTAAAG (SEQ | (SEQ ID NO: 8) | |
| (SEQ ID NO: 1124) | | | ID NO: 52) | | |
| ATTGCTGTGGGAAATAATG | 504 | GTGTCTA | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1146) | GTAAAG (SEQ | (SEQ ID NO: 8) | |
| (SEQ ID NO: 1124) | | | ID NO: 52) | | |
| ATTGCTGTGGGAAATAATG | 499 | CGTACTG | ATTGCTGTGG | GAGGATCCAAAGT | KIF5BE15GTL- |
| ATGTAAAGGAGGATCCAAA | | (SEQ ID | GAAATAATGAT | GGGAATTCCCT | RETE12DTL |
| GTGGGAATTCCCT | | NO: 1147) | GTAAAG (SEQ | (SEQ ID NO: 8) | |
| (SEQ ID NO: 1124) | | | ID NO: 52) | | |
| . . . | . . . | . . . | . . . | | . . . |

Example of probes used and results obtained during a diagnosis of carcinoma

Analysis of the sequence corresponding to PCR products makes it possible to identify the two partner genes involved in the chromosomal rearrangement, here the KIF5B and RET genes. The diagnosis of carcinoma was thus confirmed for the patient to be tested.

This rearrangement is recurrent in lung carcinomas, and makes the patient eligible for certain targeted therapies.

### Example 4: Diagnosing a Sarcoma

The sample from a subject was subjected to an RT-MLPA step according to the invention, using the probes described above (more particularly at least probes SEQ: 868 to 938 and probes SEQ ID NO: 940 to 1054).

At the end of the PCR step, 62,151 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to accurately determine the number of fusion RNA molecules present in the starting sample (in the case tested here: 505, see FIG. 11).

Table 5 shows the results obtained.

TABLE 5

| | Number | | | | Sequences |
|---|---|---|---|---|---|
| Complete sequence | of reads | Barcode | Left probe | Right probe | Identified |
| AGCAGCAGCTACGGGCAG | 472 | CATGAG | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1151) | 1148) | | |
| AGCAGCAGCTACGGGCAG | 397 | TCGCGG | AGCAGCAGCTA | GTTCACTGCTGGC | EWSR1E7-FLI1 E5 |
| CAGAGTTCACTGCTGGCCT | | C (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1152) | 1148) | | |
| AGCAGCAGCTACGGGCAG | 385 | TTTGTTT | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1153) | 1148) | | |
| AGCAGCAGCTACGGGCAG | 369 | CGTGTG | AGCAGCAGCTA | GTTCACTGCTGGC | EWSR1E7-FLI1 E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1154) | 1148) | | |
| AGCAGCAGCTACGGGCAG | 363 | CTTGGG | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1155) | 1148) | | |
| AGCAGCAGCTACGGGCAG | 357 | TAGCGAT | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1156) | 1148) | | |
| AGCAGCAGCTACGGGCAG | 354 | CGTCCTT | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1157) | 1148) | | |
| AGCAGCAGCTACGGGCAG | 344 | GTGAGT | AGCAGCAGCTA | GTTCACTGCTGGC | EWSR1E7-FLI1E5 |
| CAGAGTTCACTGCTGGCCT | | C (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1158) | 1148) | | |
| AGCAGCAGCTACGGGCAG | 336 | CGGGGG | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1159) | 1148) | | |
| AGCAGCAGCTACGGGCAG | 329 | GAGCCT | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1160) | 1148) | | |
| AGCAGCAGCTACGGGCAG | 318 | GTTTTGG | AGCAGCAGCTA | GTTCACTGCTGGC | EWSR1E7-FLI1E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1161) | 1148) | | |
| AGCAGCAGCTACGGGCAG | 312 | GTCGGG | AGCAGCAGCTA | GTTCACTGCTGGC | EWSR1E7-FLI1E5 |
| CAGAGTTCACTGCTGGCCT | | A (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1162) | 1148) | | |
| AGCAGCAGCTACGGGCAG | 304 | TTGGTCC | AGCAGCAGCTA | GTTCACTGCTGGC | EWSR1E7-FLI1E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1163) | 1148) | | |
| AGCAGCAGCTACGGGCAG | 303 | ACGGAA | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1164) | 1148) | | |
| AGCAGCAGCTACGGGCAG | 291 | AGTATTA | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1165) | 1148) | | |
| AGCAGCAGCTACGGGCAG | 289 | CATTCGC | AGCAGCAGCTA | GTTCACTGCTGGC | EWSR1E7-FLI1E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1166) | 1148) | | |
| AGCAGCAGCTACGGGCAG | 278 | TAGTAAG | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1167) | 1148) | | |
| AGCAGCAGCTACGGGCAG | 273 | TCCTACG | AGCAGCAGCTA | GTTCACTGCTGGC | EWSR1E7-FLI1 E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1168) | 1148) | | |
| AGCAGCAGCTACGGGCAG | 267 | GGTATG | AGCAGCAGCTA | GTTCACTGCTGGC | EWSR1E7-FLI1 E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1169) | 1148) | | |
| AGCAGCAGCTACGGGCAG | 261 | CGGGGT | AGCAGCAGCTA | GTTCACTGCTGGC | EWSR1E7-FLI1E5 |
| CAGAGTTCACTGCTGGCCT | | A (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1170) | 1148) | | |
| AGCAGCAGCTACGGGCAG | 258 | CTGATAG | AGCAGCAGCTA | GTTCACTGCTGGC | EWSR1E7-FLI1E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1171) | 1148) | | |
| AGCAGCAGCTACGGGCAG | 257 | TAGGGT | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1172) | 1148) | | |
| AGCAGCAGCTACGGGCAG | 251 | TGGGGA | AGCAGCAGCTA | GTTCACTGCTGGC | EWSR1E7-FLI1E5 |
| CAGAGTTCACTGCTGGCCT | | G (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1173) | 1148) | | |
| AGCAGCAGCTACGGGCAG | 251 | GCTGGT | AGCAGCAGCTA | GTTCACTGCTGGC | EWSR1E7-FLI1E5 |
| CAGAGTTCACTGCTGGCCT | | C (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1174) | 1148) | | |
| AGCAGCAGCTACGGGCAG | 242 | TATGGG | AGCAGCAGCTA | GTTCACTGCTGGC | EWSR1E7-FLI1E5 |
| CAGAGTTCACTGCTGGCCT | | C (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1175) | 1148) | | |
| AGCAGCAGCTACGGGCAG | 241 | ATACGTC | AGCAGCAGCTA | GTTCACTGCTGGC | EWSR1E7-FLI1E5 |
| CAGAGTTCACTGCTGGCCT | | (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1176) | 1148) | | |
| AGCAGCAGCTACGGGCAG | 240 | AGACAA | AGCAGCAGCTA | GTTCACTGCTGGC | EWSR1E7-FLI1E5 |
| CAGAGTTCACTGCTGGCCT | | C (SEQ ID | CGGGCAGCAGA | CTATACAACCTC | |
| ATACAACCTC | | NO: | (SEQ ID No: | (SEQ ID NO: 1149) | |
| (SEQ ID NO: 1150) | | 1177) | 1148) | | |
| . . . | . . . | . . . | . . . | | . . . |

Example of probes used and results obtained during a diagnosis of sarcoma

Analysis of the sequence corresponding to PCR products makes it possible to identify the two partner genes involved in the chromosomal rearrangement, here the EWSR1 and FLI1 genes. The diagnosis of sarcoma was thus confirmed for the patient to be tested.

This rearrangement is recurrent in Ewing sarcomas, which makes it possible to make the diagnosis.

### Example 5: Diagnosing a Sarcoma

The sample from a subject was subjected to an RT-MLPA step according to the invention, using the probes described above (more particularly at least probes SEQ: 868 to 938 and probes SEQ ID NO: 940 to 1054).

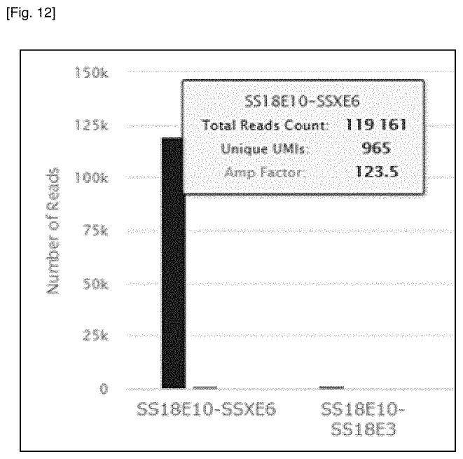

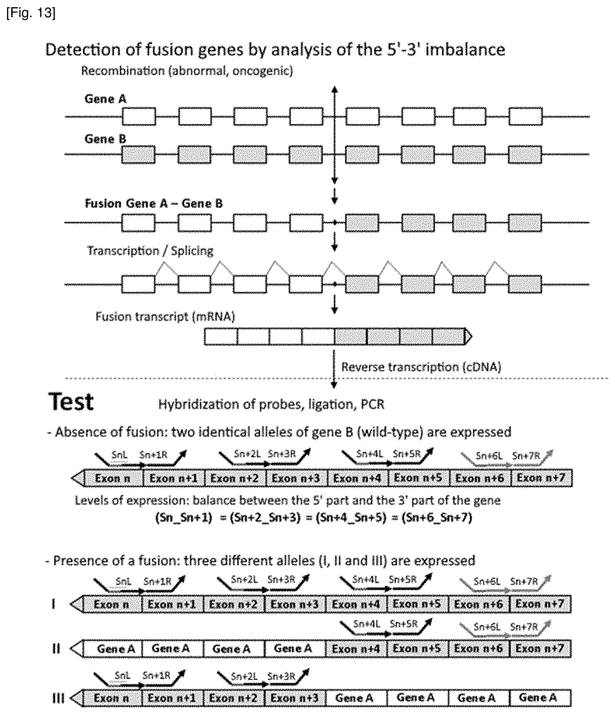

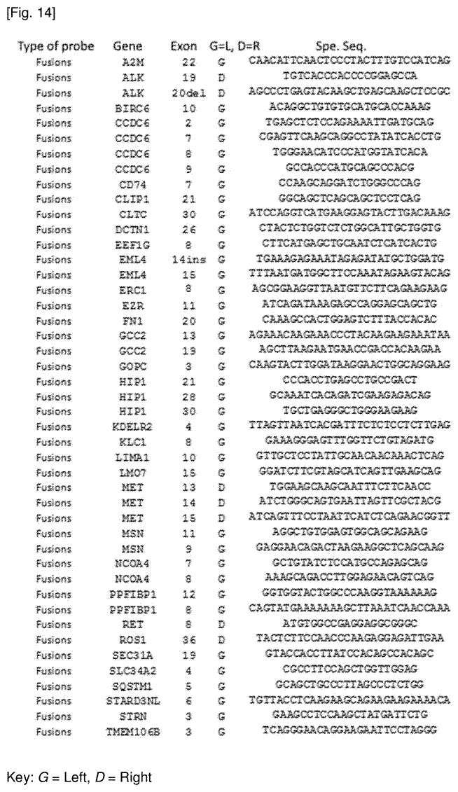

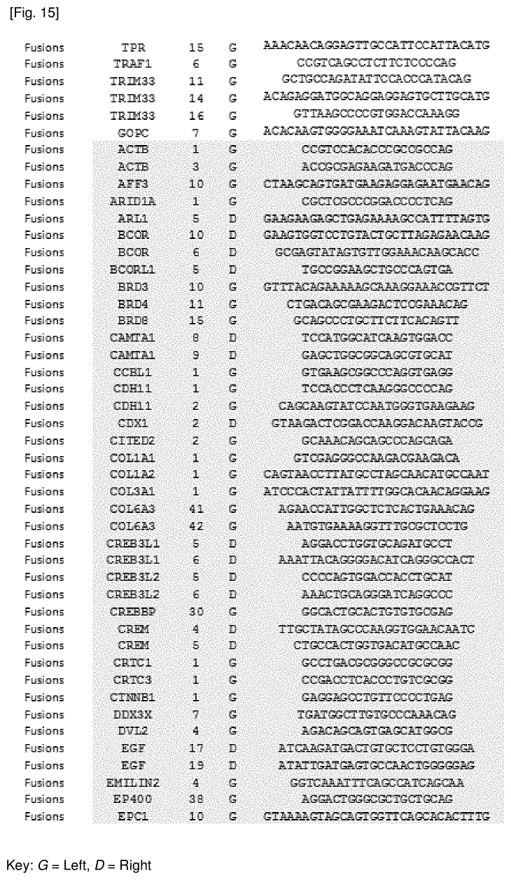

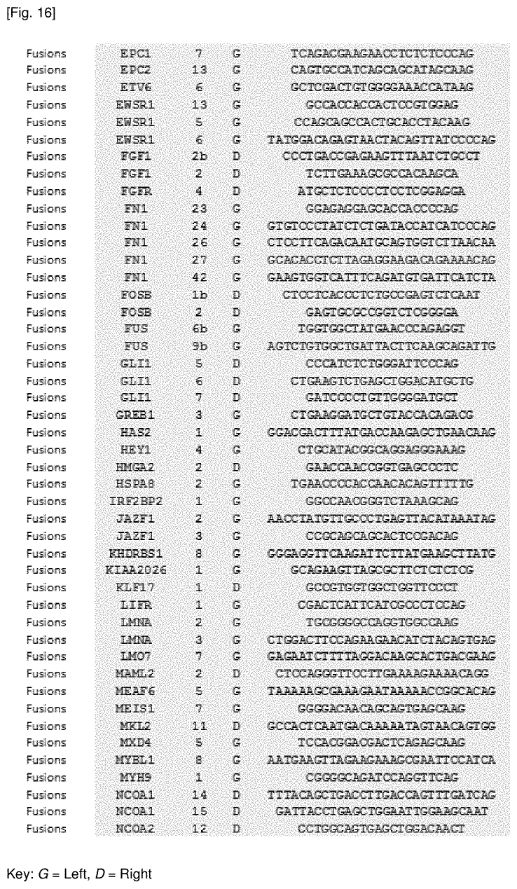

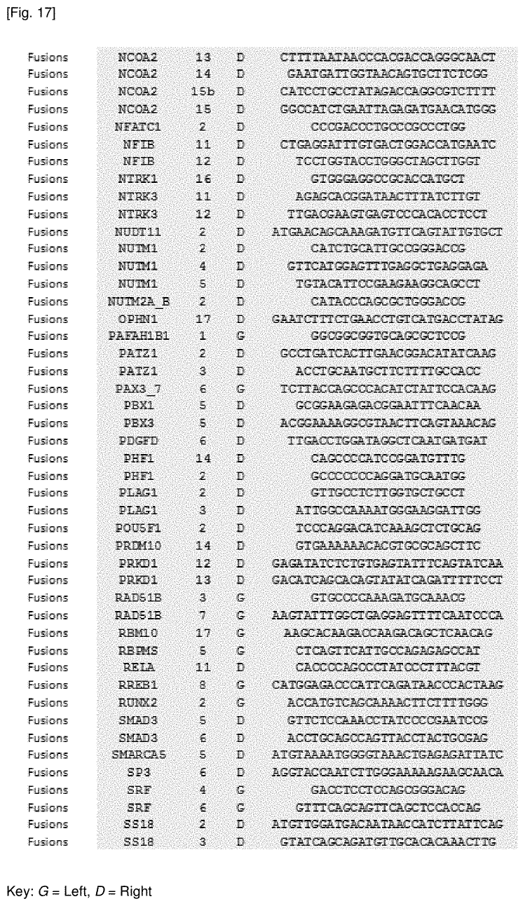

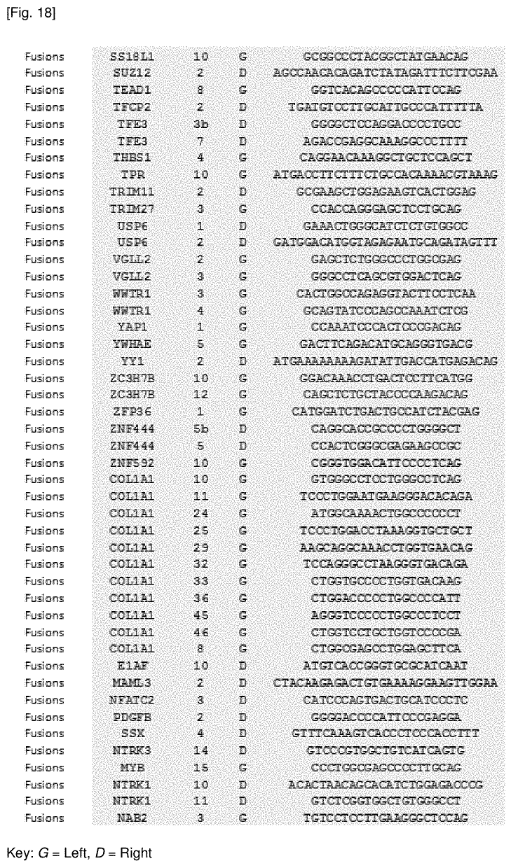

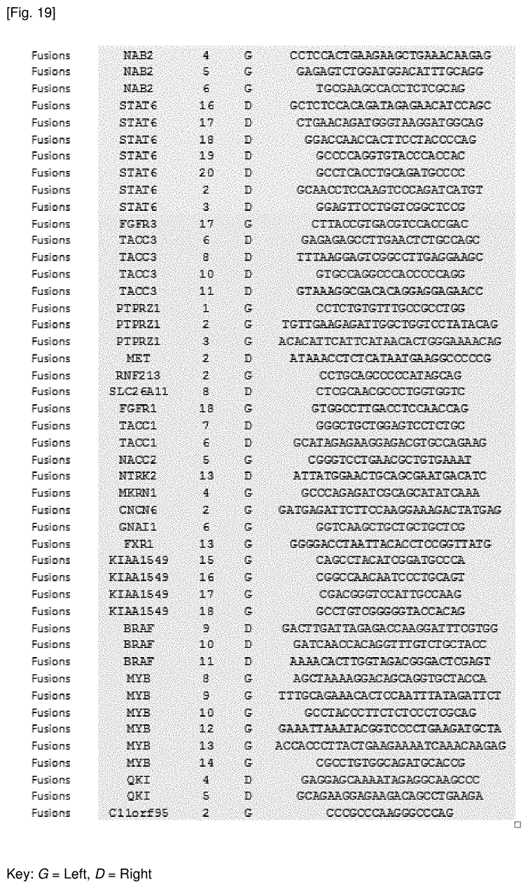

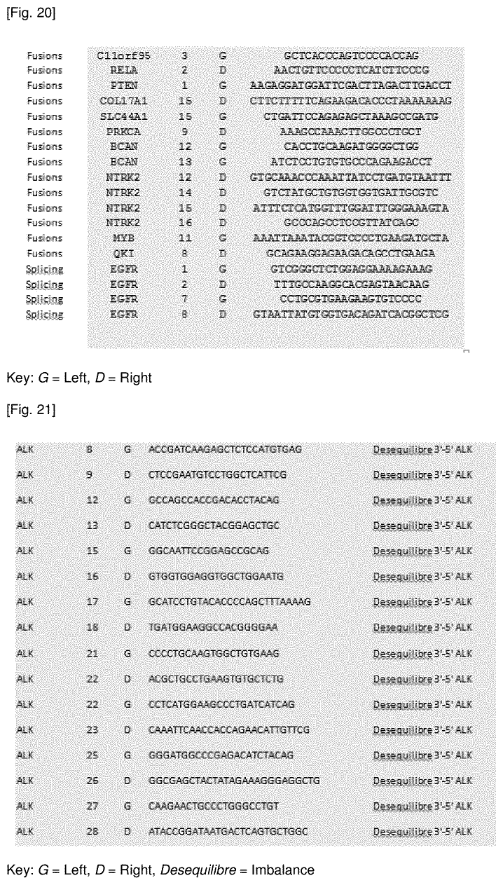

At the end of the PCR step, 119,161 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to accurately determine the number of fusion RNA molecules present in the starting sample (in the case tested here: 960, see FIG. 12).

Table 6 shows the results obtained.

TABLE 6

| | Number | | | | Sequences |
|---|---|---|---|---|---|
| Complete sequence | of reads | Barcode | Left probe | Right probe | identified |
| AGCAGAGGCCTTATGGATA | 610 | ATGTGTC | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1181) | CAG (SEQ ID NO: | (SEQ ID NO: 1179) | |
| (SEQ ID NO: 1180) | | | 1178) | | |
| AGCAGAGGCCTTATGGATA | 604 | GGGGGC | AGCAGAGGCCT | ATCATGCCCAAGA | SS18E10-SSXE6 |
| TGACCAGATCATGCCCAAG | | G (SEQ ID | TATGGATATGAC | AGCCAGCAGA | |
| AAGCCAGCAGA | | NO: 1182) | CAG (SEQ ID NO: | (SEQ ID NO: 1179) | |
| (SEQ ID NO: 1180) | | | 1178) | | |
| AGCAGAGGCCTTATGGATA | 601 | ATATTCG | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1183) | CAG (SEQ ID NO: | (SEQ ID NO: 1179) | |
| (SEQ ID NO: 1180) | | | 1178) | | |
| AGCAGAGGCCTTATGGATA | 524 | CGCGTTT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1184) | CAG (SEQ ID NO: | (SEQ ID NO: 1179) | |
| (SEQ ID NO: 1180) | | | 1178) | | |
| AGCAGAGGCCTTATGGATA | 507 | GTGGTTA | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1185) | CAG (SEQ ID NO: | (SEQ ID NO: 1179) | |
| (SEQ ID NO: 1180) | | | 1078) | | |
| AGCAGAGGCCTTATGGATA | 505 | CGGGTT | AGCAGAGGCCT | ATCATGCCCAAGA | SS18E10-SSXE6 |
| TGACCAGATCATGCCCAAG | | T (SEQ ID | TATGGATATGAC | AGCCAGCAGA | |
| AAGCCAGCAGA | | NO: 1186) | CAG (SEQ ID NO: | (SEQ ID NO: 1179) | |
| (SEQ ID NO: 1180) | | | 1178) | | |
| AGCAGAGGCCTTATGGATA | 491 | GGGAGG | AGCAGAGGCCT | ATCATGCCCAAGA | SS18E10-SSXE6 |
| TGACCAGATCATGCCCAAG | | C (SEQ ID | TATGGATATGAC | AGCCAGCAGA | |
| AAGCCAGCAGA | | NO: 1187) | CAG (SEQ ID NO: | (SEQ ID NO: 1179) | |
| (SEQ ID NO: 1180) | | | 1178) | | |
| AGCAGAGGCCTTATGGATA | 472 | GTATATG | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1188) | CAG (SEQ ID NO: | (SEQ ID NO: 1179) | |
| (SEQ ID NO: 1180) | | | 1178) | | |
| AGCAGAGGCCTTATGGATA | 439 | ACCTTGT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1189) | CAG (SEQ ID NO: | (SEQ ID NO: 1179) | |
| (SEQ ID NO: 1180) | | | 1178) | | |
| AGCAGAGGCCTTATGGATA | 425 | TTGCAGA | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1190) | CAG (SEQ ID NO: | (SEQ ID NO: 1179) | |
| (SEQ ID NO: 1180) | | | 1178) | | |
| AGCAGAGGCCTTATGGATA | 416 | GGGGCA | AGCAGAGGCCT | ATCATGCCCAAGA | SS18E10-SSXE6 |
| TGACCAGATCATGCCCAAG | | A (SEQ ID | TATGGATATGAC | AGCCAGCAGA | |
| AAGCCAGCAGA | | NO: 1191) | CAG (SEQ ID NO: | (SEQ ID NO: 1179) | |
| (SEQ ID NO: 1180) | | | 1178) | | |
| AGCAGAGGCCTTATGGATA | 409 | GAGGCT | AGCAGAGGCCT | ATCATGCCCAAGA | SS18E10-SSXE6 |
| TGACCAGATCATGCCCAAG | | T (SEQ ID | TATGGATATGAC | AGCCAGCAGA | |
| AAGCCAGCAGA | | NO: 1192) | CAG (SEQ ID NO: | (SEQ ID NO: 1179) | |
| (SEQ ID NO: 1180) | | | 1178) | | |
| AGCAGAGGCCTTATGGATA | 408 | I CAI ITT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1193) | CAG (SEQ ID NO: | (SEQ ID NO: 1179) | |
| (SEQ ID NO: 1180) | | | 1178) | | |
| AGCAGAGGCCTTATGGATA | 400 | GGTGAC | AGCAGAGGCCT | ATCATGCCCAAGA | SS18E10-SSXE6 |
| TGACCAGATCATGCCCAAG | | T (SEQ ID | TATGGATATGAC | AGCCAGCAGA | |
| AAGCCAGCAGA | | NO: 1194) | CAG (SEQ ID NO: | (SEQ ID NO: 1179) | |
| (SEQ ID NO: 1180) | | | 1178) | | |
| AGCAGAGGCCTTATGGATA | 394 | TGTGCG | AGCAGAGGCCT | ATCATGCCCAAGA | SS18E10-SSXE6 |
| TGACCAGATCATGCCCAAG | | T (SEQ ID | TATGGATATGAC | AGCCAGCAGA | |
| AAGCCAGCAGA | | NO: 1195) | CAG (SEQ ID NO: | (SEQ ID NO: 1179) | |
| (SEQ ID NO: 1180) | | | 1178) | | |
| AGCAGAGGCCTTATGGATA | 393 | GGGAGA | AGCAGAGGCCT | ATCATGCCCAAGA | SS18E10-SSXE6 |
| TGACCAGATCATGCCCAAG | | G (SEQ ID | TATGGATATGAC | AGCCAGCAGA | |
| AAGCCAGCAGA | | NO: 1196) | CAG (SEQ ID NO: | (SEQ ID NO: 1179) | |
| (SEQ ID NO: 1180) | | | 1178) | | |
| AGCAGAGGCCTTATGGATA | 391 | GCCATTT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1197) | CAG (SEQ ID NO: | (SEQ ID NO: 1179) | |
| (SEQ ID NO: 1180) | | | 1178) | | |
| AGCAGAGGCCTTATGGATA | 380 | AAGCCA | AGCAGAGGCCT | ATCATGCCCAAGA | SS18E10-SSXE6 |
| TGACCAGATCATGCCCAAG | | A (SEQ ID | TATGGATATGAC | AGCCAGCAGA | |
| AAGCCAGCAGA | | NO: 1198) | CAG (SEQ ID NO: | (SEQ ID NO: 1179) | |
| (SEQ ID NO: 1180) | | | 1178) | | |
| AGCAGAGGCCTTATGGATA | 370 | ATTAGG | AGCAGAGGCCT | ATCATGCCCAAGA | SS18E10-SSXE6 |
| TGACCAGATCATGCCCAAG | | G (SEQ ID | TATGGATATGAC | AGCCAGCAGA | |
| AAGCCAGCAGA | | NO: 1199) | CAG (SEQ ID NO: | (SEQ ID NO: 1179) | |
| (SEQ ID NO: 1180) | | | 1178) | | |
| AGCAGAGGCCTTATGGATA | 365 | CCTGGTT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1200) | CAG (SEQ ID NO: | (SEQ ID NO: 1179) | |
| (SEQ ID NO: 1180) | | | 1178) | | |
| AGCAGAGGCCTTATGGATA | 364 | GATTTGT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1201) | CAG (SEQ ID NO: | (SEQ ID NO: 1179) | |
| (SEQ ID NO: 1180) | | | 1178) | | |
| AGCAGAGGCCTTATGGATA | 359 | TAGAGTT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1202) | CAG (SEQ ID NO: | (SEQ ID NO: 1179) | |
| (SEQ ID NO: 1180) | | | 1178) | | |
| AGCAGAGGCCTTATGGATA | 359 | TGCTTTG | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1203) | CAG (SEQ ID NO: | (SEQ ID NO: 1179) | |
| (SEQ ID NO: 1080) | | | 1178) | | |
| AGCAGAGGCCTTATGGATA | 343 | TCCTAGC | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1204) | CAG (SEQ ID NO: | (SEQ ID NO: 1179) | |
| (SEQ ID NO: 1180) | | | 1178) | | |
| AGCAGAGGCCTTATGGATA | 339 | GTAATCT | AGCAGAGGCCT | ATCATGCCCAAGA | SS18E10-SSXE6 |
| TGACCAGATCATGCCCAAG | | (SEQ ID | TATGGATATGAC | AGCCAGCAGA | |
| AAGCCAGCAGA | | NO: 1205) | CAG (SEQ ID NO: | (SEQ ID NO: 1179) | |
| (SEQ ID NO: 1180) | | | 1178) | | |
| AGCAGAGGCCTTATGGATA | 338 | GAGCCT | AGCAGAGGCCT | ATCATGCCCAAGA | SS18E10-SSXE6 |
| TGACCAGATCATGCCCAAG | | G (SEQ ID | TATGGATATGAC | AGCCAGCAGA | |
| AAGCCAGCAGA | | NO: 1206) | CAG (SEQ ID NO: | (SEQ ID NO: 1179 | |
| (SEQ ID NO: 1180) | | | 1178) | | |
| AGCAGAGGCCTTATGGATA | 335 | CCGCAG | AGCAGAGGCCT | ATCATGCCCAAGA | SS18E10-SSXE6 |
| TGACCAGATCATGCCCAAG | | G (SEQ ID | TATGGATATGAC | AGCCAGCAGA | |
| AAGCCAGCAGA | | NO: 1207) | CAG (SEQ ID NO: | (SEQ ID NO: 1179 | |
| (SEQ ID NO: 1180) | | | 1178) | | |
| AGCAGAGGCCTTATGGATA | 332 | GCCGGG | AGCAGAGGCCT | ATCATGCCCAAGA | SS18E10-SSXE6 |
| TGACCAGATCATGCCCAAG | | A (SEQ ID | TATGGATATGAC | AGCCAGCAGA | |
| AAGCCAGCAGA | | NO: 1208) | CAG (SEQ ID NO: | (SEQ ID NO: 1179 | |
| (SEQ ID NO: 1180) | | | 1178) | | |
| . . . | . . . | . . . | . . . | | . . . |

Example of probes used and results obtained during a diagnosis of sarcoma

Analysis of the sequence corresponding to PCR products makes it possible to identify the two partner genes involved in the chromosomal rearrangement, here the SS18 and SSX genes. The diagnosis of sarcoma was thus confirmed for the patient to be tested.

This rearrangement is recurrent in synovial sarcomas, which makes it possible to make the diagnosis.

### Example 6: Examples of Fusion Associated with Pathologies

Table 7 shows some examples.

TABLE 7

| | | |
|---|---|---|
| EWSR1 | SMAD3 | Acral fibroblastic spindle cell neoplams |
| MYB | NFIB | Adenoid cystic carcinoma |
| MYBL1 | NFIB | Adenoid cystic carcinoma/Breast adenoid carcinoma |
| CDH11 | USP6 | Aneurysmal bone cyst |
| COL1A1 | USP6 | Aneurysmal bone cyst |
| CTNNB1 | USP6 | Aneurysmal bone cyst |
| PAFAH1B1 | USP6 | Aneurysmal bone cyst |
| RUNX2 | USP6 | Aneurysmal bone cyst |
| PAX3_7 | FKHR(FOXO1) | ARMS/Biphenotypic sinonasal sarcoma (BSNS) |
| PAX3_7 | NCOA1 | ARMS/Biphenotypic sinonasal sarcoma (BSNS) |
| BCOR | CCNB3 | BCOR round cell sarcoma |
| RREB1 | MKL2 | Biphenotypic oropharyngeal sarcoma/Ectomesenchymal |
| | | chondromyxoid tumor |
| PAX3_7 | MAML3 | Biphenotypic sinonasal sarcoma (BSNS) |
| EWSR1 | NFATC1 | Bone hemangioma |
| FN1 | EGF | Calcifying aponeurotic fibroma |
| EWSR1 | CREB1 | Clear cell sarcoma soft tissues and digestive |
| | | tract/Angiomatoid fibrous histiocytoma |
| EML4 | NTRK3 | Congenital fibrosarcoma |
| KHDRBS1 | NTRK3 | Congenital pediatric CD34+ skin tumor/dermohypodermal |
| | | spindle cell neoplasm |
| SRF | NCOA2 | Congenital spindle cell RMS |
| TEAD1 | NCOA2 | Congenital spindle cell RMS |
| VGLL2 | NCOA2 | Congenital spindle cell RMS/Small round cell sarcomas |
| ARID1A | PRKD1 | Cribriform adenocarcinoma of salivary gland origin |
| DDX3X | PRKD1 | Cribriform adenocarcinoma of salivary gland origin |
| EWSR1 | TRIM11 | Cutaneous melanocytoma |
| COL1A1 | PDGFB | Dermatofibrosarcoma protuberans |
| COL6A3 | PDGFD | Dermatofibrosarcoma protuberans |
| EMILIN2 | PDGFD | Dermatofibrosarcoma protuberans |
| EWSR1 | WT1 | Desmoplastic small round cell tumor |
| EPC1 | BCOR | Endometrial stromal sarcoma (aggressive) |
| EPC1 | SUZ12 | Endometrial stromal sarcoma (aggressive) |
| WWTR1 | CAMTA1 | Epithelioid hemangioendothelioma |
| YAP1 | TFE3 | Epithelioid hemangioendothelioma |
| WWTR1 | FOSB | Epithelioid Hemangioma |
| ZFP36 | FOSB | Epithelioid hemangioma |
| EWSR1 | TFCP2 | Epithelioid rhabdomyosarcoma |
| EWSR1 | E1AF | Ewing Sarcoma |
| FUS | ERG | Ewing Sarcoma/PNET |
| EWSR1 | ETV1 | Ewing Sarcoma/PNET |
| EWSR1 | FEV | Ewing Sarcoma/PNET |
| FUS | FEV | Ewing Sarcoma/PNET |
| EWSR1 | FLI1 | Ewing Sarcoma/PNET |
| EWSR1 | NFATC2 | Ewing Sarcoma/PNET |
| EWSR1 | SMARCA5 | Ewing Sarcoma/PNET |
| EWSR1 | ERG | Ewing Sarcoma/PNET/Desmoplastic small round cell tumor |
| EWSR1 | NR4A3 | Extraskeletal myxoid chondrosarcoma |
| TAF15_68 | NR4A3 | Extraskeletal myxoid chondrosarcoma |
| TCF12 | NR4A3 | Extraskeletal myxoid chondrosarcoma |
| TFG | NR4A3 | Extraskeletal myxoid chondrosarcoma |
| HSPA8 | NR4A3 | Extraskeletal myxoid chondrosarcoma |
| ETV6 | NTRK3 | Head and Neck analog Mammary secretory |
| | | carcinoma/Mammary secretory carcinoma/ |
| | | Papillary thyroid carcinoma |
| EWSR1 | CREM | Hyalinizing renal cell carcinoma |
| TFG | MET | Infantile spindle cell sarcoma with neural features |
| CARS | ALK | inflammatory myofibroblastic tumor |
| CLTC | ALK | inflammatory myofibroblastic tumor |
| FN1 | ALK | inflammatory myofibroblastic tumor |
| KIF5B | ALK | inflammatory myofibroblastic tumor |
| NPM | ALK | inflammatory myofibroblastic tumor |
| RANBP2 | ALK | inflammatory myofibroblastic tumor |
| RNF213 | ALK | inflammatory myofibroblastic tumor |
| SEC31A | ALK | inflammatory myofibroblastic tumor |
| TFG | ALK | inflammatory myofibroblastic tumor |
| TPM3 | ALK | inflammatory myofibroblastic tumor |
| CCDC6 | RET | inflammatory myofibroblastic tumor |
| CCDC6 | ROS | inflammatory myofibroblastic tumor |
| CD74 | ROS | inflammatory myofibroblastic tumor |
| EZR | ROS | inflammatory myofibroblastic tumor |
| LRIG3 | ROS | inflammatory myofibroblastic tumor |
| SDC4 | ROS | inflammatory myofibroblastic tumor |
| TPM3 | ROS | inflammatory myofibroblastic tumor |
| THBS1 | ALK | inflammatory myofibroblastic tumor + Uterine Inflammatory |
| | | Myofibroblastic Tumors |
| EML4 | ALK | inflammatory myofibroblastic tumours/Lung Cancer |
| ATIC | ALK | inflammatory myofibroblastic tumours/Lung Cancer |
| SLC34A2 | ROS | inflammatory myofibroblastic tumours/Lung Cancer |
| A2M | ALK | inflammatory myofibroblastic tumours/Lung Cancer |
| BIRC6 | ALK | inflammatory myofibroblastic tumours/Lung Cancer |
| CLIP1 | ALK | inflammatory myofibroblastic tumours/Lung Cancer |
| DCTN1 | ALK | inflammatory myofibroblastic tumours/Lung Cancer |
| EEF1G | ALK | inflammatory myofibroblastic tumours/Lung Cancer |
| GCC2 | ALK | inflammatory myofibroblastic tumours/Lung Cancer |
| HIP1 | ALK | inflammatory myofibroblastic tumours/Lung Cancer |
| KLC1 | ALK | inflammatory myofibroblastic tumours/Lung Cancer |
| LMO7 | ALK | inflammatory myofibroblastic tumours/Lung Cancer |
| MSN | ALK | inflammatory myofibroblastic tumours/Lung Cancer |
| PPFIBP1 | ALK | inflammatory myofibroblastic tumours/Lung Cancer |
| SQSTM1 | ALK | inflammatory myofibroblastic tumours/Lung Cancer |
| TPR | ALK | inflammatory myofibroblastic tumours/Lung Cancer |
| TRAF1 | ALK | inflammatory myofibroblastic tumours/Lung Cancer |
| KIF5B | MET | inflammatory myofibroblastic tumours/Lung Cancer |
| STARD3NL | MET | inflammatory myofibroblastic tumours/Lung Cancer |
| CLIP1 | RET | inflammatory myofibroblastic tumours/Lung Cancer |
| ERC1 | RET | inflammatory myofibroblastic tumours/Lung Cancer |
| TRIM33 | RET | inflammatory myofibroblastic tumours/Lung Cancer |
| CLIP1 | ROS | inflammatory myofibroblastic tumours/Lung Cancer |
| CLTC | ROS | inflammatory myofibroblastic tumours/Lung Cancer |
| ERC1 | ROS | inflammatory myofibroblastic tumours/Lung Cancer |
| GOPC | ROS | inflammatory myofibroblastic tumours/Lung Cancer |
| KDELR2 | ROS | inflammatory myofibroblastic tumours/Lung Cancer |
| LIMA1 | ROS | inflammatory myofibroblastic tumours/Lung Cancer |
| MSN | ROS | inflammatory myofibroblastic tumours/Lung Cancer |
| PPFIBP1 | ROS | inflammatory myofibroblastic tumours/Lung Cancer |
| TFG | ROS | inflammatory myofibroblastic tumours/Lung Cancer |
| TMEM106B | ROS | inflammatory myofibroblastic tumours/Lung Cancer |
| KIF5B | RET | inflammatory myofibroblastic tumours/Lung Cancer |
| NCOA4 | RET | Intraductal carcinomas of salivary gland |
| TRIM27 | RET | Intraductal carcinomas of salivary gland |
| COL1A2 | PLAG1 | Lipoblastoma |
| COL3A1 | PLAG1 | Lipoblastoma |
| HAS2 | PLAG1 | Lipoblastoma |
| TPR | NTRK1 | Locally agressive lipofibromatosis-like neural tumor/Uterine |
| | | sarcoma with features of fibrosarcoma |
| LMNA | NTRK1 | Locally agressive lipofibromatosis-like neural tumor/Uterine |
| | | sarcoma with features of fibrosarcoma/Pediatric |
| | | haemangiopericytoma-like sarcoma |
| BRD8 | PHF1 | Low grade endometrial stromal sarcoma |
| EPC2 | PHF1 | Low grade endometrial stromal sarcoma |
| JAZF1 | PHF1 | Low grade endometrial stromal sarcoma |
| JAZF1 | SUZ12 | Low grade endometrial stromal sarcoma |
| EPC1 | PHF1 | Low grade endometrial stromal sarcoma/Ossifying |
| | | fibromyxoid tumor |
| EWSR1 | CREB3L1 | Low grade fibromyxoid sarcoma/Sclerosing epithelioid |
| | | fibrosarcoma |
| FUS | CREB3L1 | Low grade fibromyxoid sarcoma/Sclerosing epithelioid |
| | | fibrosarcoma |
| EWSR1 | CREB3L2 | Low grade fibromyxoid sarcoma/Sclerosing epithelioid |
| | | fibrosarcoma |
| FUS | CREB3L2 | Low grade fibromyxoid sarcoma/Sclerosing epithelioid |
| | | fibrosarcoma |
| ETV6 | RET | Mammary analog secretory carcinoma |
| IRF2BP2 | CDX1 | Mesenchymal chondrosarcoma |
| HEY1 | NCOA2 | Mesenchymal chondrosarcoma |
| EWSR1 | YY1 | Mesothelioma |
| FUS | ATF1 | Mesothelioma/Angiomatoid fibrous histiocytoma |
| CRTC1 | MAML2 | Mucoepidermoid carcinoma |
| CRTC3 | MAML2 | Mucoepidermoid carcinoma |
| FUS | KLF17 | Myoepithelial carcinoma/myoepithelioma soft tissue |
| EWSR1 | PBX1 | Myoepithelial carcinoma/myoepithelioma soft tissue |
| EWSR1 | PBX3 | Myoepithelial carcinoma/myoepithelioma soft tissue |
| LIFR | PLAG1 | Myoepithelial carcinoma/myoepithelioma soft tissue |
| EWSR1 | ZNF444 | Myoepithelial carcinoma/myoepithelioma soft tissue |
| EWSR1 | ATF1 | Myoepithelial carcinoma/myoepithelioma soft |
| | | tissue/mesothelioma/Clear cell sarcoma soft tissues and |
| | | digestive tract/Angiomatoid fibrous histiocytoma |
| EWSR1 | POU5F1 | Myoepithelial carcinoma/myoepithelioma soft |
| | | tissue/Undifferenciated round cell sarcoma/Ewing |
| | | Sarcoma/PNET |
| SRF | RELA | Myofibroma/myopericytoma |
| CCBL1 | ARL1 | Myxofibrosarcoma |
| KIAA2026 | NUDT11 | Myxofibrosarcoma |
| AFF3 | PHF1 | Myxofibrosarcoma |
| EWSR1 | DDIT3(CHOP) | Myxoid/round cell liposarcoma |
| FUS | DDIT3(CHOP) | Myxoid/round cell liposarcoma |
| MYH9 | USP6 | Nodular fasciitis/Cellular fibroma of tendon sheath |
| BRD3 | NUTM1 | NUT carcinoma |
| BRD4 | NUTM1 | NUT carcinoma |
| ZNF592 | NUTM1 | NUT Carcinoma |
| FUS | TFCP2 | Osseous RMS/epithelioid rhabdomyosarcoma |
| CREBBP | BCORL1 | Ossifying fibromyxoid tumor |
| EP400 | PHF1 | Ossifying fibromyxoid tumor |
| MEAF6 | PHF1 | Ossifying fibromyxoid tumor |
| ZC3H7B | BCOR | Ossifying fibromyxoid tumor/High grade endometrial stromal |
| | | sarcoma |
| STRN | ALK | Papillary thyroid carcinoma |
| RAD51B | OPHNI | PEComa |
| DVL2 | TFE3 | PEComa/Xp11 renal cell carcinoma |
| ACTB | GLI1 | Pericytoma/Pericytoma AND Malignant Epithelioid Neoplasm |
| FN1 | FGF1 | Phosphaturic mesenchymal tumor |
| FN1 | FGFR | Phosphaturic mesenchymal tumor |
| MXD4 | NUTM1 | Primary ovarian undifferentiated small round cell sarcoma |
| YWHAE | NUTM2A_B | Primitive myxoid mesenchymal tumor of infancy |
| | | (PMMTI)/SoftTissue Undifferentiated Round Cell Sarcoma of |
| | | Infancy/Clear cell sarcoma of the kidney/High grade |
| | | endometrial stromal sarcoma |
| MEIS1 | NCOA2 | Primitive spindle cell sarcoma of the kidney |
| TMPRSS2 | ERG | Prostate Tumor |
| TMPRSS2 | ETV1 | Prostate Tumor |
| ACTB | FOSB | Pseudomyogenic hemangioendothelioma |
| ETV4 | NCOA2 | Soft tissue angiofibroma |
| NAB2 | STAT6 | Solitary fibrous tumor |
| EWSR1 | PATZ1 | Spindle round cell sarcomas/Ewing Sarcoma/PNET |
| SS18 | SSX | Synovial sarcoma |
| SS18L1 | SSX | Synovial sarcoma |
| CRTC1 | SS18 | Undifferenciated round cell sarcoma |
| EWSR1 | SP3 | Undifferenciated round cell sarcoma/Ewing Sarcoma/PNET |
| CITED2 | PRDM10 | Undifferenciated round cell sarcoma/Undifferentiated |
| | | pleomorphic sarcoma |
| RAD51B | HMGA2 | Uterine leiomyoma |
| RBPMS | NTRK3 | Uterine sarcoma with features of fibrosarcoma |
| GREB1 | NCOA2 | Uterine Tumors Resembling Ovarian Sex Cord Tumors |
| NonO | TFE3 | Xp11 renal cell carcinoma |
| PRCC | TFE3 | Xp11 renal cell carcinoma |
| RBM10 | TFE3 | Xp11 renal cell carcinoma |
| SFPQ | TFE3 | Xp11 renal cell carcinoma |
| ASPSCR1 | TFE3 | Xp11 renal cell carcinoma/Alveolar soft part sarcoma |
| FXR1 | BRAF | ganglioma |
| C11orf95 | RELA | ependymoma |
| ETV6 | NTRK3 | xanthoastrocytoma |
| FGFR1 | TACC1 | pilocytic astrocytoma |
| FGFR3 | TACC3 | glioblastoma |
| GOPC | ROS | glioblastoma |
| KIAA1549 | BRAF | glioblastoma, pilocytic astrocytoma, ganglioma |
| MYB | QKI | angiocentric glioma |
| PTEN | COL17A1 | glioblastome |
| PTPRZ1 | MET | glioblastome |
| RNF213 | SLC26A11 | glioblastome |
| SLC44A1 | PRKCA | tumeur glioneuronale papillaire |
| NACC2 | NTRK2 | pilocytic astrocytoma |
| MKRN1 | BRAF | Papillary Thyroid Carcinoma |
| BCAN | NTRK1 | Glioma |
| PTEN | COL17A1 | glioblastoma multiforme |
| X | NTRK1 | Various |
| X | NTRK2 | Various |
| X | NTRK3 | Various |

### Example 7: Diagnosing a Lung Carcinoma

The sample from a subject was subjected to an RT-MLPA step according to the invention, using the probes described above.

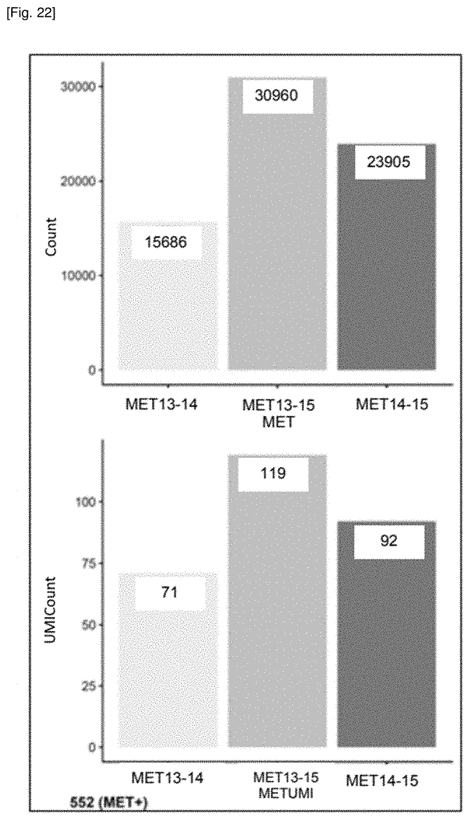

At the end of the PCR step, 70,571 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to precisely determine the number of fusion RNA molecules present in the starting sample (in the case tested here: (71 junctions between exons 13 and 14, 119 between exons 13 and 15, and 92 between exons 14 and 15 of the METgene)). These results, and in particular the detection of transcripts 13-15, indicate the presence of a splicing abnormality of the MET gene, making this patient eligible for targeted therapy (see FIG. 22).

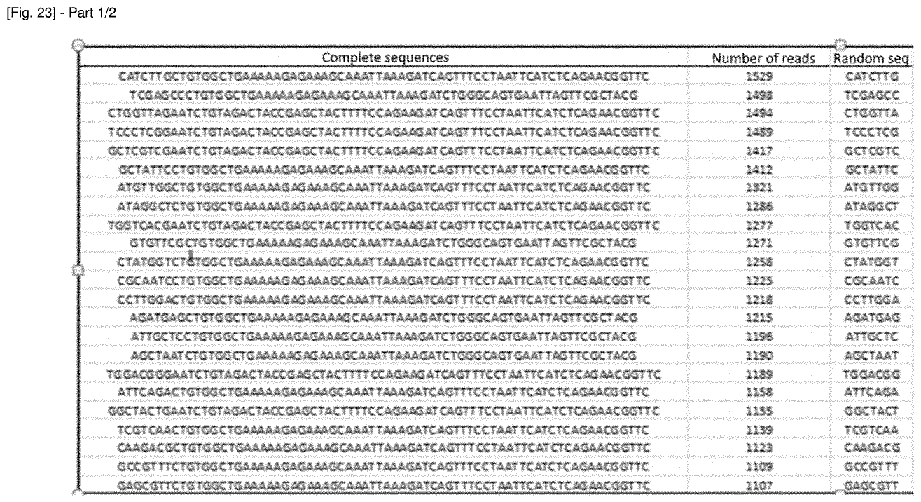

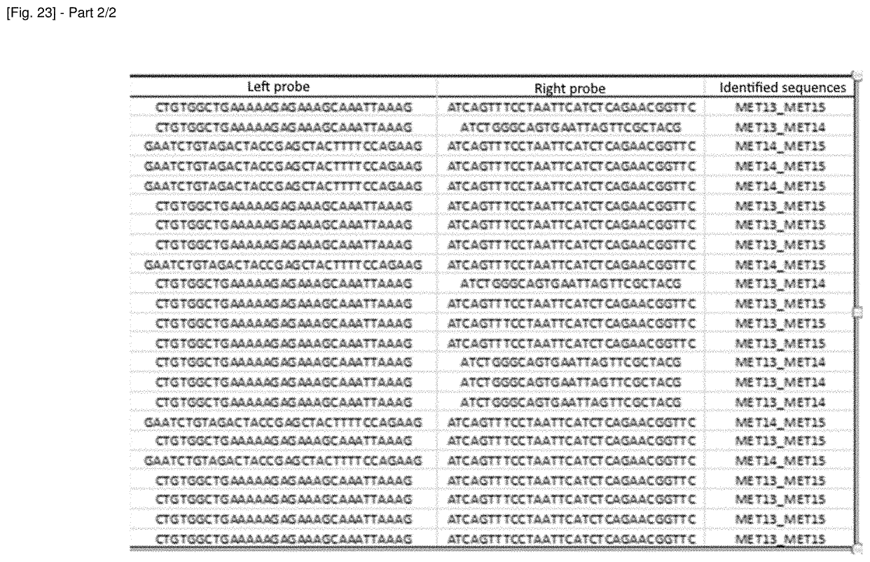

FIG. 23 shows the results obtained. The results allow making the diagnosis.

### Example 8: Diagnosing a Lung Carcinoma

The sample from a subject was subjected to an RT-MLPA step according to the invention, using the probes described above.

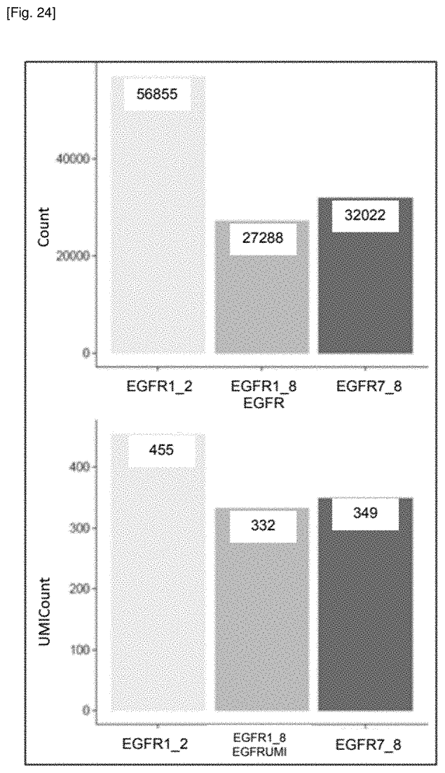

At the end of the PCR step, 116,165 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to precisely determine the number of fusion RNA molecules present in the starting sample (in the case tested here: (455 junctions between exons 1 and 2, 332 between exons 1 and 8, and 349 between exons 7 and 8 of the EGFR gene)). These results, and in particular the detection of transcripts 1-8, indicate the presence of an internal deletion of the EGFR gene, making this patient eligible for targeted therapy (see FIG. 24).

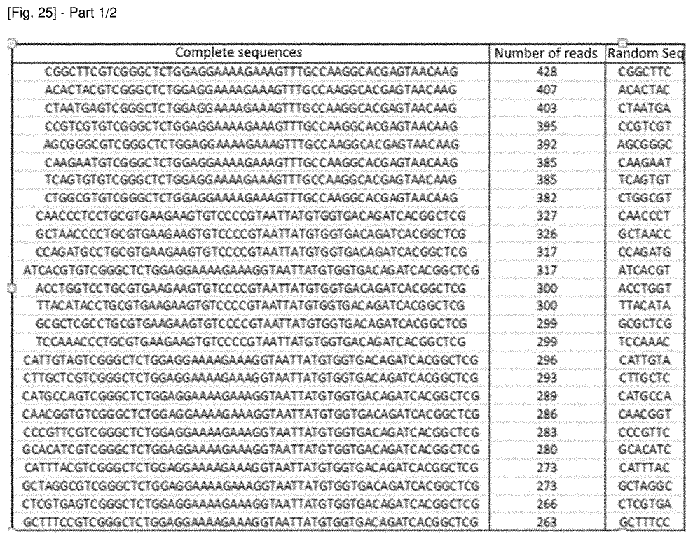

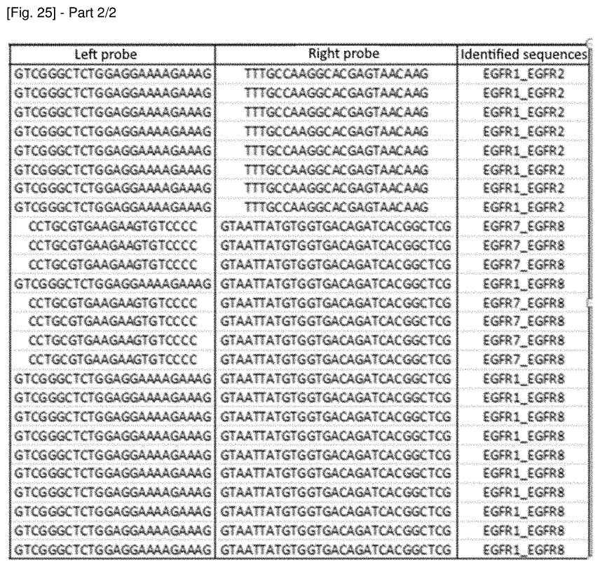

FIG. 25 shows the results obtained. The results allow making the diagnosis.

### Example 9: Diagnosing a Lung Carcinoma

The sample from a subject was subjected to an RT-MLPA step according to the invention, using the probes described above.

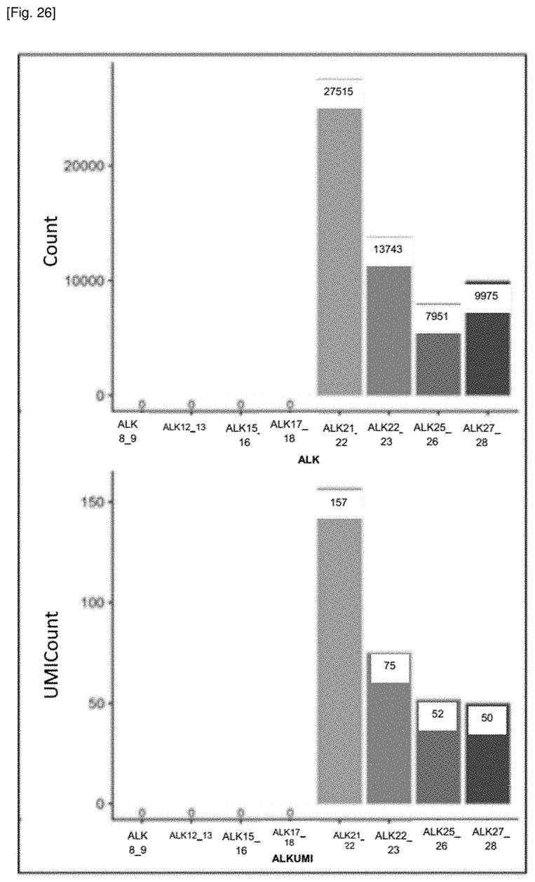

At the end of the PCR step, 59,214 sequences corresponding to unique PCR products (fusion transcripts) were read by next-generation sequencing. These sequences all carry a 7 base-pair molecular barcode sequence at 5′. Due to PCR amplification, these molecular barcode sequences are read several times (number of reads). Counting these barcodes makes it possible to precisely determine the number of fusion RNA molecules present in the starting sample (in the case tested here: 157 junctions between exons 21 and 22, 75 between exons 22 and 23, 52 between exons 25 and 26, and 50 between exons 27 and 28 of the ALK gene). These results, and in particular the demonstration of an expression imbalance between the 5′ and 3′ portions of the ALK gene, indicate that this gene is rearranged, making this patient eligible for targeted therapy (see FIG. 26).

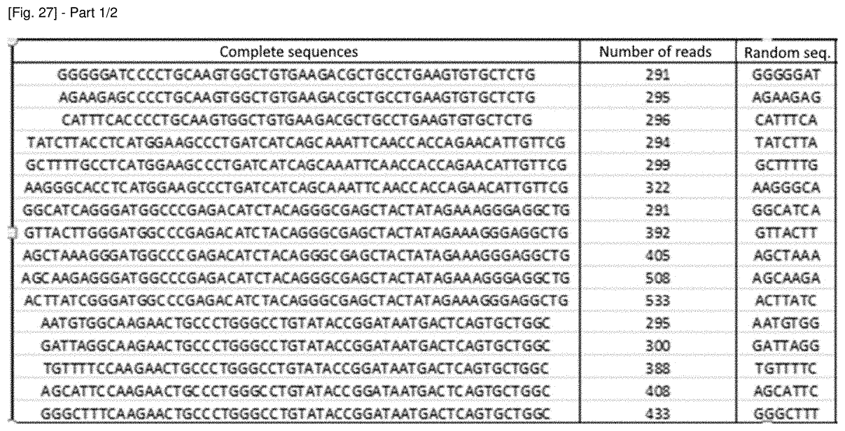

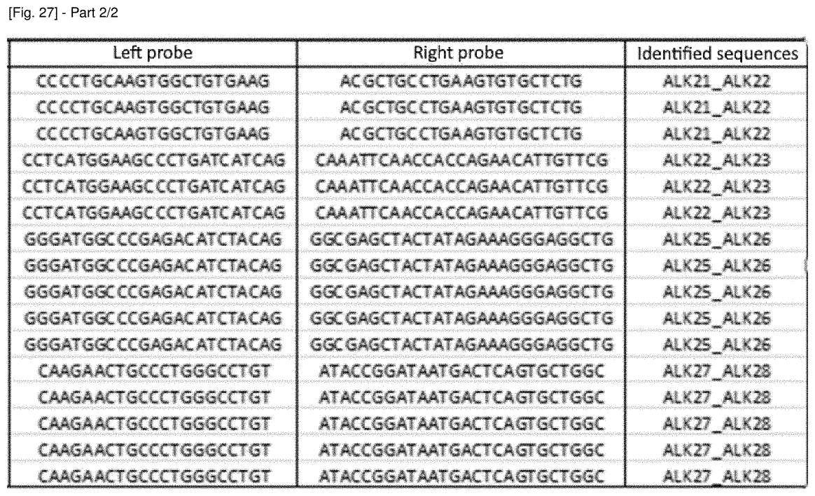

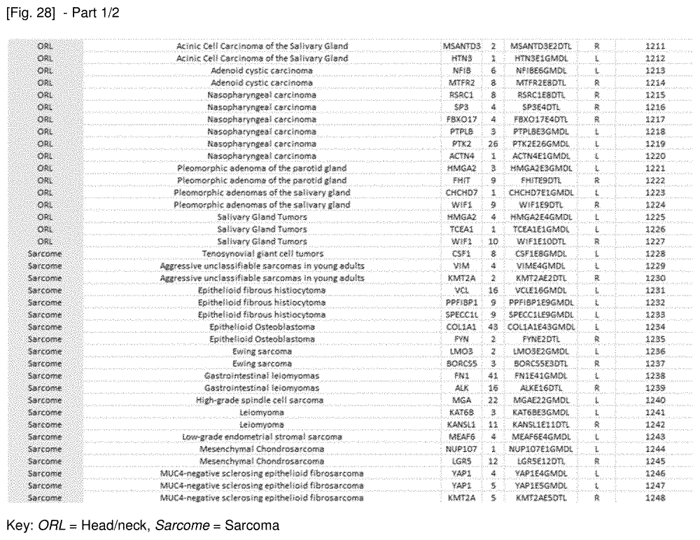

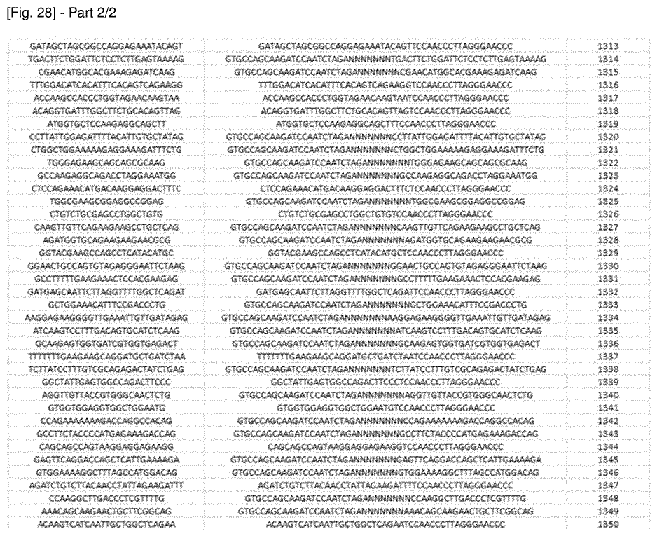

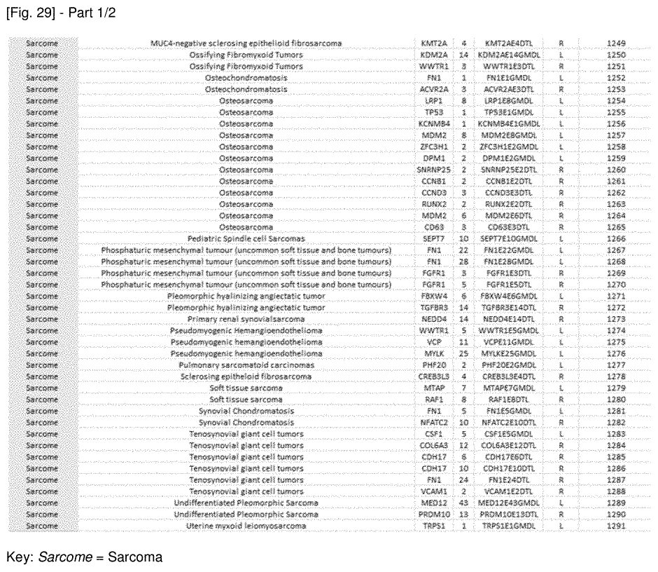

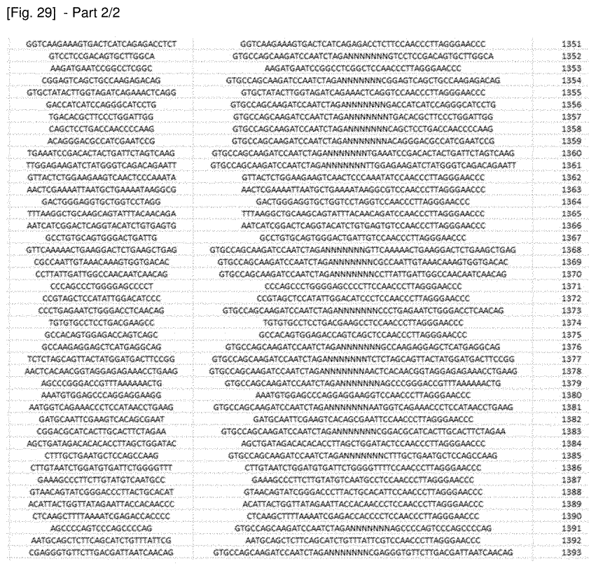

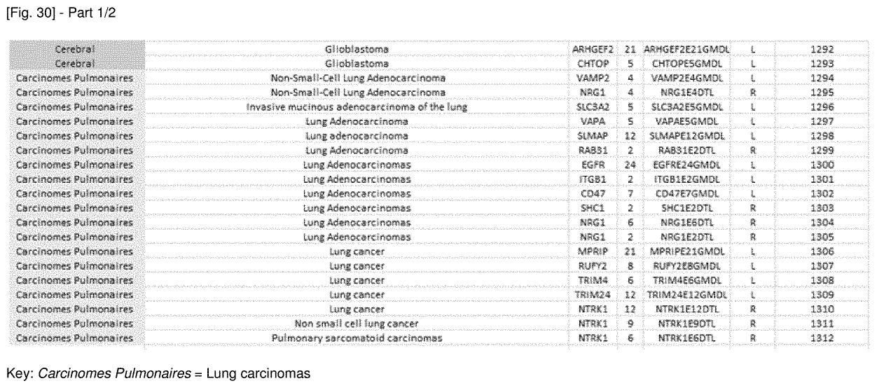

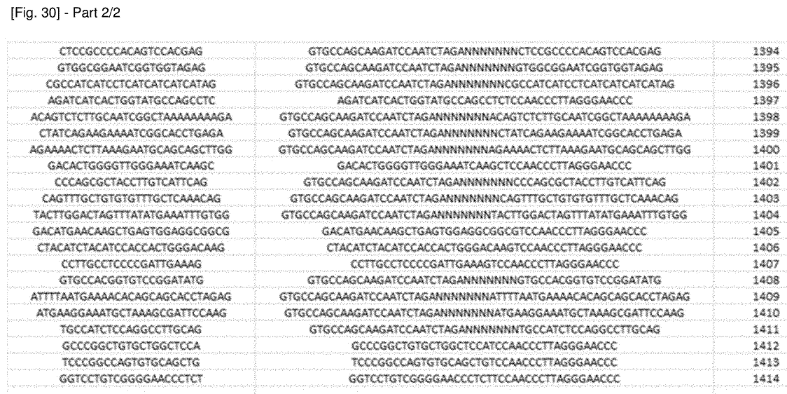

FIG. 27 shows the results obtained. The results allow making the diagnosis.

1. Method for diagnosing cancer in a subject, comprising an RT-MLPA step on a biological sample obtained from said subject, wherein:
the RT-MLPA step is carried out using at least one pair of probes comprising at least one probe selected from:
the probes SEQ ID NO: 1 to 13, and/or 866 to 938, and/or SEQ ID NO: 940 to 1104, and/or SEQ ID NO: 1211 to 1312, and/or
the probes SEQ ID NO: 96 to 99, and/or SEQ ID NO: 1105 to 1107 and/or SEQ ID NO: 939, and/or
the probes SEQ ID NO: 1108 to 1123,
each of the probes being fused, at at least one end, with a primer sequence,
and at least one of the probes of said pair comprising a molecular barcode sequence.

2. Method according to claim 1, wherein the probes SEQ ID NO: 14 to 91 are also used for the RT-MLPA step, each of the probes being fused, at at least one end, with a primer sequence, and at least one of the probes preferably comprising a molecular barcode sequence.

3. Method according to any one of claims 1 to 2, wherein the cancer is associated with formation of a fusion gene and/or an exon skipping and/or a 5′-3′ imbalance.

4. Method according to any one of claims 1 to 3, wherein the cancer involves at least one gene selected from RET, MET, ALK, EGFR and/or ROS.

5. Method according to any one of claims 1 to 3, wherein the cancer is associated with the formation of an exon skipping of the MET or EGFR gene.

6. Method according to any one of claims 1 to 3, wherein the cancer is a carcinoma, in particular a lung carcinoma, and more particularly a bronchopulmonary carcinoma.

7. Method according to any one of claims 1 to 2, wherein the cancer is a sarcoma, a brain tumor, a gynecological tumor, or a tumor of the head and neck.

8. Method according to any one of claims 1 to 4, wherein the primer sequence is selected from the sequences:
SEQ ID NO: 92 and SEQ ID NO: 93, or
SEQ ID NO: 94 and SEQ ID NO: 95.

9. Method according to any one of claims 1 to 5, wherein the molecular barcode sequence is represented by SEQ ID NO: 100.

10. Method according to any one of claims 1 to 6, wherein the cancer associated with the formation of a fusion gene is diagnosed using at least one pair of probes comprising at least one probe selected among probes SEQ ID NO: 1 to 13, SEQ ID NO: 866 to 938 and/or SEQ ID NO: 940 to 1104, and/or SEQ ID NO: 1211 to 1312, optionally the probes SEQ ID NO: 14 to 91, and wherein each of the probes is fused, at at least one end, with a primer sequence, preferably selected from the sequences SEQ ID NO: 92 and SEQ ID NO: 93,
and wherein at least one of the probes comprises a molecular barcode sequence.

11. Method according to any one of claims 1 to 6, wherein the cancer associated with an exon skipping is diagnosed using at least one pair of probes comprising at least one probe selected from probes SEQ ID NO: 96 to 99 and/or SEQ ID NO: 1105 to 1107 and/or SEQ ID NO: 939, and wherein each of the probes is fused, at at least one end, with a primer sequence, preferably selected from the sequences SEQ ID NO: 94 and SEQ ID NO: 95 and wherein at least one of the probes comprises a molecular barcode sequence.

12. Method according to any one of claims 1 to 6, wherein the cancer associated with a 5′-3′ imbalance is diagnosed using at least one pair of probes comprising at least one probe selected from probes SEQ ID NO: 1108 to 1123,
and wherein each of the probes is fused, at at least one end, with a primer sequence, preferably selected from the sequences SEQ ID NO: 94 and SEQ ID NO: 95
and wherein at least one of the probes comprises a molecular barcode sequence.

13. Method according to any one of claims 1 to 12, wherein said biological sample is selected among blood and a biopsy from said subject.

14. Method according to any one of claims 1 to 13, wherein said RT-MLPA step comprises at least the following steps:
a) extraction of RNA from the biological sample from the subject,
b) conversion of the RNA extracted in a) into cDNA by reverse transcription,
c) incubation of the cDNA obtained in b) with a pair of probes comprising at least one probe selected from:
probes SEQ ID NO: 1 to 13, and/or SEQ ID NO: 866 to 938 and/or SEQ ID NO: 940 to 1104, and/or SEQ ID NO: 1211 to 1312, and/or
probes SEQ ID NO: 96 to 99, and/or SEQ ID NO: 1105 to 1107 and/or SEQ ID NO: 939, and/or
probes SEQ ID NO: 1108 to 1123,
each of the probes being fused, at at least one end, with a primer sequence,
and at least one of the probes of said pair comprising a molecular barcode sequence,
d) addition of a DNA ligase to the mixture obtained in c), in order to establish a covalent bond between two adjacent probes,
e) PCR amplification of the adjacent covalently bound probes obtained in d), in order to obtain amplicons.

15. Method according to claim 10, wherein it comprises a step f) of analyzing the results of the PCR of step e), preferably by sequencing.

16. Method according to claim 11, wherein the sequencing step is a step of capillary sequencing or next-generation sequencing.

17. Method according to claim 15 or 16, wherein it comprises a step g) of determining the level of expression of the amplicons that are obtained at the end of the PCR step, implemented by computer.

18. Kit comprising at least probes SEQ ID NO: 1 to 13, and/or probes SEQ ID NO: 96 to 99, and/or probes SEQ ID NO: 866 to 938 and/or probes SEQ ID NO: 940 to 1104, and/or SEQ ID NO: 1211 to 1312, and/or probes SEQ ID NO: 1105 to 1107 and/or probe SEQ ID NO: 939, and/or probes SEQ ID NO: 1108 to 1123, preferably further comprising probes SEQ ID NO: 14 to 91, each of the probes preferably being fused, at at least one end, with a primer sequence, and at least one of the probes preferably comprising a molecular barcode sequence.

19. Kit comprising at least the following probes: SEQ ID NO: 1 to 13, SEQ ID NO: 14 to 91, SEQ ID NO: 96 to 99, SEQ ID NO: 103 to 127, SEQ ID NO: 128, SEQ ID NO: 129, SEQ ID NO: 130 to 137, SEQ ID NO: 138 to 168, SEQ ID NO: 169 to 194, SEQ ID NO: 195 to 198, SEQ ID NO: 199 to 245, SEQ ID NO: 246 to 344, SEQ ID NO: 345 to 403, SEQ ID NO: 404 to 428, SEQ ID NO: 429 to 436, SEQ ID NO: 437 to 479, SEQ ID NO: 480 to 504, SEQ ID NO: 505, SEQ ID NO: 506, SEQ ID NO: 507 to 514, SEQ ID NO: 515 to 546, SEQ ID NO: 547 to 582, SEQ ID NO: 583 to 586, SEQ ID NO: 587 to 633, SEQ ID NO: 634 to 732, SEQ ID NO: 733 to 791, SEQ ID NO: 792 to 816, SEQ ID NO: 817 to 824, SEQ ID NO: 825, SEQ ID NO: 826 to 835, SEQ ID NO: 866 to 938, SEQ ID NO: 940 to 1104, SEQ ID NO: 1105 to 1107, SEQ ID NO: 939, and SEQ ID NO: 1108 to 1123, and SEQ ID NO: 1211 to 1312,
each of the probes preferably being fused, at at least one end, with a primer sequence, and at least one of the probes preferably comprising a molecular barcode sequence.

20. Method for determining the level of expression of amplicons that are obtained at the end of a PCR step, said method being implemented by computer, and comprising:
(1) a step of demultiplexing the results of amplicons obtained at the end of a PCR step,
(2) a step of searching for pairs of probes used during the PCR step,
(3) a step of counting the results and molecular barcode sequences, and optionally
(4) a step of evaluating the quality of sequencing of the sample.


SEQUENCE LISTING (1414 sequences)

SEQ ID NO: 1 - DNA, 20 bp, Artificial sequence
  Primer
tgtcacccaccccggagcca

SEQ ID NO: 2 - DNA, 30 bp, Artificial sequence
  Primer
agccctgagtacaagctgagcaagctccgc

SEQ ID NO: 3 - DNA, 23 bp, Artificial sequence
  Primer
tgtaccgccggaagcaccaggag

SEQ ID NO: 4 - DNA, 24 bp, Artificial sequence
  Primer
tggaagcaagcaatttcttcaacc

SEQ ID NO: 5 - DNA, 27 bp, Artificial sequence
  Primer
atctgggcagtgaattagttcgctacg

SEQ ID NO: 6 - DNA, 30 bp, Artificial sequence
  Primer
atcagtttcctaattcatctcagaacggtt

SEQ ID NO: 7 - DNA, 23 bp, Artificial sequence
  Primer
atccactgtgcgacgagctgtgc

SEQ ID NO: 8 - DNA, 24 bp, Artificial sequence
  Primer
gaggatccaaagtgggaattccct

SEQ ID NO: 9 - DNA, 20 bp, Artificial sequence
  Primer
atgtggccgaggaggcgggc

SEQ ID NO: 10 - DNA, 25 bp, Artificial sequence
  Primer
ctggagtcccaaataaaccaggcat

SEQ ID NO: 11 - DNA, 30 bp, Artificial sequence
  Primer
atgatttttggataccagaaacaagtttca

SEQ ID NO: 12 - DNA, 30 bp, Artificial sequence
  Primer
tctggcatagaagattaaagaatcaaaaaa

SEQ ID NO: 13 - DNA, 28 bp, Artificial sequence
  Primer
tactcttccaacccaagaggagattgaa

SEQ ID NO: 14 - DNA, 30 bp, Artificial sequence
  Primer
caacattcaactccctactttgtccatcag

SEQ ID NO: 15 - DNA, 21 bp, Artificial sequence
  Primer
agcccaagcttcccatcacag

SEQ ID NO: 16 - DNA, 24 bp, Artificial sequence
  Primer
acaggctgtgtgcatgcaccaaag

SEQ ID NO: 17 - DNA, 23 bp, Artificial sequence
  Primer
gaagattgcccgagagcaaaaag

SEQ ID NO: 18 - DNA, 20 bp, Artificial sequence
  Primer
gcaaagccagcgtgaccatc

SEQ ID NO: 19 - DNA, 25 bp, Artificial sequence
  Primer
tgagctctccagaaaattgatgcag

SEQ ID NO: 20 - DNA, 27 bp, Artificial sequence
  Primer
cgagttcaagcaggcctatatcacctg

SEQ ID NO: 21 - DNA, 23 bp, Artificial sequence
  Primer
tgggaacatcccatggtatcaca

SEQ ID NO: 22 - DNA, 19 bp, Artificial sequence
  Primer
gccacccatgcagcccacg

SEQ ID NO: 23 - DNA, 23 bp, Artificial sequence
  Primer
gcccactgacgctccaccgaaag

SEQ ID NO: 24 - DNA, 21 bp, Artificial sequence
  Primer
ccaagcaggatctgggcccag

SEQ ID NO: 25 - DNA, 21 bp, Artificial sequence
  Primer
ggcagctcagcagctcctcag

SEQ ID NO: 26 - DNA, 28 bp, Artificial sequence
  Primer
tggccaatgtgatctggaacttattaat

SEQ ID NO: 27 - DNA, 30 bp, Artificial sequence
  Primer
atccaggtcatgaaggagtacttgacaaag

SEQ ID NO: 28 - DNA, 27 bp, Artificial sequence
  Primer
ctacagagacacaacccattgtttatg

SEQ ID NO: 29 - DNA, 27 bp, Artificial sequence
  Primer
ctactctggtctctggcattgctggtg

SEQ ID NO: 30 - DNA, 26 bp, Artificial sequence
  Primer
cttcatgagctgcaatctcatcactg

SEQ ID NO: 31 - DNA, 25 bp, Artificial sequence
  Primer
cccacacctgggaaaggacctaaag

SEQ ID NO: 32 - DNA, 27 bp, Artificial sequence
  Primer
gatctgaatcctgaaagagaaatagag

SEQ ID NO: 33 - DNA, 28 bp, Artificial sequence
  Primer
tgaaagagaaatagagatatgctggatg

SEQ ID NO: 34 - DNA, 30 bp, Artificial sequence
  Primer
tttaatgatggcttccaaatagaagtacag

SEQ ID NO: 35 - DNA, 23 bp, Artificial sequence
  Primer
gccataggaacgcactcaggcag

SEQ ID NO: 36 - DNA, 27 bp, Artificial sequence
  Primer
agctctctgtgatgcgctactcaatag

SEQ ID NO: 37 - DNA, 27 bp, Artificial sequence
  Primer
actcgggagactatgaaatattgtact

SEQ ID NO: 38 - DNA, 27 bp, Artificial sequence
  Primer
cagtgaaaaaatcagtctcaagtaaag

SEQ ID NO: 39 - DNA, 27 bp, Artificial sequence
  Primer
agcataaagatgtcatcatcaaccaag

SEQ ID NO: 40 - DNA, 29 bp, Artificial sequence
  Primer
agcggaaggttaatgttcttcagaagaag

SEQ ID NO: 41 - DNA, 26 bp, Artificial sequence
  Primer
ggagaagacaaagaaggcagagagag

SEQ ID NO: 42 - DNA, 26 bp, Artificial sequence
  Primer
atcagataaagagccaggagcagctg

SEQ ID NO: 43 - DNA, 26 bp, Artificial sequence
  Primer
caaagccactggagtctttaccacac

SEQ ID NO: 44 - DNA, 30 bp, Artificial sequence
  Primer
agaaacaagaaaccctacaagaagaaataa

SEQ ID NO: 45 - DNA, 27 bp, Artificial sequence
  Primer
agcttaagaatgaaccgaccacaagaa

SEQ ID NO: 46 - DNA, 30 bp, Artificial sequence
  Primer
caagtacttggataaggaactggcaggaag

SEQ ID NO: 47 - DNA, 30 bp, Artificial sequence
  Primer
acacaagtggggaaatcaaagtattacaag

SEQ ID NO: 48 - DNA, 20 bp, Artificial sequence
  Primer
cccacctgagcctgccgact

SEQ ID NO: 49 - DNA, 24 bp, Artificial sequence
  Primer
gcaaatcacagatcgaagagacag

SEQ ID NO: 50 - DNA, 20 bp, Artificial sequence
  Primer
tgctgagggctgggaagaag

SEQ ID NO: 51 - DNA, 30 bp, Artificial sequence
  Primer
ttagttaatcacgatttctctcctcttgag

SEQ ID NO: 52 - DNA, 27 bp, Artificial sequence
  Primer
attgctgtgggaaataatgatgtaaag

SEQ ID NO: 53 - DNA, 27 bp, Artificial sequence
  Primer
gcagcatgtcagcttcgtatctctcaa

SEQ ID NO: 54 - DNA, 27 bp, Artificial sequence
  Primer
aagaactagtccagcttcgagcacaag

SEQ ID NO: 55 - DNA, 27 bp, Artificial sequence
  Primer
caggacctggctacaagagttaaaaag

SEQ ID NO: 56 - DNA, 27 bp, Artificial sequence
  Primer
gaacagctcactaaagtgcacaaacag

SEQ ID NO: 57 - DNA, 25 bp, Artificial sequence
  Primer
agaagagggcattctgcacagattg

SEQ ID NO: 58 - DNA, 25 bp, Artificial sequence
  Primer
gaaagggagtttggttctgtagatg

SEQ ID NO: 59 - DNA, 28 bp, Artificial sequence
  Primer
gttgctcctattgcaacaacaaactcag

SEQ ID NO: 60 - DNA, 27 bp, Artificial sequence
  Primer
ggatcttcgtagcatcagttgaagcag

SEQ ID NO: 61 - DNA, 27 bp, Artificial sequence
  Primer
ttttcttaccacaacatgacagtagtg

SEQ ID NO: 62 - DNA, 23 bp, Artificial sequence
  Primer
aggctgtggagtggcagcagaag

SEQ ID NO: 63 - DNA, 28 bp, Artificial sequence
  Primer
gaggaacagactaagaaggctcagcaag

SEQ ID NO: 64 - DNA, 23 bp, Artificial sequence
  Primer
gctgtatctccatgccagagcag

SEQ ID NO: 65 - DNA, 25 bp, Artificial sequence
  Primer
aaagcagaccttggagaacagtcag

SEQ ID NO: 66 - DNA, 30 bp, Artificial sequence
  Primer
cagtgcatattagtggacagcacttagtag

SEQ ID NO: 67 - DNA, 26 bp, Artificial sequence
  Primer
ggtggtactggcccaaggtaaaaaag

SEQ ID NO: 68 - DNA, 30 bp, Artificial sequence
  Primer
cagtatgaaaaaaagcttaaatcaaccaaa

SEQ ID NO: 69 - DNA, 25 bp, Artificial sequence
  Primer
acatttcatggggctccactaacag

SEQ ID NO: 70 - DNA, 27 bp, Artificial sequence
  Primer
gtgggaacgtgaaacatctgatacaag

SEQ ID NO: 71 - DNA, 24 bp, Artificial sequence
  Primer
agctgtctggctctggagatctgg

SEQ ID NO: 72 - DNA, 23 bp, Artificial sequence
  Primer
tgagagaacggaggtcctggcag

SEQ ID NO: 73 - DNA, 25 bp, Artificial sequence
  Primer
gtaccaccttatccacagccacagc

SEQ ID NO: 74 - DNA, 23 bp, Artificial sequence
  Primer
gctgcctgcgtcccaaagaacag

SEQ ID NO: 75 - DNA, 27 bp, Artificial sequence
  Primer
acataaccattagcagagaggctcagg

SEQ ID NO: 76 - DNA, 20 bp, Artificial sequence
  Primer
cgccttccagctggttggag

SEQ ID NO: 77 - DNA, 22 bp, Artificial sequence
  Primer
gcagctgcccttagccctctgg

SEQ ID NO: 78 - DNA, 30 bp, Artificial sequence
  Primer
tgttacctcaagaagcagaagaagaaaaca

SEQ ID NO: 79 - DNA, 23 bp, Artificial sequence
  Primer
gaagcctccaagctatgattctg

SEQ ID NO: 80 - DNA, 27 bp, Artificial sequence
  Primer
gaccttccaccaatattcctgaaaatg

SEQ ID NO: 81 - DNA, 27 bp, Artificial sequence
  Primer
ttggcttaacagatgatcaggtttcag

SEQ ID NO: 82 - DNA, 27 bp, Artificial sequence
  Primer
ctcagactcaagcaggtcagattgaag

SEQ ID NO: 83 - DNA, 30 bp, Artificial sequence
  Primer
agcctcaacagtatggtattcagtattcag

SEQ ID NO: 84 - DNA, 26 bp, Artificial sequence
  Primer
tcagggaacaggaagaattcctaggg

SEQ ID NO: 85 - DNA, 27 bp, Artificial sequence
  Primer
tggaaaagacaattgatgacctggaag

SEQ ID NO: 86 - DNA, 30 bp, Artificial sequence
  Primer
aaacaacaggagttgccattccattacatg

SEQ ID NO: 87 - DNA, 21 bp, Artificial sequence
  Primer
ccgtcagcctcttctccccag

SEQ ID NO: 88 - DNA, 25 bp, Artificial sequence
  Primer
gctgccagatattccacccatacag

SEQ ID NO: 89 - DNA, 30 bp, Artificial sequence
  Primer
acagaggatggcaggaggagtgcttgcatg

SEQ ID NO: 90 - DNA, 22 bp, Artificial sequence
  Primer
gttaagccccgtggaccaaagg

SEQ ID NO: 91 - DNA, 22 bp, Artificial sequence
  Primer
gctggaaacatttccgaccctg

SEQ ID NO: 92 - DNA, 23 bp, Artificial sequence
  Priming sequence
gtgccagcaagatccaatctaga

SEQ ID NO: 93 - DNA, 19 bp, Artificial sequence
  Priming sequence
tccaacccttagggaaccc

SEQ ID NO: 94 - DNA, 21 bp, Artificial sequence
  Priming sequence - Additional queue
gccattgcggtgacactatag

SEQ ID NO: 95 - DNA, 22 bp, Artificial sequence
  Priming sequence - Additional queue
ccctatagtgagtcgtcgtcgc

SEQ ID NO: 96 - DNA, 31 bp, Artificial sequence
  Primer
ctgtggctgaaaaagagaaagcaaattaaag

SEQ ID NO: 97 - DNA, 27 bp, Artificial sequence
  Primer
atctgggcagtgaattagttcgctacg

SEQ ID NO: 98 - DNA, 34 bp, Artificial sequence
  Primer
gaatctgtagactaccgagctacttttccagaag

SEQ ID NO: 99 - DNA, 31 bp, Artificial sequence
  Primer
atcagtttcctaattcatctcagaacggttc

SEQ ID NO: 100 - DNA, 10 bp, Artificial sequence
  Sequence of molecular barcode
  misc_feature (1)..(10): n -if present- represents A, T, C or G
  misc_feature (6)..(10): n is present or absent
nnnnnnnnnn

SEQ ID NO: 101 - DNA, 19 bp, Artificial sequence
  Primer
gggttccctaagggttgga

SEQ ID NO: 102 - DNA, 22 bp, Artificial sequence
  Primer
gcgacgacgactcactataggg

SEQ ID NO: 103 - DNA, 22 bp, Artificial sequence
  Primer
aagcccttcagcggccagtagc

SEQ ID NO: 104 - DNA, 23 bp, Artificial sequence
  Primer
gtgaaaagctccgggtcttaggc

SEQ ID NO: 105 - DNA, 22 bp, Artificial sequence
  Primer
ggcgccttccatggagacgcag

SEQ ID NO: 106 - DNA, 25 bp, Artificial sequence
  Primer
attccgctgaccatcaataaggaag

SEQ ID NO: 107 - DNA, 26 bp, Artificial sequence
  Primer
agccactggatttaagcagagttcaa

SEQ ID NO: 108 - DNA, 23 bp, Artificial sequence
  Primer
actgaaggcagccttcgacgtca

SEQ ID NO: 109 - DNA, 24 bp, Artificial sequence
  Primer
gctcttgcatcacccaggggaaag

SEQ ID NO: 110 - DNA, 20 bp, Artificial sequence
  Primer
cagtggcgccggggaggcag

SEQ ID NO: 111 - DNA, 25 bp, Artificial sequence
  Primer
ccattgagacccagagcagcagttc

SEQ ID NO: 112 - DNA, 24 bp, Artificial sequence
  Primer
gagtttgatgaggagcgagcccag

SEQ ID NO: 113 - DNA, 25 bp, Artificial sequence
  Primer
caggtctcatcgggaggaaatggag

SEQ ID NO: 114 - DNA, 26 bp, Artificial sequence
  Primer
agtttcacagctgctggcagtaactg

SEQ ID NO: 115 - DNA, 27 bp, Artificial sequence
  Primer
gccaaggcgaacctagacaagaataag

SEQ ID NO: 116 - DNA, 26 bp, Artificial sequence
  Primer
aatgaagttgagagcgtcacagggat

SEQ ID NO: 117 - DNA, 25 bp, Artificial sequence
  Primer
gagctgcttcaagaagaaacccggc

SEQ ID NO: 118 - DNA, 25 bp, Artificial sequence
  Primer
ctctccgactcgaagaagaagctgc

SEQ ID NO: 119 - DNA, 29 bp, Artificial sequence
  Primer
ttgttagccgaggagaaaaacatctcttc

SEQ ID NO: 120 - DNA, 25 bp, Artificial sequence
  Primer
gtccatgagctggagaagtccaagc

SEQ ID NO: 121 - DNA, 28 bp, Artificial sequence
  Primer
cttcacgagtatgagacggaactggaag

SEQ ID NO: 122 - DNA, 25 bp, Artificial sequence
  Primer
tctcggcctcccgactcctacagtg

SEQ ID NO: 123 - DNA, 26 bp, Artificial sequence
  Primer
ttttgagtatccgaggagcccaggag

SEQ ID NO: 124 - DNA, 29 bp, Artificial sequence
  Primer
ggtcatactgcatcagaaccatgaagaag

SEQ ID NO: 125 - DNA, 24 bp, Artificial sequence
  Primer
ccatgcccattgggagaatagcag

SEQ ID NO: 126 - DNA, 30 bp, Artificial sequence
  Primer
aatgcatacttggaatgaatccttctagag

SEQ ID NO: 127 - DNA, 23 bp, Artificial sequence
  Primer
atgccagcacgagccgccgcttc

SEQ ID NO: 128 - DNA, 24 bp, Artificial sequence
  Primer
tggatgggccccgagaacctcgaa

SEQ ID NO: 129 - DNA, 27 bp, Artificial sequence
  Primer
atcgtactgagaagcactccacaatgc

SEQ ID NO: 130 - DNA, 30 bp, Artificial sequence
  Primer
atccctgtaaaacaaaaaccaaaagaaaag

SEQ ID NO: 131 - DNA, 28 bp, Artificial sequence
  Primer
agtccacaggatcagagtggactttaag

SEQ ID NO: 132 - DNA, 27 bp, Artificial sequence
  Primer
ctctgtgccagtagtgggcatgtagag

SEQ ID NO: 133 - DNA, 26 bp, Artificial sequence
  Primer
gtggaaggcaacatcaggctacaaag

SEQ ID NO: 134 - DNA, 26 bp, Artificial sequence
  Primer
cagacctactccaatgaagtccattg

SEQ ID NO: 135 - DNA, 23 bp, Artificial sequence
  Primer
gaaatgacccattcatggccgcc

SEQ ID NO: 136 - DNA, 26 bp, Artificial sequence
  Primer
gactctcagcatgtcagttctgtaac

SEQ ID NO: 137 - DNA, 26 bp, Artificial sequence
  Primer
cctgagcctccaacaacaaacaaatg

SEQ ID NO: 138 - DNA, 22 bp, Artificial sequence
  Primer
agcagcagctacgggcagcaga

SEQ ID NO: 139 - DNA, 22 bp, Artificial sequence
  Primer
gaggaggacgcggtggaatggg

SEQ ID NO: 140 - DNA, 24 bp, Artificial sequence
  Primer
gaggtggcttcaataagcctggtg

SEQ ID NO: 141 - DNA, 27 bp, Artificial sequence
  Primer
tggatgaaggaccagatcttgatctag

SEQ ID NO: 142 - DNA, 28 bp, Artificial sequence
  Primer
gttcactgctggcctataatacaacctc

SEQ ID NO: 143 - DNA, 30 bp, Artificial sequence
  Primer
acccttcttatgactcagtcagaagaggag

SEQ ID NO: 144 - DNA, 23 bp, Artificial sequence
  Primer
gtcctccccttggaggggcacaa

SEQ ID NO: 145 - DNA, 26 bp, Artificial sequence
  Primer
gttattccaggatctttggagacccg

SEQ ID NO: 146 - DNA, 28 bp, Artificial sequence
  Primer
gaagccttatcagttgtgagtgaggacc

SEQ ID NO: 147 - DNA, 24 bp, Artificial sequence
  Primer
atttaccatatgagccccccagga

SEQ ID NO: 148 - DNA, 25 bp, Artificial sequence
  Primer
ctgctcaaccatctccttccacagt

SEQ ID NO: 149 - DNA, 30 bp, Artificial sequence
  Primer
atccttatcagattcttggaccaacaagta

SEQ ID NO: 150 - DNA, 22 bp, Artificial sequence
  Primer
gcagtggccagatccagctttg

SEQ ID NO: 151 - DNA, 23 bp, Artificial sequence
  Primer
atcccgtcggagacggtctcttc

SEQ ID NO: 152 - DNA, 28 bp, Artificial sequence
  Primer
ctcaggtacctgacaatgatgagcagtt

SEQ ID NO: 153 - DNA, 29 bp, Artificial sequence
  Primer
gagacatcaaacaagagccaggaatgtat

SEQ ID NO: 154 - DNA, 22 bp, Artificial sequence
  Primer
atgtcaccgggtgcgcatcaat

SEQ ID NO: 155 - DNA, 24 bp, Artificial sequence
  Primer
attggcaatggcctctcacctcag

SEQ ID NO: 156 - DNA, 30 bp, Artificial sequence
  Primer
aattcaattcgtcataatctgtccctacac

SEQ ID NO: 157 - DNA, 29 bp, Artificial sequence
  Primer
gtgagaaaccataccagtgtgacttcaag

SEQ ID NO: 158 - DNA, 23 bp, Artificial sequence
  Primer
cccaggacagcagcagggctacg

SEQ ID NO: 159 - DNA, 26 bp, Artificial sequence
  Primer
agcagaggccttatggatatgaccag

SEQ ID NO: 160 - DNA, 23 bp, Artificial sequence
  Primer
atcatgcccaagaagccagcaga

SEQ ID NO: 161 - DNA, 25 bp, Artificial sequence
  Primer
gtttcaaagtcaccctcccaccttt

SEQ ID NO: 162 - DNA, 30 bp, Artificial sequence
  Primer
tgttcaagaaggaagtgtatcttcatacat

SEQ ID NO: 163 - DNA, 27 bp, Artificial sequence
  Primer
atgtgcttttccagactgatccaactg

SEQ ID NO: 164 - DNA, 23 bp, Artificial sequence
  Primer
tggaggtggaggtggaggtggag

SEQ ID NO: 165 - DNA, 23 bp, Artificial sequence
  Primer
cgtggaggcagaggtggcatggg

SEQ ID NO: 166 - DNA, 25 bp, Artificial sequence
  Primer
cgtggtggcttcaataaatttggtg

SEQ ID NO: 167 - DNA, 24 bp, Artificial sequence
  Primer
accaaggatcacgtcatgactccg

SEQ ID NO: 168 - DNA, 23 bp, Artificial sequence
  Primer
ggctgccgtggaatggtttgatg

SEQ ID NO: 169 - DNA, 26 bp, Artificial sequence
  Primer
gttattccaggatctttggagacccg

SEQ ID NO: 170 - DNA, 28 bp, Artificial sequence
  Primer
gaagccttatcagttgtgagtgaggacc

SEQ ID NO: 171 - DNA, 24 bp, Artificial sequence
  Primer
atttaccatatgagccccccagga

SEQ ID NO: 172 - DNA, 25 bp, Artificial sequence
  Primer
ctgctcaaccatctccttccacagt

SEQ ID NO: 173 - DNA, 30 bp, Artificial sequence; identical to SEQ ID NO: 149
  Primer

SEQ ID NO: 174 - DNA, 22 bp, Artificial sequence
  Primer
gcagtggccagatccagctttg

SEQ ID NO: 175 - DNA, 22 bp, Artificial sequence
  Primer
agcgccgcctggagcgcggcag

SEQ ID NO: 176 - DNA, 27 bp, Artificial sequence
  Primer
gataacagcaagatggctttgaactca

SEQ ID NO: 177 - DNA, 23 bp, Artificial sequence
  Primer
tgtaccgccggaagcaccaggag

SEQ ID NO: 178 - DNA, 23 bp, Artificial sequence
  Primer
ggctggaaacatttccgaccctg

SEQ ID NO: 179 - DNA, 27 bp, Artificial sequence
  Primer
tggaaaagacaattgatgacctggaag

SEQ ID NO: 180 - DNA, 27 bp, Artificial sequence
  Primer
cagtgaaaaaatcagtctcaagtaaag

SEQ ID NO: 181 - DNA, 27 bp, Artificial sequence
  Primer
agcataaagatgtcatcatcaaccaag

SEQ ID NO: 182 - DNA, 25 bp, Artificial sequence
  Primer
cccacacctgggaaaggacctaaag

SEQ ID NO: 183 - DNA, 27 bp, Artificial sequence
  Primer
gatctgaatcctgaaagagaaatagag

SEQ ID NO: 184 - DNA, 23 bp, Artificial sequence
  Primer
gccataggaacgcactcaggcag

SEQ ID NO: 185 - DNA, 27 bp, Artificial sequence
  Primer
agctctctgtgatgcgctactcaatag

SEQ ID NO: 186 - DNA, 27 bp, Artificial sequence
  Primer
actcgggagactatgaaatattgtact

SEQ ID NO: 187 - DNA, 25 bp, Artificial sequence
  Primer
ctggagtcccaaataaaccaggcat

SEQ ID NO: 188 - DNA, 30 bp, Artificial sequence; identical to SEQ ID NO: 11
  Primer

SEQ ID NO: 189 - DNA, 30 bp, Artificial sequence; identical to SEQ ID NO: 12
  Primer

SEQ ID NO: 190 - DNA, 24 bp, Artificial sequence
  Primer
agctgtctggctctggagatctgg

SEQ ID NO: 191 - DNA, 23 bp, Artificial sequence
  Primer
tgagagaacggaggtcctggcag

SEQ ID NO: 192 - DNA, 27 bp, Artificial sequence
  Primer
acataaccattagcagagaggctcagg

SEQ ID NO: 193 - DNA, 23 bp, Artificial sequence
  Primer
gcccactgacgctccaccgaaag

SEQ ID NO: 194 - DNA, 26 bp, Artificial sequence
  Primer
ggagaagacaaagaaggcagagagag

SEQ ID NO: 195 - DNA, 23 bp, Artificial sequence
  Primer
acctcagctccgcggaagttgcg

SEQ ID NO: 196 - DNA, 22 bp, Artificial sequence
  Primer
atcgcccaggaccacaccgcag

SEQ ID NO: 197 - DNA, 22 bp, Artificial sequence
  Primer
gatgaccgagcggccgccgagc

SEQ ID NO: 198 - DNA, 22 bp, Artificial sequence
  Primer
gtccccacaccaaagttgtgcg

SEQ ID NO: 199 - DNA, 23 bp, Artificial sequence
  Primer
gcagatggccagtcaggcaccag

SEQ ID NO: 200 - DNA, 29 bp, Artificial sequence
  Primer
tcagttatcatctggtgacaaagcttcag

SEQ ID NO: 201 - DNA, 27 bp, Artificial sequence
  Primer
cgtcttctaatttcactgctgcacaag

SEQ ID NO: 202 - DNA, 24 bp, Artificial sequence
  Primer
ggttcagcttttgccaagcttcag

SEQ ID NO: 203 - DNA, 25 bp, Artificial sequence
  Primer
ggctttggatccacagctacctcaa

SEQ ID NO: 204 - DNA, 26 bp, Artificial sequence
  Primer
tgggttttcctctccaaacaaaacag

SEQ ID NO: 205 - DNA, 23 bp, Artificial sequence
  Primer
tggttttggatcaggcacaggag

SEQ ID NO: 206 - DNA, 30 bp, Artificial sequence
  Primer
attatgaactattaacagaaaatgacatgt

SEQ ID NO: 207 - DNA, 28 bp, Artificial sequence
  Primer
ttcttcaggagagaataccatgggtacc

SEQ ID NO: 208 - DNA, 30 bp, Artificial sequence
  Primer
gaaattgaacttagctcattaagggaagct

SEQ ID NO: 209 - DNA, 30 bp, Artificial sequence
  Primer
cgagaaaatgtcattgaatataaacactgt

SEQ ID NO: 210 - DNA, 30 bp, Artificial sequence
  Primer
cctagtgagagccttgctactactgatgat

SEQ ID NO: 211 - DNA, 30 bp, Artificial sequence
  Primer
actgaatctccagtgttagtgaatgactat

SEQ ID NO: 212 - DNA, 30 bp, Artificial sequence; identical to SEQ ID NO: 66
  Primer

SEQ ID NO: 213 - DNA, 28 bp, Artificial sequence
  Primer
ctgagaatgcacttactggctcattcag

SEQ ID NO: 214 - DNA, 26 bp, Artificial sequence
  Primer
ggagacacacaggcagacccatactg

SEQ ID NO: 215 - DNA, 28 bp, Artificial sequence
  Primer
atcaccattgcttggaagtttgattctc

SEQ ID NO: 216 - DNA, 26 bp, Artificial sequence
  Primer
accagttccctgcgagtctgctactg

SEQ ID NO: 217 - DNA, 27 bp, Artificial sequence
  Primer
attacctggtcatgatcattgtccgtg

SEQ ID NO: 218 - DNA, 26 bp, Artificial sequence
  Primer
tgctacagttgaaactccagcagcgc

SEQ ID NO: 219 - DNA, 26 bp, Artificial sequence
  Primer
ctttgaaaagtccagccgcatttcat

SEQ ID NO: 220 - DNA, 30 bp, Artificial sequence
  Primer
gaagtcacaatgaaacagatttgcaaaaag

SEQ ID NO: 221 - DNA, 25 bp, Artificial sequence
  Primer
gaaattcggcgccttcatcagtatg

SEQ ID NO: 222 - DNA, 30 bp, Artificial sequence
  Primer
aagaagatgaagagtcagatgatgctgatg

SEQ ID NO: 223 - DNA, 30 bp, Artificial sequence
  Primer
attttggatcattgtttgacttggaaaatg

SEQ ID NO: 224 - DNA, 26 bp, Artificial sequence
  Primer
atgggaataactgggaacacaagtcc

SEQ ID NO: 225 - DNA, 23 bp, Artificial sequence
  Primer
gcttggtgcaggatttggaacag

SEQ ID NO: 226 - DNA, 22 bp, Artificial sequence
  Primer
ggagccccccaggccccagtag

SEQ ID NO: 227 - DNA, 28 bp, Artificial sequence
  Primer
gatgtcagaccctaagaagaaggaagag

SEQ ID NO: 228 - DNA, 24 bp, Artificial sequence
  Primer
agccaatggagcattcatgcccaa

SEQ ID NO: 229 - DNA, 23 bp, Artificial sequence
  Primer
ctgtgcggtcagagaagaaacgc

SEQ ID NO: 230 - DNA, 27 bp, Artificial sequence
  Primer
ttgatagagaaaaacaacccagcgaag

SEQ ID NO: 231 - DNA, 24 bp, Artificial sequence
  Primer
ataacccagcagccaactggcttc

SEQ ID NO: 232 - DNA, 27 bp, Artificial sequence
  Primer
gatggtaaattgaaaaaacccaagaat

SEQ ID NO: 233 - DNA, 27 bp, Artificial sequence
  Primer
ttcttgatgaagcagatagaatcttgg

SEQ ID NO: 234 - DNA, 23 bp, Artificial sequence
  Primer
cacccctgccactttggaacaga

SEQ ID NO: 235 - DNA, 29 bp, Artificial sequence
  Primer
ataatctcagtgataccttgaagaagctg

SEQ ID NO: 236 - DNA, 28 bp, Artificial sequence
  Primer
atacggaaacaagtgaaaaaatccaagc

SEQ ID NO: 237 - DNA, 29 bp, Artificial sequence
  Primer
accaagaggctattcaagatctctgtctg

SEQ ID NO: 238 - DNA, 29 bp, Artificial sequence
  Primer
accaagaggctattcaagatctctgcatg

SEQ ID NO: 239 - DNA, 30 bp, Artificial sequence
  Primer
gcagtggaggaagtctctttaagaaaatag

SEQ ID NO: 240 - DNA, 23 bp, Artificial sequence
  Primer
cttctgccgctgcttctgcacag

SEQ ID NO: 241 - DNA, 23 bp, Artificial sequence
  Primer
gcatggggcggctggttctgctg

SEQ ID NO: 242 - DNA, 24 bp, Artificial sequence
  Primer
ctgaggacatctggaggaaggctg

SEQ ID NO: 243 - DNA, 21 bp, Artificial sequence
  Primer
cagcaggaggactccagcgag

SEQ ID NO: 244 - DNA, 24 bp, Artificial sequence
  Primer
gcttcctgctgaactccaagttcc

SEQ ID NO: 245 - DNA, 23 bp, Artificial sequence
  Primer
gacttccagccactgcgctattt

SEQ ID NO: 246 - DNA, 27 bp, Artificial sequence
  Primer
gatgagcaattcttaggttttggctca

SEQ ID NO: 247 - DNA, 25 bp, Artificial sequence
  Primer
gaagaacctaggaaagtccgctttg

SEQ ID NO: 248 - DNA, 27 bp, Artificial sequence
  Primer
gaccctaataggagtattcataccagc

SEQ ID NO: 249 - DNA, 23 bp, Artificial sequence
  Primer
tctgaacaacccagtcctgccag

SEQ ID NO: 250 - DNA, 31 bp, Artificial sequence
  Primer
attcttgaagtgaaaagtccaataaagcaaa

SEQ ID NO: 251 - DNA, 25 bp, Artificial sequence
  Primer
gcatacctagatgaactggtagagc

SEQ ID NO: 252 - DNA, 23 bp, Artificial sequence
  Primer
tgcaccgtccaggtgaggttaga

SEQ ID NO: 253 - DNA, 23 bp, Artificial sequence
  Primer
gaggagccgaggaaggtctgctt

SEQ ID NO: 254 - DNA, 23 bp, Artificial sequence
  Primer
gtgatggtaatgcccgaaggagc

SEQ ID NO: 255 - DNA, 23 bp, Artificial sequence
  Primer
gacgccaacaaggagagcagcaa

SEQ ID NO: 256 - DNA, 23 bp, Artificial sequence
  Primer
tctgcccagtcaagcccgtccaa

SEQ ID NO: 257 - DNA, 27 bp, Artificial sequence
  Primer
agatgtgaactttgtccccataaggat

SEQ ID NO: 258 - DNA, 29 bp, Artificial sequence
  Primer
acttgctacatttgtgatgaacaaggaag

SEQ ID NO: 259 - DNA, 31 bp, Artificial sequence
  Primer
aaatataaagagaaggacaaacacaaacaga

SEQ ID NO: 260 - DNA, 30 bp, Artificial sequence
  Primer
acttatacaagcactagcaacaactctata

SEQ ID NO: 261 - DNA, 27 bp, Artificial sequence
  Primer
gcaaatactctatctggatcttctctc

SEQ ID NO: 262 - DNA, 26 bp, Artificial sequence
  Primer
gatttggagttccatggagtgatgag

SEQ ID NO: 263 - DNA, 27 bp, Artificial sequence
  Primer
gattctgtttcactgaggccatctatc

SEQ ID NO: 264 - DNA, 23 bp, Artificial sequence
  Primer
cacatctccatcccccagcctga

SEQ ID NO: 265 - DNA, 24 bp, Artificial sequence
  Primer
gtggccaacatgagtgctaaggac

SEQ ID NO: 266 - DNA, 31 bp, Artificial sequence
  Primer
ttatcaagtgggaatcctgtatatgaaaaat

SEQ ID NO: 267 - DNA, 27 bp, Artificial sequence
  Primer
catgataccagtagtcctttgctaatc

SEQ ID NO: 268 - DNA, 27 bp, Artificial sequence
  Primer
agtcgaaaggacaaagaacgccttaag

SEQ ID NO: 269 - DNA, 23 bp, Artificial sequence
  Primer
gtctcctcctcggcttcctcttc

SEQ ID NO: 270 - DNA, 24 bp, Artificial sequence
  Primer
gtgttttctctggctggctctacc

SEQ ID NO: 271 - DNA, 23 bp, Artificial sequence
  Primer
ggtgaaggttgccgaactgtccc

SEQ ID NO: 272 - DNA, 23 bp, Artificial sequence
  Primer
gaagctatgagggaccctgtgag

SEQ ID NO: 273 - DNA, 24 bp, Artificial sequence
  Primer
gcacggacacttgctagtatgttg

SEQ ID NO: 274 - DNA, 24 bp, Artificial sequence
  Primer
gcatcagcttctggtgatgtgagc

SEQ ID NO: 275 - DNA, 23 bp, Artificial sequence
  Primer
gagatgacgcattcatggcctcc

SEQ ID NO: 276 - DNA, 24 bp, Artificial sequence
  Primer
gagtctcagcagtccaattttggc

SEQ ID NO: 277 - DNA, 26 bp, Artificial sequence
  Primer
atatttaacaccgtgcccgatatgcc

SEQ ID NO: 278 - DNA, 27 bp, Artificial sequence
  Primer
aagatggcagtgaacgtatactcaacg

SEQ ID NO: 279 - DNA, 30 bp, Artificial sequence
  Primer
ataattcctgtggacaaattagtaaaagga

SEQ ID NO: 280 - DNA, 30 bp, Artificial sequence
  Primer
gtcaacgtattgaaacttactgttgaagac

SEQ ID NO: 281 - DNA, 24 bp, Artificial sequence
  Primer
gacattgacaagcagtacgtgggc

SEQ ID NO: 282 - DNA, 23 bp, Artificial sequence
  Primer
gaggcacgcggacctccagtggc

SEQ ID NO: 283 - DNA, 27 bp, Artificial sequence
  Primer
ccttgaaaagatcttttgaggtcgagg

SEQ ID NO: 284 - DNA, 31 bp, Artificial sequence
  Primer
gaaaaaaaccttgaagataacttacagagtt

SEQ ID NO: 285 - DNA, 26 bp, Artificial sequence
  Primer
gagagtagatctggagaaaccaacag

SEQ ID NO: 286 - DNA, 23 bp, Artificial sequence
  Primer
gagatgacctggcttccaccact

SEQ ID NO: 287 - DNA, 23 bp, Artificial sequence
  Primer
caggcagctcagagaacggctct

SEQ ID NO: 288 - DNA, 31 bp, Artificial sequence
  Primer
atttttgatcaccatactgaagaggatatag

SEQ ID NO: 289 - DNA, 23 bp, Artificial sequence
  Primer
aactccatccggcacaacctgtc

SEQ ID NO: 290 - DNA, 27 bp, Artificial sequence
  Primer
ctccagggttccttgaaaagaaaacag

SEQ ID NO: 291 - DNA, 24 bp, Artificial sequence
  Primer
gatcaacactctgtggtaggccag

SEQ ID NO: 292 - DNA, 28 bp, Artificial sequence
  Primer
caagttggaattgacagaggtgatatac

SEQ ID NO: 293 - DNA, 23 bp, Artificial sequence
  Primer
gcccctagcagtcttcttgatgc

SEQ ID NO: 294 - DNA, 26 bp, Artificial sequence
  Primer
caacctcataatattctgcagaggcg

SEQ ID NO: 295 - DNA, 23 bp, Artificial sequence
  Primer
gacatgcggaagcacgtggccat

SEQ ID NO: 296 - DNA, 23 bp, Artificial sequence
  Primer
ggctacatgcagccgctgaagca

SEQ ID NO: 297 - DNA, 27 bp, Artificial sequence
  Primer
ttctccaaggatgtcctagtaaacatc

SEQ ID NO: 298 - DNA, 24 bp, Artificial sequence
  Primer
caaagcatgcgtgagaacaaggag

SEQ ID NO: 299 - DNA, 25 bp, Artificial sequence
  Primer
ccagaagtcattggatctgtgtcac

SEQ ID NO: 300 - DNA, 30 bp, Artificial sequence
  Primer
gtaaccatggagcttattacagataacaaa

SEQ ID NO: 301 - DNA, 24 bp, Artificial sequence
  Primer
gtgattcctgtctctctgtcttcc

SEQ ID NO: 302 - DNA, 23 bp, Artificial sequence
  Primer
gcagaggaccgaggaaatggact

SEQ ID NO: 303 - DNA, 24 bp, Artificial sequence
  Primer
gatggagctgtagttacaccctcc

SEQ ID NO: 304 - DNA, 24 bp, Artificial sequence
  Primer
agtcccaagagtggcccaaaagag

SEQ ID NO: 305 - DNA, 25 bp, Artificial sequence
  Primer
gacactcaatcacttgtcggaagtc

SEQ ID NO: 306 - DNA, 23 bp, Artificial sequence
  Primer
gtggaacggccgccttctccatt

SEQ ID NO: 307 - DNA, 23 bp, Artificial sequence
  Primer
gtgaatgaggcctctggggatgg

SEQ ID NO: 308 - DNA, 23 bp, Artificial sequence
  Primer
aagcctggaatggtcccccctcc

SEQ ID NO: 309 - DNA, 23 bp, Artificial sequence
  Primer
aactcgatccgccacaacctgtc

SEQ ID NO: 310 - DNA, 28 bp, Artificial sequence
  Primer
caacagccaactcagtttataaatccag

SEQ ID NO: 311 - DNA, 25 bp, Artificial sequence
  Primer
ctgcagaagaaagatcagcaactgg

SEQ ID NO: 312 - DNA, 26 bp, Artificial sequence
  Primer
atgggaataactgggaacacaagtcc

SEQ ID NO: 313 - DNA, 27 bp, Artificial sequence
  Primer
tctcagatgcaaacatcagtgggaatt

SEQ ID NO: 314 - DNA, 25 bp, Artificial sequence
  Primer
atgatggaggaggatttgcaaggag

SEQ ID NO: 315 - DNA, 25 bp, Artificial sequence
  Primer
gtgagtacccagaacatgaagatgg

SEQ ID NO: 316 - DNA, 23 bp, Artificial sequence
  Primer
ggcctggagcaggatgtcctcca

SEQ ID NO: 317 - DNA, 27 bp, Artificial sequence
  Primer
aatcagatgggtgactcaaatatctcc

SEQ ID NO: 318 - DNA, 24 bp, Artificial sequence
  Primer
tctgctccatctggacacaagcat

SEQ ID NO: 319 - DNA, 23 bp, Artificial sequence
  Primer
ctggagaatgctggaggagacct

SEQ ID NO: 320 - DNA, 26 bp, Artificial sequence
  Primer
aatactctggagcagtgcaatgtgtg

SEQ ID NO: 321 - DNA, 26 bp, Artificial sequence
  Primer
aatactctggagcagtgcaatgtgtg

SEQ ID NO: 322 - DNA, 27 bp, Artificial sequence
  Primer
agagaaatagcccggaaacttgcaaat

SEQ ID NO: 323 - DNA, 29 bp, Artificial sequence
  Primer
aatgaagagcttcgaaacttgtctttgtc

SEQ ID NO: 324 - DNA, 24 bp, Artificial sequence
  Primer
ccacagcgtcctgtgtttactcat

SEQ ID NO: 325 - DNA, 30 bp, Artificial sequence
  Primer
ttccaaaagctgagacaagatcttgaaatg

SEQ ID NO: 326 - DNA, 24 bp, Artificial sequence
  Primer
gtgacgacgtcatcaggaagcaag

SEQ ID NO: 327 - DNA, 23 bp, Artificial sequence
  Primer
gacaacagccggcgtgtggagca

SEQ ID NO: 328 - DNA, 24 bp, Artificial sequence
  Primer
gacttcctgacagacctgatgatg

SEQ ID NO: 329 - DNA, 30 bp, Artificial sequence
  Primer
gatcagtttgacaacttagaaaaacacaca

SEQ ID NO: 330 - DNA, 27 bp, Artificial sequence
  Primer
gctacagacaagagaaaagctttagag

SEQ ID NO: 331 - DNA, 28 bp, Artificial sequence
  Primer
actgtggatattcataaggagaaagtgg

SEQ ID NO: 332 - DNA, 30 bp, Artificial sequence
  Primer
gtcaagtacaaaagagattttgaagaaagc

SEQ ID NO: 333 - DNA, 31 bp, Artificial sequence
  Primer
gtaaaataccatgaagattttgaaaaaacaa

SEQ ID NO: 334 - DNA, 23 bp, Artificial sequence
  Primer
aagcggtaccgcgcggtgtatga

SEQ ID NO: 335 - DNA, 23 bp, Artificial sequence
  Primer
ctggtcagtgagaaggtcggagg

SEQ ID NO: 336 - DNA, 27 bp, Artificial sequence
  Primer
ttctccaatatccccttcttcatcttc

SEQ ID NO: 337 - DNA, 23 bp, Artificial sequence
  Primer
ggccctcctcaggacctgtctgt

SEQ ID NO: 338 - DNA, 23 bp, Artificial sequence
  Primer
ggcagcaaggagcgcttccactg

SEQ ID NO: 339 - DNA, 24 bp, Artificial sequence
  Primer
acctacatcggctctgtgctcatc

SEQ ID NO: 340 - DNA, 23 bp, Artificial sequence
  Primer
accttcacggcatggtgcaactc

SEQ ID NO: 341 - DNA, 23 bp, Artificial sequence
  Primer
cctatgggctatgggcctcgtat

SEQ ID NO: 342 - DNA, 25 bp, Artificial sequence
  Primer
gcacaaatgtctagttcttcctgcc

SEQ ID NO: 343 - DNA, 24 bp, Artificial sequence
  Primer
ctttcccagccagctgtaagcatt

SEQ ID NO: 344 - DNA, 28 bp, Artificial sequence
  Primer
gaaacaatgaccgataaaacagagaagg

SEQ ID NO: 345 - DNA, 23 bp, Artificial sequence
  Primer
ctctacgtctcctccgagagccg

SEQ ID NO: 346 - DNA, 23 bp, Artificial sequence
  Primer
gtctcctcctcggcttcctcttc

SEQ ID NO: 347 - DNA, 29 bp, Artificial sequence
  Primer
ataatctcagtgataccttgaagaagctg

SEQ ID NO: 348 - DNA, 28 bp, Artificial sequence
  Primer
aaatgacatcagatgtaccatcactggg

SEQ ID NO: 349 - DNA, 29 bp, Artificial sequence
  Primer
tcgcttcatggagatataagtagcctgaa

SEQ ID NO: 350 - DNA, 27 bp, Artificial sequence
  Primer
gaaattcggcgccttcatcagtatgtg

SEQ ID NO: 351 - DNA, 30 bp, Artificial sequence
  Primer
atcgtactgagaagcactccacaatgccag

SEQ ID NO: 352 - DNA, 33 bp, Artificial sequence
  Primer
caggaacgaatttcatatacacctccagagagc

SEQ ID NO: 353 - DNA, 26 bp, Artificial sequence
  Primer
gcttgcagccaatttactggagcagg

SEQ ID NO: 354 - DNA, 33 bp, Artificial sequence
  Primer
atcttagacgaattttacaatgtgaagttctgc

SEQ ID NO: 355 - DNA, 24 bp, Artificial sequence
  Primer
gtgtctgctgactccagtgcatcc

SEQ ID NO: 356 - DNA, 28 bp, Artificial sequence
  Primer
attgtcagtaaacgggcaggtactcagt

SEQ ID NO: 357 - DNA, 27 bp, Artificial sequence
  Primer
gatgacagcatggaagagaaaccacta

SEQ ID NO: 358 - DNA, 23 bp, Artificial sequence
  Primer
tgagtccattcttgcacaccgag

SEQ ID NO: 359 - DNA, 27 bp, Artificial sequence
  Primer
attttggctctctatttgacttggagc

SEQ ID NO: 360 - DNA, 24 bp, Artificial sequence
  Primer
atgggaatgatgaacaaccccaat

SEQ ID NO: 361 - DNA, 23 bp, Artificial sequence
  Primer
ccttgccctttaaggtggtggtg

SEQ ID NO: 362 - DNA, 28 bp, Artificial sequence
  Primer
ctgccttatgactcaagatgggagtttc

SEQ ID NO: 363 - DNA, 22 bp, Artificial sequence
  Primer
gggccccgagaacctcgaaatc

SEQ ID NO: 364 - DNA, 22 bp, Artificial sequence
  Primer
gggcgacctcttccagaagctg

SEQ ID NO: 365 - DNA, 27 bp, Artificial sequence
  Primer
atccaacgaccaagaactctctggaaa

SEQ ID NO: 366 - DNA, 28 bp, Artificial sequence
  Primer
gccagttatttgcctcagtaaaagacag

SEQ ID NO: 367 - DNA, 30 bp, Artificial sequence
  Primer
cagtatgagtacacggagctcaagaaacag

SEQ ID NO: 368 - DNA, 28 bp, Artificial sequence
  Primer
cacaatgaaacagatttgcaaaaaggaa

SEQ ID NO: 369 - DNA, 20 bp, Artificial sequence
  Primer
ccgcctgcctgcgcacctgc

SEQ ID NO: 370 - DNA, 22 bp, Artificial sequence
  Primer
cgggcagaaggctggtgacaag

SEQ ID NO: 371 - DNA, 20 bp, Artificial sequence
  Primer
aagggccaaccactggggaa

SEQ ID NO: 372 - DNA, 30 bp, Artificial sequence
  Primer
aaaacaagtgcacagacaacaccaagtaag

SEQ ID NO: 373 - DNA, 26 bp, Artificial sequence
  Primer
gggcaaatgagaacagcaacatacag

SEQ ID NO: 374 - DNA, 26 bp, Artificial sequence
  Primer
gcacacctctcaatgcagctttacag

SEQ ID NO: 375 - DNA, 29 bp, Artificial sequence
  Primer
tgaggacaagttctacagccacaagaaaa

SEQ ID NO: 376 - DNA, 24 bp, Artificial sequence
  Primer
agaaccccaacagcaaagaaggct

SEQ ID NO: 377 - DNA, 25 bp, Artificial sequence
  Primer
cctaagatgcccgacttcaactgct

SEQ ID NO: 378 - DNA, 24 bp, Artificial sequence
  Primer
aatgctggacttccgtagtgacca

SEQ ID NO: 379 - DNA, 30 bp, Artificial sequence
  Primer
cagtgccaggaaagagaattagagatcagt

SEQ ID NO: 380 - DNA, 23 bp, Artificial sequence
  Primer
caaagcccacgctgaagcgaaag

SEQ ID NO: 381 - DNA, 26 bp, Artificial sequence
  Primer
agctaaaaggacagcaggtgctacca

SEQ ID NO: 382 - DNA, 28 bp, Artificial sequence
  Primer
gttcagagactgaaggatgaagccagag

SEQ ID NO: 383 - DNA, 29 bp, Artificial sequence
  Primer
gatgatgatgaagaggatgatgatgaaga

SEQ ID NO: 384 - DNA, 24 bp, Artificial sequence
  Primer
cagcttccagtcacagcataggct

SEQ ID NO: 385 - DNA, 28 bp, Artificial sequence
  Primer
gatgtcagcatacccagatccacattag

SEQ ID NO: 386 - DNA, 24 bp, Artificial sequence
  Primer
accgcttggaatgctgcaacaatg

SEQ ID NO: 387 - DNA, 27 bp, Artificial sequence
  Primer
ccaacaggcatgataggatatggaatt

SEQ ID NO: 388 - DNA, 25 bp, Artificial sequence
  Primer
ctttggccctgtatcaggagcacag

SEQ ID NO: 389 - DNA, 21 bp, Artificial sequence
  Primer
ctgggccagaatggtgaggag

SEQ ID NO: 390 - DNA, 23 bp, Artificial sequence
  Primer
ggctggcgcacctgggcgtgcag

SEQ ID NO: 391 - DNA, 25 bp, Artificial sequence
  Primer
tgatgaaggggaggaaggagaggaa

SEQ ID NO: 392 - DNA, 28 bp, Artificial sequence
  Primer
cgtgatctttgatagatccagggaagag

SEQ ID NO: 393 - DNA, 27 bp, Artificial sequence
  Primer
ttcttggctcaacaagccataaaacag

SEQ ID NO: 394 - DNA, 29 bp, Artificial sequence
  Primer
ggtcttataatcttccctctcttccggat

SEQ ID NO: 395 - DNA, 25 bp, Artificial sequence
  Primer
ccagcaaccttccatctctcatcag

SEQ ID NO: 396 - DNA, 24 bp, Artificial sequence
  Primer
ccgcagagcactgtatcacgaaaa

SEQ ID NO: 397 - DNA, 30 bp, Artificial sequence
  Primer
attcctgttcctactacagttcctgttcct

SEQ ID NO: 398 - DNA, 29 bp, Artificial sequence
  Primer
accaagaggctattcaagatctctgcctg

SEQ ID NO: 399 - DNA, 29 bp, Artificial sequence
  Primer
accaagaggctattcaagatctctgtatg

SEQ ID NO: 400 - DNA, 30 bp, Artificial sequence
  Primer
agcatttggttttaaattatggagtatgtt

SEQ ID NO: 401 - DNA, 26 bp, Artificial sequence
  Primer
tctgtggagacgagaatattctggtt

SEQ ID NO: 402 - DNA, 27 bp, Artificial sequence
  Primer
gatttgtgattttggtctagccagagt

SEQ ID NO: 403 - DNA, 31 bp, Artificial sequence
  Primer
catcaagaatgattctaattatgtggttaaa

SEQ ID NO: 404 - DNA, 22 bp, Artificial sequence
  Primer
catcccagtgactgcatccctc

SEQ ID NO: 405 - DNA, 20 bp, Artificial sequence
  Primer
ggggaccccattcccgagga

SEQ ID NO: 406 - DNA, 27 bp, Artificial sequence
  Primer
gctctccacagatagagaacatccagc

SEQ ID NO: 407 - DNA, 25 bp, Artificial sequence
  Primer
ctgaacagatgggtaaggatggcag

SEQ ID NO: 408 - DNA, 23 bp, Artificial sequence
  Primer
ggaccaaccacttcctaccccag

SEQ ID NO: 409 - DNA, 20 bp, Artificial sequence
  Primer
gccccaggtgtacccaccac

SEQ ID NO: 410 - DNA, 20 bp, Artificial sequence
  Primer
gcctcacctgcagatgcccc

SEQ ID NO: 411 - DNA, 25 bp, Artificial sequence
  Primer
gcaacctccaagtcccagatcatgt

SEQ ID NO: 412 - DNA, 20 bp, Artificial sequence
  Primer
ggagttcctggtcggctccg

SEQ ID NO: 413 - DNA, 27 bp, Artificial sequence
  Primer
cgagttcaagcaggcctatatcacctg

SEQ ID NO: 414 - DNA, 20 bp, Artificial sequence
  Primer
gtgggcctcctgggcctcag

SEQ ID NO: 415 - DNA, 23 bp, Artificial sequence
  Primer
tccctggaatgaagggacacaga

SEQ ID NO: 416 - DNA, 20 bp, Artificial sequence
  Primer
atggcaaaactggcccccct

SEQ ID NO: 417 - DNA, 23 bp, Artificial sequence
  Primer
tccctggacctaaaggtgctgct

SEQ ID NO: 418 - DNA, 23 bp, Artificial sequence
  Primer
aagcaggcaaacctggtgaacag

SEQ ID NO: 419 - DNA, 22 bp, Artificial sequence
  Primer
tccagggcctaagggtgacaga

SEQ ID NO: 420 - DNA, 20 bp, Artificial sequence
  Primer
ctggtgcccctggtgacaag

SEQ ID NO: 421 - DNA, 20 bp, Artificial sequence
  Primer
ctggaccccctggccccatt

SEQ ID NO: 422 - DNA, 20 bp, Artificial sequence
  Primer
agggtccccctggccctcct

SEQ ID NO: 423 - DNA, 20 bp, Artificial sequence
  Primer
ctggtcctgctggtccccga

SEQ ID NO: 424 - DNA, 20 bp, Artificial sequence
  Primer
ctggcgagcctggagcttca

SEQ ID NO: 425 - DNA, 22 bp, Artificial sequence
  Primer
tgtcctccttgaagggctccag

SEQ ID NO: 426 - DNA, 27 bp, Artificial sequence
  Primer
cctccactgaagaagctgaaacaagag

SEQ ID NO: 427 - DNA, 25 bp, Artificial sequence
  Primer
gagagtctggatggacatttgcagg

SEQ ID NO: 428 - DNA, 20 bp, Artificial sequence
  Primer
tgcgaagccacctctcgcag

SEQ ID NO: 429 - DNA, 30 bp, Artificial sequence
  Primer
ggagcggacatggactacgactcgtaccag

SEQ ID NO: 430 - DNA, 30 bp, Artificial sequence
  Primer
agaatgaagaaattgatgttgtgacagtag

SEQ ID NO: 431 - DNA, 26 bp, Artificial sequence
  Primer
aggaaggcatcaaccacgagtgtaag

SEQ ID NO: 432 - DNA, 28 bp, Artificial sequence
  Primer
cagctctctgtaatgcgatactcaccag

SEQ ID NO: 433 - DNA, 30 bp, Artificial sequence
  Primer
aagagaggctgtatctccatgccagagcag

SEQ ID NO: 434 - DNA, 30 bp, Artificial sequence
  Primer
acccaaaagcagaccttggagaacagtcag

SEQ ID NO: 435 - DNA, 30 bp, Artificial sequence
  Primer
tctttgaactactgccggagcttctgccag

SEQ ID NO: 436 - DNA, 24 bp, Artificial sequence
  Primer
caccgaggaccagctcaatgacag

SEQ ID NO: 437 - DNA, 25 bp, Artificial sequence
  Primer
acatttcatggggctccactaacag

SEQ ID NO: 438 - DNA, 23 bp, Artificial sequence
  Primer
gctgcctgcgtcccaaagaacag

SEQ ID NO: 439 - DNA, 27 bp, Artificial sequence
  Primer
ctacagagacacaacccattgtttatg

SEQ ID NO: 440 - DNA, 27 bp, Artificial sequence
  Primer
gtgggaacgtgaaacatctgatacaag

SEQ ID NO: 441 - DNA, 24 bp, Artificial sequence
  Primer
ggaagattgcccgagagcaaaaag

SEQ ID NO: 442 - DNA, 27 bp, Artificial sequence
  Primer
tgcatattagtggacagcacttagtag

SEQ ID NO: 443 - DNA, 23 bp, Artificial sequence
  Primer
gcagcccaagcttcccatcacag

SEQ ID NO: 444 - DNA, 27 bp, Artificial sequence
  Primer
gaccttccaccaatattcctgaaaatg

SEQ ID NO: 445 - DNA, 27 bp, Artificial sequence
  Primer
ttggcttaacagatgatcaggtttcag

SEQ ID NO: 446 - DNA, 27 bp, Artificial sequence
  Primer
ctcagactcaagcaggtcagattgaag

SEQ ID NO: 447 - DNA, 27 bp, Artificial sequence
  Primer
ctcaacagtatggtattcagtattcag

SEQ ID NO: 448 - DNA, 23 bp, Artificial sequence
  Primer
atatgccctgcgtccaagcccaa

SEQ ID NO: 449 - DNA, 23 bp, Artificial sequence
  Primer
agcccactgcggaagagggcagc

SEQ ID NO: 450 - DNA, 30 bp, Artificial sequence
  Primer
ctgcatcaggagatatgcaaacatatcaga

SEQ ID NO: 451 - DNA, 22 bp, Artificial sequence
  Primer
ttgccattgccccaaatggagc

SEQ ID NO: 452 - DNA, 30 bp, Artificial sequence
  Primer
aaaaattttgaaagacttatcttctgaaga

SEQ ID NO: 453 - DNA, 30 bp, Artificial sequence
  Primer
gtatcatctttatcagaaagtgaggagtcc

SEQ ID NO: 454 - DNA, 23 bp, Artificial sequence
  Primer
ttgccattacccagggaggagca

SEQ ID NO: 455 - DNA, 27 bp, Artificial sequence
  Primer
ctgcctctggagacgtacaaacatacc

SEQ ID NO: 456 - DNA, 28 bp, Artificial sequence
  Primer
atgtgcagcacattaagaggagagacat

SEQ ID NO: 457 - DNA, 23 bp, Artificial sequence
  Primer
gtgcagacccatctggagaaccc

SEQ ID NO: 458 - DNA, 29 bp, Artificial sequence
  Primer
attgatgatgtcattgatgagatcatcag

SEQ ID NO: 459 - DNA, 24 bp, Artificial sequence
  Primer
ctgcctgtgtcagggaatctgctt

SEQ ID NO: 460 - DNA, 26 bp, Artificial sequence
  Primer
ggaaaactacagccaccacacacaag

SEQ ID NO: 461 - DNA, 28 bp, Artificial sequence
  Primer
acacctttcatgaactcaaatctgatgg

SEQ ID NO: 462 - DNA, 29 bp, Artificial sequence
  Primer
ggtcatactgcatcagaaccatgaagaag

SEQ ID NO: 463 - DNA, 24 bp, Artificial sequence
  Primer
ccatgcccattgggagaatagcag

SEQ ID NO: 464 - DNA, 23 bp, Artificial sequence
  Primer
agcaggagcaggagcgggagcgg

SEQ ID NO: 465 - DNA, 23 bp, Artificial sequence
  Primer
cgccggagttgcataagggagat

SEQ ID NO: 466 - DNA, 24 bp, Artificial sequence
  Primer
ggttccatgatgggaagtgacatg

SEQ ID NO: 467 - DNA, 28 bp, Artificial sequence
  Primer
tggccaatgtgatctggaacttattaat

SEQ ID NO: 468 - DNA, 23 bp, Artificial sequence
  Primer
tcaagggaaccttccctgatgcg

SEQ ID NO: 469 - DNA, 27 bp, Artificial sequence
  Primer
cagtgatctggcctcagacaactactg

SEQ ID NO: 470 - DNA, 29 bp, Artificial sequence
  Primer
gcataagctggaagtcacaccagtagtag

SEQ ID NO: 471 - DNA, 27 bp, Artificial sequence
  Primer
aggttgaaattgggtcttcaaaaccag

SEQ ID NO: 472 - DNA, 23 bp, Artificial sequence
  Primer
tcaggatgggaaaattgcaccag

SEQ ID NO: 473 - DNA, 35 bp, Artificial sequence
  Primer
gagaatatataaaaaactggaggccaagatacttc

SEQ ID NO: 474 - DNA, 30 bp, Artificial sequence; identical to SEQ ID NO: 210
  Primer

SEQ ID NO: 475 - DNA, 30 bp, Artificial sequence; identical to SEQ ID NO: 211
  Primer

SEQ ID NO: 476 - DNA, 30 bp, Artificial sequence; identical to SEQ ID NO: 450
  Primer

SEQ ID NO: 477 - DNA, 22 bp, Artificial sequence
  Primer
ttgccattgccccaaatggagc

SEQ ID NO: 478 - DNA, 30 bp, Artificial sequence; identical to SEQ ID NO: 452
  Primer

SEQ ID NO: 479 - DNA, 30 bp, Artificial sequence; identical to SEQ ID NO: 453
  Primer

SEQ ID NO: 480 - DNA, 41 bp, Artificial sequence
  Primer
aagcccttcagcggccagtagctccaacccttagggaaccc

SEQ ID NO: 481 - DNA, 42 bp, Artificial sequence
  Primer
gtgaaaagctccgggtcttaggctccaacccttagggaaccc

SEQ ID NO: 482 - DNA, 45 bp, Artificial sequence
  Primer
gtgccagcaagatccaatctagaggcgccttccatggagacgcag

SEQ ID NO: 483 - DNA, 48 bp, Artificial sequence
  Primer
gtgccagcaagatccaatctagaattccgctgaccatcaataaggaag

SEQ ID NO: 484 - DNA, 49 bp, Artificial sequence
  Primer
gtgccagcaagatccaatctagaagccactggatttaagcagagttcaa

SEQ ID NO: 485 - DNA, 46 bp, Artificial sequence
  Primer
gtgccagcaagatccaatctagaactgaaggcagccttcgacgtca

SEQ ID NO: 486 - DNA, 47 bp, Artificial sequence
  Primer
gtgccagcaagatccaatctagagctcttgcatcacccaggggaaag

SEQ ID NO: 487 - DNA, 43 bp, Artificial sequence
  Primer
gtgccagcaagatccaatctagacagtggcgccggggaggcag

SEQ ID NO: 488 - DNA, 44 bp, Artificial sequence
  Primer
ccattgagacccagagcagcagttctccaacccttagggaaccc

SEQ ID NO: 489 - DNA, 47 bp, Artificial sequence
  Primer
gtgccagcaagatccaatctagagagtttgatgaggagcgagcccag

SEQ ID NO: 490 - DNA, 48 bp, Artificial sequence
  Primer
gtgccagcaagatccaatctagacaggtctcatcgggaggaaatggag

SEQ ID NO: 491 - DNA, 49 bp, Artificial sequence
  Primer
gtgccagcaagatccaatctagaagtttcacagctgctggcagtaactg

SEQ ID NO: 492 - DNA, 46 bp, Artificial sequence
  Primer
gccaaggcgaacctagacaagaataagtccaacccttagggaaccc

SEQ ID NO: 493 - DNA, 45 bp, Artificial sequence
  Primer
aatgaagttgagagcgtcacagggattccaacccttagggaaccc

SEQ ID NO: 494 - DNA, 44 bp, Artificial sequence
  Primer
gagctgcttcaagaagaaacccggctccaacccttagggaaccc

SEQ ID NO: 495 - DNA, 44 bp, Artificial sequence
  Primer
ctctccgactcgaagaagaagctgctccaacccttagggaaccc

SEQ ID NO: 496 - DNA, 48 bp, Artificial sequence
  Primer
ttgttagccgaggagaaaaacatctcttctccaacccttagggaaccc

SEQ ID NO: 497 - DNA, 44 bp, Artificial sequence
  Primer
gtccatgagctggagaagtccaagctccaacccttagggaaccc

SEQ ID NO: 498 - DNA, 47 bp, Artificial sequence
  Primer
cttcacgagtatgagacggaactggaagtccaacccttagggaaccc

SEQ ID NO: 499 - DNA, 48 bp, Artificial sequence
  Primer
gtgccagcaagatccaatctagatctcggcctcccgactcctacagtg

SEQ ID NO: 500 - DNA, 45 bp, Artificial sequence
  Primer
ttttgagtatccgaggagcccaggagtccaacccttagggaaccc

SEQ ID NO: 501 - DNA, 52 bp, Artificial sequence
  Primer
gtgccagcaagatccaatctagaggtcatactgcatcagaaccatgaagaag

SEQ ID NO: 502 - DNA, 47 bp, Artificial sequence
  Primer
gtgccagcaagatccaatctagaccatgcccattgggagaatagcag

SEQ ID NO: 503 - DNA, 49 bp, Artificial sequence
  Primer
aatgcatacttggaatgaatccttctagagtccaacccttagggaaccc

SEQ ID NO: 504 - DNA, 42 bp, Artificial sequence
  Primer
atgccagcacgagccgccgcttctccaacccttagggaaccc

SEQ ID NO: 505 - DNA, 47 bp, Artificial sequence
  Primer
gtgccagcaagatccaatctagatggatgggccccgagaacctcgaa

SEQ ID NO: 506 - DNA, 46 bp, Artificial sequence
  Primer
atcgtactgagaagcactccacaatgctccaacccttagggaaccc

SEQ ID NO: 507 - DNA, 53 bp, Artificial sequence
  Primer
gtgccagcaagatccaatctagaatccctgtaaaacaaaaaccaaaagaaaag

SEQ ID NO: 508 - DNA, 51 bp, Artificial sequence
  Primer
gtgccagcaagatccaatctagaagtccacaggatcagagtggactttaag

SEQ ID NO: 509 - DNA, 50 bp, Artificial sequence
  Primer
gtgccagcaagatccaatctagactctgtgccagtagtgggcatgtagag

SEQ ID NO: 510 - DNA, 49 bp, Artificial sequence
  Primer
gtgccagcaagatccaatctagagtggaaggcaacatcaggctacaaag

SEQ ID NO: 511 - DNA, 45 bp, Artificial sequence
  Primer
cagacctactccaatgaagtccattgtccaacccttagggaaccc

SEQ ID NO: 512 - DNA, 42 bp, Artificial sequence
  Primer
gaaatgacccattcatggccgcctccaacccttagggaaccc

SEQ ID NO: 513 - DNA, 45 bp, Artificial sequence
  Primer
gactctcagcatgtcagttctgtaactccaacccttagggaaccc

SEQ ID NO: 514 - DNA, 45 bp, Artificial sequence
  Primer
cctgagcctccaacaacaaacaaatgtccaacccttagggaaccc

SEQ ID NO: 515 - DNA, 45 bp, Artificial sequence
  Primer
gtgccagcaagatccaatctagaagcagcagctacgggcagcaga

SEQ ID NO: 516 - DNA, 45 bp, Artificial sequence
  Primer
gtgccagcaagatccaatctagagaggaggacgcggtggaatggg

SEQ ID NO: 517 - DNA, 47 bp, Artificial sequence
  Primer
gtgccagcaagatccaatctagagaggtggcttcaataagcctggtg

SEQ ID NO: 518 - DNA, 50 bp, Artificial sequence
  Primer
gtgccagcaagatccaatctagatggatgaaggaccagatcttgatctag

SEQ ID NO: 519 - DNA, 47 bp, Artificial sequence
  Primer
gttcactgctggcctataatacaacctctccaacccttagggaaccc

SEQ ID NO: 520 - DNA, 49 bp, Artificial sequence
  Primer
acccttcttatgactcagtcagaagaggagtccaacccttagggaaccc

SEQ ID NO: 521 - DNA, 42 bp, Artificial sequence
  Primer
gtcctccccttggaggggcacaatccaacccttagggaaccc

SEQ ID NO: 522 - DNA, 45 bp, Artificial sequence
  Primer
gttattccaggatctttggagacccgtccaacccttagggaaccc

SEQ ID NO: 523 - DNA, 47 bp, Artificial sequence
  Primer
gaagccttatcagttgtgagtgaggacctccaacccttagggaaccc

SEQ ID NO: 524 - DNA, 43 bp, Artificial sequence
  Primer
atttaccatatgagccccccaggatccaacccttagggaaccc

SEQ ID NO: 525 - DNA, 44 bp, Artificial sequence
  Primer
ctgctcaaccatctccttccacagttccaacccttagggaaccc

SEQ ID NO: 526 - DNA, 49 bp, Artificial sequence
  Primer
atccttatcagattcttggaccaacaagtatccaacccttagggaaccc

SEQ ID NO: 527 - DNA, 41 bp, Artificial sequence
  Primer
gcagtggccagatccagctttgtccaacccttagggaaccc

SEQ ID NO: 528 - DNA, 42 bp, Artificial sequence
  Primer
atcccgtcggagacggtctcttctccaacccttagggaaccc

SEQ ID NO: 529 - DNA, 47 bp, Artificial sequence
  Primer
ctcaggtacctgacaatgatgagcagtttccaacccttagggaaccc

SEQ ID NO: 530 - DNA, 48 bp, Artificial sequence
  Primer
gagacatcaaacaagagccaggaatgtattccaacccttagggaaccc

SEQ ID NO: 531 - DNA, 41 bp, Artificial sequence
  Primer
atgtcaccgggtgcgcatcaattccaacccttagggaaccc

SEQ ID NO: 532 - DNA, 47 bp, Artificial sequence
  Primer
gtgccagcaagatccaatctagaattggcaatggcctctcacctcag

SEQ ID NO: 533 - DNA, 49 bp, Artificial sequence
  Primer
aattcaattcgtcataatctgtccctacactccaacccttagggaaccc

SEQ ID NO: 534 - DNA, 48 bp, Artificial sequence
  Primer
gtgagaaaccataccagtgtgacttcaagtccaacccttagggaaccc

SEQ ID NO: 535 - DNA, 46 bp, Artificial sequence
  Primer
gtgccagcaagatccaatctagacccaggacagcagcagggctacg

SEQ ID NO: 536 - DNA, 49 bp, Artificial sequence
  Primer
gtgccagcaagatccaatctagaagcagaggccttatggatatgaccag

SEQ ID NO: 537 - DNA, 42 bp, Artificial sequence
  Primer
atcatgcccaagaagccagcagatccaacccttagggaaccc

SEQ ID NO: 538 - DNA, 44 bp, Artificial sequence
  Primer
gtttcaaagtcaccctcccaccttttccaacccttagggaaccc

SEQ ID NO: 539 - DNA, 49 bp, Artificial sequence
  Primer
tgttcaagaaggaagtgtatcttcatacattccaacccttagggaaccc

SEQ ID NO: 540 - DNA, 46 bp, Artificial sequence
  Primer
atgtgcttttccagactgatccaactgtccaacccttagggaaccc

SEQ ID NO: 541 - DNA, 46 bp, Artificial sequence
  Primer
gtgccagcaagatccaatctagatggaggtggaggtggaggtggag

SEQ ID NO: 542 - DNA, 46 bp, Artificial sequence
  Primer
gtgccagcaagatccaatctagacgtggaggcagaggtggcatggg

SEQ ID NO: 543 - DNA, 48 bp, Artificial sequence
  Primer
gtgccagcaagatccaatctagacgtggtggcttcaataaatttggtg

SEQ ID NO: 544 - DNA, 47 bp, Artificial sequence
  Primer
gtgccagcaagatccaatctagaaccaaggatcacgtcatgactccg

SEQ ID NO: 545 - DNA, 46 bp, Artificial sequence
  Primer
gtgccagcaagatccaatctagaggctgccgtggaatggtttgatg

SEQ ID NO: 546 - DNA, 51 bp, Artificial sequence
  Primer
gtgccagcaagatccaatctagacttctttaagcagtgtggggttgttaag

SEQ ID NO: 547 - DNA, 45 bp, Artificial sequence; identical to SEQ ID NO: 522
  Primer

SEQ ID NO: 548 - DNA, 47 bp, Artificial sequence; identical to SEQ ID NO: 523
  Primer

SEQ ID NO: 549 - DNA, 43 bp, Artificial sequence; identical to SEQ ID NO: 524
  Primer

SEQ ID NO: 550 - DNA, 44 bp, Artificial sequence; identical to SEQ ID NO: 525
  Primer

SEQ ID NO: 551 - DNA, 49 bp, Artificial sequence; identical to SEQ ID NO: 526
  Primer

SEQ ID NO: 552 - DNA, 41 bp, Artificial sequence; identical to SEQ ID NO: 527
  Primer

SEQ ID NO: 553 - DNA, 45 bp, Artificial sequence
  Primer
gtgccagcaagatccaatctagaagcgccgcctggagcgcggcag

SEQ ID NO: 554 - DNA, 50 bp, Artificial sequence
  Primer
gtgccagcaagatccaatctagagataacagcaagatggctttgaactca

SEQ ID NO: 555 - DNA, 42 bp, Artificial sequence
  Primer
tgtaccgccggaagcaccaggagtccaacccttagggaaccc

SEQ ID NO: 556 - DNA, 46 bp, Artificial sequence
  Primer
gtgccagcaagatccaatctagaggctggaaacatttccgaccctg

SEQ ID NO: 557 - DNA, 50 bp, Artificial sequence
  Primer
gtgccagcaagatccaatctagatggaaaagacaattgatgacctggaag

SEQ ID NO: 558 - DNA, 50 bp, Artificial sequence
  Primer
gtgccagcaagatccaatctagacagtgaaaaaatcagtctcaagtaaag

SEQ ID NO: 559 - DNA, 50 bp, Artificial sequence
  Primer
gtgccagcaagatccaatctagaagcataaagatgtcatcatcaaccaag

SEQ ID NO: 560 - DNA, 48 bp, Artificial sequence
  Primer
gtgccagcaagatccaatctagacccacacctgggaaaggacctaaag

SEQ ID NO: 561 - DNA, 50 bp, Artificial sequence
  Primer
gtgccagcaagatccaatctagagatctgaatcctgaaagagaaatagag

SEQ ID NO: 562 - DNA, 46 bp, Artificial sequence
  Primer
gtgccagcaagatccaatctagagccataggaacgcactcaggcag

SEQ ID NO: 563 - DNA, 50 bp, Artificial sequence
  Primer
gtgccagcaagatccaatctagaagctctctgtgatgcgctactcaatag

SEQ ID NO: 564 - DNA, 50 bp, Artificial sequence
  Primer
gtgccagcaagatccaatctagaactcgggagactatgaaatattgtact

SEQ ID NO: 565 - DNA, 44 bp, Artificial sequence
  Primer
ctggagtcccaaataaaccaggcattccaacccttagggaaccc

SEQ ID NO: 566 - DNA, 49 bp, Artificial sequence
  Primer
atgatttttggataccagaaacaagtttcatccaacccttagggaaccc

SEQ ID NO: 567 - DNA, 49 bp, Artificial sequence
  Primer
tctggcatagaagattaaagaatcaaaaaatccaacccttagggaaccc

SEQ ID NO: 568 - DNA, 46 bp, Artificial sequence
  Primer
gtgccagcaagatccaatctagaagctgtctggctctggagatctg

SEQ ID NO: 569 - DNA, 46 bp, Artificial sequence
  Primer
gtgccagcaagatccaatctagatgagagaacggaggtcctggcag

SEQ ID NO: 570 - DNA, 50 bp, Artificial sequence
  Primer
gtgccagcaagatccaatctagaacataaccattagcagagaggctcagg

SEQ ID NO: 571 - DNA, 46 bp, Artificial sequence
  Primer
gtgccagcaagatccaatctagagcccactgacgctccaccgaaag

SEQ ID NO: 572 - DNA, 49 bp, Artificial sequence
  Primer
gtgccagcaagatccaatctagaggagaagacaaagaaggcagagagag

SEQ ID NO: 573 - DNA, 50 bp, Artificial sequence
  Primer
gtgccagcaagatccaatctagattttcttaccacaacatgacagtagtg

SEQ ID NO: 574 - DNA, 42 bp, Artificial sequence
  Primer
atccactgtgcgacgagctgtgctccaacccttagggaaccc

SEQ ID NO: 575 - DNA, 43 bp, Artificial sequence
  Primer
gaggatccaaagtgggaattcccttccaacccttagggaaccc

SEQ ID NO: 576 - DNA, 50 bp, Artificial sequence
  Primer
gtgccagcaagatccaatctagaattgctgtgggaaataatgatgtaaag

SEQ ID NO: 577 - DNA, 50 bp, Artificial sequence
  Primer
gtgccagcaagatccaatctagagcagcatgtcagcttcgtatctctcaa

SEQ ID NO: 578 - DNA, 50 bp, Artificial sequence
  Primer
gtgccagcaagatccaatctagaaagaactagtccagcttcgagcacaag

SEQ ID NO: 579 - DNA, 50 bp, Artificial sequence
  Primer
gtgccagcaagatccaatctagacaggacctggctacaagagttaaaaag

SEQ ID NO: 580 - DNA, 50 bp, Artificial sequence
  Primer
gtgccagcaagatccaatctagagaacagctcactaaagtgcacaaacag

SEQ ID NO: 581 - DNA, 48 bp, Artificial sequence
  Primer
gtgccagcaagatccaatctagaagaagagggcattctgcacagattg

SEQ ID NO: 582 - DNA, 46 bp, Artificial sequence
  Primer
gtgccagcaagatccaatctagatgcgcaaagccagcgtgaccatc

SEQ ID NO: 583 - DNA, 46 bp, Artificial sequence
  Primer
gtgccagcaagatccaatctagaacctcagctccgcggaagttgcg

SEQ ID NO: 584 - DNA, 41 bp, Artificial sequence
  Primer
atcgcccaggaccacaccgcagtccaacccttagggaaccc

SEQ ID NO: 585 - DNA, 41 bp, Artificial sequence
  Primer
gatgaccgagcggccgccgagctccaacccttagggaaccc

SEQ ID NO: 586 - DNA, 41 bp, Artificial sequence
  Primer
gtccccacaccaaagttgtgcgtccaacccttagggaaccc

SEQ ID NO: 587 - DNA, 46 bp, Artificial sequence
  Primer
gtgccagcaagatccaatctagagcagatggccagtcaggcaccag

SEQ ID NO: 588 - DNA, 52 bp, Artificial sequence
  Primer
gtgccagcaagatccaatctagatcagttatcatctggtgacaaagcttcag

SEQ ID NO: 589 - DNA, 50 bp, Artificial sequence
  Primer
gtgccagcaagatccaatctagacgtcttctaatttcactgctgcacaag

SEQ ID NO: 590 - DNA, 47 bp, Artificial sequence
  Primer
gtgccagcaagatccaatctagaggttcagcttttgccaagcttcag

SEQ ID NO: 591 - DNA, 48 bp, Artificial sequence
  Primer
gtgccagcaagatccaatctagaggctttggatccacagctacctcaa

SEQ ID NO: 592 - DNA, 49 bp, Artificial sequence
  Primer
gtgccagcaagatccaatctagatgggttttcctctccaaacaaaacag

SEQ ID NO: 593 - DNA, 46 bp, Artificial sequence
  Primer
gtgccagcaagatccaatctagatggttttggatcaggcacaggag

SEQ ID NO: 594 - DNA, 49 bp, Artificial sequence
  Primer
attatgaactattaacagaaaatgacatgttccaacccttagggaaccc

SEQ ID NO: 595 - DNA, 47 bp, Artificial sequence
  Primer
ttcttcaggagagaataccatgggtacctccaacccttagggaaccc

SEQ ID NO: 596 - DNA, 49 bp, Artificial sequence
  Primer
gaaattgaacttagctcattaagggaagcttccaacccttagggaaccc

SEQ ID NO: 597 - DNA, 49 bp, Artificial sequence
  Primer
cgagaaaatgtcattgaatataaacactgttccaacccttagggaaccc

SEQ ID NO: 598 - DNA, 53 bp, Artificial sequence
  Primer
gtgccagcaagatccaatctagacctagtgagagccttgctactactgatgat

SEQ ID NO: 599 - DNA, 53 bp, Artificial sequence
  Primer
gtgccagcaagatccaatctagaactgaatctccagtgttagtgaatgactat

SEQ ID NO: 600 - DNA, 53 bp, Artificial sequence
  Primer
gtgccagcaagatccaatctagacagtgcatattagtggacagcacttagtag

SEQ ID NO: 601 - DNA, 51 bp, Artificial sequence
  Primer
gtgccagcaagatccaatctagactgagaatgcacttactggctcattcag

SEQ ID NO: 602 - DNA, 49 bp, Artificial sequence
  Primer
gtgccagcaagatccaatctagaggagacacacaggcagacccatactg

SEQ ID NO: 603 - DNA, 51 bp, Artificial sequence
  Primer
gtgccagcaagatccaatctagaatcaccattgcttggaagtttgattctc

SEQ ID NO: 604 - DNA, 49 bp, Artificial sequence
  Primer
gtgccagcaagatccaatctagaaccagttccctgcgagtctgctactg

SEQ ID NO: 605 - DNA, 50 bp, Artificial sequence
  Primer
gtgccagcaagatccaatctagaattacctggtcatgatcattgtccgtg

SEQ ID NO: 606 - DNA, 45 bp, Artificial sequence
  Primer
tgctacagttgaaactccagcagcgctccaacccttagggaaccc

SEQ ID NO: 607 - DNA, 45 bp, Artificial sequence
  Primer
ctttgaaaagtccagccgcatttcattccaacccttagggaaccc

SEQ ID NO: 608 - DNA, 53 bp, Artificial sequence
  Primer
gtgccagcaagatccaatctagagaagtcacaatgaaacagatttgcaaaaag

SEQ ID NO: 609 - DNA, 44 bp, Artificial sequence
  Primer
gaaattcggcgccttcatcagtatgtccaacccttagggaaccc

SEQ ID NO: 610 - DNA, 53 bp, Artificial sequence
  Primer
gtgccagcaagatccaatctagaaagaagatgaagagtcagatgatgctgatg

SEQ ID NO: 611 - DNA, 49 bp, Artificial sequence
  Primer
attttggatcattgtttgacttggaaaatgtccaacccttagggaaccc

SEQ ID NO: 612 - DNA, 45 bp, Artificial sequence
  Primer
atgggaataactgggaacacaagtcctccaacccttagggaaccc

SEQ ID NO: 613 - DNA, 46 bp, Artificial sequence
  Primer
gtgccagcaagatccaatctagagcttggtgcaggatttggaacag

SEQ ID NO: 614 - DNA, 45 bp, Artificial sequence
  Primer
gtgccagcaagatccaatctagaggagccccccaggccccagtag

SEQ ID NO: 615 - DNA, 51 bp, Artificial sequence
  Primer
gtgccagcaagatccaatctagagatgtcagaccctaagaagaaggaagag

SEQ ID NO: 616 - DNA, 47 bp, Artificial sequence
  Primer
gtgccagcaagatccaatctagaagccaatggagcattcatgcccaa

SEQ ID NO: 617 - DNA, 42 bp, Artificial sequence
  Primer
ctgtgcggtcagagaagaaacgctccaacccttagggaaccc

SEQ ID NO: 618 - DNA, 46 bp, Artificial sequence
  Primer
ttgatagagaaaaacaacccagcgaagtccaacccttagggaaccc

SEQ ID NO: 619 - DNA, 43 bp, Artificial sequence
  Primer
ataacccagcagccaactggcttctccaacccttagggaaccc

SEQ ID NO: 620 - DNA, 46 bp, Artificial sequence
  Primer
gatggtaaattgaaaaaacccaagaattccaacccttagggaaccc

SEQ ID NO: 621 - DNA, 46 bp, Artificial sequence
  Primer
ttcttgatgaagcagatagaatcttggtccaacccttagggaaccc

SEQ ID NO: 622 - DNA, 42 bp, Artificial sequence
  Primer
cacccctgccactttggaacagatccaacccttagggaaccc

SEQ ID NO: 623 - DNA, 48 bp, Artificial sequence
  Primer
ataatctcagtgataccttgaagaagctgtccaacccttagggaaccc

SEQ ID NO: 624 - DNA, 47 bp, Artificial sequence
  Primer
atacggaaacaagtgaaaaaatccaagctccaacccttagggaaccc

SEQ ID NO: 625 - DNA, 52 bp, Artificial sequence
  Primer
gtgccagcaagatccaatctagaaccaagaggctattcaagatctctgtctg

SEQ ID NO: 626 - DNA, 52 bp, Artificial sequence
  Primer
gtgccagcaagatccaatctagaaccaagaggctattcaagatctctgcatg

SEQ ID NO: 627 - DNA, 49 bp, Artificial sequence
  Primer
gcagtggaggaagtctctttaagaaaatagtccaacccttagggaaccc

SEQ ID NO: 628 - DNA, 46 bp, Artificial sequence
  Primer
gtgccagcaagatccaatctagacttctgccgctgcttctgcacag

SEQ ID NO: 629 - DNA, 42 bp, Artificial sequence
  Primer
gcatggggcggctggttctgctgtccaacccttagggaaccc

SEQ ID NO: 630 - DNA, 47 bp, Artificial sequence
  Primer
gtgccagcaagatccaatctagactgaggacatctggaggaaggctg

SEQ ID NO: 631 - DNA, 44 bp, Artificial sequence
  Primer
gtgccagcaagatccaatctagacagcaggaggactccagcgag

SEQ ID NO: 632 - DNA, 43 bp, Artificial sequence
  Primer
gcttcctgctgaactccaagttcctccaacccttagggaaccc

SEQ ID NO: 633 - DNA, 42 bp, Artificial sequence
  Primer
gacttccagccactgcgctattttccaacccttagggaaccc

SEQ ID NO: 634 - DNA, 46 bp, Artificial sequence
  Primer
gatgagcaattcttaggttttggctcatccaacccttagggaaccc

SEQ ID NO: 635 - DNA, 44 bp, Artificial sequence
  Primer
gaagaacctaggaaagtccgctttgtccaacccttagggaaccc

SEQ ID NO: 636 - DNA, 46 bp, Artificial sequence
  Primer
gaccctaataggagtattcataccagctccaacccttagggaaccc

SEQ ID NO: 637 - DNA, 42 bp, Artificial sequence
  Primer
tctgaacaacccagtcctgccagtccaacccttagggaaccc

SEQ ID NO: 638 - DNA, 50 bp, Artificial sequence
  Primer
attcttgaagtgaaaagtccaataaagcaaatccaacccttagggaaccc

SEQ ID NO: 639 - DNA, 44 bp, Artificial sequence
  Primer
gcatacctagatgaactggtagagctccaacccttagggaaccc

SEQ ID NO: 640 - DNA, 42 bp, Artificial sequence
  Primer
tgcaccgtccaggtgaggttagatccaacccttagggaaccc

SEQ ID NO: 641 - DNA, 42 bp, Artificial sequence
  Primer
gaggagccgaggaaggtctgctttccaacccttagggaaccc

SEQ ID NO: 642 - DNA, 42 bp, Artificial sequence
  Primer
gtgatggtaatgcccgaaggagctccaacccttagggaaccc

SEQ ID NO: 643 - DNA, 42 bp, Artificial sequence
  Primer
gacgccaacaaggagagcagcaatccaacccttagggaaccc

SEQ ID NO: 644 - DNA, 42 bp, Artificial sequence
  Primer
tctgcccagtcaagcccgtccaatccaacccttagggaaccc

SEQ ID NO: 645 - DNA, 46 bp, Artificial sequence
  Primer
agatgtgaactttgtccccataaggattccaacccttagggaaccc

SEQ ID NO: 646 - DNA, 48 bp, Artificial sequence
  Primer
acttgctacatttgtgatgaacaaggaagtccaacccttagggaaccc

SEQ ID NO: 647 - DNA, 50 bp, Artificial sequence
  Primer
aaatataaagagaaggacaaacacaaacagatccaacccttagggaaccc

SEQ ID NO: 648 - DNA, 49 bp, Artificial sequence
  Primer
acttatacaagcactagcaacaactctatatccaacccttagggaaccc

SEQ ID NO: 649 - DNA, 46 bp, Artificial sequence
  Primer
gcaaatactctatctggatcttctctctccaacccttagggaaccc

SEQ ID NO: 650 - DNA, 45 bp, Artificial sequence
  Primer
gatttggagttccatggagtgatgagtccaacccttagggaaccc

SEQ ID NO: 651 - DNA, 46 bp, Artificial sequence
  Primer
gattctgtttcactgaggccatctatctccaacccttagggaaccc

SEQ ID NO: 652 - DNA, 42 bp, Artificial sequence
  Primer
cacatctccatcccccagcctgatccaacccttagggaaccc

SEQ ID NO: 653 - DNA, 43 bp, Artificial sequence
  Primer
gtggccaacatgagtgctaaggactccaacccttagggaaccc

SEQ ID NO: 654 - DNA, 50 bp, Artificial sequence
  Primer
ttatcaagtgggaatcctgtatatgaaaaattccaacccttagggaaccc

SEQ ID NO: 655 - DNA, 46 bp, Artificial sequence
  Primer
catgataccagtagtcctttgctaatctccaacccttagggaaccc

SEQ ID NO: 656 - DNA, 46 bp, Artificial sequence
  Primer
agtcgaaaggacaaagaacgccttaagtccaacccttagggaaccc

SEQ ID NO: 657 - DNA, 41 bp, Artificial sequence
  Primer
tctcctcctcggcttcctcttctccaacccttagggaaccc

SEQ ID NO: 658 - DNA, 43 bp, Artificial sequence
  Primer
gtgttttctctggctggctctacctccaacccttagggaaccc

SEQ ID NO: 659 - DNA, 42 bp, Artificial sequence
  Primer
ggtgaaggttgccgaactgtccctccaacccttagggaaccc

SEQ ID NO: 660 - DNA, 42 bp, Artificial sequence
  Primer
gaagctatgagggaccctgtgagtccaacccttagggaaccc

SEQ ID NO: 661 - DNA, 43 bp, Artificial sequence
  Primer
gcacggacacttgctagtatgttgtccaacccttagggaaccc

SEQ ID NO: 662 - DNA, 43 bp, Artificial sequence
  Primer
gcatcagcttctggtgatgtgagctccaacccttagggaaccc

SEQ ID NO: 663 - DNA, 42 bp, Artificial sequence
  Primer
gagatgacgcattcatggcctcctccaacccttagggaaccc

SEQ ID NO: 664 - DNA, 43 bp, Artificial sequence
  Primer
gagtctcagcagtccaattttggctccaacccttagggaaccc

SEQ ID NO: 665 - DNA, 45 bp, Artificial sequence
  Primer
atatttaacaccgtgcccgatatgcctccaacccttagggaaccc

SEQ ID NO: 666 - DNA, 46 bp, Artificial sequence
  Primer
aagatggcagtgaacgtatactcaacgtccaacccttagggaaccc

SEQ ID NO: 667 - DNA, 49 bp, Artificial sequence
  Primer
ataattcctgtggacaaattagtaaaaggatccaacccttagggaaccc

SEQ ID NO: 668 - DNA, 49 bp, Artificial sequence
  Primer
gtcaacgtattgaaacttactgttgaagactccaacccttagggaaccc

SEQ ID NO: 669 - DNA, 43 bp, Artificial sequence
  Primer
gacattgacaagcagtacgtgggctccaacccttagggaaccc

SEQ ID NO: 670 - DNA, 42 bp, Artificial sequence
  Primer
gaggcacgcggacctccagtggctccaacccttagggaaccc

SEQ ID NO: 671 - DNA, 46 bp, Artificial sequence
  Primer
ccttgaaaagatcttttgaggtcgaggtccaacccttagggaaccc

SEQ ID NO: 672 - DNA, 50 bp, Artificial sequence
  Primer
gaaaaaaaccttgaagataacttacagagtttccaacccttagggaaccc

SEQ ID NO: 673 - DNA, 45 bp, Artificial sequence
  Primer
gagagtagatctggagaaaccaacagtccaacccttagggaaccc

SEQ ID NO: 674 - DNA, 42 bp, Artificial sequence
  Primer
gagatgacctggcttccaccacttccaacccttagggaaccc

SEQ ID NO: 675 - DNA, 42 bp, Artificial sequence
  Primer
caggcagctcagagaacggctcttccaacccttagggaaccc

SEQ ID NO: 676 - DNA, 50 bp, Artificial sequence
  Primer
atttttgatcaccatactgaagaggatatagtccaacccttagggaaccc

SEQ ID NO: 677 - DNA, 42 bp, Artificial sequence
  Primer
aactccatccggcacaacctgtctccaacccttagggaaccc

SEQ ID NO: 678 - DNA, 46 bp, Artificial sequence
  Primer
ctccagggttccttgaaaagaaaacagtccaacccttagggaaccc

SEQ ID NO: 679 - DNA, 43 bp, Artificial sequence
  Primer
gatcaacactctgtggtaggccagtccaacccttagggaaccc

SEQ ID NO: 680 - DNA, 47 bp, Artificial sequence
  Primer
caagttggaattgacagaggtgatatactccaacccttagggaaccc

SEQ ID NO: 681 - DNA, 42 bp, Artificial sequence
  Primer
gcccctagcagtcttcttgatgctccaacccttagggaaccc

SEQ ID NO: 682 - DNA, 45 bp, Artificial sequence
  Primer
caacctcataatattctgcagaggcgtccaacccttagggaaccc

SEQ ID NO: 683 - DNA, 42 bp, Artificial sequence
  Primer
gacatgcggaagcacgtggccattccaacccttagggaaccc

SEQ ID NO: 684 - DNA, 42 bp, Artificial sequence
  Primer
ggctacatgcagccgctgaagcatccaacccttagggaaccc

SEQ ID NO: 685 - DNA, 46 bp, Artificial sequence
  Primer
ttctccaaggatgtcctagtaaacatctccaacccttagggaaccc

SEQ ID NO: 686 - DNA, 43 bp, Artificial sequence
  Primer
caaagcatgcgtgagaacaaggagtccaacccttagggaaccc

SEQ ID NO: 687 - DNA, 44 bp, Artificial sequence
  Primer
ccagaagtcattggatctgtgtcactccaacccttagggaaccc

SEQ ID NO: 688 - DNA, 49 bp, Artificial sequence
  Primer
gtaaccatggagcttattacagataacaaatccaacccttagggaaccc

SEQ ID NO: 689 - DNA, 43 bp, Artificial sequence
  Primer
gtgattcctgtctctctgtcttcctccaacccttagggaaccc

SEQ ID NO: 690 - DNA, 42 bp, Artificial sequence
  Primer
gcagaggaccgaggaaatggacttccaacccttagggaaccc

SEQ ID NO: 691 - DNA, 43 bp, Artificial sequence
  Primer
gatggagctgtagttacaccctcctccaacccttagggaaccc

SEQ ID NO: 692 - DNA, 43 bp, Artificial sequence
  Primer
agtcccaagagtggcccaaaagagtccaacccttagggaaccc

SEQ ID NO: 693 - DNA, 44 bp, Artificial sequence
  Primer
gacactcaatcacttgtcggaagtctccaacccttagggaaccc

SEQ ID NO: 694 - DNA, 42 bp, Artificial sequence
  Primer
gtggaacggccgccttctccatttccaacccttagggaaccc

SEQ ID NO: 695 - DNA, 42 bp, Artificial sequence
  Primer
gtgaatgaggcctctggggatggtccaacccttagggaaccc

SEQ ID NO: 696 - DNA, 42 bp, Artificial sequence
  Primer
aagcctggaatggtcccccctcctccaacccttagggaaccc

SEQ ID NO: 697 - DNA, 42 bp, Artificial sequence
  Primer
aactcgatccgccacaacctgtctccaacccttagggaaccc

SEQ ID NO: 698 - DNA, 47 bp, Artificial sequence
  Primer
caacagccaactcagtttataaatccagtccaacccttagggaaccc

SEQ ID NO: 699 - DNA, 44 bp, Artificial sequence
  Primer
ctgcagaagaaagatcagcaactggtccaacccttagggaaccc

SEQ ID NO: 700 - DNA, 45 bp, Artificial sequence; identical to SEQ ID NO: 612
  Primer

SEQ ID NO: 701 - DNA, 46 bp, Artificial sequence
  Primer
tctcagatgcaaacatcagtgggaatttccaacccttagggaaccc

SEQ ID NO: 702 - DNA, 44 bp, Artificial sequence
  Primer
atgatggaggaggatttgcaaggagtccaacccttagggaaccc

SEQ ID NO: 703 - DNA, 44 bp, Artificial sequence
  Primer
gtgagtacccagaacatgaagatggtccaacccttagggaaccc

SEQ ID NO: 704 - DNA, 42 bp, Artificial sequence
  Primer
ggcctggagcaggatgtcctccatccaacccttagggaaccc

SEQ ID NO: 705 - DNA, 46 bp, Artificial sequence
  Primer
aatcagatgggtgactcaaatatctcctccaacccttagggaaccc

SEQ ID NO: 706 - DNA, 43 bp, Artificial sequence
  Primer
tctgctccatctggacacaagcattccaacccttagggaaccc

SEQ ID NO: 707 - DNA, 42 bp, Artificial sequence
  Primer
ctggagaatgctggaggagaccttccaacccttagggaaccc

SEQ ID NO: 708 - DNA, 45 bp, Artificial sequence
  Primer
aatactctggagcagtgcaatgtgtgtccaacccttagggaaccc

SEQ ID NO: 709 - DNA, 45 bp, Artificial sequence; identical to SEQ ID NO: 708
  Primer

SEQ ID NO: 710 - DNA, 46 bp, Artificial sequence
  Primer
agagaaatagcccggaaacttgcaaattccaacccttagggaaccc

SEQ ID NO: 711 - DNA, 48 bp, Artificial sequence
  Primer
aatgaagagcttcgaaacttgtctttgtctccaacccttagggaaccc

SEQ ID NO: 712 - DNA, 43 bp, Artificial sequence
  Primer
ccacagcgtcctgtgtttactcattccaacccttagggaaccc

SEQ ID NO: 713 - DNA, 49 bp, Artificial sequence
  Primer
ttccaaaagctgagacaagatcttgaaatgtccaacccttagggaaccc

SEQ ID NO: 714 - DNA, 43 bp, Artificial sequence
  Primer
gtgacgacgtcatcaggaagcaagtccaacccttagggaaccc

SEQ ID NO: 715 - DNA, 42 bp, Artificial sequence
  Primer
gacaacagccggcgtgtggagcatccaacccttagggaaccc

SEQ ID NO: 716 - DNA, 43 bp, Artificial sequence
  Primer
gacttcctgacagacctgatgatgtccaacccttagggaaccc

SEQ ID NO: 717 - DNA, 49 bp, Artificial sequence
  Primer
gatcagtttgacaacttagaaaaacacacatccaacccttagggaaccc

SEQ ID NO: 718 - DNA, 46 bp, Artificial sequence
  Primer
gctacagacaagagaaaagctttagagtccaacccttagggaaccc

SEQ ID NO: 719 - DNA, 47 bp, Artificial sequence
  Primer
actgtggatattcataaggagaaagtggtccaacccttagggaaccc

SEQ ID NO: 720 - DNA, 49 bp, Artificial sequence
  Primer
gtcaagtacaaaagagattttgaagaaagctccaacccttagggaaccc

SEQ ID NO: 721 - DNA, 50 bp, Artificial sequence
  Primer
gtaaaataccatgaagattttgaaaaaacaatccaacccttagggaaccc

SEQ ID NO: 722 - DNA, 42 bp, Artificial sequence
  Primer
aagcggtaccgcgcggtgtatgatccaacccttagggaaccc

SEQ ID NO: 723 - DNA, 42 bp, Artificial sequence
  Primer
ctggtcagtgagaaggtcggaggtccaacccttagggaaccc

SEQ ID NO: 724 - DNA, 46 bp, Artificial sequence
  Primer
ttctccaatatccccttcttcatcttctccaacccttagggaaccc

SEQ ID NO: 725 - DNA, 42 bp, Artificial sequence
  Primer
ggccctcctcaggacctgtctgttccaacccttagggaaccc

SEQ ID NO: 726 - DNA, 42 bp, Artificial sequence
  Primer
ggcagcaaggagcgcttccactgtccaacccttagggaaccc

SEQ ID NO: 727 - DNA, 43 bp, Artificial sequence
  Primer
acctacatcggctctgtgctcatctccaacccttagggaaccc

SEQ ID NO: 728 - DNA, 42 bp, Artificial sequence
  Primer
accttcacggcatggtgcaactctccaacccttagggaaccc

SEQ ID NO: 729 - DNA, 42 bp, Artificial sequence
  Primer
cctatgggctatgggcctcgtattccaacccttagggaaccc

SEQ ID NO: 730 - DNA, 44 bp, Artificial sequence
  Primer
gcacaaatgtctagttcttcctgcctccaacccttagggaaccc

SEQ ID NO: 731 - DNA, 43 bp, Artificial sequence
  Primer
ctttcccagccagctgtaagcatttccaacccttagggaaccc

SEQ ID NO: 732 - DNA, 47 bp, Artificial sequence
  Primer
gaaacaatgaccgataaaacagagaaggtccaacccttagggaaccc

SEQ ID NO: 733 - DNA, 42 bp, Artificial sequence
  Primer
ctctacgtctcctccgagagccgtccaacccttagggaaccc

SEQ ID NO: 734 - DNA, 42 bp, Artificial sequence
  Primer
gtctcctcctcggcttcctcttctccaacccttagggaaccc

SEQ ID NO: 735 - DNA, 48 bp, Artificial sequence; identical to SEQ ID NO: 623
  Primer

SEQ ID NO: 736 - DNA, 47 bp, Artificial sequence
  Primer
aaatgacatcagatgtaccatcactgggtccaacccttagggaaccc

SEQ ID NO: 737 - DNA, 48 bp, Artificial sequence
  Primer
tcgcttcatggagatataagtagcctgaatccaacccttagggaaccc

SEQ ID NO: 738 - DNA, 46 bp, Artificial sequence
  Primer
gaaattcggcgccttcatcagtatgtgtccaacccttagggaaccc

SEQ ID NO: 739 - DNA, 49 bp, Artificial sequence
  Primer
atcgtactgagaagcactccacaatgccagtccaacccttagggaaccc

SEQ ID NO: 740 - DNA, 52 bp, Artificial sequence
  Primer
caggaacgaatttcatatacacctccagagagctccaacccttagggaaccc

SEQ ID NO: 741 - DNA, 45 bp, Artificial sequence
  Primer
gcttgcagccaatttactggagcaggtccaacccttagggaaccc

SEQ ID NO: 742 - DNA, 52 bp, Artificial sequence
  Primer
atcttagacgaattttacaatgtgaagttctgctccaacccttagggaaccc

SEQ ID NO: 743 - DNA, 43 bp, Artificial sequence
  Primer
gtgtctgctgactccagtgcatcctccaacccttagggaaccc

SEQ ID NO: 744 - DNA, 47 bp, Artificial sequence
  Primer
attgtcagtaaacgggcaggtactcagttccaacccttagggaaccc

SEQ ID NO: 745 - DNA, 46 bp, Artificial sequence
  Primer
gatgacagcatggaagagaaaccactatccaacccttagggaaccc

SEQ ID NO: 746 - DNA, 46 bp, Artificial sequence
  Primer
attttggctctctatttgacttggagctccaacccttagggaaccc

SEQ ID NO: 747 - DNA, 43 bp, Artificial sequence
  Primer
atgggaatgatgaacaaccccaattccaacccttagggaaccc

SEQ ID NO: 748 - DNA, 42 bp, Artificial sequence
  Primer
ccttgccctttaaggtggtggtgtccaacccttagggaaccc

SEQ ID NO: 749 - DNA, 47 bp, Artificial sequence
  Primer
ctgccttatgactcaagatgggagtttctccaacccttagggaaccc

SEQ ID NO: 750 - DNA, 42 bp, Artificial sequence
  Primer
gtgccagcaagatccaatctagagggccccgagaacctcgaa

SEQ ID NO: 751 - DNA, 45 bp, Artificial sequence
  Primer
gtgccagcaagatccaatctagagggcgacctcttccagaagctg

SEQ ID NO: 752 - DNA, 50 bp, Artificial sequence
  Primer
gtgccagcaagatccaatctagaatccaacgaccaagaactctctggaaa

SEQ ID NO: 753 - DNA, 48 bp, Artificial sequence
  Primer
gtgccagcaagatccaatctagagccagttatttgcctcagtaaaaga

SEQ ID NO: 754 - DNA, 53 bp, Artificial sequence
  Primer
gtgccagcaagatccaatctagacagtatgagtacacggagctcaagaaacag

SEQ ID NO: 755 - DNA, 48 bp, Artificial sequence
  Primer
gtgccagcaagatccaatctagacacaatgaaacagatttgcaaaaag

SEQ ID NO: 756 - DNA, 43 bp, Artificial sequence
  Primer
gtgccagcaagatccaatctagaccgcctgcctgcgcacctgc

SEQ ID NO: 757 - DNA, 45 bp, Artificial sequence
  Primer
gtgccagcaagatccaatctagacgggcagaaggctggtgacaag

SEQ ID NO: 758 - DNA, 43 bp, Artificial sequence
  Primer
gtgccagcaagatccaatctagaaagggccaaccactggggaa

SEQ ID NO: 759 - DNA, 53 bp, Artificial sequence
  Primer
gtgccagcaagatccaatctagaaaaacaagtgcacagacaacaccaagtaag

SEQ ID NO: 760 - DNA, 49 bp, Artificial sequence
  Primer
gtgccagcaagatccaatctagagggcaaatgagaacagcaacatacag

SEQ ID NO: 761 - DNA, 49 bp, Artificial sequence
  Primer
gtgccagcaagatccaatctagagcacacctctcaatgcagctttacag

SEQ ID NO: 762 - DNA, 52 bp, Artificial sequence
  Primer
gtgccagcaagatccaatctagatgaggacaagttctacagccacaagaaaa

SEQ ID NO: 763 - DNA, 44 bp, Artificial sequence
  Primer
gtgccagcaagatccaatctagaagaaccccaacagcaaagaag

SEQ ID NO: 764 - DNA, 45 bp, Artificial sequence
  Primer
gtgccagcaagatccaatctagacctaagatgcccgacttcaact

SEQ ID NO: 765 - DNA, 45 bp, Artificial sequence; identical to SEQ ID NO: 764
  Primer

SEQ ID NO: 766 - DNA, 53 bp, Artificial sequence
  Primer
gtgccagcaagatccaatctagacagtgccaggaaagagaattagagatcagt

SEQ ID NO: 767 - DNA, 53 bp, Artificial sequence; identical to SEQ ID NO: 766
  Primer

SEQ ID NO: 768 - DNA, 46 bp, Artificial sequence
  Primer
gtgccagcaagatccaatctagacaaagcccacgctgaagcgaaag

SEQ ID NO: 769 - DNA, 49 bp, Artificial sequence
  Primer
gtgccagcaagatccaatctagaagctaaaaggacagcaggtgctacca

SEQ ID NO: 770 - DNA, 51 bp, Artificial sequence
  Primer
gtgccagcaagatccaatctagagttcagagactgaaggatgaagccagag

SEQ ID NO: 771 - DNA, 52 bp, Artificial sequence
  Primer
gtgccagcaagatccaatctagagatgatgatgaagaggatgatgatgaaga

SEQ ID NO: 772 - DNA, 44 bp, Artificial sequence
  Primer
gtgccagcaagatccaatctagacagcttccagtcacagcatag

SEQ ID NO: 773 - DNA, 51 bp, Artificial sequence
  Primer
gtgccagcaagatccaatctagagatgtcagcatacccagatccacattag

SEQ ID NO: 774 - DNA, 47 bp, Artificial sequence
  Primer
gtgccagcaagatccaatctagaaccgcttggaatgctgcaacaatg

SEQ ID NO: 775 - DNA, 50 bp, Artificial sequence
  Primer
gtgccagcaagatccaatctagaccaacaggcatgataggatatggaatt

SEQ ID NO: 776 - DNA, 48 bp, Artificial sequence
  Primer
gtgccagcaagatccaatctagactttggccctgtatcaggagcacag

SEQ ID NO: 777 - DNA, 44 bp, Artificial sequence
  Primer
gtgccagcaagatccaatctagactgggccagaatggtgaggag

SEQ ID NO: 778 - DNA, 46 bp, Artificial sequence
  Primer
gtgccagcaagatccaatctagaggctggcgcacctgggcgtgcag

SEQ ID NO: 779 - DNA, 45 bp, Artificial sequence
  Primer
gtgccagcaagatccaatctagatgatgaaggggaggaaggagag

SEQ ID NO: 780 - DNA, 51 bp, Artificial sequence
  Primer
gtgccagcaagatccaatctagacgtgatctttgatagatccagggaagag

SEQ ID NO: 781 - DNA, 50 bp, Artificial sequence
  Primer
gtgccagcaagatccaatctagattcttggctcaacaagccataaaacag

SEQ ID NO: 782 - DNA, 52 bp, Artificial sequence
  Primer
gtgccagcaagatccaatctagaggtcttataatcttccctctcttccggat

SEQ ID NO: 783 - DNA, 48 bp, Artificial sequence
  Primer
gtgccagcaagatccaatctagaccagcaaccttccatctctcatcag

SEQ ID NO: 784 - DNA, 47 bp, Artificial sequence
  Primer
gtgccagcaagatccaatctagaccgcagagcactgtatcacgaaaa

SEQ ID NO: 785 - DNA, 53 bp, Artificial sequence
  Primer
gtgccagcaagatccaatctagaattcctgttcctactacagttcctgttcct

SEQ ID NO: 786 - DNA, 52 bp, Artificial sequence
  Primer
gtgccagcaagatccaatctagaaccaagaggctattcaagatctctgcctg

SEQ ID NO: 787 - DNA, 52 bp, Artificial sequence
  Primer
gtgccagcaagatccaatctagaaccaagaggctattcaagatctctgtatg

SEQ ID NO: 788 - DNA, 53 bp, Artificial sequence
  Primer
gtgccagcaagatccaatctagaagcatttggttttaaattatggagtatgtt

SEQ ID NO: 789 - DNA, 45 bp, Artificial sequence
  Primer
tctgtggagacgagaatattctggtttccaacccttagggaaccc

SEQ ID NO: 790 - DNA, 50 bp, Artificial sequence
  Primer
gtgccagcaagatccaatctagagatttgtgattttggtctagccagagt

SEQ ID NO: 791 - DNA, 50 bp, Artificial sequence
  Primer
catcaagaatgattctaattatgtggttaaatccaacccttagggaaccc

SEQ ID NO: 792 - DNA, 41 bp, Artificial sequence
  Primer
catcccagtgactgcatccctctccaacccttagggaaccc

SEQ ID NO: 793 - DNA, 39 bp, Artificial sequence
  Primer
ggggaccccattcccgaggatccaacccttagggaaccc

SEQ ID NO: 794 - DNA, 46 bp, Artificial sequence
  Primer
gctctccacagatagagaacatccagctccaacccttagggaaccc

SEQ ID NO: 795 - DNA, 44 bp, Artificial sequence
  Primer
ctgaacagatgggtaaggatggcagtccaacccttagggaaccc

SEQ ID NO: 796 - DNA, 42 bp, Artificial sequence
  Primer
ggaccaaccacttcctaccccagtccaacccttagggaaccc

SEQ ID NO: 797 - DNA, 39 bp, Artificial sequence
  Primer
gccccaggtgtacccaccactccaacccttagggaaccc

SEQ ID NO: 798 - DNA, 44 bp, Artificial sequence
  Primer
gcaacctccaagtcccagatcatgttccaacccttagggaaccc

SEQ ID NO: 799 - DNA, 39 bp, Artificial sequence
  Primer
ggagttcctggtcggctccgtccaacccttagggaaccc

SEQ ID NO: 800 - DNA, 50 bp, Artificial sequence
  Primer
gtgccagcaagatccaatctagacgagttcaagcaggcctatatcacctg

SEQ ID NO: 801 - DNA, 43 bp, Artificial sequence
  Primer
gtgccagcaagatccaatctagagtgggcctcctgggcctcag

SEQ ID NO: 802 - DNA, 46 bp, Artificial sequence
  Primer
gtgccagcaagatccaatctagatccctggaatgaagggacacaga

SEQ ID NO: 803 - DNA, 43 bp, Artificial sequence
  Primer
gtgccagcaagatccaatctagaatggcaaaactggcccccct

SEQ ID NO: 804 - DNA, 46 bp, Artificial sequence
  Primer
gtgccagcaagatccaatctagatccctggacctaaaggtgctgct

SEQ ID NO: 805 - DNA, 46 bp, Artificial sequence
  Primer
gtgccagcaagatccaatctagaaagcaggcaaacctggtgaacag

SEQ ID NO: 806 - DNA, 45 bp, Artificial sequence
  Primer
gtgccagcaagatccaatctagatccagggcctaagggtgacaga

SEQ ID NO: 807 - DNA, 43 bp, Artificial sequence
  Primer
gtgccagcaagatccaatctagactggtgcccctggtgacaag

SEQ ID NO: 808 - DNA, 43 bp, Artificial sequence
  Primer
gtgccagcaagatccaatctagactggaccccctggccccatt

SEQ ID NO: 809 - DNA, 43 bp, Artificial sequence
  Primer
gtgccagcaagatccaatctagaagggtccccctggccctcct

SEQ ID NO: 810 - DNA, 43 bp, Artificial sequence
  Primer
gtgccagcaagatccaatctagactggtcctgctggtccccga

SEQ ID NO: 811 - DNA, 43 bp, Artificial sequence
  Primer
gtgccagcaagatccaatctagactggcgagcctggagcttca

SEQ ID NO: 812 - DNA, 45 bp, Artificial sequence
  Primer
gtgccagcaagatccaatctagatgtcctccttgaagggctccag

SEQ ID NO: 813 - DNA, 50 bp, Artificial sequence
  Primer
gtgccagcaagatccaatctagacctccactgaagaagctgaaacaagag

SEQ ID NO: 814 - DNA, 50 bp, Artificial sequence; identical to SEQ ID NO: 813
  Primer

SEQ ID NO: 815 - DNA, 48 bp, Artificial sequence
  Primer
gtgccagcaagatccaatctagagagagtctggatggacatttgcagg

SEQ ID NO: 816 - DNA, 43 bp, Artificial sequence
  Primer
gtgccagcaagatccaatctagatgcgaagccacctctcgcag

SEQ ID NO: 817 - DNA, 49 bp, Artificial sequence
  Primer
ggagcggacatggactacgactcgtaccagtccaacccttagggaaccc

SEQ ID NO: 818 - DNA, 49 bp, Artificial sequence
  Primer
agaatgaagaaattgatgttgtgacagtagtccaacccttagggaaccc

SEQ ID NO: 819 - DNA, 45 bp, Artificial sequence
  Primer
aggaaggcatcaaccacgagtgtaagtccaacccttagggaaccc

SEQ ID NO: 820 - DNA, 51 bp, Artificial sequence
  Primer
gtgccagcaagatccaatctagacagctctctgtaatgcgatactcaccag

SEQ ID NO: 821 - DNA, 53 bp, Artificial sequence
  Primer
gtgccagcaagatccaatctagaaagagaggctgtatctccatgccagagcag

SEQ ID NO: 822 - DNA, 53 bp, Artificial sequence
  Primer
gtgccagcaagatccaatctagaacccaaaagcagaccttggagaacagtcag

SEQ ID NO: 823 - DNA, 53 bp, Artificial sequence
  Primer
gtgccagcaagatccaatctagatctttgaactactgccggagcttctgccag

SEQ ID NO: 824 - DNA, 47 bp, Artificial sequence
  Primer
gtgccagcaagatccaatctagacaccgaggaccagctcaatgacag

SEQ ID NO: 825 - DNA, 28 bp, Artificial sequence
  Primer
cttctttaagcagtgtggggttgttaag

SEQ ID NO: 826 - DNA, 27 bp, Artificial sequence
  Primer
ttttcttaccacaacatgacagtagtg

SEQ ID NO: 827 - DNA, 23 bp, Artificial sequence
  Primer
atccactgtgcgacgagctgtgc

SEQ ID NO: 828 - DNA, 24 bp, Artificial sequence
  Primer
gaggatccaaagtgggaattccct

SEQ ID NO: 829 - DNA, 27 bp, Artificial sequence
  Primer
attgctgtgggaaataatgatgtaaag

SEQ ID NO: 830 - DNA, 27 bp, Artificial sequence
  Primer
gcagcatgtcagcttcgtatctctcaa

SEQ ID NO: 831 - DNA, 27 bp, Artificial sequence
  Primer
aagaactagtccagcttcgagcacaag

SEQ ID NO: 832 - DNA, 27 bp, Artificial sequence
  Primer
caggacctggctacaagagttaaaaag

SEQ ID NO: 833 - DNA, 27 bp, Artificial sequence
  Primer
gaacagctcactaaagtgcacaaacag

SEQ ID NO: 834 - DNA, 25 bp, Artificial sequence
  Primer
agaagagggcattctgcacagattg

SEQ ID NO: 835 - DNA, 23 bp, Artificial sequence
  Primer
tgcgcaaagccagcgtgaccatc

SEQ ID NO: 836 - DNA, 10 bp, artificial sequence
  sequence index
  misc_feature (1)..(10): n is a, c, g, or t
  misc_feature (1)..(10): n is a, c, g, or t
  misc_feature (6)..(10): n is present or absent
nnnnnnnnnn

SEQ ID NO: 837 - DNA, 55 bp, Artificial sequence
  Fusion transcript
aaaaatacccacacctgggaaaggacctaaagtgtaccgccggaagcaccaggag

SEQ ID NO: 838 - DNA, 55 bp, Artificial sequence
  Fusion transcript
aaaatgacccacacctgggaaaggacctaaagtgtaccgccggaagcaccaggag

SEQ ID NO: 839 - DNA, 55 bp, Artificial sequence
  Fusion transcript
aaaatgccccacacctgggaaaggacctaaagtgtaccgccggaagcaccaggag

SEQ ID NO: 840 - DNA, 55 bp, Artificial sequence
  Fusion transcript
aaacactcccacacctgggaaaggacctaaagtgtaccgccggaagcaccaggag

SEQ ID NO: 841 - DNA, 55 bp, Artificial sequence; identical to SEQ ID NO: 840
  Fusion transcript

SEQ ID NO: 842 - DNA, 55 bp, Artificial sequence
  Fusion transcript
aaactgccccacacctgggaaaggacctaaagtgtaccgccggaagcaccaggag

SEQ ID NO: 843 - DNA, 55 bp, Artificial sequence
  Fusion transcript
aaactgtcccacacctgggaaaggacctaaagtgtaccgccggaagcaccaggag

SEQ ID NO: 844 - DNA, 55 bp, Artificial sequence
  Fusion transcript
aaagagacccacacctgggaaaggacctaaagtgtaccgccggaagcaccaggag

SEQ ID NO: 845 - DNA, 55 bp, Artificial sequence
  Fusion transcript
aaagatgcccacacctgggaaaggacctaaagtgtaccgccggaagcaccaggag

SEQ ID NO: 846 - DNA, 55 bp, Artificial sequence
  Fusion transcript
aaaggctcccacacctgggaaaggacctaaagtgtaccgccggaagcaccaggag

SEQ ID NO: 847 - DNA, 55 bp, Artificial sequence
  Fusion transcript
aaaggtacccacacctgggaaaggacctaaagtgtaccgccggaagcaccaggag

SEQ ID NO: 848 - DNA, 55 bp, Artificial sequence; identical to SEQ ID NO: 847
  Fusion transcript

SEQ ID NO: 849 - DNA, 55 bp, Artificial sequence
  Fusion transcript
aaagtgtcccacacctgggaaaggacctaaagtgtaccgccggaagcaccaggag

SEQ ID NO: 850 - DNA, 55 bp, Artificial sequence
  Fusion transcript
aaagttccccacacctgggaaaggacctaaagtgtaccgccggaagcaccaggag

SEQ ID NO: 851 - DNA, 7 bp, Artificial sequence
  Sequence of molecular barcode
aaaaata

SEQ ID NO: 852 - DNA, 7 bp, Artificial sequence
  Sequence of molecular barcode
aaaatga

SEQ ID NO: 853 - DNA, 7 bp, Artificial sequence
  Sequence of molecular barcode
aaaatgc

SEQ ID NO: 854 - DNA, 7 bp, Artificial sequence
  Sequence of molecular barcode
aaacact

SEQ ID NO: 855 - DNA, 7 bp, Artificial sequence
  Sequence of molecular barcode
aaacgag

SEQ ID NO: 856 - DNA, 7 bp, Artificial sequence
  Sequence of molecular barcode
aaacgag

SEQ ID NO: 857 - DNA, 7 bp, Artificial sequence
  Sequence of molecular barcode
aaactgt

SEQ ID NO: 858 - DNA, 7 bp, Artificial sequence
  Sequence of molecular barcode
aaactgt

SEQ ID NO: 859 - DNA, 7 bp, Artificial sequence
  Sequence of molecular barcode
aaagatg

SEQ ID NO: 860 - DNA, 7 bp, Artificial sequence
  Sequence of molecular barcode
aaaggct

SEQ ID NO: 861 - DNA, 7 bp, Artificial sequence
  Sequence of molecular barcode
aaaggta

SEQ ID NO: 862 - DNA, 7 bp, Artificial sequence
  Sequence of molecular barcode
aaagtca

SEQ ID NO: 863 - DNA, 7 bp, Artificial sequence
  Sequence of molecular barcode
aaagtgt

SEQ ID NO: 864 - DNA, 7 bp, Artificial sequence
  Sequence of molecular barcode
aaagttc

SEQ ID NO: 865 - DNA, 20 bp, Artificial sequence
  Extension, for example P5 or P7 65
  misc_feature (1)..(20): n -if present- represents A, T, C or G
  misc_feature (6)..(20): n is present or absent
nnnnnnnnnnnnnnnnnnnn

SEQ ID NO: 866 - DNA, 20 bp, Artificial Sequence
  Primer
ccgtccacacccgccgccag

SEQ ID NO: 867 - DNA, 20 bp, Artificial sequence
  Primer
accgcgagaagatgacccag

SEQ ID NO: 868 - DNA, 30 bp, Artificial sequence
  Primer
ctaagcagtgatgaagaggagaatgaacag

SEQ ID NO: 869 - DNA, 20 bp, Artificial sequence
  Primer
cgctcgcccggacccctcag

SEQ ID NO: 870 - DNA, 30 bp, Artificial sequence
  Primer
gaagaagagctgagaaaagccattttagtg

SEQ ID NO: 871 - DNA, 30 bp, Artificial sequence
  Primer
gaagtggtcctgtactgcttagagaacaag

SEQ ID NO: 872 - DNA, 27 bp, Artificial sequence
  Primer
gcgagtatagtgttggaaacaagcacc

SEQ ID NO: 873 - DNA, 20 bp, Artificial sequence
  Primer
tgccggaagctgcccagtga

SEQ ID NO: 874 - DNA, 30 bp, Artificial sequence
  Primer
gtttacagaaaaagcaaaggaaaccgttct

SEQ ID NO: 875 - DNA, 24 bp, Artificial sequence
  Primer
ctgacagcgaagactccgaaacag

SEQ ID NO: 876 - DNA, 22 bp, Artificial sequence
  Primer
gcagccctgcttcttcacagtt

SEQ ID NO: 877 - DNA, 20 bp, Artificial sequence
  Primer
tccatggcatcaagtggacc

SEQ ID NO: 878 - DNA, 20 bp, Artificial sequence
  Primer
gagctggcggcagcgtgcat

SEQ ID NO: 879 - DNA, 20 bp, Artificial sequence
  Primer
gtgaagcggcccaggtgagg

SEQ ID NO: 880 - DNA, 20 bp, Artificial sequence
  Primer
tccaccctcaagggccccag

SEQ ID NO: 881 - DNA, 26 bp, Artificial sequence
  Primer
cagcaagtatccaatgggtgaagaag

SEQ ID NO: 882 - DNA, 28 bp, Artificial sequence
  Primer
gtaagactcggaccaaggacaagtaccg

SEQ ID NO: 883 - DNA, 20 bp, Artificial sequence
  Primer
gcaaacagcagcccagcaga

SEQ ID NO: 884 - DNA, 22 bp, Artificial sequence
  Primer
gtcgagggccaagacgaagaca

SEQ ID NO: 885 - DNA, 30 bp, Artificial sequence
  Primer
cagtaaccttatgcctagcaacatgccaat

SEQ ID NO: 886 - DNA, 30 bp, Artificial sequence
  Primer
atcccactattattttggcacaacaggaag

SEQ ID NO: 887 - DNA, 26 bp, Artificial sequence
  Primer
agaaccattggctctcactgaaacag

SEQ ID NO: 888 - DNA, 24 bp, Artificial sequence
  Primer
aatgtgaaaaggtttgcgctcctg

SEQ ID NO: 889 - DNA, 20 bp, Artificial sequence
  Primer
aggacctggtgcagatgcct

SEQ ID NO: 890 - DNA, 26 bp, Artificial sequence
  Primer
aaattacaggggacatcagggccact

SEQ ID NO: 891 - DNA, 20 bp, Artificial sequence
  Primer
ccccagtggaccacctgcat

SEQ ID NO: 892 - DNA, 20 bp, Artificial sequence
  Primer
aaactgcagggatcaggccc

SEQ ID NO: 893 - DNA, 20 bp, Artificial sequence
  Primer
ggcactgcactgtgtgcgag

SEQ ID NO: 894 - DNA, 26 bp, Artificial sequence
  Primer
ttgctatagcccaaggtggaacaatc

SEQ ID NO: 895 - DNA, 22 bp, Artificial sequence
  Primer
ctgccactggtgacatgccaac

SEQ ID NO: 896 - DNA, 20 bp, Artificial sequence
  Primer
gcctgacgcgggccgcgcgg

SEQ ID NO: 897 - DNA, 20 bp, Artificial sequence
  Primer
ccgacctcaccctgtcgcgg

SEQ ID NO: 898 - DNA, 20 bp, Artificial sequence
  Primer
gaggagcctgttcccctgag

SEQ ID NO: 899 - DNA, 21 bp, Artificial sequence
  Primer
tgatggcttgtgcccaaacag

SEQ ID NO: 900 - DNA, 20 bp, Artificial sequence
  Primer
agacagcagtgagcatggcg

SEQ ID NO: 901 - DNA, 26 bp, Artificial sequence
  Primer
atcaagatgactgtgctcctgtggga

SEQ ID NO: 902 - DNA, 26 bp, Artificial sequence
  Primer
atattgatgagtgccaactgggggag

SEQ ID NO: 903 - DNA, 23 bp, Artificial sequence
  Primer
ggtcaaatttcagccatcagcaa

SEQ ID NO: 904 - DNA, 20 bp, Artificial sequence
  Primer
aggactgggcgctgctgcag

SEQ ID NO: 905 - DNA, 30 bp, Artificial sequence
  Primer
gtaaaagtagcagtggttcagcacactttg

SEQ ID NO: 906 - DNA, 24 bp, Artificial sequence
  Primer
tcagacgaagaacctctctcccag

SEQ ID NO: 907 - DNA, 24 bp, Artificial sequence
  Primer
cagtgccatcagcagcatagcaag

SEQ ID NO: 908 - DNA, 24 bp, Artificial sequence
  Primer
gctcgactgtggggaaaccataag

SEQ ID NO: 909 - DNA, 20 bp, Artificial sequence
  Primer
gccaccaccactccgtggag

SEQ ID NO: 910 - DNA, 23 bp, Artificial sequence
  Primer
ccagcagccactgcacctacaag

SEQ ID NO: 911 - DNA, 30 bp, Artificial sequence
  Primer
tatggacagagtaactacagttatccccag

SEQ ID NO: 912 - DNA, 26 bp, Artificial sequence
  Primer
ccctgaccgagaagtttaatctgcct

SEQ ID NO: 913 - DNA, 20 bp, Artificial sequence
  Primer
tcttgaaagcgccacaagca

SEQ ID NO: 914 - DNA, 22 bp, Artificial sequence
  Primer
atgctctcccctcctcggagga

SEQ ID NO: 915 - DNA, 20 bp, Artificial sequence
  Primer
ggagaggagcaccaccccag

SEQ ID NO: 916 - DNA, 30 bp, Artificial sequence
  Primer
gtgtccctatctctgataccatcatcccag

SEQ ID NO: 917 - DNA, 30 bp, Artificial sequence
  Primer
ctccttcagacaatgcagtggtcttaacaa

SEQ ID NO: 918 - DNA, 30 bp, Artificial sequence
  Primer
gcacacctcttagaggaagacagaaaacag

SEQ ID NO: 919 - DNA, 30 bp, Artificial sequence
  Primer
gaagtggtcatttcagatgtgattcatcta

SEQ ID NO: 920 - DNA, 26 bp, Artificial sequence
  Primer
ctcctcaccctctgccgagtctcaat

SEQ ID NO: 921 - DNA, 20 bp, Artificial sequence
  Primer
gagtgcgccggtctcgggga

SEQ ID NO: 922 - DNA, 22 bp, Artificial sequence
  Primer
tggtggctatgaacccagaggt

SEQ ID NO: 923 - DNA, 30 bp, Artificial sequence
  Primer
agtctgtggctgattacttcaagcagattg

SEQ ID NO: 924 - DNA, 20 bp, Artificial sequence
  Primer
cccatctctgggattcccag

SEQ ID NO: 925 - DNA, 24 bp, Artificial sequence
  Primer
ctgaagtctgagctggacatgctg

SEQ ID NO: 926 - DNA, 20 bp, Artificial sequence
  Primer
gatcccctgttggggatgct

SEQ ID NO: 927 - DNA, 24 bp, Artificial sequence
  Primer
ctgaaggatgctgtaccacagacg

SEQ ID NO: 928 - DNA, 30 bp, Artificial sequence
  Primer
ggacgactttatgaccaagagctgaacaag

SEQ ID NO: 929 - DNA, 22 bp, Artificial sequence
  Primer
ctgcatacggcaggagggaaag

SEQ ID NO: 930 - DNA, 20 bp, Artificial sequence
  Primer
gaaccaaccggtgagccctc

SEQ ID NO: 931 - DNA, 24 bp, Artificial sequence
  Primer
tgaaccccaccaacacagtttttg

SEQ ID NO: 932 - DNA, 20 bp, Artificial sequence
  Primer
ggccaacgggtctaaagcag

SEQ ID NO: 933 - DNA, 30 bp, Artificial sequence
  Primer
aacctatgttgccctgagttacataaatag

SEQ ID NO: 934 - DNA, 20 bp, Artificial sequence
  Primer
ccgcagcagcactccgacag

SEQ ID NO: 935 - DNA, 30 bp, Artificial sequence
  Primer
gggaggttcaagattcttatgaagcttatg

SEQ ID NO: 936 - DNA, 24 bp, Artificial sequence
  Primer
gcagaagttagcgcttctctctcg

SEQ ID NO: 937 - DNA, 20 bp, Artificial sequence
  Primer
gccgtggtggctggttccct

SEQ ID NO: 938 - DNA, 22 bp, Artificial sequence
  Primer
cgactcattcatcgccctccag

SEQ ID NO: 939 - DNA, 29 bp, Artificial sequence
  Primer
gtaattatgtggtgacagatcacggctcg

SEQ ID NO: 940 - DNA, 20 bp, Artificial sequence
  Primer
tgcggggccaggtggccaag

SEQ ID NO: 941 - DNA, 30 bp, Artificial sequence
  Primer
ctggacttccagaagaacatctacagtgag

SEQ ID NO: 942 - DNA, 30 bp, Artificial sequence
  Primer
gagaatcttttaggacaagcactgacgaag

SEQ ID NO: 943 - DNA, 28 bp, Artificial sequence
  Primer
ctccagggttccttgaaaagaaaacagg

SEQ ID NO: 944 - DNA, 30 bp, Artificial sequence
  Primer
taaaaagcgaaagaataaaaaccggcacag

SEQ ID NO: 945 - DNA, 22 bp, Artificial sequence
  Primer
ggggacaacagcagtgagcaag

SEQ ID NO: 946 - DNA, 30 bp, Artificial sequence
  Primer
gccactcaatgacaaaaatagtaacagtgg

SEQ ID NO: 947 - DNA, 22 bp, Artificial sequence
  Primer
tccacggacgactcagagcaag

SEQ ID NO: 948 - DNA, 30 bp, Artificial sequence
  Primer
aatgaagttagaagaaagcgaattccatca

SEQ ID NO: 949 - DNA, 20 bp, Artificial sequence
  Primer
cggggcagatccaggttcag

SEQ ID NO: 950 - DNA, 30 bp, Artificial sequence
  Primer
tttacagctgaccttgaccagtttgatcag

SEQ ID NO: 951 - DNA, 28 bp, Artificial sequence
  Primer
gattacctgagctggaattggaagcaat

SEQ ID NO: 952 - DNA, 22 bp, Artificial sequence
  Primer
cctggcagtgagctggacaact

SEQ ID NO: 953 - DNA, 28 bp, Artificial sequence
  Primer
cttttaataacccacgaccagggcaact

SEQ ID NO: 954 - DNA, 26 bp, Artificial sequence
  Primer
gaatgattggtaacagtgcttctcgg

SEQ ID NO: 955 - DNA, 28 bp, Artificial sequence
  Primer
catcctgcctatagaccaggcgtctttt

SEQ ID NO: 956 - DNA, 28 bp, Artificial sequence
  Primer
ggccatctgaattagagatgaacatggg

SEQ ID NO: 957 - DNA, 20 bp, Artificial sequence
  Primer
cccgaccctgcccgccctgg

SEQ ID NO: 958 - DNA, 28 bp, Artificial sequence
  Primer
ctgaggatttgtgactggaccatgaatc

SEQ ID NO: 959 - DNA, 24 bp, Artificial sequence
  Primer
tcctggtacctgggctagcttggt

SEQ ID NO: 960 - DNA, 20 bp, Artificial sequence
  Primer
gtgggaggccgcaccatgct

SEQ ID NO: 961 - DNA, 24 bp, Artificial sequence
  Primer
agagcacggataactttatcttgt

SEQ ID NO: 962 - DNA, 26 bp, Artificial sequence
  Primer
ttgacgaagtgagtcccacacctcct

SEQ ID NO: 963 - DNA, 30 bp, Artificial sequence
  Primer
atgaacagcaaagatgttcagtattgtgct

SEQ ID NO: 964 - DNA, 20 bp, Artificial sequence
  Primer
catctgcattgccgggaccg

SEQ ID NO: 965 - DNA, 26 bp, Artificial sequence
  Primer
gttcatggagtttgaggctgaggaga

SEQ ID NO: 966 - DNA, 24 bp, Artificial sequence
  Primer
tgtacattccgaagaaggcagcct

SEQ ID NO: 967 - DNA, 20 bp, Artificial sequence
  Primer
catacccagcgctgggaccg

SEQ ID NO: 968 - DNA, 30 bp, Artificial sequence
  Primer
gaatctttctgaacctgtcatgacctatag

SEQ ID NO: 969 - DNA, 20 bp, Artificial sequence
  Primer
ggcggcggtgcagcgctccg

SEQ ID NO: 970 - DNA, 28 bp, Artificial sequence
  Primer
gcctgatcacttgaacggacatatcaag

SEQ ID NO: 971 - DNA, 24 bp, Artificial sequence
  Primer
acctgcaatgcttcttttgccacc

SEQ ID NO: 972 - DNA, 28 bp, Artificial sequence
  Primer
tcttaccagcccacatctattccacaag

SEQ ID NO: 973 - DNA, 24 bp, Artificial sequence
  Primer
gcggaagagacggaatttcaacaa

SEQ ID NO: 974 - DNA, 28 bp, Artificial sequence
  Primer
acggaaaaggcgtaacttcagtaaacag

SEQ ID NO: 975 - DNA, 26 bp, Artificial sequence
  Primer
ttgacctggataggctcaatgatgat

SEQ ID NO: 976 - DNA, 20 bp, Artificial sequence
  Primer
cagccccatccggatgtttg

SEQ ID NO: 977 - DNA, 20 bp, Artificial sequence
  Primer
gcccccccaggatgcaatgg

SEQ ID NO: 978 - DNA, 20 bp, Artificial sequence
  Primer
gttgcctcttggtgctgcct

SEQ ID NO: 979 - DNA, 24 bp, Artificial sequence
  Primer
attggccaaaatgggaaggattgg

SEQ ID NO: 980 - DNA, 24 bp, Artificial sequence
  Primer
tcccaggacatcaaagctctgcag

SEQ ID NO: 981 - DNA, 24 bp, Artificial sequence
  Primer
gtgaaaaaacacgtgcgcagcttc

SEQ ID NO: 982 - DNA, 30 bp, Artificial sequence
  Primer
gagatatctctgtgagtatttcagtatcaa

SEQ ID NO: 983 - DNA, 30 bp, Artificial sequence
  Primer
gacatcagcacagtatatcagatttttcct

SEQ ID NO: 984 - DNA, 20 bp, Artificial sequence
  Primer
gtgccccaaagatgcaaacg

SEQ ID NO: 985 - DNA, 30 bp, Artificial sequence
  Primer
aagtatttggctgaggagttttcaatccca

SEQ ID NO: 986 - DNA, 27 bp, Artificial sequence
  Primer
aagcacaagaccaagacagctcaacag

SEQ ID NO: 987 - DNA, 24 bp, Artificial sequence
  Primer
ctcagttcattgccagagagccat

SEQ ID NO: 988 - DNA, 24 bp, Artificial sequence
  Primer
caccccagccctatccctttacgt

SEQ ID NO: 989 - DNA, 30 bp, Artificial sequence
  Primer
catggagacccattcagataacccactaag

SEQ ID NO: 990 - DNA, 26 bp, Artificial sequence
  Primer
accatgtcagcaaaacttcttttggg

SEQ ID NO: 991 - DNA, 26 bp, Artificial sequence
  Primer
gttctccaaacctatccccgaatccg

SEQ ID NO: 992 - DNA, 26 bp, Artificial sequence
  Primer
acctgcagccagttacctactgcgag

SEQ ID NO: 993 - DNA, 30 bp, Artificial sequence
  Primer
atgtaaaatggggtaaactgagagattatc

SEQ ID NO: 994 - DNA, 30 bp, Artificial sequence
  Primer
aggtaccaatcttgggaaaaagaagcaaca

SEQ ID NO: 995 - DNA, 20 bp, Artificial sequence
  Primer
gacctcctccagcgggacag

SEQ ID NO: 996 - DNA, 24 bp, Artificial sequence
  Primer
gtttcagcagttcagctccaccag

SEQ ID NO: 997 - DNA, 30 bp, Artificial sequence
  Primer
atgttggatgacaataaccatcttattcag

SEQ ID NO: 998 - DNA, 28 bp, Artificial sequence
  Primer
gtatcagcagatgttgcacacaaacttg

SEQ ID NO: 999 - DNA, 22 bp, Artificial sequence
  Primer
gcggccctacggctatgaacag

SEQ ID NO: 1000 - DNA, 30 bp, Artificial sequence
  Primer
agccaacacagatctatagatttcttcgaa

SEQ ID NO: 1001 - DNA, 20 bp, Artificial sequence
  Primer
ggtcacagcccccattccag

SEQ ID NO: 1002 - DNA, 26 bp, Artificial sequence
  Primer
tgatgtccttgcattgcccattttta

SEQ ID NO: 1003 - DNA, 20 bp, Artificial sequence
  Primer
ggggctccaggacccctgcc

SEQ ID NO: 1004 - DNA, 22 bp, Artificial sequence
  Primer
agaccgaggcaaaggccctttt

SEQ ID NO: 1005 - DNA, 23 bp, Artificial sequence
  Primer
caggaacaaaggctgctccagct

SEQ ID NO: 1006 - DNA, 30 bp, Artificial sequence
  Primer
atgaccttctttctgccacaaaacgtaaag

SEQ ID NO: 1007 - DNA, 24 bp, Artificial sequence
  Primer
gcgaagctggagaagtcactggag

SEQ ID NO: 1008 - DNA, 20 bp, Artificial sequence
  Primer
ccaccagggagctcctgcag

SEQ ID NO: 1009 - DNA, 22 bp, Artificial sequence
  Primer
gaaactgggcatctctgtggcc

SEQ ID NO: 1010 - DNA, 30 bp, Artificial sequence
  Primer
gatggacatggtagagaatgcagatagttt

SEQ ID NO: 1011 - DNA, 20 bp, Artificial sequence
  Primer
gagctctgggccctggcgag

SEQ ID NO: 1012 - DNA, 20 bp, Artificial sequence
  Primer
gggcctcagcgtggactcag

SEQ ID NO: 1013 - DNA, 24 bp, Artificial sequence
  Primer
cactggccagaggtacttcctcaa

SEQ ID NO: 1014 - DNA, 22 bp, Artificial sequence
  Primer
gcagtatcccagccaaatctcg

SEQ ID NO: 1015 - DNA, 20 bp, Artificial sequence
  Primer
ccaaatcccactcccgacag

SEQ ID NO: 1016 - DNA, 23 bp, Artificial sequence
  Primer
gacttcagacatgcagggtgacg

SEQ ID NO: 1017 - DNA, 30 bp, Artificial sequence
  Primer
atgaaaaaaaagatattgaccatgagacag

SEQ ID NO: 1018 - DNA, 23 bp, Artificial sequence
  Primer
ggacaaacctgactccttcatgg

SEQ ID NO: 1019 - DNA, 22 bp, Artificial sequence
  Primer
cagctctgctaccccaagacag

SEQ ID NO: 1020 - DNA, 25 bp, Artificial sequence
  Primer
catggatctgactgccatctacgag

SEQ ID NO: 1021 - DNA, 20 bp, Artificial sequence
  Primer
caggcaccgcccctggggct

SEQ ID NO: 1022 - DNA, 20 bp, Artificial sequence
  Primer
ccactcgggcgagaagccgc

SEQ ID NO: 1023 - DNA, 20 bp, Artificial sequence
  Primer
cgggtggacattcccctcag

SEQ ID NO: 1024 - DNA, 20 bp, Artificial sequence
  Primer
gtgggcctcctgggcctcag

SEQ ID NO: 1025 - DNA, 23 bp, Artificial sequence
  Primer
tccctggaatgaagggacacaga

SEQ ID NO: 1026 - DNA, 20 bp, Artificial sequence
  Primer
atggcaaaactggcccccct

SEQ ID NO: 1027 - DNA, 23 bp, Artificial sequence
  Primer
tccctggacctaaaggtgctgct

SEQ ID NO: 1028 - DNA, 23 bp, Artificial sequence
  Primer
aagcaggcaaacctggtgaacag

SEQ ID NO: 1029 - DNA, 22 bp, Artificial sequence
  Primer
tccagggcctaagggtgacaga

SEQ ID NO: 1030 - DNA, 20 bp, Artificial sequence
  Primer
ctggtgcccctggtgacaag

SEQ ID NO: 1031 - DNA, 20 bp, Artificial sequence
  Primer
ctggaccccctggccccatt

SEQ ID NO: 1032 - DNA, 20 bp, Artificial sequence
  Primer
agggtccccctggccctcct

SEQ ID NO: 1033 - DNA, 20 bp, Artificial sequence
  Primer
ctggtcctgctggtccccga

SEQ ID NO: 1034 - DNA, 20 bp, Artificial sequence
  Primer
ctggcgagcctggagcttca

SEQ ID NO: 1035 - DNA, 22 bp, Artificial sequence
  Primer
atgtcaccgggtgcgcatcaat

SEQ ID NO: 1036 - DNA, 30 bp, Artificial sequence
  Primer
ctacaagagactgtgaaaaggaagttggaa

SEQ ID NO: 1037 - DNA, 22 bp, Artificial sequence
  Primer
catcccagtgactgcatccctc

SEQ ID NO: 1038 - DNA, 20 bp, Artificial sequence
  Primer
ggggaccccattcccgagga

SEQ ID NO: 1039 - DNA, 25 bp, Artificial sequence
  Primer
gtttcaaagtcaccctcccaccttt

SEQ ID NO: 1040 - DNA, 21 bp, Artificial sequence
  Primer
gtcccgtggctgtcatcagtg

SEQ ID NO: 1041 - DNA, 20 bp, Artificial sequence
  Primer
ccctggcgagccccttgcag

SEQ ID NO: 1042 - DNA, 26 bp, Artificial sequence
  Primer
acactaacagcacatctggagacccg

SEQ ID NO: 1043 - DNA, 20 bp, Artificial sequence
  Primer
gtctcggtggctgtgggcct

SEQ ID NO: 1044 - DNA, 22 bp, Artificial sequence
  Primer
tgtcctccttgaagggctccag

SEQ ID NO: 1045 - DNA, 27 bp, Artificial sequence
  Primer
cctccactgaagaagctgaaacaagag

SEQ ID NO: 1046 - DNA, 25 bp, Artificial sequence
  Primer
gagagtctggatggacatttgcagg

SEQ ID NO: 1047 - DNA, 20 bp, Artificial sequence
  Primer
tgcgaagccacctctcgcag

SEQ ID NO: 1048 - DNA, 27 bp, Artificial sequence
  Primer
gctctccacagatagagaacatccagc

SEQ ID NO: 1049 - DNA, 25 bp, Artificial sequence
  Primer
ctgaacagatgggtaaggatggcag

SEQ ID NO: 1050 - DNA, 23 bp, Artificial sequence
  Primer
ggaccaaccacttcctaccccag

SEQ ID NO: 1051 - DNA, 20 bp, Artificial sequence
  Primer
gccccaggtgtacccaccac

SEQ ID NO: 1052 - DNA, 20 bp, Artificial sequence
  Primer
gcctcacctgcagatgcccc

SEQ ID NO: 1053 - DNA, 25 bp, Artificial sequence
  Primer
gcaacctccaagtcccagatcatgt

SEQ ID NO: 1054 - DNA, 20 bp, Artificial sequence
  Primer
ggagttcctggtcggctccg

SEQ ID NO: 1055 - DNA, 21 bp, Artificial sequence
  Primer
cttaccgtgacgtccaccgac

SEQ ID NO: 1056 - DNA, 24 bp, Artificial sequence
  Primer
gagagagccttgaactctgccagc

SEQ ID NO: 1057 - DNA, 25 bp, Artificial sequence
  Primer
tttaaggagtcggccttgaggaagc

SEQ ID NO: 1058 - DNA, 20 bp, Artificial sequence
  Primer
gtgccaggcccacccccagg

SEQ ID NO: 1059 - DNA, 25 bp, Artificial sequence
  Primer
gtaaaggcgacacaggaggagaacc

SEQ ID NO: 1060 - DNA, 20 bp, Artificial sequence
  Primer
cctctgtgtttgccgcctgg

SEQ ID NO: 1061 - DNA, 29 bp, Artificial sequence
  Primer
tgttgaagagattggctggtcctatacag

SEQ ID NO: 1062 - DNA, 30 bp, Artificial sequence
  Primer
acacattcattcataacactgggaaaacag

SEQ ID NO: 1063 - DNA, 27 bp, Artificial sequence
  Primer
ataaacctctcataatgaaggcccccg

SEQ ID NO: 1064 - DNA, 19 bp, Artificial sequence
  Primer
cctgcagcccccatagcag

SEQ ID NO: 1065 - DNA, 20 bp, Artificial sequence
  Primer
ctcgcaacgccctggtggtc

SEQ ID NO: 1066 - DNA, 21 bp, Artificial sequence
  Primer
gtggccttgacctccaaccag

SEQ ID NO: 1067 - DNA, 20 bp, Artificial sequence
  Primer
gggctgctggagtcctctgc

SEQ ID NO: 1068 - DNA, 26 bp, Artificial sequence
  Primer
gcatagagaaggagacgtgccagaag

SEQ ID NO: 1069 - DNA, 22 bp, Artificial sequence
  Primer
cgggtcctgaacgctgtgaaat

SEQ ID NO: 1070 - DNA, 26 bp, Artificial sequence
  Primer
attatggaactgcagcgaatgacatc

SEQ ID NO: 1071 - DNA, 24 bp, Artificial sequence
  Primer
gcccagagatcgcagcatatcaaa

SEQ ID NO: 1072 - DNA, 30 bp, Artificial sequence
  Primer
gatgagattcttccaaggaaagactatgag

SEQ ID NO: 1073 - DNA, 20 bp, Artificial sequence
  Primer
ggtcaagctgctgctgctcg

SEQ ID NO: 1074 - DNA, 27 bp, Artificial sequence
  Primer
ggggacctaattacacctccggttatg

SEQ ID NO: 1075 - DNA, 20 bp, Artificial sequence
  Primer
cagcctacatcggatgccca

SEQ ID NO: 1076 - DNA, 20 bp, Artificial sequence
  Primer
cggccaacaatccctgcagt

SEQ ID NO: 1077 - DNA, 19 bp, Artificial sequence
  Primer
cgacgggtccattgccaag

SEQ ID NO: 1078 - DNA, 20 bp, Artificial sequence
  Primer
gcctgtcgggggtaccacag

SEQ ID NO: 1079 - DNA, 29 bp, Artificial sequence
  Primer
gacttgattagagaccaaggatttcgtgg

SEQ ID NO: 1080 - DNA, 26 bp, Artificial sequence
  Primer
gatcaaccacaggtttgtctgctacc

SEQ ID NO: 1081 - DNA, 27 bp, Artificial sequence
  Primer
aaaacacttggtagacgggactcgagt

SEQ ID NO: 1082 - DNA, 26 bp, Artificial sequence
  Primer
agctaaaaggacagcaggtgctacca

SEQ ID NO: 1083 - DNA, 30 bp, Artificial sequence
  Primer
tttgcagaaacactccaatttatagattct

SEQ ID NO: 1084 - DNA, 23 bp, Artificial sequence
  Primer
gcctacccttctctccctcgcag

SEQ ID NO: 1085 - DNA, 30 bp, Artificial sequence
  Primer
gaaattaaatacggtcccctgaagatgcta

SEQ ID NO: 1086 - DNA, 31 bp, Artificial sequence
  Primer
accacccttactgaagaaaatcaaacaagag

SEQ ID NO: 1087 - DNA, 20 bp, Artificial sequence
  Primer
cgcctgtggcagatgcaccg

SEQ ID NO: 1088 - DNA, 24 bp, Artificial sequence
  Primer
gaggagcaaaatagaggcaagccc

SEQ ID NO: 1089 - DNA, 25 bp, Artificial sequence
  Primer
gcagaaggagaagacagcctgaaga

SEQ ID NO: 1090 - DNA, 17 bp, Artificial sequence
  Primer
cccgcccaagggcccag

SEQ ID NO: 1091 - DNA, 20 bp, Artificial sequence
  Primer
gctcacccagtccccaccag

SEQ ID NO: 1092 - DNA, 23 bp, Artificial sequence
  Primer
aactgttccccctcatcttcccg

SEQ ID NO: 1093 - DNA, 30 bp, Artificial sequence
  Primer
aagaggatggattcgacttagacttgacct

SEQ ID NO: 1094 - DNA, 30 bp, Artificial sequence
  Primer
cttctttttcagaagacaccctaaaaaaag

SEQ ID NO: 1095 - DNA, 26 bp, Artificial sequence
  Primer
ctgattccagagagctaaagccgatg

SEQ ID NO: 1096 - DNA, 21 bp, Artificial sequence
  Primer
aaagccaaacttggccctgct

SEQ ID NO: 1097 - DNA, 20 bp, Artificial sequence
  Primer
cacctgcaagatggggctgg

SEQ ID NO: 1098 - DNA, 24 bp, Artificial sequence
  Primer
atctcctgtgtgcccagaagacct

SEQ ID NO: 1099 - DNA, 30 bp, Artificial sequence
  Primer
gtgcaaacccaaattatcctgatgtaattt

SEQ ID NO: 1100 - DNA, 26 bp, Artificial sequence
  Primer
gtctatgctgtggtggtgattgcgtc

SEQ ID NO: 1101 - DNA, 29 bp, Artificial sequence
  Primer
atttctcatggtttggatttgggaaagta

SEQ ID NO: 1102 - DNA, 20 bp, Artificial sequence
  Primer
gcccagcctccgttatcagc

SEQ ID NO: 1103 - DNA, 29 bp, Artificial sequence
  Primer
aaattaaatacggtcccctgaagatgcta

SEQ ID NO: 1104 - DNA, 25 bp, Artificial sequence
  Primer
gcagaaggagaagacagcctgaaga

SEQ ID NO: 1105 - DNA, 24 bp, Artificial sequence
  Primer
gtcgggctctggaggaaaagaaag

SEQ ID NO: 1106 - DNA, 23 bp, Artificial sequence
  Primer
tttgccaaggcacgagtaacaag

SEQ ID NO: 1107 - DNA, 21 bp, Artificial sequence
  Primer
cctgcgtgaagaagtgtcccc

SEQ ID NO: 1108 - DNA, 25 bp, Artificial sequence
  Primer
accgatcaagagctctccatgtgag

SEQ ID NO: 1109 - DNA, 23 bp, Artificial sequence
  Primer
ctccgaatgtcctggctcattcg

SEQ ID NO: 1110 - DNA, 21 bp, Artificial sequence
  Primer
gccagccaccgacacctacag

SEQ ID NO: 1111 - DNA, 21 bp, Artificial sequence
  Primer
catctcgggctacggagctgc

SEQ ID NO: 1112 - DNA, 19 bp, Artificial sequence
  Primer
ggcaattccggagccgcag

SEQ ID NO: 1113 - DNA, 21 bp, Artificial sequence
  Primer
gtggtggaggtggctggaatg

SEQ ID NO: 1114 - DNA, 27 bp, Artificial sequence
  Primer
gcatcctgtacaccccagctttaaaag

SEQ ID NO: 1115 - DNA, 20 bp, Artificial sequence
  Primer
tgatggaaggccacggggaa

SEQ ID NO: 1116 - DNA, 21 bp, Artificial sequence
  Primer
cccctgcaagtggctgtgaag

SEQ ID NO: 1117 - DNA, 22 bp, Artificial sequence
  Primer
acgctgcctgaagtgtgctctg

SEQ ID NO: 1118 - DNA, 24 bp, Artificial sequence
  Primer
cctcatggaagccctgatcatcag

SEQ ID NO: 1119 - DNA, 27 bp, Artificial sequence
  Primer
caaattcaaccaccagaacattgttcg

SEQ ID NO: 1120 - DNA, 23 bp, Artificial sequence
  Primer
gggatggcccgagacatctacag

SEQ ID NO: 1121 - DNA, 27 bp, Artificial sequence
  Primer
ggcgagctactatagaaagggaggctg

SEQ ID NO: 1122 - DNA, 21 bp, Artificial sequence
  Primer
caagaactgccctgggcctgt

SEQ ID NO: 1123 - DNA, 26 bp, Artificial sequence
  Primer
ataccggataatgactcagtgctggc

SEQ ID NO: 1124 - DNA, 51 bp, Artificial sequence
  Fusion transcript
attgctgtgggaaataatgatgtaaaggaggatccaaagtgggaattccct

SEQ ID NO: 1125 - DNA, 7 bp, Artificial sequence
  Sequence of molecular barcode
gtgctca

SEQ ID NO: 1126 - DNA, 7 bp, Artificial sequence
  Sequence of molecular barcode
ctagggc

SEQ ID NO: 1127 - DNA, 7 bp, Artificial sequence
  Sequence of molecular barcode
atgctat

SEQ ID NO: 1128 - DNA, 7 bp, Artificial sequence
  Sequence of molecular barcode
ctttgta

SEQ ID NO: 1129 - DNA, 7 bp, Artificial sequence
  Sequence of molecular barcode
tgaccaa

SEQ ID NO: 1130 - DNA, 7 bp, Artificial sequence
  Sequence of molecular barcode
aggtctt

SEQ ID NO: 1131 - DNA, 7 bp, Artificial sequence
  Sequence of molecular barcode
tccattt

SEQ ID NO: 1132 - DNA, 7 bp, Artificial sequence
  Sequence of molecular barcode
tcgttga

SEQ ID NO: 1133 - DNA, 7 bp, Artificial sequence
  Sequence of molecular barcode
gaaaata

SEQ ID NO: 1134 - DNA, 7 bp, Artificial sequence
  Sequence of molecular barcode
gcgagta

SEQ ID NO: 1135 - DNA, 7 bp, Artificial sequence
  Sequence of molecular barcode
gggggta

SEQ ID NO: 1136 - DNA, 7 bp, Artificial sequence
  Sequence of molecular barcode
tccagcc

SEQ ID NO: 1137 - DNA, 7 bp, Artificial sequence
  Sequence of molecular barcode
acgctta

SEQ ID NO: 1138 - DNA, 7 bp, Artificial sequence
  Sequence of molecular barcode
tcctgcg

SEQ ID NO: 1139 - DNA, 7 bp, Artificial sequence
  Sequence of molecular barcode
gtgggct

SEQ ID NO: 1140 - DNA, 7 bp, Artificial sequence
  Sequence of molecular barcode
ggccggc

SEQ ID NO: 1141 - DNA, 7 bp, Artificial sequence
  Sequence of molecular barcode
gggtcac

SEQ ID NO: 1142 - DNA, 7 bp, Artificial sequence
  Sequence of molecular barcode
cgagatt

SEQ ID NO: 1143 - DNA, 7 bp, Artificial sequence
  Sequence of molecular barcode
acctgat

SEQ ID NO: 1144 - DNA, 7 bp, Artificial sequence
  Sequence of molecular barcode
gcggcta

SEQ ID NO: 1145 - DNA, 7 bp, Artificial sequence
  Sequence of molecular barcode
gacgtct

SEQ ID NO: 1146 - DNA, 7 bp, Artificial sequence
  Sequence of molecular barcode
gtgtcta

SEQ ID NO: 1147 - DNA, 7 bp, Artificial sequence
  Sequence of molecular barcode
cgtactg

SEQ ID NO: 1148 - DNA, 22 bp, Artificial sequence
  Primer
agcagcagctacgggcagcaga

SEQ ID NO: 1149 - DNA, 25 bp, Artificial sequence
  Primer
gttcactgctggcctatacaacctc

SEQ ID NO: 1150 - DNA, 47 bp, Artificial sequence
  Fusion transcript
agcagcagctacgggcagcagagttcactgctggcctatacaacctc

SEQ ID NO: 1151 - DNA, 7 bp, Artificial sequence
  Sequence of molecular barcode
catgagg

SEQ ID NO: 1152 - DNA, 7 bp, Artificial sequence
  Sequence of molecular barcode
tcgcggc

SEQ ID NO: 1153 - DNA, 7 bp, Artificial sequence
  Sequence of molecular barcode
tttgttt

SEQ ID NO: 1154 - DNA, 7 bp, Artificial sequence
  Sequence of molecular barcode
cgtgtgg

SEQ ID NO: 1155 - DNA, 7 bp, Artificial sequence
  Sequence of molecular barcode
cttgggg

SEQ ID NO: 1156 - DNA, 7 bp, Artificial sequence
  Sequence of molecular barcode
tagcgat

SEQ ID NO: 1157 - DNA, 7 bp, Artificial sequence
  Sequence of molecular barcode
cgtcctt

SEQ ID NO: 1158 - DNA, 7 bp, Artificial sequence
  Sequence of molecular barcode
gtgagtc

SEQ ID NO: 1159 - DNA, 7 bp, Artificial sequence
  Sequence of molecular barcode
cgggggg

SEQ ID NO: 1160 - DNA, 7 bp, Artificial sequence
  Sequence of molecular barcode
gagcctg

SEQ ID NO: 1161 - DNA, 7 bp, Artificial sequence
  Sequence of molecular barcode
gttttgg

SEQ ID NO: 1162 - DNA, 7 bp, Artificial sequence
  Sequence of molecular barcode
gtcggga

SEQ ID NO: 1163 - DNA, 7 bp, Artificial sequence
  Sequence of molecular barcode
ttggtcc

SEQ ID NO: 1164 - DNA, 7 bp, Artificial sequence
  Sequence of molecular barcode
acggaag

SEQ ID NO: 1165 - DNA, 7 bp, Artificial sequence
  Sequence of molecular barcode
agtatta

SEQ ID NO: 1166 - DNA, 7 bp, Artificial sequence
  Sequence of molecular barcode
cattcgc

SEQ ID NO: 1167 - DNA, 7 bp, Artificial sequence
  Sequence of molecular barcode
tagtaag

SEQ ID NO: 1168 - DNA, 7 bp, Artificial sequence
  Sequence of molecular barcode
tcctacg

SEQ ID NO: 1169 - DNA, 7 bp, Artificial sequence
  Sequence of molecular barcode
ggtatgg

SEQ ID NO: 1170 - DNA, 7 bp, Artificial sequence
  Sequence of molecular barcode
cggggta

SEQ ID NO: 1171 - DNA, 7 bp, Artificial sequence
  Sequence of molecular barcode
ctgatag

SEQ ID NO: 1172 - DNA, 7 bp, Artificial sequence
  Sequence of molecular barcode
tagggtg

SEQ ID NO: 1173 - DNA, 7 bp, Artificial sequence
  Sequence of molecular barcode
tggggag

SEQ ID NO: 1174 - DNA, 7 bp, Artificial sequence
  Sequence of molecular barcode
gctggtc

SEQ ID NO: 1175 - DNA, 7 bp, Artificial sequence
  Sequence of molecular barcode
tatgggc

SEQ ID NO: 1176 - DNA, 7 bp, Artificial sequence
  Sequence of molecular barcode
atacgtc

SEQ ID NO: 1177 - DNA, 7 bp, Artificial sequence
  Sequence of molecular barcode
agacaac

SEQ ID NO: 1178 - DNA, 26 bp, Artificial sequence
  Primer
agcagaggccttatggatatgaccag

SEQ ID NO: 1179 - DNA, 23 bp, Artificial sequence
  Primer
atcatgcccaagaagccagcaga

SEQ ID NO: 1180 - DNA, 49 bp, Artificial sequence
  Fusion transcript
agcagaggccttatggatatgaccagatcatgcccaagaagccagcaga

SEQ ID NO: 1181 - DNA, 7 bp, Artificial sequence
  Sequence of molecular barcode
atgtgtc

SEQ ID NO: 1182 - DNA, 7 bp, Artificial sequence
  Sequence of molecular barcode
gggggcg

SEQ ID NO: 1183 - DNA, 7 bp, Artificial sequence
  Sequence of molecular barcode
atattcg

SEQ ID NO: 1184 - DNA, 7 bp, Artificial sequence
  Sequence of molecular barcode
cgcgttt

SEQ ID NO: 1185 - DNA, 7 bp, Artificial sequence
  Sequence of molecular barcode
gtggtta

SEQ ID NO: 1186 - DNA, 7 bp, Artificial sequence
  Sequence of molecular barcode
cgggttt

SEQ ID NO: 1187 - DNA, 7 bp, Artificial sequence
  Sequence of molecular barcode
gggaggc

SEQ ID NO: 1188 - DNA, 7 bp, Artificial sequence
  Sequence of molecular barcode
gtatatg

SEQ ID NO: 1189 - DNA, 7 bp, Artificial sequence
  Sequence of molecular barcode
accttgt

SEQ ID NO: 1190 - DNA, 7 bp, Artificial sequence
  Sequence of molecular barcode
ttgcaga

SEQ ID NO: 1191 - DNA, 7 bp, Artificial sequence
  Sequence of molecular barcode
ggggcaa

SEQ ID NO: 1192 - DNA, 7 bp, Artificial sequence
  Sequence of molecular barcode
gaggctt

SEQ ID NO: 1193 - DNA, 7 bp, Artificial sequence
  Sequence of molecular barcode
tcatttt

SEQ ID NO: 1194 - DNA, 7 bp, Artificial sequence
  Sequence of molecular barcode
ggtgact

SEQ ID NO: 1195 - DNA, 7 bp, Artificial sequence
  Sequence of molecular barcode
tgtgcgt

SEQ ID NO: 1196 - DNA, 7 bp, Artificial sequence
  Sequence of molecular barcode
gggagag

SEQ ID NO: 1197 - DNA, 7 bp, Artificial sequence
  Sequence of molecular barcode
gccattt

SEQ ID NO: 1198 - DNA, 7 bp, Artificial sequence
  Sequence of molecular barcode
aagccaa

SEQ ID NO: 1199 - DNA, 7 bp, Artificial sequence
  Sequence of molecular barcode
attaggg

SEQ ID NO: 1200 - DNA, 7 bp, Artificial sequence
  Sequence of molecular barcode
cctggtt

SEQ ID NO: 1201 - DNA, 7 bp, Artificial sequence
  Sequence of molecular barcode
gatttgt

SEQ ID NO: 1202 - DNA, 7 bp, Artificial sequence
  Sequence of molecular barcode
tagagtt

SEQ ID NO: 1203 - DNA, 7 bp, Artificial sequence
  Sequence of molecular barcode
tgctttg

SEQ ID NO: 1204 - DNA, 7 bp, Artificial sequence
  Sequence of molecular barcode
tcctagc

SEQ ID NO: 1205 - DNA, 7 bp, Artificial sequence
  Sequence of molecular barcode
gtaatct

SEQ ID NO: 1206 - DNA, 7 bp, Artificial sequence
  Sequence of molecular barcode
gagcctg

SEQ ID NO: 1207 - DNA, 7 bp, Artificial sequence
  Sequence of molecular barcode
ccgcagg

SEQ ID NO: 1208 - DNA, 7 bp, Artificial sequence
  Sequence of molecular barcode
gccggga

SEQ ID NO: 1209 - DNA, 30 bp, Artificial sequence; identical to SEQ ID NO: 12
  Primer

SEQ ID NO: 1210 - DNA, 27 bp, Artificial sequence
  Primer
tggaaaagacaattgatgacctggaag

SEQ ID NO: 1211 - DNA, 28 bp, Artificial sequence
  Primer
gatagctagcggccaggagaaatacagt

SEQ ID NO: 1212 - DNA, 30 bp, Artificial sequence
  Primer
tgacttctggattctcctcttgagtaaaag

SEQ ID NO: 1213 - DNA, 25 bp, Artificial sequence
  Primer
cgaacatggcacgaaagagatcaag

SEQ ID NO: 1214 - DNA, 29 bp, Artificial sequence
  Primer
tttggacatcacatttcacagtcagaagg

SEQ ID NO: 1215 - DNA, 27 bp, Artificial sequence
  Primer
accaagccaccctggtagaacaagtaa

SEQ ID NO: 1216 - DNA, 28 bp, Artificial sequence
  Primer
acaggtgatttggcttctgcacagttag

SEQ ID NO: 1217 - DNA, 22 bp, Artificial sequence
  Primer
atggtgctccaagaggcagctt

SEQ ID NO: 1218 - DNA, 30 bp, Artificial sequence
  Primer
ccttattggagattttacattgtgctatag

SEQ ID NO: 1219 - DNA, 28 bp, Artificial sequence
  Primer
ctggctggaaaaagaggaaagatttctg

SEQ ID NO: 1220 - DNA, 21 bp, Artificial sequence
  Primer
tgggagaagcagcagcgcaag

SEQ ID NO: 1221 - DNA, 25 bp, Artificial sequence
  Primer
gccaagaggcagacctaggaaatgg

SEQ ID NO: 1222 - DNA, 28 bp, Artificial sequence
  Primer
ctccagaaacatgacaaggaggactttc

SEQ ID NO: 1223 - DNA, 20 bp, Artificial sequence
  Primer
tggcgaagcggaggccggag

SEQ ID NO: 1224 - DNA, 21 bp, Artificial sequence
  Primer
ctgtctgcgagcctggctgtg

SEQ ID NO: 1225 - DNA, 27 bp, Artificial sequence
  Primer
caagttgttcagaagaagcctgctcag

SEQ ID NO: 1226 - DNA, 23 bp, Artificial sequence
  Primer
agatggtgcagaagaagaacgcg

SEQ ID NO: 1227 - DNA, 25 bp, Artificial sequence
  Primer
ggtacgaagccagcctcatacatgc

SEQ ID NO: 1228 - DNA, 29 bp, Artificial sequence
  Primer
ggaactgccagtgtagagggaattctaag

SEQ ID NO: 1229 - DNA, 27 bp, Artificial sequence
  Primer
gcctttttgaagaaactccacgaagag

SEQ ID NO: 1230 - DNA, 30 bp, Artificial sequence
  Primer
gatgagcaattcttaggttttggctcagat

SEQ ID NO: 1231 - DNA, 22 bp, Artificial sequence
  Primer
gctggaaacatttccgaccctg

SEQ ID NO: 1232 - DNA, 30 bp, Artificial sequence
  Primer
aaggagaaggggttgaaattgttgatagag

SEQ ID NO: 1233 - DNA, 28 bp, Artificial sequence
  Primer
atcaagtcctttgacagtgcatctcaag

SEQ ID NO: 1234 - DNA, 26 bp, Artificial sequence
  Primer
gcaagagtggtgatcgtggtgagact

SEQ ID NO: 1235 - DNA, 30 bp, Artificial sequence
  Primer
tttttttgaagaagcaggatgctgatctaa

SEQ ID NO: 1236 - DNA, 30 bp, Artificial sequence
  Primer
tcttatcctttgtcgcagagactatctgag

SEQ ID NO: 1237 - DNA, 24 bp, Artificial sequence
  Primer
ggctattgagtggccagacttccc

SEQ ID NO: 1238 - DNA, 24 bp, Artificial sequence
  Primer
aggttgttaccgtgggcaactctg

SEQ ID NO: 1239 - DNA, 21 bp, Artificial sequence
  Primer
gtggtggaggtggctggaatg

SEQ ID NO: 1240 - DNA, 24 bp, Artificial sequence
  Primer
ccagaaaaaaagaccaggccacag

SEQ ID NO: 1241 - DNA, 26 bp, Artificial sequence
  Primer
gccttctaccccatgagaaagaccag

SEQ ID NO: 1242 - DNA, 24 bp, Artificial sequence
  Primer
cagcagccagtaaggaggagaagg

SEQ ID NO: 1243 - DNA, 27 bp, Artificial sequence
  Primer
gagttcaggaccagctcattgaaaaga

SEQ ID NO: 1244 - DNA, 26 bp, Artificial sequence
  Primer
gtggaaaaggctttagccatggacag

SEQ ID NO: 1245 - DNA, 30 bp, Artificial sequence
  Primer
agatctgtcttacaacctattagaagattt

SEQ ID NO: 1246 - DNA, 22 bp, Artificial sequence
  Primer
ccaaggcttgaccctcgttttg

SEQ ID NO: 1247 - DNA, 24 bp, Artificial sequence
  Primer
aaacagcaagaactgcttcggcag

SEQ ID NO: 1248 - DNA, 26 bp, Artificial sequence
  Primer
acaagtcatcaattgctggctcagaa

SEQ ID NO: 1249 - DNA, 30 bp, Artificial sequence
  Primer
ggtcaagaaagtgactcatcagagacctct

SEQ ID NO: 1250 - DNA, 21 bp, Artificial sequence
  Primer
gtcctccgacagtgcttggca

SEQ ID NO: 1251 - DNA, 20 bp, Artificial sequence
  Primer
aagatgaatccggcctcggc

SEQ ID NO: 1252 - DNA, 23 bp, Artificial sequence
  Primer
cggagtcagctgccaagagacag

SEQ ID NO: 1253 - DNA, 30 bp, Artificial sequence
  Primer
gtgctatacttggtagatcagaaactcagg

SEQ ID NO: 1254 - DNA, 22 bp, Artificial sequence
  Primer
gaccatcatccagggcatcctg

SEQ ID NO: 1255 - DNA, 21 bp, Artificial sequence
  Primer
tgacacgcttccctggattgg

SEQ ID NO: 1256 - DNA, 21 bp, Artificial sequence
  Primer
cagctcctgaccaaccccaag

SEQ ID NO: 1257 - DNA, 21 bp, Artificial sequence
  Primer
acagggacgccatcgaatccg

SEQ ID NO: 1258 - DNA, 30 bp, Artificial sequence
  Primer
tgaaatccgacactactgattctagtcaag

SEQ ID NO: 1259 - DNA, 30 bp, Artificial sequence
  Primer
ttggagaagatctatgggtcagacagaatt

SEQ ID NO: 1260 - DNA, 30 bp, Artificial sequence
  Primer
gttactctggaagaagtcaactcccaaata

SEQ ID NO: 1261 - DNA, 30 bp, Artificial sequence
  Primer
aactcgaaaattaatgctgaaaataaggcg

SEQ ID NO: 1262 - DNA, 23 bp, Artificial sequence
  Primer
gactgggaggtgctggtcctagg

SEQ ID NO: 1263 - DNA, 30 bp, Artificial sequence
  Primer
tttaaggctgcaagcagtatttacaacaga

SEQ ID NO: 1264 - DNA, 30 bp, Artificial sequence
  Primer
aatcatcggactcaggtacatctgtgagtg

SEQ ID NO: 1265 - DNA, 22 bp, Artificial sequence
  Primer
gcctgtgcagtgggactgattg

SEQ ID NO: 1266 - DNA, 30 bp, Artificial sequence
  Primer
gttcaaaaactgaaggactctgaagctgag

SEQ ID NO: 1267 - DNA, 27 bp, Artificial sequence
  Primer
cgccaattgtaaacaaagtggtgacac

SEQ ID NO: 1268 - DNA, 27 bp, Artificial sequence
  Primer
ccttattgattggccaacaatcaacag

SEQ ID NO: 1269 - DNA, 20 bp, Artificial sequence
  Primer
cccagccctggggagcccct

SEQ ID NO: 1270 - DNA, 24 bp, Artificial sequence
  Primer
ccgtagctccatattggacatccc

SEQ ID NO: 1271 - DNA, 25 bp, Artificial sequence
  Primer
ccctgagaatctgggacctcaacag

SEQ ID NO: 1272 - DNA, 21 bp, Artificial sequence
  Primer
tgtgtgcctcctgacgaagcc

SEQ ID NO: 1273 - DNA, 22 bp, Artificial sequence
  Primer
gccacagtggagaccagtcagc

SEQ ID NO: 1274 - DNA, 23 bp, Artificial sequence
  Primer
gccaagaggagctcatgaggcag

SEQ ID NO: 1275 - DNA, 30 bp, Artificial sequence
  Primer
tctctagcagttactatggatgacttccgg

SEQ ID NO: 1276 - DNA, 30 bp, Artificial sequence
  Primer
aactcacaacggtaggagagaaacctgaag

SEQ ID NO: 1277 - DNA, 24 bp, Artificial sequence
  Primer
agcccgggaccgtttaaaaaactg

SEQ ID NO: 1278 - DNA, 23 bp, Artificial sequence
  Primer
aaatgtggagcccaggaggaagg

SEQ ID NO: 1279 - DNA, 29 bp, Artificial sequence
  Primer
aatggtcagaaaccctccataacctgaag

SEQ ID NO: 1280 - DNA, 25 bp, Artificial sequence
  Primer
gatgcaattcgaagtcacagcgaat

SEQ ID NO: 1281 - DNA, 26 bp, Artificial sequence
  Primer
cggacgcatcacttgcacttctagaa

SEQ ID NO: 1282 - DNA, 30 bp, Artificial sequence
  Primer
agctgatagacacacaccttagctggatac

SEQ ID NO: 1283 - DNA, 23 bp, Artificial sequence
  Primer
ctttgctgaatgctccagccaag

SEQ ID NO: 1284 - DNA, 29 bp, Artificial sequence
  Primer
cttgtaatctggatgtgattctggggttt

SEQ ID NO: 1285 - DNA, 26 bp, Artificial sequence
  Primer
gaaagcccttcttgtatgtcaatgcc

SEQ ID NO: 1286 - DNA, 29 bp, Artificial sequence
  Primer
gtaacagtatcgggacccttactgcacat

SEQ ID NO: 1287 - DNA, 30 bp, Artificial sequence
  Primer
acattactggttatagaattaccacaaccc

SEQ ID NO: 1288 - DNA, 28 bp, Artificial sequence
  Primer
ctcaagcttttaaaatcgagaccacccc

SEQ ID NO: 1289 - DNA, 20 bp, Artificial sequence
  Primer
agccccagtcccagccccag

SEQ ID NO: 1290 - DNA, 29 bp, Artificial sequence
  Primer
aatgcagctcttcagcatctgtttattcg

SEQ ID NO: 1291 - DNA, 29 bp, Artificial sequence
  Primer
cgagggtgttcttgacgattaatcaacag

SEQ ID NO: 1292 - DNA, 21 bp, Artificial sequence
  Primer
ctccgccccacagtccacgag

SEQ ID NO: 1293 - DNA, 21 bp, Artificial sequence
  Primer
gtggcggaatcggtggtagag

SEQ ID NO: 1294 - DNA, 26 bp, Artificial sequence
  Primer
cgccatcatcctcatcatcatcatag

SEQ ID NO: 1295 - DNA, 25 bp, Artificial sequence
  Primer
agatcatcactggtatgccagcctc

SEQ ID NO: 1296 - DNA, 30 bp, Artificial sequence
  Primer
acagtctcttgcaatcggctaaaaaaaaga

SEQ ID NO: 1297 - DNA, 27 bp, Artificial sequence
  Primer
ctatcagaagaaaatcggcacctgaga

SEQ ID NO: 1298 - DNA, 30 bp, Artificial sequence
  Primer
agaaaactcttaaagaatgcagcagcttgg

SEQ ID NO: 1299 - DNA, 24 bp, Artificial sequence
  Primer
gacactggggttgggaaatcaagc

SEQ ID NO: 1300 - DNA, 23 bp, Artificial sequence
  Primer
cccagcgctaccttgtcattcag

SEQ ID NO: 1301 - DNA, 27 bp, Artificial sequence
  Primer
cagtttgctgtgtgtttgctcaaacag

SEQ ID NO: 1302 - DNA, 30 bp, Artificial sequence
  Primer
tacttggactagtttatatgaaatttgtgg

SEQ ID NO: 1303 - DNA, 28 bp, Artificial sequence
  Primer
gacatgaacaagctgagtggaggcggcg

SEQ ID NO: 1304 - DNA, 28 bp, Artificial sequence
  Primer
ctacatctacatccaccactgggacaag

SEQ ID NO: 1305 - DNA, 21 bp, Artificial sequence
  Primer
ccttgcctccccgattgaaag

SEQ ID NO: 1306 - DNA, 21 bp, Artificial sequence
  Primer
gtgccacggtgtccggatatg

SEQ ID NO: 1307 - DNA, 30 bp, Artificial sequence
  Primer
attttaatgaaaacacagcagcacctagag

SEQ ID NO: 1308 - DNA, 28 bp, Artificial sequence
  Primer
atgaaggaaatgctaaagcgattccaag

SEQ ID NO: 1309 - DNA, 21 bp, Artificial sequence
  Primer
tgccatctccaggccttgcag

SEQ ID NO: 1310 - DNA, 20 bp, Artificial sequence
  Primer
gcccggctgtgctggctcca

SEQ ID NO: 1311 - DNA, 20 bp, Artificial sequence
  Primer
tcccggccagtgtgcagctg

SEQ ID NO: 1312 - DNA, 21 bp, Artificial sequence
  Primer
ggtcctgtcggggaaccctct

SEQ ID NO: 1313 - DNA, 47 bp, Artificial sequence
  Amorgage + (Barcode) + Primer
gatagctagcggccaggagaaatacagttccaacccttagggaaccc

SEQ ID NO: 1314 - DNA, 60 bp, Artificial sequence
  Amorgage + (Barcode) + Primer
  misc_feature (24)..(30): n is a, c, g, or t
gtgccagcaagatccaatctagannnnnnntgacttctggattctcctcttgagtaaaag

SEQ ID NO: 1315 - DNA, 55 bp, Artificial sequence
  Amorgage + (Barcode) + Primer
  misc_feature (24)..(30): n is a, c, g, or t
gtgccagcaagatccaatctagannnnnnncgaacatggcacgaaagagatcaag

SEQ ID NO: 1316 - DNA, 48 bp, Artificial sequence
  Amorgage + (Barcode) + Primer
tttggacatcacatttcacagtcagaaggtccaacccttagggaaccc

SEQ ID NO: 1317 - DNA, 46 bp, Artificial sequence
  Amorgage + (Barcode) + Primer
accaagccaccctggtagaacaagtaatccaacccttagggaaccc

SEQ ID NO: 1318 - DNA, 47 bp, Artificial sequence
  Amorgage + (Barcode) + Primer
acaggtgatttggcttctgcacagttagtccaacccttagggaaccc

SEQ ID NO: 1319 - DNA, 41 bp, Artificial sequence
  Amorgage + (Barcode) + Primer
atggtgctccaagaggcagctttccaacccttagggaaccc

SEQ ID NO: 1320 - DNA, 60 bp, Artificial sequence
  Amorgage + (Barcode) + Primer
  misc_feature (24)..(30): n is a, c, g, or t
gtgccagcaagatccaatctagannnnnnnccttattggagattttacattgtgctatag

SEQ ID NO: 1321 - DNA, 58 bp, Artificial sequence
  Amorgage + (Barcode) + Primer
  misc_feature (24)..(30): n is a, c, g, or t
gtgccagcaagatccaatctagannnnnnnctggctggaaaaagaggaaagatttctg

SEQ ID NO: 1322 - DNA, 51 bp, Artificial sequence
  Amorgage + (Barcode) + Primer
  misc_feature (24)..(30): n is a, c, g, or t
gtgccagcaagatccaatctagannnnnnntgggagaagcagcagcgcaag

SEQ ID NO: 1323 - DNA, 55 bp, Artificial sequence
  Amorgage + (Barcode) + Primer
  misc_feature (24)..(30): n is a, c, g, or t
gtgccagcaagatccaatctagannnnnnngccaagaggcagacctaggaaatgg

SEQ ID NO: 1324 - DNA, 47 bp, Artificial sequence
  Amorgage + (Barcode) + Primer
ctccagaaacatgacaaggaggactttctccaacccttagggaaccc

SEQ ID NO: 1325 - DNA, 50 bp, Artificial sequence
  Amorgage + (Barcode) + Primer
  misc_feature (24)..(30): n is a, c, g, or t
gtgccagcaagatccaatctagannnnnnntggcgaagcggaggccggag

SEQ ID NO: 1326 - DNA, 40 bp, Artificial sequence
  Amorgage + (Barcode) + Primer
ctgtctgcgagcctggctgtgtccaacccttagggaaccc

SEQ ID NO: 1327 - DNA, 57 bp, Artificial sequence
  Amorgage + (Barcode) + Primer
  misc_feature (24)..(30): n is a, c, g, or t
gtgccagcaagatccaatctagannnnnnncaagttgttcagaagaagcctgctcag

SEQ ID NO: 1328 - DNA, 53 bp, Artificial sequence
  Amorgage + (Barcode) + Primer
  misc_feature (24)..(30): n is a, c, g, or t
gtgccagcaagatccaatctagannnnnnnagatggtgcagaagaagaacgcg

SEQ ID NO: 1329 - DNA, 44 bp, Artificial sequence
  Amorgage + (Barcode) + Primer
ggtacgaagccagcctcatacatgctccaacccttagggaaccc

SEQ ID NO: 1330 - DNA, 59 bp, Artificial sequence
  Amorgage + (Barcode) + Primer
  misc_feature (24)..(30): n is a, c, g, or t
gtgccagcaagatccaatctagannnnnnnggaactgccagtgtagagggaattctaag

SEQ ID NO: 1331 - DNA, 57 bp, Artificial sequence
  Amorgage + (Barcode) + Primer
  misc_feature (24)..(30): n is a, c, g, or t
gtgccagcaagatccaatctagannnnnnngcctttttgaagaaactccacgaagag

SEQ ID NO: 1332 - DNA, 49 bp, Artificial sequence
  Amorgage + (Barcode) + Primer
gatgagcaattcttaggttttggctcagattccaacccttagggaaccc

SEQ ID NO: 1333 - DNA, 52 bp, Artificial sequence
  Amorgage + (Barcode) + Primer
  misc_feature (24)..(30): n is a, c, g, or t
gtgccagcaagatccaatctagannnnnnngctggaaacatttccgaccctg

SEQ ID NO: 1334 - DNA, 60 bp, Artificial sequence
  Amorgage + (Barcode) + Primer
  misc_feature (24)..(30): n is a, c, g, or t
gtgccagcaagatccaatctagannnnnnnaaggagaaggggttgaaattgttgatagag

SEQ ID NO: 1335 - DNA, 58 bp, Artificial sequence
  Amorgage + (Barcode) + Primer
  misc_feature (24)..(30): n is a, c, g, or t
gtgccagcaagatccaatctagannnnnnnatcaagtcctttgacagtgcatctcaag

SEQ ID NO: 1336 - DNA, 56 bp, Artificial sequence
  Amorgage + (Barcode) + Primer
  misc_feature (24)..(30): n is a, c, g, or t
gtgccagcaagatccaatctagannnnnnngcaagagtggtgatcgtggtgagact

SEQ ID NO: 1337 - DNA, 49 bp, Artificial sequence
  Amorgage + (Barcode) + Primer
tttttttgaagaagcaggatgctgatctaatccaacccttagggaaccc

SEQ ID NO: 1338 - DNA, 60 bp, Artificial sequence
  Amorgage + (Barcode) + Primer
  misc_feature (24)..(30): n is a, c, g, or t
gtgccagcaagatccaatctagannnnnnntcttatcctttgtcgcagagactatctgag

SEQ ID NO: 1339 - DNA, 43 bp, Artificial sequence
  Amorgage + (Barcode) + Primer
ggctattgagtggccagacttccctccaacccttagggaaccc

SEQ ID NO: 1340 - DNA, 54 bp, Artificial sequence
  Amorgage + (Barcode) + Primer
  misc_feature (24)..(30): n is a, c, g, or t
gtgccagcaagatccaatctagannnnnnnaggttgttaccgtgggcaactctg

SEQ ID NO: 1341 - DNA, 40 bp, Artificial sequence
  Amorgage + (Barcode) + Primer
gtggtggaggtggctggaatgtccaacccttagggaaccc

SEQ ID NO: 1342 - DNA, 54 bp, Artificial sequence
  Amorgage + (Barcode) + Primer
  misc_feature (24)..(30): n is a, c, g, or t
gtgccagcaagatccaatctagannnnnnnccagaaaaaaagaccaggccacag

SEQ ID NO: 1343 - DNA, 56 bp, Artificial sequence
  Amorgage + (Barcode) + Primer
  misc_feature (24)..(30): n is a, c, g, or t
gtgccagcaagatccaatctagannnnnnngccttctaccccatgagaaagaccag

SEQ ID NO: 1344 - DNA, 43 bp, Artificial sequence
  Amorgage + (Barcode) + Primer
cagcagccagtaaggaggagaaggtccaacccttagggaaccc

SEQ ID NO: 1345 - DNA, 57 bp, Artificial sequence
  Amorgage + (Barcode) + Primer
  misc_feature (24)..(30): n is a, c, g, or t
gtgccagcaagatccaatctagannnnnnngagttcaggaccagctcattgaaaaga

SEQ ID NO: 1346 - DNA, 56 bp, Artificial sequence
  Amorgage + (Barcode) + Primer
  misc_feature (24)..(30): n is a, c, g, or t
gtgccagcaagatccaatctagannnnnnngtggaaaaggctttagccatggacag

SEQ ID NO: 1347 - DNA, 49 bp, Artificial sequence
  Amorgage + (Barcode) + Primer
agatctgtcttacaacctattagaagattttccaacccttagggaaccc

SEQ ID NO: 1348 - DNA, 52 bp, Artificial sequence
  Amorgage + (Barcode) + Primer
  misc_feature (24)..(30): n is a, c, g, or t
gtgccagcaagatccaatctagannnnnnnccaaggcttgaccctcgttttg

SEQ ID NO: 1349 - DNA, 54 bp, Artificial sequence
  Amorgage + (Barcode) + Primer
  misc_feature (24)..(30): n is a, c, g, or t
gtgccagcaagatccaatctagannnnnnnaaacagcaagaactgcttcggcag

SEQ ID NO: 1350 - DNA, 45 bp, Artificial sequence
  Amorgage + (Barcode) + Primer
acaagtcatcaattgctggctcagaatccaacccttagggaaccc

SEQ ID NO: 1351 - DNA, 49 bp, Artificial sequence
  Amorgage + (Barcode) + Primer
ggtcaagaaagtgactcatcagagacctcttccaacccttagggaaccc

SEQ ID NO: 1352 - DNA, 51 bp, Artificial sequence
  Amorgage + (Barcode) + Primer
  misc_feature (24)..(30): n is a, c, g, or t
gtgccagcaagatccaatctagannnnnnngtcctccgacagtgcttggca

SEQ ID NO: 1353 - DNA, 39 bp, Artificial sequence
  Amorgage + (Barcode) + Primer
aagatgaatccggcctcggctccaacccttagggaaccc

SEQ ID NO: 1354 - DNA, 53 bp, Artificial sequence
  Amorgage + (Barcode) + Primer
  misc_feature (24)..(30): n is a, c, g, or t
gtgccagcaagatccaatctagannnnnnncggagtcagctgccaagagacag

SEQ ID NO: 1355 - DNA, 49 bp, Artificial sequence
  Amorgage + (Barcode) + Primer
gtgctatacttggtagatcagaaactcaggtccaacccttagggaaccc

SEQ ID NO: 1356 - DNA, 52 bp, Artificial sequence
  Amorgage + (Barcode) + Primer
  misc_feature (24)..(30): n is a, c, g, or t
gtgccagcaagatccaatctagannnnnnngaccatcatccagggcatcctg

SEQ ID NO: 1357 - DNA, 51 bp, Artificial sequence
  Amorgage + (Barcode) + Primer
  misc_feature (24)..(30): n is a, c, g, or t
gtgccagcaagatccaatctagannnnnnntgacacgcttccctggattgg

SEQ ID NO: 1358 - DNA, 51 bp, Artificial sequence
  Amorgage + (Barcode) + Primer
  misc_feature (24)..(30): n is a, c, g, or t
gtgccagcaagatccaatctagannnnnnncagctcctgaccaaccccaag

SEQ ID NO: 1359 - DNA, 51 bp, Artificial sequence
  Amorgage + (Barcode) + Primer
  misc_feature (24)..(30): n is a, c, g, or t
gtgccagcaagatccaatctagannnnnnnacagggacgccatcgaatccg

SEQ ID NO: 1360 - DNA, 60 bp, Artificial sequence
  Amorgage + (Barcode) + Primer
  misc_feature (24)..(30): n is a, c, g, or t
gtgccagcaagatccaatctagannnnnnntgaaatccgacactactgattctagtcaag

SEQ ID NO: 1361 - DNA, 60 bp, Artificial sequence
  Amorgage + (Barcode) + Primer
  misc_feature (24)..(30): n is a, c, g, or t
gtgccagcaagatccaatctagannnnnnnttggagaagatctatgggtcagacagaatt

SEQ ID NO: 1362 - DNA, 49 bp, Artificial sequence
  Amorgage + (Barcode) + Primer
gttactctggaagaagtcaactcccaaatatccaacccttagggaaccc

SEQ ID NO: 1363 - DNA, 49 bp, Artificial sequence
  Amorgage + (Barcode) + Primer
aactcgaaaattaatgctgaaaataaggcgtccaacccttagggaaccc

SEQ ID NO: 1364 - DNA, 42 bp, Artificial sequence
  Amorgage + (Barcode) + Primer
gactgggaggtgctggtcctaggtccaacccttagggaaccc

SEQ ID NO: 1365 - DNA, 49 bp, Artificial sequence
  Amorgage + (Barcode) + Primer
tttaaggctgcaagcagtatttacaacagatccaacccttagggaaccc

SEQ ID NO: 1366 - DNA, 49 bp, Artificial sequence
  Amorgage + (Barcode) + Primer
aatcatcggactcaggtacatctgtgagtgtccaacccttagggaaccc

SEQ ID NO: 1367 - DNA, 41 bp, Artificial sequence
  Amorgage + (Barcode) + Primer
gcctgtgcagtgggactgattgtccaacccttagggaaccc

SEQ ID NO: 1368 - DNA, 60 bp, Artificial sequence
  Amorgage + (Barcode) + Primer
  misc_feature (24)..(30): n is a, c, g, or t
gtgccagcaagatccaatctagannnnnnngttcaaaaactgaaggactctgaagctgag

SEQ ID NO: 1369 - DNA, 57 bp, Artificial sequence
  Amorgage + (Barcode) + Primer
  misc_feature (24)..(30): n is a, c, g, or t
gtgccagcaagatccaatctagannnnnnncgccaattgtaaacaaagtggtgacac

SEQ ID NO: 1370 - DNA, 57 bp, Artificial sequence
  Amorgage + (Barcode) + Primer
  misc_feature (24)..(30): n is a, c, g, or t
gtgccagcaagatccaatctagannnnnnnccttattgattggccaacaatcaacag

SEQ ID NO: 1371 - DNA, 39 bp, Artificial sequence
  Amorgage + (Barcode) + Primer
cccagccctggggagccccttccaacccttagggaaccc

SEQ ID NO: 1372 - DNA, 43 bp, Artificial sequence
  Amorgage + (Barcode) + Primer
ccgtagctccatattggacatccctccaacccttagggaaccc

SEQ ID NO: 1373 - DNA, 55 bp, Artificial sequence
  Amorgage + (Barcode) + Primer
  misc_feature (24)..(30): n is a, c, g, or t
gtgccagcaagatccaatctagannnnnnnccctgagaatctgggacctcaacag

SEQ ID NO: 1374 - DNA, 40 bp, Artificial sequence
  Amorgage + (Barcode) + Primer
tgtgtgcctcctgacgaagcctccaacccttagggaaccc

SEQ ID NO: 1375 - DNA, 41 bp, Artificial sequence
  Amorgage + (Barcode) + Primer
gccacagtggagaccagtcagctccaacccttagggaaccc

SEQ ID NO: 1376 - DNA, 53 bp, Artificial sequence
  Amorgage + (Barcode) + Primer
  misc_feature (24)..(30): n is a, c, g, or t
gtgccagcaagatccaatctagannnnnnngccaagaggagctcatgaggcag

SEQ ID NO: 1377 - DNA, 60 bp, Artificial sequence
  Amorgage + (Barcode) + Primer
  misc_feature (24)..(30): n is a, c, g, or t
gtgccagcaagatccaatctagannnnnnntctctagcagttactatggatgacttccgg

SEQ ID NO: 1378 - DNA, 60 bp, Artificial sequence
  Amorgage + (Barcode) + Primer
  misc_feature (24)..(30): n is a, c, g, or t
gtgccagcaagatccaatctagannnnnnnaactcacaacggtaggagagaaacctgaag

SEQ ID NO: 1379 - DNA, 54 bp, Artificial sequence
  Amorgage + (Barcode) + Primer
  misc_feature (24)..(30): n is a, c, g, or t
gtgccagcaagatccaatctagannnnnnnagcccgggaccgtttaaaaaactg

SEQ ID NO: 1380 - DNA, 42 bp, Artificial sequence
  Amorgage + (Barcode) + Primer
aaatgtggagcccaggaggaaggtccaacccttagggaaccc

SEQ ID NO: 1381 - DNA, 59 bp, Artificial sequence
  Amorgage + (Barcode) + Primer
  misc_feature (24)..(30): n is a, c, g, or t
gtgccagcaagatccaatctagannnnnnnaatggtcagaaaccctccataacctgaag

SEQ ID NO: 1382 - DNA, 44 bp, Artificial sequence
  Amorgage + (Barcode) + Primer
gatgcaattcgaagtcacagcgaattccaacccttagggaaccc

SEQ ID NO: 1383 - DNA, 56 bp, Artificial sequence
  Amorgage + (Barcode) + Primer
  misc_feature (24)..(30): n is a, c, g, or t
gtgccagcaagatccaatctagannnnnnncggacgcatcacttgcacttctagaa

SEQ ID NO: 1384 - DNA, 49 bp, Artificial sequence
  Amorgage + (Barcode) + Primer
agctgatagacacacaccttagctggatactccaacccttagggaaccc

SEQ ID NO: 1385 - DNA, 53 bp, Artificial sequence
  Amorgage + (Barcode) + Primer
  misc_feature (24)..(30): n is a, c, g, or t
gtgccagcaagatccaatctagannnnnnnctttgctgaatgctccagccaag

SEQ ID NO: 1386 - DNA, 48 bp, Artificial sequence
  Amorgage + (Barcode) + Primer
cttgtaatctggatgtgattctggggttttccaacccttagggaaccc

SEQ ID NO: 1387 - DNA, 45 bp, Artificial sequence
  Amorgage + (Barcode) + Primer
gaaagcccttcttgtatgtcaatgcctccaacccttagggaaccc

SEQ ID NO: 1388 - DNA, 48 bp, Artificial sequence
  Amorgage + (Barcode) + Primer
gtaacagtatcgggacccttactgcacattccaacccttagggaaccc

SEQ ID NO: 1389 - DNA, 49 bp, Artificial sequence
  Amorgage + (Barcode) + Primer
acattactggttatagaattaccacaaccctccaacccttagggaaccc

SEQ ID NO: 1390 - DNA, 47 bp, Artificial sequence
  Amorgage + (Barcode) + Primer
ctcaagcttttaaaatcgagaccacccctccaacccttagggaaccc

SEQ ID NO: 1391 - DNA, 50 bp, Artificial sequence
  Amorgage + (Barcode) + Primer
  misc_feature (24)..(30): n is a, c, g, or t
gtgccagcaagatccaatctagannnnnnnagccccagtcccagccccag

SEQ ID NO: 1392 - DNA, 48 bp, Artificial sequence
  Amorgage + (Barcode) + Primer
aatgcagctcttcagcatctgtttattcgtccaacccttagggaaccc

SEQ ID NO: 1393 - DNA, 59 bp, Artificial sequence
  Amorgage + (Barcode) + Primer
  misc_feature (24)..(30): n is a, c, g, or t
gtgccagcaagatccaatctagannnnnnncgagggtgttcttgacgattaatcaacag

SEQ ID NO: 1394 - DNA, 51 bp, Artificial sequence
  Amorgage + (Barcode) + Primer
  misc_feature (24)..(30): n is a, c, g, or t
gtgccagcaagatccaatctagannnnnnnctccgccccacagtccacgag

SEQ ID NO: 1395 - DNA, 51 bp, Artificial sequence
  Amorgage + (Barcode) + Primer
  misc_feature (24)..(30): n is a, c, g, or t
gtgccagcaagatccaatctagannnnnnngtggcggaatcggtggtagag

SEQ ID NO: 1396 - DNA, 56 bp, Artificial sequence
  Amorgage + (Barcode) + Primer
  misc_feature (24)..(30): n is a, c, g, or t
gtgccagcaagatccaatctagannnnnnncgccatcatcctcatcatcatcatag

SEQ ID NO: 1397 - DNA, 44 bp, Artificial sequence
  Amorgage + (Barcode) + Primer
agatcatcactggtatgccagcctctccaacccttagggaaccc

SEQ ID NO: 1398 - DNA, 60 bp, Artificial sequence
  Amorgage + (Barcode) + Primer
  misc_feature (24)..(30): n is a, c, g, or t
gtgccagcaagatccaatctagannnnnnnacagtctcttgcaatcggctaaaaaaaaga

SEQ ID NO: 1399 - DNA, 57 bp, Artificial sequence
  Amorgage + (Barcode) + Primer
  misc_feature (24)..(30): n is a, c, g, or t
gtgccagcaagatccaatctagannnnnnnctatcagaagaaaatcggcacctgaga

SEQ ID NO: 1400 - DNA, 60 bp, Artificial sequence
  Amorgage + (Barcode) + Primer
  misc_feature (24)..(30): n is a, c, g, or t
gtgccagcaagatccaatctagannnnnnnagaaaactcttaaagaatgcagcagcttgg

SEQ ID NO: 1401 - DNA, 43 bp, Artificial sequence
  Amorgage + (Barcode) + Primer
gacactggggttgggaaatcaagctccaacccttagggaaccc

SEQ ID NO: 1402 - DNA, 53 bp, Artificial sequence
  Amorgage + (Barcode) + Primer
  misc_feature (24)..(30): n is a, c, g, or t
gtgccagcaagatccaatctagannnnnnncccagcgctaccttgtcattcag

SEQ ID NO: 1403 - DNA, 57 bp, Artificial sequence
  Amorgage + (Barcode) + Primer
  misc_feature (24)..(30): n is a, c, g, or t
gtgccagcaagatccaatctagannnnnnncagtttgctgtgtgtttgctcaaacag

SEQ ID NO: 1404 - DNA, 60 bp, Artificial sequence
  Amorgage + (Barcode) + Primer
  misc_feature (24)..(30): n is a, c, g, or t
gtgccagcaagatccaatctagannnnnnntacttggactagtttatatgaaatttgtgg

SEQ ID NO: 1405 - DNA, 47 bp, Artificial sequence
  Amorgage + (Barcode) + Primer
gacatgaacaagctgagtggaggcggcgtccaacccttagggaaccc

SEQ ID NO: 1406 - DNA, 47 bp, Artificial sequence
  Amorgage + (Barcode) + Primer
ctacatctacatccaccactgggacaagtccaacccttagggaaccc

SEQ ID NO: 1407 - DNA, 40 bp, Artificial sequence
  Amorgage + (Barcode) + Primer
ccttgcctccccgattgaaagtccaacccttagggaaccc

SEQ ID NO: 1408 - DNA, 51 bp, Artificial sequence
  Amorgage + (Barcode) + Primer
  misc_feature (24)..(30): n is a, c, g, or t
gtgccagcaagatccaatctagannnnnnngtgccacggtgtccggatatg

SEQ ID NO: 1409 - DNA, 60 bp, Artificial sequence
  Amorgage + (Barcode) + Primer
  misc_feature (24)..(30): n is a, c, g, or t
gtgccagcaagatccaatctagannnnnnnattttaatgaaaacacagcagcacctagag

SEQ ID NO: 1410 - DNA, 58 bp, Artificial sequence
  Amorgage + (Barcode) + Primer
  misc_feature (24)..(30): n is a, c, g, or t
gtgccagcaagatccaatctagannnnnnnatgaaggaaatgctaaagcgattccaag

SEQ ID NO: 1411 - DNA, 51 bp, Artificial sequence
  Amorgage + (Barcode) + Primer
  misc_feature (24)..(30): n is a, c, g, or t
gtgccagcaagatccaatctagannnnnnntgccatctccaggccttgcag

SEQ ID NO: 1412 - DNA, 39 bp, Artificial sequence
  Amorgage + (Barcode) + Primer
gcccggctgtgctggctccatccaacccttagggaaccc

SEQ ID NO: 1413 - DNA, 39 bp, Artificial sequence
  Amorgage + (Barcode) + Primer
tcccggccagtgtgcagctgtccaacccttagggaaccc

SEQ ID NO: 1414 - DNA, 40 bp, Artificial sequence
  Amorgage + (Barcode) + Primer
ggtcctgtcggggaaccctcttccaacccttagggaaccc